(19) United States
(12) Patent Application Publication
(10) Pub. No.: US 2011/0150833 A1
(43) Pub. Date: Jun. 23, 2011

(54) BENZOPYRANS AND ANALOGS AS RHO KINASE INHIBITORS

(75) Inventors: Yangbo Feng, Palm Beach Gardens, FL (US); Philip LoGrasso, Jupiter, FL (US); Thomas Bannister, Palm Beach Gardens, FL (US); Thomas Schroeter, Riviera Beach, FL (US); Yen Ting Chen, Palm Beach Gardens, FL (US); Yan Yin, Jupiter, FL (US); Hampton Sessions, Orlando, FL (US); Michael P. Smolinski, Port St. Lucie, FL (US); Lei Yao, Tianjin (CN); Bo Wang, Collegeville, PA (US); Bozena Frackowiak-Wojtasek, Wielki (PL)

(73) Assignee: The Scripps Research Institute, La Jolla, CA (US)

(21) Appl. No.: 12/746,776

(22) PCT Filed: Dec. 18, 2008
(86) PCT No.: PCT/US08/13844

§ 371 Date: Mar. 14, 2011

Related U.S. Application Data
(60) Provisional application No. 61/008,493, filed on Dec. 19, 2007.

(51) Int. Cl. A61K 31/4155; C07D 405/12; A61K 31/4433; C07D 405/14; A61K 31/496; A61K 31/506; A61K 31/519; C07D 471/02; A61K 31/4375; A61K 31/501; A61K 38/21; A61K 39/395; A61K 31/56; A61P 9/00; A61P 15/10; A61P 29/00; A61P 25/00; A61P 19/02; A61P 11/00; A61P 35/00; A61P 31/04

(52) U.S. Cl. 424/85.6; 548/364.4; 514/406; 546/282.4; 514/338; 544/364; 514/253.11; 544/332; 514/275; 544/280; 514/265.1; 546/122; 514/300; 544/238; 514/252.03; 424/133.1; 514/171

(57) ABSTRACT

Compounds useful as Rho kinase inhibitors of formula (1) wherein the variables are as defined herein are provided. Methods of treatment of malconditions mediated by Rho kinase, and methods of preparation of the compounds, are also provided.

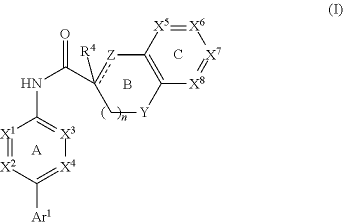

### CROSS-REFERENCE TO RELATED APPLICATIONS

This application claims the priority of U.S. Ser. No. 61/016,132, filed Dec. 21, 2007, which is incorporated herein by reference in its entirety.

### BACKGROUND

Rho kinases, also known as Rho-associated kinases, are serine/threonine kinases that function downstream of Rho which is a low molecular GTP-binding protein. Two Rho kinase isoforms, termed ROCK I and ROCK II, have been identified. The enzymes are believed to be involved in a variety of biological events such as smooth muscle contraction, apoptosis, cell growth, cell migration, cell proliferation, cytokinesis, cytoskeletal control, and inflammation, and to be involved in pathology of various diseases including cardiovascular disease, tumor infiltration, osteogenesis, chondrocyte differentiation and neurogenic pain. See, e.g., H. Satoh, et al., Jpn. J. Pharmacol., 1999, 79, Suppl I, 211, K. Kuwahara, et al., FEBS Lett., 1999, 452, 314-18; N. Sawada, et al., Circulation, 2000, 101, 2030-33; C. Kataoka, et al., Hypertension, 2002, 39(2), 245-50; F. Imamura, et al., Jpn. J. Cancer Res., 2000, 91, 811-16, K. Itoh et al, Nature Medicine, 1999, 5, 221-5, M. Nakajima, et al., Clin. Exp. Pharmacol. Physiol., 2003; 30(7): 457-63; W. Guoyan, et al., J. Biol. Chem., 2004, 279(13), 13205-14; S. Tatsumi, Neuroscience, 2005, 131(2) 491-98.

It is therefore believed that Rho kinase inhibitors have utility in the treatment of diseases and conditions such as hypertension, atherosclerosis, stroke, angina, arterial obstruction, peripheral arterial disease, peripheral circulation disorder, erectile dysfunction, acute and chronic pain, dementia, Alzheimer's disease, Parkinson's disease, neuronal degeneration, asthma, amyotrophic lateral sclerosis, spinal cord injury, rheumatoid arthritis, osteoarthritis, osteoporosis, psoriasis, multiple sclerosis, diabetes, urinary organ diseases such as overactive bladder (OAB) and benign prostatic hypertrophy (BPH), metastasis, cancer, glaucoma, ocular hypertension, retinopathy, autoimmune disease and viral infection, and myocardial protection.

Various compounds have been described in the literature as Rho kinase inhibitors. See, e.g. WO98/06433; WO00/09162; WO00/78351; WO01/17562; WO02/076976; EP1256574; WO02/100833; WO03/082808; WO2004/009555; WO2004/024717; WO2004/041813; WO2004/108724; WO2005/003101; WO2005/035501; WO2005/035503; WO2005/035506; WO2005/037198; WO2005/058891; WO2005/061463; WO2005/074642; WO2005/074643; WO2005/080934; WO2005/082367; WO2005/082890; WO2005/097790; WO2005/100342; WO2005/103050; WO2005/105780; WO2005/108397; WO2006/044753; WO2006/051311; WO2006/057270; WO2006/058120; WO2006/065946; WO2006/099268; WO2006/072792; WO2006/127587; WO2006/136829; WO2006/136837; WO2007/026920; WO2008/110846; A. Takami, et al., Bioorg. Med. Chem., 2004, 12, 2115-37; M. Iwakubo, et al., Bioorg. Med. Chem., 2007, 15, 350-64; M. Iwakubo, et al., Bioorg. Med. Chem., 2007, 15, 1022-33.

### SUMMARY

The present invention is directed to certain compounds and compositions that are effective Rho kinase inhibitors, to methods of their use in the treatment of diseases for which inhibition of Rho kinase is therapeutically indicated, and to methods for their preparation.

In various embodiments, the invention is directed to a compound of formula (I)

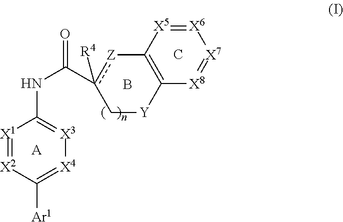

wherein

X¹, X², X³, and X⁴ are each independently N, CH, or CR¹ such that ring A comprises a phenyl, pyridyl, pyridazinyl, or pyrimidinyl ring, provided that 0-2 of X¹, X², X³ and X⁴ are CR¹, the remainder being independently N or CH;

R¹ comprises independently at each occurrence F, Cl, Br, I, CF₃, OCF₃, (C₁₋₆)-alkyl substituted with 0-2 R^{a}, (C₂₋₆)-alkenyl substituted with 0-2 R^{a}, (C₂₋₆)-alkynyl substituted with 0-2 R^{a}, (CH₂)ₚNO₂, (CH₂)ₚCN, (CH₂)ₚOR, (CH₂)ₚNR₂, (CH₂)ₚCOR, (CH₂)ₚOCOR, (CH₂)ₚCO₂R, (CH₂)ₚCONR₂, (CH₂)ₚOCONR₂, (CH₂)ₚNRCOR, (CH₂)ₚNRCO₂R, (CH₂)ₚNRCONR₂, (CH₂)ₚC(═NH)NH₂, (CH₂)ₚS(O)_{q}R, (CH₂)ₚSO₂NR₂, (CH₂)ₚNRSO₂R, (CH₂)ₚNRSO₂NR₂, (CH₂)ₚ-(3-10 membered)-cycloalkyl substituted with 0-2 R^{a}, or (CH₂)ₚ-(4-10 membered)-heterocyclyl substituted with 0-2 R^{a} comprising 1-4 heteroatoms selected from O, S(O)_{q}, and N; or two adjacent R¹ substituents can form a fused phenyl or a 5-6 membered heteroaryl comprising carbon atoms and 1-2 heteroatoms selected from O, S(O)_{q}, and N, and substituted with 0-3 R^{a}, wherein p is 0-4 and q is 0-2;

R is independently at each occurrence H, (C₁₋₆)-alkyl substituted with 0-2 R^{a}, (C₂₋₆)-alkenyl substituted with 0-2 R^{a}, (C₂₋₆)-alkynyl substituted with 0-2 R^{a}, (3-10 membered)-cycloalkyl substituted with 0-2 R^{a}, or (3-10 membered)-heterocyclyl substituted with 0-2 R^{a} comprising 1-4 heteroatoms selected from O, S(O)_{q}, and N; or, an NR₂ forms a (3-10 membered)-heterocyclyl substituted with 0-2 R^{a} and comprising 0-1 additional ring heteroatoms selected from N, O, and S(O)_{q};

R^{a} is independently at each occurrence oxo, F, Cl, Br, I, CF₃, OCF₃, (C₁₋₆)-alkyl substituted with 0-2 R^{a}, (C₂₋₆)-alkenyl substituted with 0-2 R^{a}, (C₂₋₆)-alkynyl substituted with 0-2 R^{a}, (CH₂)ₚNO₂, (CH₂)ₚCN, (CH₂)ₚOR, (CH₂)ₚNR₂, (CH₂)ₚCOR, (CH₂)ₚOCOR, (CH₂)ₚCO₂R, (CH₂)ₚCONR₂, (CH₂)ₚOCONR₂, (CH₂)ₚNRCOR, (CH₂)ₚNRCO₂R, (CH₂)ₚNRCONR₂, (CH₂)ₚC(═NH)NH₂, (CH₂)ₚS(O)_{q}R, (CH₂)ₚSO₂NR₂, (CH₂)ₚNRSO₂R, (CH₂)ₚNRSO₂NR₂, (CH₂)ₚ-(3-10 membered)-cycloalkyl substituted with 0-2 R^{a}, or (CH₂)ₚ-(3-10 membered)-heterocyclyl substituted with 0-2 R^{a} comprising 1-4 heteroatoms selected from O, S(O)_{q}, and N;

Ar¹ comprises a 5- or 6-membered heteroaryl comprising at least one nitrogen atom and 0-3 additional heteroatoms selected from O, S(O)_{q}, and N; when Ar¹ is a 5-membered heteroaryl, a nitrogen atom is disposed one atom away from an atom of the heteroaryl bonded to ring A, and when Ar¹ is a 6-membered heteroaryl, a nitrogen atom is disposed two atoms away from an atom of the heteroaryl bonded to ring A; wherein Ar¹ is optionally fused with phenyl or a 5-6 membered heteroaryl comprising 1-2 heteroatoms selected from O, S(O)_{q}, and N, wherein the fused phenyl or 5-6 membered heteroaryl is substituted with 0-3 R^{a};

n is 0 or 1;

wherein a dotted line indicates a single bond or a double bond;

Y is O, CH(R³), S(O)_{q}, N(R³), or C(O);

Z is O, CH(R³), CR³, S(O)_{q}, N(R³), or C(O), provided that when the double bond is absent, R⁴ is present, Z is O, CH(R³), or C(O) and when the double bond is present R⁴ is absent and Z is CR³;

X⁵, X⁶, X⁷ and X⁸ are each independently N, CH, or CR², such that ring C comprises a phenyl, pyridyl, pyrazinyl, pyridazinyl, or pyrimidinyl ring;

R² is independently at each occurrence hydrogen, (C₁-C₆)alkyl, hydroxy(C₁-C₆)alkyl, (C₂-C₆)alkenyl; (C₂-C₆)alkynyl; halogen; —C≡N; —NO₂; —C(═O)R; —C(═O)OR; (C₁-C₆)alkylene-C(═O)OR; —C(═O)NR₂; (C₁-C₆)alkylene-C(═O)NR₂; —C(═NR)NR₂; —OR; (C₁-C₆)alkylene-OR; —OC(═O)(C₁-C₆)alkyl; —OC(═O)O(C₁-C₆)alkyl; —OC(═O)NR₂; —NR₂; —NRC(═O)R; —NRC(═O)O(C₁-C₆)alkyl; —NRC(═O)NR₂; —NR(C₁-C₆)alkylene-NR₂; —NR(C₁-C₆)alkylene-OR; —NR(C₁-C₆)alkylene-Ar²; —NRSO₂R; —SR; —S(O)R; —SO₂R; —OSO₂(C₁-C₆)alkyl; —SO₂NR₂; (C₁-C₃)perfluoroalkyl; —O(C₁-C₃)perfluoroalkyl; pyrazolyl; triazolyl; and tetrazolyl; or two R² groups taken together form a fused cycloalkyl, heterocyclyl, aryl or heteroaryl ring which is substituted with 0-3 R^{a};

R³ is H, CF₃, OCF₃, OCH₃, (C₁₋₆)-alkyl substituted with 0-3 R^{a}, or NR₂;

R⁴ is H or methyl;

or any salt, stereoisomer, tautomer, hydrate, solvate, or prodrug thereof.

In various embodiments, the invention provides methods of synthesis of compounds of the invention.

In various embodiments, the invention provides a pharmaceutical composition comprising a compound of the invention and a suitable excipient.

In various embodiments, the invention provides a pharmaceutical combination comprising a compound of the invention and a second medicament.

In various embodiments, the invention provides a method of treatment of a malcondition in a patient comprising administering a therapeutically effective amount of a compound, pharmaceutical composition, or pharmaceutical combination of the invention to the patient at a frequency of administration and for a duration of time sufficient to provide a beneficial effect to the patient.

In various embodiments, the inventive method can further comprise administration of an effective a second medicament to the patient at a frequency and for a duration sufficient to provide a beneficial effect to the patient. The second medicament can be an anti-proliferative agent, an anti-glaucoma agent, an anti-hypertensive agent, an anti-atherosclerotic agent, an anti-multiple sclerosis agent, an anti-angina agent, an anti-erectile dysfunction agent, an anti-stroke agent, or an anti-asthma agent.

In various embodiments, the invention provides a method of treatment of a malcondition in a patient, comprising administering to the patient the pharmaceutical combination of the invention or a pharmaceutical composition comprising the inventive combination in a therapeutically effective amount at a frequency of administration and for a duration of time sufficient to provide a beneficial effect to the patient.

The malcondition can comprise cardiovascular disease, neurogenic pain, hypertension, atherosclerosis, angina, stroke, arterial obstruction, peripheral arterial disease, peripheral circulation disorder, erectile dysfunction, acute or chronic pain, dementia, Alzheimer's disease, Parkinson's disease, neuronal degeneration, asthma, amyotrophic lateral sclerosis, spinal cord injury, rheumatoid arthritis, osteoarthritis, osteoporosis, psoriasis, cerebral vasospasm, glaucoma, multiple sclerosis, pulmonary hypertension, acute respiratory distress syndrome, inflammation, diabetes, urinary organ diseases such as overactive bladder (OAB) and benign prostatic hypertrophy (BPH), metastasis, cancer, glaucoma, ocular hypertension, retinopathy, autoimmune disease and viral infection, or myocardial pathology, or any combination thereof. The malcondition can be one for the treatment of which binding of a ligand to a Rho kinase or inhibition of a bioactivity of a Rho kinase, or both, is medically indicated.

In various embodiments, the invention provides a use of a compound, composition, or combination of the invention in the preparation of a medicament for treatment of a malcondition. The malcondition can be one wherein binding of a ligand to a Rho kinase or inhibition of a bioactivity of a Rho kinase, or both, is medically indicated. The malcondition can include cardiovascular disease, neurogenic pain, hypertension, atherosclerosis, angina, stroke, arterial obstruction, peripheral arterial disease, peripheral circulation disorder, erectile dysfunction, acute or chronic pain, dementia, Alzheimer's disease, Parkinson's disease, neuronal degeneration, asthma, amyotrophic lateral sclerosis, spinal cord injury, rheumatoid arthritis, osteoarthritis, osteoporosis, psoriasis, cerebral vasospasm, glaucoma, multiple sclerosis, pulmonary hypertension, acute respiratory distress syndrome, inflammation, diabetes, urinary organ diseases such as overactive bladder (OAB) and benign prostatic hypertrophy (BPH), metastasis, cancer, glaucoma, ocular hypertension, retinopathy, autoimmune disease and viral infection, or myocardial pathology, or any combination thereof.

In various embodiments, the invention provides a compound of the invention for use in treatment of cardiovascular disease, neurogenic pain, hypertension, atherosclerosis, angina, stroke, arterial obstruction, peripheral arterial disease, peripheral circulation disorder, erectile dysfunction, acute or chronic pain, dementia, Alzheimer's disease, Parkinson's disease, neuronal degeneration, asthma, amyotrophic lateral sclerosis, spinal cord injury, rheumatoid arthritis, osteoarthritis, osteoporosis, psoriasis, cerebral vasospasm, glaucoma, multiple sclerosis, pulmonary hypertension, acute respiratory distress syndrome, inflammation, diabetes, urinary organ diseases such as overactive bladder (OAB) and benign prostatic hypertrophy (BPH), metastasis, cancer, glaucoma, ocular hypertension, retinopathy, autoimmune disease and viral infection, or myocardial pathology, or any combination thereof.

In various embodiments, the invention provides a compound of any the invention for use in combination with an effective amount of a second bioactive agent in treatment of cardiovascular disease, neurogenic pain, hypertension, atherosclerosis, angina, stroke, arterial obstruction, peripheral arterial disease, peripheral circulation disorder, erectile dysfunction, acute or chronic pain, dementia, Alzheimer's disease, Parkinson's disease, neuronal degeneration, asthma, amyotrophic lateral sclerosis, spinal cord injury, rheumatoid arthritis, osteoarthritis, osteoporosis, psoriasis, cerebral vasospasm, glaucoma, multiple sclerosis, pulmonary hypertension, acute respiratory distress syndrome, inflammation, diabetes, urinary organ diseases such as overactive bladder (OAB) and benign prostatic hypertrophy (BPH), metastasis, cancer, glaucoma, ocular hypertension, retinopathy, autoimmune disease and viral infection, or myocardial pathology, or any combination thereof. The second medicament can be an anti-proliferative agent, an anti-glaucoma agent, an anti-hypertensive agent, an anti-atherosclerotic agent, an anti-multiple sclerosis agent, an anti-angina agent, an anti-erectile dysfunction agent, an anti-stroke agent, or an anti-asthma agent.

### DETAILED DESCRIPTION

### Definitions

As used in the specification and the appended claims, the singular forms “a,” “an” and “the” include plural referents unless the context clearly dictates otherwise.

As used herein, “individual” (as in the subject of the treatment) means both mammals and non-mammals. Mammals include, for example, humans; non-human primates, e.g. apes and monkeys; cattle; horses; sheep; and goats. Non-mammals include, for example, fish and birds.

The term “Rho-kinase-mediated disease” or “Rho-kinase-mediated disorder” are used interchangeably, and are used to refer to diseases or conditions wherein a Rho-kinase (ROCK) plays a role in the biochemical mechanisms involved in the diseases such that a therapeutically beneficial effect can be achieved by inhibiting a Rho-kinase.

The expression “effective amount”, when used to describe therapy to an individual suffering from Rho-kinase-mediated disorder, refers to the amount of a compound of the invention that is effective to inhibit or otherwise act on a Rho kinase in the individual's tissues wherein the Rho-kinase involved in the disorder is active, wherein such inhibition or other action occurs to an extent sufficient to produce a beneficial therapeutic effect.

“Treating” or “treatment” within the meaning herein refers to an alleviation of symptoms associated with a disorder or disease, or inhibition of further progression or worsening of those symptoms, or prevention or prophylaxis of the disease or disorder. Similarly, as used herein, an “effective amount” or a “therapeutically effective amount” of a compound of the invention refers to an amount of the compound that alleviates, in whole or in part, symptoms associated with the disorder or condition, or halts or slows further progression or worsening of those symptoms, or prevents or provides prophylaxis for the disorder or condition. In particular, a “therapeutically effective amount” refers to an amount effective, at dosages and for periods of time necessary, to achieve the desired therapeutic result. A therapeutically effective amount is also one in which any toxic or detrimental effects of compounds of the invention are outweighed by the therapeutically beneficial effects.

By “chemically feasible” is meant a bonding arrangement or a compound where the generally understood rules of organic structure are not violated; for example a structure within a definition of a claim that would contain in certain situations a pentavalent carbon atom that would not exist in nature would be understood to not be within the claim.

When a substituent is specified to be an atom or atoms of specified identity, “or a bond”, a configuration is referred to when the substituent is “a bond” that the groups that are immediately adjacent to the specified substituent are directly connected to each other by a chemically feasible bonding configuration.

All chiral, diastereomeric, racemic forms of a structure are intended, unless a particular stereochemistry or isomeric form is specifically indicated. Compounds used in the present invention can include enriched or resolved optical isomers at any or all asymmetric atoms as are apparent from the depictions, at any degree of enrichment. Both racemic and diastereomeric mixtures, as well as the individual optical isomers can be isolated or synthesized so as to be substantially free of their enantiomeric or diastereomeric partners, and these are all within the scope of the invention.

The term “amino protecting group” or “N-protected” as used herein refers to those groups intended to protect an amino group against undesirable reactions during synthetic procedures and which can later be removed to reveal the amine. Commonly used amino protecting groups are disclosed in Protective Groups in Organic Synthesis, Greene, T. W.; Wuts, P. G. M., John Wiley & Sons, New York, N.Y., (3rd Edition, 1999). Amino protecting groups include acyl groups such as formyl, acetyl, propionyl, pivaloyl, t-butylacetyl, 2-chloroacetyl, 2-bromoacetyl, trifluoroacetyl, trichloroacetyl, o-nitrophenoxyacetyl, α-chlorobutyryl, benzoyl, 4-chlorobenzoyl, 4-bromobenzoyl, 4-nitrobenzoyl, and the like; sulfonyl groups such as benzenesulfonyl, p-toluenesulfonyl and the like; alkoxy- or aryloxy-carbonyl groups (which form urethanes with the protected amine) such as benzyloxycarbonyl (Cbz), p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-methoxybenzyloxy-carbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxycarbonyl, t-butyloxycarbonyl (Boc), diisopropylmethoxycarbonyl, isopropyloxycarbonyl, ethoxycarbonyl, methoxycarbonyl, allyloxycarbonyl (Alloc), 2,2,2-trichloroethoxycarbonyl, 2-trimethylsilylethyloxycarbonyl (Teoc), phenoxycarbonyl, 4-nitrophenoxycarbonyl, fluorenyl-9-methoxycarbonyl (Fmoc), cyclopentyloxycarbonyl, adamantyloxycarbonyl, cyclohexyloxycarbonyl, phenylthiocarbonyl and the like; aralkyl groups such as benzyl, triphenylmethyl, benzyloxymethyl and the like; and silyl groups such as trimethylsilyl and the like. Amine protecting groups also include cyclic amino protecting groups such as phthaloyl and dithiosuccinimidyl, which incorporate the amino nitrogen into a heterocycle. Typically, amino protecting groups include formyl, acetyl, benzoyl, pivaloyl, t-butylacetyl, phenylsulfonyl, Alloc, Teoc, benzyl, Fmoc, Boc and Cbz. It is well within the skill of the ordinary artisan to select and use the appropriate amino protecting group for the synthetic task at hand.

The term “hydroxyl protecting group” or “O-protected” as used herein refers to those groups intended to protect an OH group against undesirable reactions during synthetic procedures and which can later be removed to reveal the amine. Commonly used hydroxyl protecting groups are disclosed in Protective Groups in Organic Synthesis, Greene, T. W.; Wuts, P. G. M., John Wiley & Sons, New York, N.Y., (3rd Edition, 1999). Hydroxyl protecting groups include acyl groups such as formyl, acetyl, propionyl, pivaloyl, t-butylacetyl, 2-chloroacetyl, 2-bromoacetyl, trifluoroacetyl, trichloroacetyl, o-nitrophenoxy-acetyl, α-chlorobutyryl, benzoyl, 4-chlorobenzoyl, 4-bromobenzoyl, 4-nitrobenzoyl, and the like; sulfonyl groups such as benzenesulfonyl, p-toluenesulfonyl and the like; acyloxy groups (which form urethanes with the protected amine) such as benzyloxycarbonyl (Cbz), p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-methoxybenzyloxy-carbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxycarbonyl, t-butyloxycarbonyl (Boc), diisopropylmethoxycarbonyl, isopropyloxycarbonyl, ethoxycarbonyl, methoxycarbonyl, allyloxycarbonyl (Alloc), 2,2,2-trichloroethoxycarbonyl, 2-trimethylsilylethyloxycarbonyl (Teoc), phenoxycarbonyl, 4-nitrophenoxycarbonyl, fluorenyl-9-methoxycarbonyl (Fmoc), cyclopentyloxycarbonyl, adamantyloxycarbonyl, cyclohexyloxycarbonyl, phenylthiocarbonyl and the like; aralkyl groups such as benzyl, triphenylmethyl, benzyloxymethyl and the like; and silyl groups such as trimethylsilyl and the like. It is well within the skill of the ordinary artisan to select and use the appropriate hydroxyl protecting group for the synthetic task at hand.

In general, “substituted” refers to an organic group as defined herein in which one or more bonds to a hydrogen atom contained therein are replaced by one or more bonds to a non-hydrogen atom such as, but not limited to, a halogen (i.e., F, Cl, Br, and I); an oxygen atom in groups such as hydroxyl groups, alkoxy groups, aryloxy groups, aralkyloxy groups, oxo(carbonyl) groups, carboxyl groups including carboxylic acids, carboxylates, and carboyxlate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxylamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR′, OC(O)N(R′)₂, CN, CF₃, OCF₃, R′, O, S, C(O), S(O), methylenedioxy, ethylenedioxy, N(R′)₂, SR′, SOR′, SO₂R′, SO₂N(R′)₂, SO₃R′, C(O)R′, C(O)C(O)R′, C(O)CH₂C(O)R′, C(S)R′, C(O)OR′, OC(O)R′, C(O)N(R′)₂, OC(O)N(R′)₂, C(S)N(R′)₂, (CH₂)₀₋₂NHC(O)R′, N(R′)N(R′)C(O)R′, N(R′)N(R′)C(O)OR′, N(R′)N(R′)CON(R′)₂, N(R′)SO₂R′, N(R′)SO₂N(R′)₂, N(R′)C(O)OR′, N(R′)C(O)R′, N(R′)C(S)R′, N(R′)C(O)N(R′)₂, N(R′)C(S)N(R′)₂, N(COR′)COR′, N(OR′)R′, C(═NH)N(R′)₂, C(O)N(OR′)R′, or C(═NOR′)R′ wherein R′ can be hydrogen or a carbon-based moiety, and wherein the carbon-based moiety can itself be further substituted. When a substituent is monovalent, such as, for example, F or Cl, it is bonded to the atom it is substituting by a single bond. When a substituent is more than monovalent, such as O, which is divalent, it can be bonded to the atom it is substituting by more than one bond, i.e., a divalent substituent is bonded by a double bond; for example, a C substituted with O forms a carbonyl group, C═O, which can also be written as “CO”, “C(O)”, or “C(═O)”, wherein the C and the O are double bonded. When a carbon atom is substituted with a double-bonded oxygen (═O) group, the oxygen substituent is termed an “oxo” group. Alternatively, a divalent substituent such as O, S, C(O), S(O), or S(O)₂ can be connected by two single bonds to two different carbon atoms. For example, O, a divalent substituent, can be bonded to each of two adjacent carbon atoms to provide an epoxide group, or the O can form a bridging ether group, termed an “oxy” group, between adjacent or non-adjacent carbon atoms, for example bridging the 1,4-carbons of a cyclohexyl group to form a [2.2.1]-oxabicyclo system. Further, any substituent can be bonded to a carbon or other atom by a linker, such as (CH₂), or (CR′₂)ₙ wherein n is 1, 2, 3, or more, and each R′ is independently selected.

Substituted alkyl, alkenyl, alkynyl, cycloalkyl, and cycloalkenyl groups as well as other substituted groups also include groups in which one or more bonds to a hydrogen atom are replaced by one or more bonds, including double or triple bonds, to a carbon atom, or to a heteroatom such as, but not limited to, oxygen in carbonyl (oxo), carboxyl, ester, amide, imide, urethane, and urea groups; and nitrogen in imines, hydroxyimines, oximes, hydrazones, amidines, guanidines, and nitriles.

Substituted ring groups such as substituted cycloalkyl, aryl, heterocyclyl and heteroaryl groups also include rings and fused ring systems in which a bond to a hydrogen atom is replaced with a bond to a carbon atom. Therefore, substituted cycloalkyl, aryl, heterocyclyl and heteroaryl groups can also be substituted with alkyl, alkenyl, and alkynyl groups as defined herein.

By a “ring system” as the term is used herein is meant a moiety comprising one, two, three or more rings, which can be substituted with non-ring groups or with other ring systems, or both, which can be fully saturated, partially unsaturated, fully unsaturated, or aromatic, and when the ring system includes more than a single ring, the rings can be fused, bridging, or spirocyclic. By “spirocyclic” is meant the class of structures wherein two rings are fused at a single tetrahedral carbon atom, as is well known in the art.

Alkyl groups include straight chain and branched alkyl groups and cycloalkyl groups having from 1 to about 20 carbon atoms, and typically from 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. Representative substituted alkyl groups can be substituted one or more times with any of the groups listed above, for example, amino, hydroxy, cyano, carboxy, nitro, thio, alkoxy, and halogen groups.

Cycloalkyl groups are cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined above. Representative substituted cycloalkyl groups can be mono-substituted or substituted more than once, such as, but not limited to, 2,2-, 2,3-, 2,4-2,5- or 2,6-disubstituted cyclohexyl groups or mono-, di- or tri-substituted norbornyl or cycloheptyl groups, which can be substituted with, for example, amino, hydroxy, cyano, carboxy, nitro, thio, alkoxy, and halogen groups. The term “cycloalkenyl” alone or in combination denotes a cyclic alkenyl group.

The terms “carbocyclic” and “carbocycle” denote a ring structure wherein the atoms of the ring are carbon. In some embodiments, the carbocycle has 3 to 8 ring members, whereas in other embodiments the number of ring carbon atoms is 4, 5, 6, or 7. Unless specifically indicated to the contrary, the carbocyclic ring can be substituted with as many as N−1 substituents wherein N is the size of the carbocyclic ring with, for example, alkyl, alkenyl, alkynyl, amino, aryl, hydroxy, cyano, carboxy, heteroaryl, heterocyclyl, nitro, thio, alkoxy, and halogen groups, or other groups as are listed above.

(Cycloalkyl)alkyl groups, also denoted cycloalkylalkyl, are alkyl groups as defined above in which a hydrogen or carbon bond of the alkyl group is replaced with a bond to a cycloalkyl group as defined above.

Alkenyl groups include straight and branched chain and cyclic alkyl groups as defined above, except that at least one double bond exists between two carbon atoms. Thus, alkenyl groups have from 2 to about 20 carbon atoms, and typically from 2 to 12 carbons or, in some embodiments, from 2 to 8 carbon atoms. Examples include, but are not limited to vinyl, —CH═CH(CH₃), —CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl among others.

Cycloalkenyl groups include cycloalkyl groups having at least one double bond between 2 carbons. Thus for example, cycloalkenyl groups include but are not limited to cyclohexenyl, cyclopentenyl, and cyclohexadienyl groups. Cycloalkenyl groups can have from 3 to about 8-12 ring members, whereas in other embodiments the number of ring carbon atoms range from 3 to 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like, provided they include at least one double bond within a ring. Cycloalkenyl groups also include rings that are substituted with straight or branched chain alkyl groups as defined above.

(Cycloalkenyl)alkyl groups are alkyl groups as defined above in which a hydrogen or carbon bond of the alkyl group is replaced with a bond to a cycloalkenyl group as defined above.

Alkynyl groups include straight and branched chain alkyl groups, except that at least one triple bond exists between two carbon atoms. Thus, alkynyl groups have from 2 to about 20 carbon atoms, and typically from 2 to 12 carbons or, in some embodiments, from 2 to 8 carbon atoms. Examples include, but are not limited to —C≡CH, —C≡C(CH₃), —C≡C(CH₂CH₃), —CH₂C≡CH, —CH₂C≡C(CH₃), and —CH₂C≡C(CH₂CH₃) among others.

The term “heteroalkyl” by itself or in combination with another term means, unless otherwise stated, a stable straight or branched chain alkyl group consisting of the stated number of carbon atoms and one or two heteroatoms selected from the group consisting of O, N, and S, and wherein the nitrogen and sulfur atoms may be optionally oxidized and the nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group. Examples include: —O—CH₂—CH₂—CH₃, —CH₂—CH₂CH₂—OH, —CH₂—CH₂—NH—CH₃, —CH₂—S—CH₂—CH₃, —CH₂CH₂—S(═O)—CH₃, and —CH₂CH₂—O—CH₂CH₂—O—CH₃. Up to two heteroatoms may be consecutive, such as, for example, —CH₂—NH—OCH₃, or —CH₂—CH₂—S—S—CH₃.

The term “heteroalkenyl” by itself or in combination with another term means, unless otherwise stated, a stable straight or branched chain monounsaturated or di-unsaturated hydrocarbon group consisting of the stated number of carbon atoms and one or two heteroatoms selected from the group consisting of O, N, and S, and wherein the nitrogen and sulfur atoms may optionally be oxidized and the nitrogen heteroatom may optionally be quaternized. Up to two heteroatoms may be placed consecutively. Examples include —CH═CH—O—CH₃, —CH═CH—CH₂—OH, —CH₂—CH═N—OCH₃, —CH═CH—N(CH₃)—CH₃, —CH₂—CH═CH—CH₂—SH, and —CH═CH—O—CH₂CH₂—O—CH₃.

Aryl groups are cyclic aromatic hydrocarbons that do not contain heteroatoms. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons in the ring portions of the groups. Aryl groups can be unsubstituted or substituted, as defined above. Representative substituted aryl groups can be mono-substituted or substituted more than once, such as, but not limited to, 2-, 3-, 4-, 5-, or 6-substituted phenyl or 2-8 substituted naphthyl groups, which can be substituted with carbon or non-carbon groups such as those listed above.

Aralkyl groups are alkyl groups as defined above in which a hydrogen or carbon bond of an alkyl group is replaced with a bond to an aryl group as defined above. Representative aralkyl groups include benzyl and phenylethyl groups and fused (cycloalkylaryl)alkyl groups such as 4-ethyl-indanyl. Aralkenyl group are alkenyl groups as defined above in which a hydrogen or carbon bond of an alkyl group is replaced with a bond to an aryl group as defined above.

Heterocyclyl groups include aromatic and non-aromatic ring compounds containing 3 or more ring members, of which, one or more is a heteroatom such as, but not limited to, N, O, and S. In some embodiments, heterocyclyl groups include 3 to about 20 ring members, whereas other such groups have 3 to about 15 ring members. A heterocyclyl group designated as a C₂-heterocyclyl can be a 5-ring with two carbon atoms and three heteroatoms, a 6-ring with two carbon atoms and four heteroatoms and so forth. Likewise a C₄-heterocyclyl can be a 5-ring with one heteroatom, a 6-ring with two heteroatoms, and so forth. The number of carbon atoms plus the number of heteroatoms sums up to equal the total number of ring atoms. A heterocyclyl ring can also include one or more double bonds. A heteroaryl ring is an embodiment of a heterocyclyl group. The phrase “heterocyclyl group” includes fused ring species including those comprising fused aromatic and non-aromatic groups. For example, a dioxolanyl ring and a benzdioxolanyl ring system (methylenedioxyphenyl ring system) are both heterocyclyl groups within the meaning herein. The phrase also includes polycyclic ring systems containing a heteroatom such as, but not limited to, quinuclidyl. Heterocyclyl groups can be unsubstituted, or can be substituted as discussed above. Heterocyclyl groups include, but are not limited to, pyrrolidinyl, piperidinyl, piperazinyl, morpholinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Representative substituted heterocyclyl groups can be mono-substituted or substituted more than once, such as, but not limited to, piperidinyl or quinolinyl groups, which are 2-, 3-, 4-, 5-, or 6-substituted, or disubstituted with groups such as those listed above.

Heteroaryl groups are aromatic ring compounds containing 5 or more ring members, of which, one or more is a heteroatom such as, but not limited to, N, O, and S; for instance, heteroaryl rings can have 5 to about 8-12 ring members. A heteroaryl group designated as a C₂-heteroaryl can be a 5-ring with two carbon atoms and three heteroatoms, a 6-ring with two carbon atoms and four heteroatoms and so forth. Likewise a C₄-heteroaryl can be a 5-ring with one heteroatom, a 6-ring with two heteroatoms, and so forth. The number of carbon atoms plus the number of heteroatoms sums up to equal the total number of ring atoms. Heteroaryl groups include, but are not limited to, groups such as pyrrolyl, pyrazolyl, triazolyl, tetrazolyl, oxazolyl, isoxazolyl, thiazolyl, pyridinyl, thiophenyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Heteroaryl groups can be unsubstituted, or can be substituted with groups as is discussed above. Representative substituted heteroaryl groups can be substituted one or more times with groups such as those listed above.

Additional examples of aryl and heteroaryl groups include but are not limited to phenyl, biphenyl, indenyl, naphthyl (1-naphthyl, 2-naphthyl), N-hydroxytetrazolyl, N-hydroxytriazolyl, N-hydroxyimidazolyl, anthracenyl (1-anthracenyl, 2-anthracenyl, 3-anthracenyl), thiophenyl (2-thienyl, 3-thienyl), furyl (2-furyl, 3-furyl), indolyl, oxadiazolyl, isoxazolyl, quinazolinyl, fluorenyl, xanthenyl, isoindanyl, benzhydryl, acridinyl, thiazolyl, pyrrolyl (2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl 1,2,3-triazol-4-yl, 1,2,4-triazol-3-yl), oxazolyl (2-oxazolyl, 4-oxazolyl, 5-oxazolyl), thiazolyl (2-thiazolyl, 4-thiazolyl, 5-thiazolyl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyrazinyl, pyridazinyl (3-pyridazinyl, 4-pyridazinyl, 5-pyridazinyl), quinolyl (2-quinolyl, 3-quinolyl, 4-quinolyl, 5-quinolyl, 6-quinolyl, 7-quinolyl, 8-quinolyl), isoquinolyl (1-isoquinolyl, 3-isoquinolyl, 4-isoquinolyl, 5-isoquinolyl, 6-isoquinolyl, 7-isoquinolyl, 8-isoquinolyl), benzo[b]furanyl (2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl (2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl, (2-(2,3-dihydro-benzo[b]thiophenyl), 3-(2,3-dihydro-benzo[b]thiophenyl), 4-(2,3-dihydro-benzo[b]thiophenyl), 5-(2,3-dihydro-benzo[b]thiophenyl), 6-(2,3-dihydro-benzo[b]thiophenyl), 7-(2,3-dihydro-benzo[b]thiophenyl), indolyl (1-indolyl, 2-indolyl, 3-indolyl, 4-indolyl, 5-indolyl, 6-indolyl, 7-indolyl), indazole (1-indazolyl, 3-indazolyl, 4-indazolyl, 5-indazolyl, 6-indazolyl, 7-indazolyl), benzimidazolyl (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl, 3-carbazolyl, 4-carbazolyl), 5H-dibenz[b,f]azepine (5H-dibenz[b,f]azepin-1-yl, 5H-dibenz[b,f]azepine-2-yl, 5H-dibenz[b,f]azepine-3-yl, 5H-dibenz[b,f]azepine-4-yl, 5H-dibenz[b,f]azepine-5-yl), 10,11-dihydro-5H-dibenz[b,f]azepine (10,11-dihydro-5H-dibenz[b,f]azepine-1-yl, 10,11-dihydro-5H-dibenz[b,f]azepine-2-yl, 10,11-dihydro-5H-dibenz[b,f]azepine-3-yl, 10,11-dihydro-5H-dibenz[b,f]azepine-4-yl, 10,11-dihydro-5H-dibenz[b,f]azepine-5-yl), and the like.

Heterocyclylalkyl groups are alkyl groups as defined above in which a hydrogen or carbon bond of an alkyl group as defined above is replaced with a bond to a heterocyclyl group as defined above. Representative heterocyclyl alkyl groups include, but are not limited to, furan-2-yl methyl, furan-3-yl methyl, pyridine-3-yl methyl, tetrahydrofuran-2-yl ethyl, and indol-2-yl propyl.

Heteroarylalkyl groups are alkyl groups as defined above in which a hydrogen or carbon bond of an alkyl group is replaced with a bond to a heteroaryl group as defined above.

The term “alkoxy” refers to an oxygen atom connected to an alkyl group, including a cycloalkyl group, as are defined above. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include one to about 12-20 carbon atoms bonded to the oxygen atom, and can further include double or triple bonds, and can also include heteroatoms. For example, an allyloxy group is an alkoxy group within the meaning herein. A methoxyethoxy group is also an alkoxy group within the meaning herein.

“Halo” as the term is used herein includes fluoro, chloro, bromo, and iodo. A “haloalkyl” group includes mono-halo alkyl groups, and poly-halo alkyl groups wherein all halo atoms can be the same or different. Examples of haloalkyl include trifluoromethyl, 1,1-dichloroethyl, 1,2-dichloroethyl, 1,3-dibromo-3,3-difluoropropyl and the like.

The terms “halo” or “halogen” or “halide” by themselves or as part of another substituent mean, unless otherwise stated, a fluorine, chlorine, bromine, or iodine atom, preferably, fluorine, chlorine, or bromine.

The term “(Cₓ-C_{y})perfluoroalkyl,” wherein x<y, means an alkyl group with a minimum of x carbon atoms and a maximum of y carbon atoms, wherein all hydrogen atoms are replaced by fluorine atoms. Preferred is —(C₁-C₆)perfluoroalkyl, more preferred is —(C₁-C₃)perfluoroalkyl, most preferred is —CF₃.

The term “(Cₓ-C_{y})perfluoroalkylene,” wherein x<y, means an alkyl group with a minimum of x carbon atoms and a maximum of y carbon atoms, wherein all hydrogen atoms are replaced by fluorine atoms. Preferred is —(C₁-C₆)perfluoroalkylene, more preferred is —(C₁-C₃)perfluoroalkylene, most preferred is —CF₂—.

The terms “aryloxy” and “arylalkoxy” refer to, respectively, an aryl group bonded to an oxygen atom and an aralkyl group bonded to the oxygen atom at the alkyl moiety. Examples include but are not limited to phenoxy, naphthyloxy, and benzyloxy.

An “acyl” group as the term is used herein refers to a group containing a carbonyl moiety wherein the group is bonded via the carbonyl carbon atom. The carbonyl carbon atom is also bonded to another carbon atom, which can be part of an alkyl, aryl, aralkyl cycloalkyl, cycloalkylalkyl, heterocyclyl, heterocyclylalkyl, heteroaryl, heteroarylalkyl group or the like. In the special case wherein the carbonyl carbon atom is bonded to a hydrogen, the group is a “formyl” group, an acyl group as the term is defined herein. An acyl group can include 0 to about 12-20 additional carbon atoms bonded to the carbonyl group. An acyl group can include double or triple bonds within the meaning herein. An acryloyl group is an example of an acyl group. An acyl group can also include heteroatoms within the meaning here. A nicotinoyl group (pyridyl-3-carbonyl) group is an example of an acyl group within the meaning herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “amine” includes primary, secondary, and tertiary amines having, e.g., the formula N(group)₃ wherein each group can independently be H or non-H, such as alkyl, aryl, and the like. Amines include but are not limited to R—NH₂, for example, alkylamines, arylamines, alkylarylamines; R₂NH wherein each R is independently selected, such as dialkylamines, diarylamines, aralkylamines, heterocyclylamines and the like; and R₃N wherein each R is independently selected, such as trialkylamines, dialkylarylamines, alkyldiarylamines, triarylamines, and the like. The term “amine” also includes ammonium ions as used herein.

An “amino” group is a substituent of the form —NH₂, —NHR, —NR₂, —NR₃⁺, wherein each R is independently selected, and protonated forms of each. Accordingly, any compound substituted with an amino group can be viewed as an amine.

An “ammonium” ion includes the unsubstituted ammonium ion NH₄⁺, but unless otherwise specified, it also includes any protonated or quaternarized forms of amines. Thus, trimethylammonium hydrochloride and tetramethylammonium chloride are both ammonium ions, and amines, within the meaning herein.

The term “amide” (or “amido”) includes C- and N-amide groups, i.e., —C(O)NR₂, and —NRC(O)R groups, respectively. Amide groups therefore include but are not limited to carbamoyl groups (—C(O)NH₂) and formamide groups (—NHC(O)H). A “carboxamido” group is a group of the formula C(O)NR₂, wherein R can be H, alkyl, aryl, etc.

The term “urethane” (or “carbamyl”) includes N- and O-urethane groups, i.e., —NRC(O)OR and —OC(O)NR₂ groups, respectively.

The term “sulfonamide” (or “sulfonamido”) includes S- and N-sulfonamide groups, i.e., —SO₂NR₂ and —NRSO₂R groups, respectively. Sulfonamide groups therefore include but are not limited to sulfamoyl groups (—SO₂NH₂). An organosulfur structure represented by the formula —S(O)(NR)— is understood to refer to a sulfoximine, wherein both the oxygen and the nitrogen atoms are bonded to the sulfur atom, which is also bonded to two carbon atoms.

The term “amidine” or “amidino” includes groups of the formula —C(NR)NR₂. Typically, an amidino group is —C(NH)NH₂.

The term “guanidine” or “guanidino” includes groups of the formula —NRC(NR)NR₂. Typically, a guanidino group is —NHC(NH)NH₂.

A “salt” as is well known in the art includes an organic compound such as a carboxylic acid, a sulfonic acid, or an amine, in ionic form, in combination with a counterion. For example, acids in their anionic form can form salts with cations such as metal cations, for example sodium, potassium, and the like; with ammonium salts such as NH₄⁺ or the cations of various amines, including tetraalkyl ammonium salts such as tetramethylammonium, or other cations such as trimethylsulfonium, and the like. A “pharmaceutically acceptable” or “pharmacologically acceptable” salt is a salt formed from an ion that has been approved for human consumption and is generally non-toxic, such as a chloride salt or a sodium salt. A “zwitterion” is an internal salt such as can be formed in a molecule that has at least two ionizable groups, one forming an anion and the other a cation, which serve to balance each other. For example, amino acids such as glycine can exist in a zwitterionic form. A “zwitterion” is a salt within the meaning herein.

A “hydrate” is a compound that exists in a composition with water molecules. The composition can include water in stoichiometic quantities, such as a monohydrate or a dihydrate, or can include water in random amounts.

A “solvate” is a similar composition except that a solvent other that water replaces the water. For example, methanol or ethanol can form an “alcoholate”, which can again be stoichiometic or non-stoichiometric.

“Tautomers” are two forms of a substance differing only by the position of a hydrogen atom in the molecular structures.

A “prodrug” as is well known in the art is a substance that can be administered to a patient where the substance is converted in vivo by the action of biochemicals within the patient body, such as enzymes, to the active pharmaceutical ingredient. Examples of prodrugs include esters of carboxylic acid groups, which can be hydrolyzed by endogenous esterases as are found in the bloodstream of humans and other mammals.

In addition, where features or aspects of the invention are described in terms of Markush groups, those skilled in the art will recognize that the invention is also thereby described in terms of any individual member or subgroup of members of the Markush group. For example, if X is described as selected from the group consisting of bromine, chlorine, and iodine, claims for X being bromine and claims for X being bromine and chlorine are fully described. Moreover, where features or aspects of the invention are described in terms of Markush groups, those skilled in the art will recognize that the invention is also thereby described in terms of any combination of individual members or subgroups of members of Markush groups. Thus, for example, if X is described as selected from the group consisting of bromine, chlorine, and iodine, and Y is described as selected from the group consisting of methyl, ethyl, and propyl, claims for X being bromine and Y being methyl are fully described.

In various embodiments, the compound or set of compounds, such as are used in the inventive methods, can be any one of any of the combinations and/or sub-combinations of the above-listed embodiments.

### DESCRIPTION

### Compounds of the Invention

In various embodiments, the invention provides a compound of formula (I)

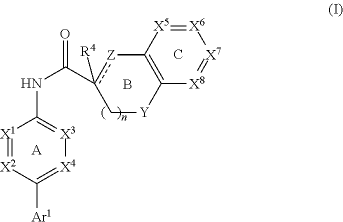

wherein

X¹, X², X³, and X⁴ are each independently N, CH, or CR¹ such that ring A comprises a phenyl, pyridyl, pyrazinyl, pyridazinyl, or pyrimidinyl ring, provided that 0-2 of X¹, X², X³ and X⁴ are CR¹, the remainder being independently N or CH;

R¹ comprises independently at each occurrence F, Cl, Br, I, CF₃, OCF₃, (C₁₋₆)-alkyl substituted with 0-2 R^{a}, (C₂₋₆)-alkenyl substituted with 0-2 R^{a}, (C₂₋₆)-alkynyl substituted with 0-2 R^{a}, (CH₂)ₚNO₂, (CH₂)ₚCN, (CH₂)ₚOR, (CH₂)ₚNR₂, (CH₂)ₚC(═O)R, (CH₂)ₚOC(═O)R, (CH₂)ₚC(═O)OR, (CH₂)ₚC(═O)NR₂, (CH₂)ₚOC(═O)NR₂, (CH₂)ₚNRC(═O)R, (CH₂)ₚNRC(═O)OR, (CH₂)ₚNRC(═O)NR₂, (CH₂)ₚC(═NH)NH₂, (CH₂)ₚS(O)_{q}R, (CH₂)ₚSO₂NR₂, (CH₂)ₚNRSO₂R, (CH₂)ₚNRSO₂NR₂, (CH₂)ₚ-(3-10 membered)-cycloalkyl substituted with 0-2 R^{a}, or (CH₂)ₚ-(4-10 membered)-heterocyclyl substituted with 0-2 R^{a} comprising 1-4 heteroatoms selected from O, S(O)_{q}, and N; or two adjacent R′ substituents can form a fused phenyl or a 5-6 membered heteroaryl comprising carbon atoms and 1-2 heteroatoms selected from O, S(O)_{q}, and N, and substituted with 0-3 R^{a}, wherein p is 0-4 and q is 0-2;

R is independently at each occurrence H, (C₁₋₆)-alkyl substituted with 0-2 R^{a}, (C₂₋₆)-alkenyl substituted with 0-2 R^{a}, (C₂₋₆)-alkynyl substituted with 0-2 R^{a}, (3-10 membered)-cycloalkyl substituted with 0-2 R^{a}, or (3-10 membered)-heterocyclyl substituted with 0-2 R^{a} comprising 1-4 heteroatoms selected from O, S(O)_{q}, and N; or, an NR₂ forms a (3-10 membered)-heterocyclyl substituted with 0-2 R^{a} and comprising 0-1 additional ring heteroatoms selected from N, O, and S(O)_{q};

R^{a} is independently at each occurrence oxo, F, Cl, Br, I, CF₃, OCF₃, (C₁₋₆)-alkyl substituted with 0-2 R^{a}, (C₂₋₆)-alkenyl substituted with 0-2 R^{a}, (C₂₋₆)-alkynyl substituted with 0-2 R^{a}, (CH₂)ₚNO₂, (CH₂)ₚCN, (CH₂)ₚOR, (CH₂)ₚNR₂, (CH₂)ₚC(═O)R, (CH₂)ₚOC(═O)R, (CH₂)ₚC(═O)OR, (CH₂)ₚC(═O)NR₂, (CH₂)ₚOC(═O)NR₂, (CH₂)ₚNRC(═O)R, (CH₂)ₚNRC(═O)OR, (CH₂)ₚNRC(═O)NR₂, (CH₂)ₚC(═NH)NH₂, (CH₂)ₚS(O)_{q}R, (CH₂)ₚSO₂NR₂, (CH₂)ₚNRSO₂R, (CH₂)ₚNRSO₂NR₂, (CH₂)ₚ-(3-10 membered)-cycloalkyl substituted with 0-2 R^{a}, or (CH₂)ₚ-(3-10 membered)-heterocyclyl substituted with 0-2 R^{a} comprising 1-4 heteroatoms selected from O, S(O)_{q}, and N;

Ar¹ comprises a 5- or 6-membered heteroaryl comprising at least one nitrogen atom and 0-3 additional heteroatoms selected from O, S(O)_{q}, and N; when Ar¹ is a 5-membered heteroaryl, a nitrogen atom is disposed one atom away from an atom of the heteroaryl bonded to ring A, and when Ar¹ is a 6-membered heteroaryl, a nitrogen atom is disposed two atoms away from an atom of the heteroaryl bonded to ring A; wherein Ar¹ is optionally fused with phenyl or a 5-6 membered heteroaryl comprising 1-2 heteroatoms selected from O, S(O)_{q}, and N, wherein the fused phenyl or 5-6 membered heteroaryl is substituted with 0-3 R^{a};

n is 0 or 1;

wherein a dotted line indicates a single bond or a double bond;

Y is O, CH(R³), S(O)_{q}, N(R³), or C(═O);

Z is O, CH(R³), CR³, S(O)_{q}, N(R³), or C(═O), provided that when the double bond is absent, R⁴ is present, Z is O, CH(R³), or C(═O) and when the double bond is present R⁴ is absent and Z is CR³;

X⁵, X⁶, X⁷ and X⁸ are each independently N, CH, or CR², such that ring C comprises a phenyl, pyridyl, pyrazinyl, pyridazinyl, or pyrimidinyl ring;

R² is independently at each occurrence hydrogen, (C₁-C₆)alkyl, hydroxy(C₁-C₆)alkyl, (C₂-C₆)alkenyl; (C₂-C₆)alkynyl; halogen; —C≡N; —NO₂; —C(═O)R; —C(═O)OR; (C₁-C₆)alkylene-C(═O)OR; —C(═O)NR₂; (C₁-C₆)alkylene-C(═O)NR₂; —C(═NR)NR₂; —OR; (C₁-C₆)alkylene-OR; —OC(═O)(C₁-C₆)alkyl; —OC(═O)O(C₁-C₆)alkyl; —OC(═O)NR₂; —NR₂; —NRC(═O)R; —NRC(═O)O(C₁-C₆)alkyl; —NRC(═O)NR₂; —NR(C₁-C₆)alkylene-NR₂; —NR(C₁-C₆)alkylene-OR; —NR(C₁-C₆)alkylene-Ar²; —NRSO₂R; —SR; —S(O)R; —SO₂R; —OSO₂(C₁-C₆)alkyl; —SO₂NR₂; (C₁-C₃)perfluoroalkyl; —O(C₁-C₃)perfluoroalkyl; pyrazolyl; triazolyl; and tetrazolyl; or two adjacent R² groups taken together form a fused cycloalkyl, heterocyclyl, aryl or heteroaryl ring which is substituted with 0-3 R^{a};

R³ is H, CF₃, OCF₃, OCH₃, (C₁₋₆)-alkyl substituted with 0-3 R^{a}, or NR₂;

R⁴ is H or (C₁₋₆)alkyl substituted with 0-3 R^{a};

or any salt, stereoisomer, tautomer, hydrate, solvate, or prodrug thereof.

In various embodiments, ring B and ring C together form a chroman ring system, that is, a ring system having a ring nucleus of the formula

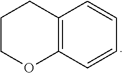

In various embodiments, ring B and ring C together form a 2H-chromene ring system, that is, a ring system having a ring nucleus of the formula

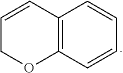

In various embodiments, ring B and ring C together form a 2,3-dihydrobenzo[b][1,4]dioxine ring system, that is, a ring system having a ring nucleus of the formula

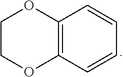

In various embodiments, ring B and ring C together form a 2,3-dihydrobenzofuran ring system, that is, a ring system having a ring nucleus of the formula

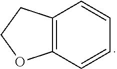

For example, in various embodiments, Y can be O and n=1.

For example, in various embodiments, Z can be CH₂.

For example, in various embodiments, Y can be O and n=1 and Z can be CH₂.

For example, in various embodiments, Z can be O.

For example, in various embodiments, Z can be O and n=1, or Z can be O and n=0.

For example, in various embodiments, Y can be CH₂.

For example, in various embodiments, Z can be O and n=1 and Y can be CH₂.

For example, in various embodiments, both Y and Z can be CH₂.

For example, in various embodiments, Ar¹ can comprise a pyrididyl, pyrimidinyl, amino-substituted pyridyl, halo-substituted pyridyl, pyridyl substituted with both amino and halo, of amino-substituted pyrimidinyl, and R¹ is other than unsubstituted alkyl.

For example, in various embodiments, Ar¹ can comprise a pyrazolyl, methylpyrazolyl, dimethylpyrazolyl, pyridinyl, methylpyridyl, pyrrolopyridyl, pyrrolopyrimidinyl, naphthyridinyl, pyridopyrimidinyl, imidazolyl, triazolyl, or oxazolyl ring system.

For example, in various embodiments, Ar¹ can comprise a pyrazol-4-yl, 3-methylpyrazol-4-yl, 5-methylpyrazol-4-yl, 3-aminopyridazol-4-yl, 3,5-dimethylpyrazol-4-yl ring system, imidazol-4-yl, 3-methylimidazol-4-yl, 1,2,3-triazol-4-yl, or 5-aminooxazol-4-yl ring system.

For example, in various embodiments, Ar¹ can comprise a pyridin-4-yl, 2-methylpyridin-y-yl ring system, 3-fluoropyridin-4-yl, 3-chloropyridin-4-yl, 3-cyano-pyridin-4-yl, 2-NR₂-substituted-pyridin-4-yl, 1,3-pyrimidin-4-yl, 2-NR₂-substituted-1,3-pyrimidin-4-yl, or 5-NR₂-substituted-1,3-pyrimidin-4-yl ring system.

For example, in various embodiments, Ar¹ can comprise a 1,8-naphthyridin-4-yl

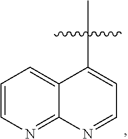

a 1,8-naphthyridin-3-yl

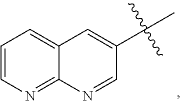

a pyrido[2,3-d]pyrimidin-4-yl

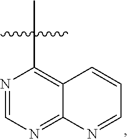

a 1H-pyrrole[2,3-b]pyridin-4-yl

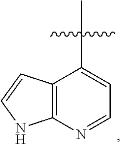

or a 7H-pyrrole[2,3-d]pyrimidin-4-yl

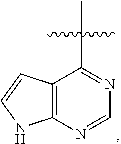

ring system, wherein a wavy line indicates a point of attachment.

For example, in various embodiments, ring A can comprise phenyl, wherein all R¹ are H or wherein ring A is mono- or independently plurisubstituted with R¹ comprising (C₁₋₆)-alkyl, fluoro, chloro, (C₁₋₄-alkoxy, carboxamidoalkoxy, aminoalkylcarboxamido, trifluoromethyl, carboxamido, aminoalkoxy, trifluoromethoxy, sulfonamido-substituted phenyl, hydroxy, heterocyclyloxy, heterocyclylalkoxy, aminoalkylthio, aralkoxy, aminoalkylamino, heterocyclylamino, heterocyclylalkylamino, heterocyclylthio, heterocyclylalkylthio, aryloxy, hydroxyalkoxy, alkoxyalkoxy, or alkenyloxyalkoxy, or any combination thereof.

For example, in various embodiments, ring A can comprise pyridyl, pyridazinyl, pyrazinyl, or pyrimidyl.

For example, in various embodiments, R⁴ can be H or methyl.

For example, in various embodiments, X⁵, X⁶, X⁷ and X⁸ can all be CH or CR².

For example, in various embodiments, X⁵ and X⁶ can both be CR² and the CR² together comprise a fused aryl ring, or X⁶ and X⁷ can both be CR² and the CR² together comprise a fused aryl ring, or X⁷ and X⁸ can both be CR² and the CR² together comprise a fused aryl ring.

For example, in various embodiments, R² can comprise chloro, fluoro, trifluoromethyl, carboxy, (C₁₋₄-alkoxycarbonyl, carboxamido, aminoalkylcarboxamido, aminoalkoxy, heterocyclyloxy, or heterocyclylalkoxy, or any combination thereof.

In various embodiments, the invention provides a compound of any of the examples 1-427, or any tautomer, salt, stereoisomer, hydrate, solvent, or prodrug thereof.

It is to be understood that other particular and preferred embodiments of the compounds of the invention will combine the features of the particular and preferred embodiments of the invention explicitly described above. Embodiments defined by such combinations are contemplated as particular embodiments of the invention.

In other preferred embodiments the compound of formula I, or any of the embodiments thereof, is an isolated compound. In other preferred embodiments, the compound of formula I, and compositions containing the compound, including pharmaceutical compositions, are substantially free of pharmaceutically unacceptable contaminants. A pharmaceutically unacceptable contaminant is a compound which, if present in more than an insubstantial amount, would render the compound unsuitable for use as a pharmaceutical for therapeutic administration.

### Methods for Preparing Compounds of the Invention

There are provided processes for preparing compounds according to formula I, intermediates that are useful in the preparation of such compounds, and processes for preparing such intermediates. The compounds can be prepared by a variety of synthetic routes. Representative procedures are shown below in the Examples in Schemes 1-11, and in the specific synthetic procedures provided in the Examples below. It will be readily apparent that the compounds can be synthesized by substitution of the appropriate starting materials, reactants, and reagents in the syntheses shown below. It will also be apparent that the selective protection and deprotection steps, as well as the order of the steps themselves, can be carried out in varying order, depending on the nature of the reactions. Precursor compounds, intermediates, and reagents are commercially available or can be prepared from commercially available starting materials. The following schemes are representative, and are in no way intended to limit the scope of the compounds in the embodiments of the present invention.

In the text, formulae and schemes that follow, unless otherwise indicated, the variables are as defined above for formula I.

The above-described reactions, unless otherwise noted, are usually conducted at a pressure of about one to about three atmospheres, preferably at ambient pressure (about one atmosphere).

The present invention further embraces isolated compounds of the invention according to formula I. The expression “isolated compound” refers to a preparation of a compound of the invention, or a mixture of compounds of the invention, wherein the isolated compound has been separated from the reagents used, and/or byproducts formed, in the synthesis of the compound or compounds. “Isolated” does not mean that the preparation is technically pure (homogeneous), but it is sufficiently pure to compound in a form in which it can be used therapeutically. Preferably an “isolated compound” refers to a preparation of a compound of the invention or a mixture of compounds of the invention, which contains the named compound or mixture of compounds of the invention in an amount of at least 10 percent by weight of the total weight. Preferably the preparation contains the named compound or mixture of compounds in an amount of at least 50 percent by weight of the total weight; more preferably at least 80 percent by weight of the total weight; and most preferably at least 90 percent, at least 95 percent or at least 98 percent by weight of the total weight of the preparation.

The compounds of the invention and intermediates may be isolated from their reaction mixtures and purified by standard techniques such as filtration, liquid-liquid extraction, solid phase extraction, distillation, recrystallization or chromatography, including flash column chromatography, or HPLC.

The synthetic methods described above reflect a convergent synthesis strategy. These convergent synthetic schemes allow for arrangement of the assembly steps of the backbone of the target compounds and derivatization of derivatizable functionalities to accommodate functional group sensitivity and/or to allow for functional groups or elements to be introduced either before or after the assembly of the backbone of the target compounds via the condensation and coupling reactions described.

It will be appreciated by one skilled in the art that certain aromatic substituents in the compounds of the invention, intermediates used in the processes described above, or precursors thereto, may be introduced by employing aromatic substitution reactions to introduce or replace a substituent, or by using functional group transformations to modify an existing substituent, or a combination thereof. Such reactions may be effected either prior to or immediately following the processes mentioned above, and are included as part of the process aspect of the invention. The reagents and reaction conditions for such procedures are known in the art. Specific examples of procedures which may be employed include, but are not limited to, electrophilic functionalization of an aromatic ring, for example via nitration, halogenation, or acylation; transformation of a nitro group to an amino group, for example via reduction, such as by metal/acid or catalytic hydrogenation; acylation, alkylation, or sulfonylation of an amino or hydroxyl group; replacement of an amino group by another functional group via conversion to an intermediate diazonium salt followed by nucleophilic or free radical substitution of the diazonium salt; or replacement of a halogen by another group, for example via nucleophilic or organometallically-catalyzed substitution reactions.

Additionally, in the aforesaid processes, certain functional groups which would be sensitive to the reaction conditions may be protected by protecting groups. A protecting group is a derivative of a chemical functional group which would otherwise be incompatible with the conditions required to perform a particular reaction which, after the reaction has been carried out, can be removed to re-generate the original functional group, which is thereby considered to have been “protected”, for example N-protected or O-protected, as defined above. Any chemical functionality that is a structural component of any of the reagents used to synthesize compounds of this invention may be optionally protected with a chemical protecting group if such a protecting group is useful in the synthesis of compounds of this invention. The person skilled in the art knows when protecting groups are indicated, how to select such groups, and processes that can be used for selectively introducing and selectively removing them, because methods of selecting and using protecting groups have been extensively documented in the chemical literature. Techniques for selecting, incorporating and removing chemical protecting groups may be found, for example, in Protective Groups in Organic Synthesis, Third Ed. by Theodora W. Greene, Peter G. M. Wuts (John Wiley & Sons, Inc., 1999), the entire disclosure of which is incorporated herein by reference.

In addition to use of a protecting group, sensitive functional groups may be introduced as synthetic precursors to the functional group desired in the intermediate or final product. An example of this is an aromatic nitro (—NO₂) group. The aromatic nitro group goes not undergo any of the nucleophilic reactions of an aromatic amino group. However, the nitro group can serve as the equivalent of a protected amino group because it is readily reduced to the amino group under mild conditions that are selective for the nitro group over most other functional groups.

It will be appreciated by one skilled in the art that the processes described are not the exclusive means by which compounds of the invention may be synthesized and that an extremely broad repertoire of synthetic organic reactions is available to be potentially employed in synthesizing compounds of the invention. The person skilled in the art knows how to select and implement appropriate synthetic routes. Suitable synthetic methods may be identified by reference to the literature, including reference sources such as Comprehensive Organic Synthesis, Ed. B. M. Trost and I. Fleming (Pergamon Press, 1991), Comprehensive Organic Functional Group Transformations, Ed. A. R. Katritzky, O. Meth-Cohn, and C. W. Rees (Pergamon Press, 1996), Comprehensive Organic Functional Group Transformations II, Ed. A. R. Katritzky and R. J. K. Taylor (Editor) (Elsevier, 2^{nd} Edition, 2004), Comprehensive Heterocyclic Chemistry, Ed. A. R. Katritzky and C. W. Rees (Pergamon Press, 1984), and Comprehensive Heterocyclic Chemistry II, Ed. A. R. Katritzky, C. W. Rees, and E. F. V. Scriven (Pergamon Press, 1996).

### Treatment of Rho-Kinase Medicated Disorders Using Compounds of the Invention

According to another embodiment of the invention, a method of treating a patient suffering from Rho-kinase-mediated disorder is provided, comprising administering to the patient an effective amount of at least one compound of the invention, or any tautomer, salt, stereoisomer, hydrate, solvent, or prodrug thereof, either alone, or in combination with a pharmaceutically acceptable carrier.

The invention is also directed to the use of a compound of the invention, or a tautomer, salt, stereoisomer, hydrate, solvent, or prodrug thereof, in the preparation of a medicament for treatment of a Rho-Kinase mediated disorder

The compounds of the present invention or a tautomer, salt, stereoisomer, hydrate, solvent, or prodrug thereof can inhibit or otherwise influence an activity of any Rho kinase such as ROCK I and/or ROCK II. Therefore, the compounds of the present invention are useful for the treatment and/or prevention of a variety of Rho-kinase-mediated diseases.

Rho-kinase-mediated diseases which can be treated and/or prevented by using the compound of the present invention include, but are not limited to, hypertension, pulmonary hypertension, atherosclerosis, stroke, angina, heart failure, arterial obstruction, peripheral arterial disease, peripheral circulation disorder, vasospasm, erectile dysfunction, acute and chronic pain, dementia, Alzheimer's disease, Parkinson's disease, neuronal degeneration, asthma, amyotrophic lateral sclerosis, spinal cord injury, rheumatoid arthritis, osteoarthritis, osteoporosis, psoriasis, multiple sclerosis, diabetes, urinary organ diseases such as overactive bladder (OAB) and benign prostatic hypertrophy (BPH), metastasis, cancer, glaucoma, ocular hypertension, retinopathy, autoimmune disease, viral infection, and myocardial protection.

Rho-kinase inhibitors of the present will also be effective for pain alleviation and cartilage protection and will therefore also be effective to treat osteoarthritis, rheumatoid arthritis. osteoporosis, and osteoarthritis.

Particular and preferred embodiments of this aspect of the invention are those wherein the compound of the invention used in the method of treatment, either alone or as part of a composition is a particular or preferred embodiment of the compound of the invention in the description of the compounds and compositions of the invention as provided herein.

The compounds according to the invention may be administered to individuals (mammals, including animals and humans) afflicted with Rho-kinase-mediated disorders as identified herein.

### Salts of Compounds According to the Invention

The compounds of the present invention may take the form of salts. The term “salts” embraces addition salts of free acids or free bases which are compounds of the invention. Salts can be “pharmaceutically-acceptable salts.” The term “pharmaceutically-acceptable salt” refers to salts which possess toxicity profiles within a range that affords utility in pharmaceutical applications. Pharmaceutically unacceptable salts may nonetheless possess properties such as high crystallinity, which have utility in the practice of the present invention, such as for example utility in process of synthesis, purification or formulation of compounds of the invention.

Suitable pharmaceutically-acceptable acid addition salts may be prepared from an inorganic acid or from an organic acid. Examples of inorganic acids include hydrochloric, hydrobromic, hydriodic, nitric, carbonic, sulfuric, and phosphoric acids. Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid. Examples of pharmaceutically unacceptable acid addition salts include, for example, perchlorates and tetrafluoroborates.

Suitable pharmaceutically acceptable base addition salts of compounds of the invention include, for example, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylenediamine, chloroprocaine, choline, diethanolamine, ethylenediamine, meglumine (N-methylglucamine) and procaine. Examples of pharmaceutically unacceptable base addition salts include lithium salts and cyanate salts. Although pharmaceutically unacceptable salts are not generally useful as medicaments, such salts may be useful, for example as intermediates in the synthesis of Formula I compounds, for example in their purification by recrystallization. All of these salts may be prepared by conventional means from the corresponding compound according to Formula I by reacting, for example, the appropriate acid or base with the compound according to Formula I.

### Isomerism and Tautomerism in Compounds of the Invention

### Tautomerism

Within the present invention it is to be understood that a compound of the formula I or a salt thereof may exhibit the phenomenon of tautomerism whereby two chemical compounds that are capable of facile interconversion by exchanging a hydrogen atom between two atoms, to either of which it forms a covalent bond. Since the tautomeric compounds exist in mobile equilibrium with each other they may be regarded as different isomeric forms of the same compound. It is to be understood that the formulae drawings within this specification can represent only one of the possible tautomeric forms. However, it is also to be understood that the invention encompasses any tautomeric form which inhibits Rho-kinase activity, and is not to be limited merely to any one tautomeric form utilized within the formulae drawings. The formulae drawings within this specification can represent only one of the possible tautomeric forms and it is to be understood that the specification encompasses all possible tautomeric forms of the compounds drawn not just those forms which it has been convenient to show graphically herein. For example, tautomerism may be exhibited by a pyrazolyl group bonded as indicated by the wavy line. While both substituents would be termed a 4-pyrazolyl group, it is evident that a different nitrogen atom bears the hydrogen atom in each structure.

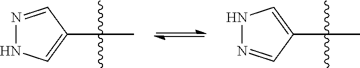

Such tautomerism can also occur with substituted pyrazoles such as 3-methyl, 5-methyl, or 3,5-dimethylpyrazoles, and the like. Indeed, tautomerization makes 3-methylpyrazole and 5-methylpyrazole equivalent structures, and it is understood that a name of a tautomerizable structure or moiety represents both possible numberings. For example, a pyrazol-4-yl moiety substituted with a group, for example an amino group, adjacent to one of the nitrogen atoms is capable of tautomerization as shown below, and depending on which tautomer is named, the moiety could be termed a 3-aminopyrazol-4-yl or a 5-aminopyrazol-4-yl group. When such is the situation, a name as used herein represents both possible variants.

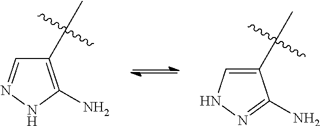

### Optical Isomerism

It will be understood that when compounds of the present invention contain one or more chiral centers, the compounds may exist in, and may be isolated as pure enantiomeric or diastereomeric forms or as racemic mixtures. The present invention therefore includes any possible enantiomers, diastereomers, racemates or mixtures thereof of the compounds of the invention which are biologically active in the treatment of Rho-kinase mediated diseases. The isomers resulting from the presence of a chiral center comprise a pair of non-superimposable isomers that are called “enantiomers.” Single enantiomers of a pure compound are optically active, i.e., they are capable of rotating the plane of plane polarized light. Single enantiomers are designated according to the Cahn-Ingold-Prelog system. Once the priority ranking of the four groups is determined, the molecule is oriented so that the lowest ranking group is pointed away from the viewer. Then, if the descending rank order of the other groups proceeds clockwise, the molecule is designated (R) and if the descending rank of the other groups proceeds counterclockwise, the molecule is designated (S). In the example in Scheme 14, the Cahn-Ingold-Prelog ranking is A>B>C>D. The lowest ranking atom, D is oriented away from the viewer.

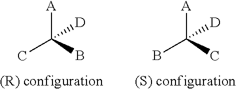

The present invention is meant to encompass diastereomers as well as their racemic and resolved, diastereomerically and enantiomerically pure forms and salts thereof.

Diastereomeric pairs may be resolved by known separation techniques including normal and reverse phase chromatography, and crystallization.

“Isolated optical isomer” means a compound which has been substantially purified from the corresponding optical isomer(s) of the same formula. Preferably, the isolated isomer is at least about 80%, more preferably at least 90% pure, even more preferably at least 98% pure, most preferably at least about 99% pure, by weight.

Isolated optical isomers may be purified from racemic mixtures by well-known chiral separation techniques. According to one such method, a racemic mixture of a compound of the invention, or a chiral intermediate thereof, is separated into 99% wt. % pure optical isomers by HPLC using a suitable chiral column, such as a member of the series of DAICEL® CHIRALPAK® family of columns (Daicel Chemical Industries, Ltd., Tokyo, Japan). The column is operated according to the manufacturer's instructions.

### Rotational Isomerism

It is understood that due to chemical properties (i.e., resonance lending some double bond character to the C—N bond) of restricted rotation about the amide bond linkage (as illustrated below) it is possible to observe separate rotamer species and even, under some circumstances, to isolate such species (Scheme 15). It is further understood that certain structural elements, including steric bulk or substituents on the amide nitrogen, may enhance the stability of a rotamer to the extent that a compound may be isolated as, and exist indefinitely, as a single stable rotamer. The present invention therefore includes any possible stable rotamers of formula I which are biologically active in the treatment of cancer or other proliferative disease states.

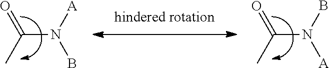

### D. Regioisomerism

The preferred compounds of the present invention have a particular spatial arrangement of substituents on the aromatic rings, which is related to the structure activity relationship demonstrated by the compound class. Often such substitution arrangement is denoted by a numbering system; however, numbering systems are often not consistent between different ring systems. In six-membered aromatic systems, the spatial arrangements are specified by the common nomenclature “para” for 1,4-substitution, “meta” for 1,3-substitution and “ortho” for 1,2-substitution as shown below (Scheme 16).

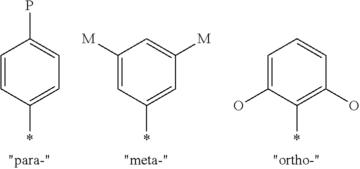

### Pharmaceutical Compositions

Another aspect of an embodiment of the invention provides compositions of the compounds of the invention, alone or in combination with another medicament. As set forth herein, compounds of the invention include stereoisomers, tautomers, solvates, prodrugs, pharmaceutically acceptable salts and mixtures thereof. Compositions containing a compound of the invention can be prepared by conventional techniques, e.g. as described in Remington: The Science and Practice of Pharmacy, 19th Ed., 1995, incorporated by reference herein. The compositions can appear in conventional forms, for example capsules, tablets, aerosols, solutions, suspensions or topical applications.

Typical compositions include a compound of the invention and a pharmaceutically acceptable excipient which can be a carrier or a diluent. For example, the active compound will usually be mixed with a carrier, or diluted by a carrier, or enclosed within a carrier which can be in the form of an ampoule, capsule, sachet, paper, or other container. When the active compound is mixed with a carrier, or when the carrier serves as a diluent, it can be solid, semi-solid, or liquid material that acts as a vehicle, excipient, or medium for the active compound. The active compound can be adsorbed on a granular solid carrier, for example contained in a sachet. Some examples of suitable carriers are water, salt solutions, alcohols, polyethylene glycols, polyhydroxyethoxylated castor oil, peanut oil, olive oil, gelatin, lactose, terra alba, sucrose, dextrin, magnesium carbonate, sugar, cyclodextrin, amylose, magnesium stearate, talc, gelatin, agar, pectin, acacia, stearic acid or lower alkyl ethers of cellulose, silicic acid, fatty acids, fatty acid amines, fatty acid monoglycerides and diglycerides, pentaerythritol fatty acid esters, polyoxyethylene, hydroxymethylcellulose and polyvinylpyrrolidone. Similarly, the carrier or diluent can include any sustained release material known in the art, such as glyceryl monostearate or glyceryl distearate, alone or mixed with a wax.

The formulations can be mixed with auxiliary agents which do not deleteriously react with the active compounds. Such additives can include wetting agents, emulsifying and suspending agents, salt for influencing osmotic pressure, buffers and/or coloring substances preserving agents, sweetening agents or flavoring agents. The compositions can also be sterilized if desired.

The route of administration can be any route which effectively transports the active compound of the invention to the appropriate or desired site of action, such as oral, nasal, pulmonary, buccal, subdermal, intradermal, transdermal or parenteral, e.g., rectal, depot, subcutaneous, intravenous, intraurethral, intramuscular, intranasal, ophthalmic solution or an ointment, the oral route being preferred.

If a solid carrier is used for oral administration, the preparation can be tabletted, placed in a hard gelatin capsule in powder or pellet form or it can be in the form of a troche or lozenge. If a liquid carrier is used, the preparation can be in the form of a syrup, emulsion, soft gelatin capsule or sterile injectable liquid such as an aqueous or non-aqueous liquid suspension or solution.

Injectable dosage forms generally include aqueous suspensions or oil suspensions which can be prepared using a suitable dispersant or wetting agent and a suspending agent Injectable forms can be in solution phase or in the form of a suspension, which is prepared with a solvent or diluent. Acceptable solvents or vehicles include sterilized water, Ringer's solution, or an isotonic aqueous saline solution. Alternatively, sterile oils can be employed as solvents or suspending agents. Preferably, the oil or fatty acid is non-volatile, including natural or synthetic oils, fatty acids, mono-, di- or tri-glycerides.

For injection, the formulation can also be a powder suitable for reconstitution with an appropriate solution as described above. Examples of these include, but are not limited to, freeze dried, rotary dried or spray dried powders, amorphous powders, granules, precipitates, or particulates. For injection, the formulations can optionally contain stabilizers, pH modifiers, surfactants, bioavailability modifiers and combinations of these. The compounds can be formulated for parenteral administration by injection such as by bolus injection or continuous infusion. A unit dosage form for injection can be in ampoules or in multi-dose containers.

The formulations of the invention can be designed to provide quick, sustained, or delayed release of the active ingredient after administration to the patient by employing procedures well known in the art. Thus, the formulations can also be formulated for controlled release or for slow release.

Compositions contemplated by the present invention can include, for example, micelles or liposomes, or some other encapsulated form, or can be administered in an extended release form to provide a prolonged storage and/or delivery effect. Therefore, the formulations can be compressed into pellets or cylinders and implanted intramuscularly or subcutaneously as depot injections. Such implants can employ known inert materials such as silicones and biodegradable polymers, e.g., polylactide-polyglycolide. Examples of other biodegradable polymers include poly(orthoesters) and poly(anhydrides).

For nasal administration, the preparation can contain a compound of the invention, dissolved or suspended in a liquid carrier, preferably an aqueous carrier, for aerosol application. The carrier can contain additives such as solubilizing agents, e.g., propylene glycol, surfactants, absorption enhancers such as lecithin (phosphatidylcholine) or cyclodextrin, or preservatives such as parabens.

For parenteral application, particularly suitable are injectable solutions or suspensions, preferably aqueous solutions with the active compound dissolved in polyhydroxylated castor oil.

Tablets, dragees, or capsules having talc and/or a carbohydrate carrier or binder or the like are particularly suitable for oral application. Preferable carriers for tablets, dragees, or capsules include lactose, corn starch, and/or potato starch. A syrup or elixir can be used in cases where a sweetened vehicle can be employed.

A typical tablet that can be prepared by conventional tabletting techniques can contain:

| Core: | | |
|---|---|---|
| Active compound (as free compound or salt thereof) | 250 | mg |
| Colloidal silicon dioxide (Aerosil) ® | 1.5 | mg |
| Cellulose, microcryst. (Avicel) ® | 70 | mg |
| Modified cellulose gum (Ac-Di-Sol) ® | 7.5 | mg |
| Magnesium stearate | Ad. | |
| Coating: | | |
| HPMC approx. | 9 | mg |
| *Mywacett 9-40 T approx. | 0.9 | mg |

*Acylated monoglyceride used as plasticizer for film coating.

A typical capsule for oral administration contains compounds of the invention (250 mg), lactose (75 mg) and magnesium stearate (15 mg). The mixture is passed through a 60 mesh sieve and packed into a No. 1 gelatin capsule. A typical injectable preparation is produced by aseptically placing 250 mg of compounds of the invention into a vial, aseptically freeze-drying and sealing. For use, the contents of the vial are mixed with 2 mL of sterile physiological saline, to produce an injectable preparation.

The compounds of the invention can be administered to a mammal, especially a human in need of such treatment, prevention, elimination, alleviation or amelioration of a malcondition. Such mammals include also animals, both domestic animals, e.g. household pets, farm animals, and non-domestic animals such as wildlife.

The compounds of the invention are effective over a wide dosage range. For example, in the treatment of adult humans, dosages from about 0.05 to about 5000 mg, preferably from about 1 to about 2000 mg, and more preferably between about 2 and about 2000 mg per day can be used. A typical dosage is about 10 mg to about 1000 mg per day. In choosing a regimen for patients it can frequently be necessary to begin with a higher dosage and when the condition is under control to reduce the dosage. The exact dosage will depend upon the activity of the compound, mode of administration, on the therapy desired, form in which administered, the subject to be treated and the body weight of the subject to be treated, and the preference and experience of the physician or veterinarian in charge.

Generally, the compounds of the invention are dispensed in unit dosage form including from about 0.05 mg to about 1000 mg of active ingredient together with a pharmaceutically acceptable carrier per unit dosage.

Usually, dosage forms suitable for oral, nasal, pulmonal or transdermal administration include from about 125 μg to about 1250 mg, preferably from about 250 μg to about 500 mg, and more preferably from about 2.5 mg to about 250 mg, of the compounds admixed with a pharmaceutically acceptable carrier or diluent.

Dosage forms can be administered daily, or more than once a day, such as twice or thrice daily. Alternatively dosage forms can be administered less frequently than daily, such as every other day, or weekly, if found to be advisable by a prescribing physician. The compounds of the invention may be administered in the form of a pharmaceutical composition, in combination with a pharmaceutically acceptable carrier. The active ingredient in such formulations may comprise from 0.1 to 99.99 weight percent.

“Pharmaceutically acceptable carrier” means any carrier, diluent or excipient which is compatible with the other ingredients of the formulation and not deleterious to the recipient. The active agent is preferably administered with a pharmaceutically acceptable carrier selected on the basis of the selected route of administration and standard pharmaceutical practice. The active agent may be formulated into dosage forms according to standard practices in the field of pharmaceutical preparations. See Alphonso Gennaro, ed., Remington's Pharmaceutical Sciences, 18th Edition (1990), Mack Publishing Co., Easton, Pa. Suitable dosage forms may comprise, for example, tablets, capsules, solutions, parenteral solutions, troches, suppositories, or suspensions.

The pharmaceutical compositions of the present invention may also be formulated so as to provide slow or controlled release of the active ingredient therein using, for example, hydropropylmethyl cellulose in varying proportions to provide the desired release profile, other polymer matrices, gels, permeable membranes, osmotic systems, multilayer coatings, microparticles, liposomes and/or microspheres.

In general, a controlled-release preparation is a pharmaceutical composition capable of releasing the active ingredient at the required rate to maintain constant pharmacological activity for a desirable period of time. Such dosage forms provide a supply of a drug to the body during a predetermined period of time and thus maintain drug levels in the therapeutic range for longer periods of time than conventional non-controlled formulations. U.S. Pat. No. 5,674,533 discloses controlled-release pharmaceutical compositions in liquid dosage forms for the administration of moguisteine, a potent peripheral antitussive. U.S. Pat. No. 5,059,595 describes the controlled-release of active agents by the use of a gastro-resistant tablet for the therapy of organic mental disturbances. U.S. Pat. No. 5,591,767 describes a liquid reservoir transdermal patch for the controlled administration of ketorolac, a non-steroidal anti-inflammatory agent with potent analgesic properties. U.S. Pat. No. 5,120,548 discloses a controlled-release drug delivery device comprised of swellable polymers. U.S. Pat. No. 5,073,543 describes controlled-release formulations containing a trophic factor entrapped by a ganglioside-liposome vehicle. U.S. Pat. No. 5,639,476 discloses a stable solid controlled-release formulation having a coating derived from an aqueous dispersion of a hydrophobic acrylic polymer. Biodegradable microparticles are known for use in controlled-release formulations. U.S. Pat. No. 5,354,566 discloses a controlled-release powder that contains the active ingredient. U.S. Pat. No. 5,733,566, describes the use of polymeric microparticles that release antiparasitic compositions. The controlled-release of the active ingredient may be stimulated by various inducers, for example pH, temperature, enzymes, water, or other physiological conditions or compounds.

Various mechanisms of drug release exist. For example, in one embodiment, the controlled-release component may swell and form porous openings large enough to release the active ingredient after administration to a patient. The term “controlled-release component” in the context of the present invention is defined herein as a compound or compounds, such as polymers, polymer matrices, gels, permeable membranes, liposomes and/or microspheres, that facilitate the controlled-release of the active ingredient in the pharmaceutical composition. In another embodiment, the controlled-release component is biodegradable, induced by exposure to the aqueous environment, pH, temperature, or enzymes in the body. In another embodiment, sol-gels may be used, wherein the active ingredient is incorporated into a sol-gel matrix that is a solid at room temperature. This matrix is implanted into a patient, preferably a mammal, having a body temperature high enough to induce gel formation of the sol-gel matrix, thereby releasing the active ingredient into the patient.

One or more compounds useful in the practice of the present inventions may be administered simultaneously, by the same or different routes, or at different times during treatment. The compounds may be administered before, along with, or after other medications. The treatment may be carried out for as long a period as necessary, either in a single, uninterrupted session, or in discrete sessions. The treating physician will know how to increase, decrease, or interrupt treatment based on patient response. The treatment schedule may be repeated as required.

### Pharmaceutical Combinations

In various embodiments, a pharmaceutical combination comprising a compound of the invention in a therapeutically effective dose and a second medicament in a therapeutically effective dose is provided. More specifically, the second medicament can comprise an anti-proliferative agent, an anti-glaucoma agent, an anti-hypertensive agent, an anti-atherosclerotic agent, an anti-multiple sclerosis agent, an anti-angina agent, an anti-erectile dysfunction agent, an anti-stroke agent, or an anti-asthma agent. For example, the anti-proliferative agent can comprise an alkylating agent, an anti-metabolite, a vinca alkaloid, a terpenoid, a topoisomerase inhibitor, a monoclonal antibody, a kinase inhibitor, carboplatin, cisplatin, taxol, leucovorin, 5-fluorouracil, eloxatin, cyclophosphamide, chlorambucil, avastin, or imatinib mesylate. For example, the anti-glaucoma agent can comprise a beta receptor-blocker, a prostaglandin, an alpha-adrenergic agonist, a parasympathomimetic (cholinergic agonist), or a carbonic anhydrase inhibitor. For example, the anti-hypertensive agent can comprise a beta receptor-blocker, a calcium channel blocker, a diueretic, an angiotensin converting enzyme (ACE) inhibitor, a renin inhibitor, or an angiotensin receptor antagonist. For example, the anti-atherosclerotic agent can comprise a 3-HMG-coA-reductase inhibitor, a statin, atorvastatin, simvastatin, niacin, or a combination drug such as vytorin. For example, the anti-multiple sclerosis agent can comprise beta-inteferon, tysabri, or glatirimar acetate. For example, the anti-angina agent can comprise a beta receptor-blocker, a calcium channel blocker, nitroglycerin, isosorbide mononitrate, nicorandil, or ranolanzine. For example, the anti-erectile dysfunction agent can comprise a phosphodiesterase-5 inhibitor. For example, the anti-stroke agent can comprise tissue plasminogen activator. For example, the anti-asthma agent can comprise a bronchodilator, an inhaled corticosteroid, a leukotrine blockers, cromolyn, nedocromil, or theophylline.

In various embodiments, a pharmaceutical combination of the invention can further comprise a suitable excipient as outlined above to provide a pharmaceutical composition comprising both medicaments.

In various embodiments, a method of treatment of a malcondition is provided comprising administering an effective amount of a compound of the invention and co-administering an effective amount of an additional medicament. The malcondition can comprise cardiovascular disease, neurogenic pain, hypertension, atherosclerosis, angina, stroke, arterial obstruction, peripheral arterial disease, peripheral circulation disorder, erectile dysfunction, acute and chronic pain, dementia, Alzheimer's disease, Parkinson's disease, neuronal degeneration, asthma, amyotrophic lateral sclerosis, spinal cord injury, rheumatoid arthritis, osteoarthritis, osteoporosis, psoriasis, cerebral vasospasm, glaucoma, multiple sclerosis, pulmonary hypertension, acute respiratory distress syndrome, inflammation, diabetes, urinary organ diseases such as overactive bladder (OAB) and benign prostatic hypertrophy (BPH), metastasis, cancer, glaucoma, ocular hypertension, retinopathy, autoimmune disease and viral infection, or myocardial pathology, or any combination thereof.

In various embodiments, the additional medicament that can be co-administered can comprise an anti-proliferative agent, an anti-glaucoma agent, an anti-hypertensive agent, an anti-atherosclerotic agent, an anti-multiple sclerosis agent, an anti-angina agent, an anti-erectile dysfunction agent, an anti-stroke agent, or an anti-asthma agent. By “co-administered” is meant that the patient is provided with an effective dose of an inventive compound and with an effective dose of the second medicament during the course of treatment, such as concurrently, consecutively, intermittently, or in other regimens. The compound of the invention and the second medicament can be administered in separate dosage forms. For example, the anti-proliferative agent can comprise an alkylating agent, an anti-metabolite, a vinca alkaloid, a terpenoid, a topoisomerase inhibitor, a monoclonal antibody, a kinase inhibitor, carboplatin, cisplatin, taxol, leucovorin, 5-fluorouracil, eloxatin, cyclophosphamide, chlorambucil, avastin, or imatinib mesylate. For example, the anti-glaucoma agent can comprise a beta receptor-blocker, a prostaglandin, an alpha-adrenergic agonist, a parasympathomimetic (cholinergic agonist), or a carbonic anhydrase inhibitor. For example, the anti-hypertensive agent can comprise a beta receptor-blocker, a calcium channel blocker, a diueretic, an angiotensin converting enzyme (ACE) inhibitor, a renin inhibitor, or an angiotensin receptor antagonist. For example, the anti-atherosclerotic agent can comprise a 3-HMG-coA-reductase inhibitor, a statin, atorvastatin, simvastatin, niacin, or a combination drug such as vytorin. For example, the anti-multiple sclerosis agent can comprise beta-inteferon, tysaberai, or glatirimar acetate. For example, the anti-angina agent can comprise a beta receptor-blocker, a calcium channel blocker, nitroglycerin, isosorbide mononitrate, nicorandil, or ranolanzine. For example, the anti-erectile dysfunction agent can comprise a phosphodiesterase-5 inhibitor. For example, the anti-stroke agent can comprise tissue plasminogen activator. For example, the anti-asthma agent can comprise a bronchodilator, an inhaled corticosteroid, a leukotrine blockers, cromolyn, nedocromil, or theophylline.

### EXAMPLES

Unless specifically stated otherwise, the experimental procedures were performed under the following conditions. All operations were carried out at room or ambient temperature—that is, at a temperature in the range of 18-25° C. Evaporation of solvent was carried out using a rotary evaporator under reduced pressure (600-4000 pascals: 4.5-30 mm. Hg) with a bath temperature of up to 60° C. The course of reactions was followed by thin layer chromatography (TLC) and reaction times are given for illustration only. The structure and purity of all final products were assured by at least one of the following techniques: TLC, mass spectrometry, nuclear magnetic resonance (NMR) spectrometry or HPLC analysis. When given, yields are for illustration only. When given, NMR data is in the form of delta (δ) values for major diagnostic protons, given in parts per million (ppm) relative to tetramethylsilane (TMS) as internal standard, determined at 400 MHz using the indicated solvent. Conventional abbreviations used for signal shape are: s. singlet; d. doublet; t. triplet; m. multiplet; br. broad; etc. Chemical symbols have their usual meanings; the following abbreviations are used: v (volume), w (weight), b.p. (boiling point), m.p. (melting point), L (liter(s)), mL (milliliters), g (gram(s)), mg (milligrams(s)), mol (moles), mmol (millimoles), eq (equivalent(s)).

### General Procedures:

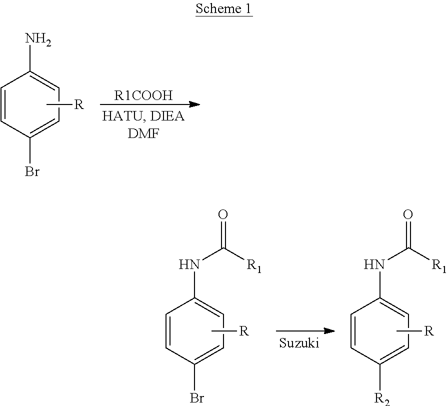

A substituted or unsubstituted 4-bromoaniline (1 equiv) was added to a stirred solution of an acid R₁COOH (1.2 equiv), HATU (1.3 equiv), and DIEA (3 equiv) in DMF at 23° C. After the amide coupling was finished (monitored by TLC or LC-MS or LC), the DMF was removed under reduced pressure. The residue was suspended in EtOAc, washed by brine, saturated NaHCO₃ (2×), brine (2×), 1N HCl (2×), and brine (2×) again, dried over Na₂SO₄.

The solvents were then evaporated in a Rotovapor to give the crude amide, which was then used directly in the next step without further purification. Standard Suzuki coupling conditions were utilized to prepare the final product. Thus, Pd[P(Ph)₃]₄ (15%) was added to a degassed (with argon) solution of the bromophenyl amide (1 equiv) obtained above, a boronic acid or ester (1.5 equiv), K₂CO₃ (4 equiv) in dioxane/H₂O (4:1 by volume). The resulting suspension was sealed in Microwave reactor (from Biotage).

This solution was then either subjected to microwave conditions (90° C., 2 h) or thermal conditions (95° C., 10 h) to do the Suzuki couplings. After the coupling was complete (monitored by LC-MS), the solvents were removed via a Rotovapor, and the resulting residue was directly subjected to preparative HPLC to give the final product as a TFA salt (after drying).

### Example 1

### Synthesis of N-(4-(1H-pyrazol-4-yl)phenyl)-2,3-dihydrobenzo[b][1,4]dioxine-2-carboxamide

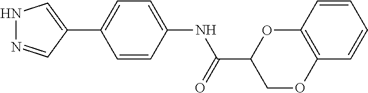

Procedures in Scheme 1 were used to prepare the title compound. ¹H NMR (DMSO, 400 MHz) δ 4.30-4.40 (dd, J=5.6, 12.0 Hz, 1H), 4.47 (dd, J=2.8, 12.0 Hz, 1H), 4.96 (dd, J=2.4, 5.6 Hz, 1H), 6.83-6.92 (m, 3H), 7.02-7.06 (m, 1H), 7.56 (d, J=8.8 Hz, 2H), 7.62 (dd, J=2.0, 6.8 Hz, 2H), 8.01 (m, 2H), 10.10 (s, 1H), 13.0 (b, 1H); Single peak in analytical HPLC, LC-MS: C₁₈H₁₅N₃O₃(M⁺+1) 322.

### Example 2

### Synthesis of N-(4-(pyridin-4-yl)phenyl)-2,3-dihydrobenzo[b][1,4]dioxine-2-carboxamide

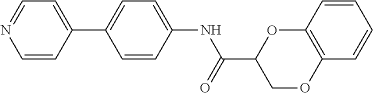

Procedures in Scheme 1 were used to prepare the title compound. ¹H NMR (DMSO, 400 MHz) δ 4.35-4.42 (dd, J=5.6, 12.0 Hz, 1H), 4.47 (dd, J=2.8, 12.0 Hz, 1H), 5.04 (dd, J=3.2, 5.6 Hz, 1H), 6.84-6.93 (m, 3H), 7.04-7.07 (m, 1H), 7.86 (d, J=8.8 Hz, 2H), 7.99 (d, J=8.8 Hz, 2H), 8.12 (d, J=6.8 Hz, 2H), 8.80 (d, J=6.8 Hz, 2H), 10.4 (s, 1H); Single peak in analytical HPLC; LC-MS: C₂₀H₁₆N₂O₃(M⁺+1) 333.

### Example 3

### Synthesis of N-(4-(pyridin-4-yl)phen yl)-2,3-dihydronaphtho[2,3-b][1,4]dioxine-2-carboxamide

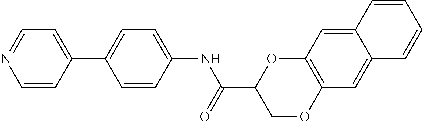

Procedures in Scheme 1 were used to prepare the title compound. Single peak in analytical HPLC; LC-MS: C₂₄H₁₈N₂O₃(M⁺+1) 383.

### Example 4

### Synthesis of a mixture of N-(4-(pyridin-4-yl)phenyl)-2,3-dihydronaphtho[2,1-b][1,4]dioxine-2-carboxamide and N-(4-(pyridin-4-yl)phenyl)-2,3-dihydronaphtho[1,2-b][1,4]dioxine-3-carboxamide

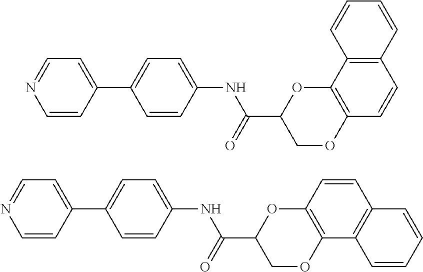

Procedures in Scheme 1 were used to prepare the title compound. Single peak in analytical HPLC; LC-MS: C₁₈H₁₅N₃O₃ (M⁺+1) 322.

### Example 5

### Synthesis of N-(4-(pyridin-4-yl)phenyl)-1,2-dihydronaphtho[2,1-b]furan-2-carboxamide

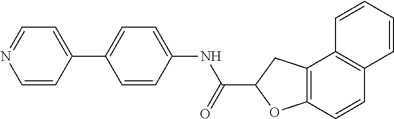

Procedures in Scheme 1 were used to prepare the title compound. Single peak in analytical HPLC; LC-MS: C₂₄H₁₈N₂O₂(M⁺+1) 367.

### Example 6

### Synthesis of N-(4-(pyridin-4-yl)phenyl)-2,3-dihydronaphtho[1,2-b]furan-2-carboxamide

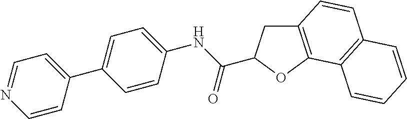

Procedures in Scheme 1 were used to prepare the title compound. Single peak in analytical HPLC; LC-MS: C₂₄H₁₈N₂O₂(M⁺+1) 367.

### Example 7

### Synthesis of (R)—N-(4-(pyridin-4-yl)phenyl)-2,3-dihydrobenzo[b][1,4]dioxine-2-carboxamide

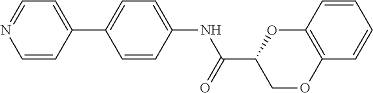

Procedures in Scheme 1 were used to prepare the title compound. ¹H NMR (DMSO, 400 MHz) δ 4.35-4.50 (m, 2H), 5.05 (dd, J=2.8, 5.2 Hz, 1H), 6.84-6.93 (m, 3H), 7.03-7.07 (m, 1H), 7.86-7.89 (dd, J=2.0, 6.8 Hz, 2H), 8.03 (d, J=8.4 Hz, 2H), 8.23 (d, J=6.8 Hz, 2H), 8.84 (dd, J=1.6, 5.2 Hz, 2H), 10.5 (s, 1H); Single peak in analytical HPLC; LC-MS: C₂₀H₁₆N₂O₃ (M⁺+1) 333.

### Example 8

### Synthesis of (R)—N-(4-(1H-pyrazol-4-yl)phenyl)-2,3-dihydrobenzo[b][1,4]dioxine-2-carboxamide

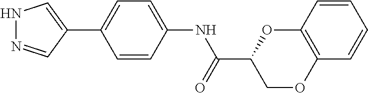

Procedures in Scheme 1 were used to prepare the title compound. ¹H NMR (DMSO, 400 MHz) δ 4.33-4.38 (dd, J=6.0, 12.0 Hz, 1H), 4.47 (dd, J=2.4, 12.0 Hz, 1H), 4.97 (dd, J=2.8, 6.0 Hz, 1H), 6.84-6.93 (m, 3H), 7.04-7.07 (m, 1H), 7.56-7.59 (dd, J=2.0, 6.8 Hz, 2H), 7.62-7.65 (dd, J=2.0, 6.8 Hz, 2H), 8.02 (s, 2H), 10.11 (s, 1H); Single peak in analytical HPLC; LC-MS: C₁₈H₁₅N₃O₃(M⁺+1) 322.

### Example 9

### Synthesis of (R)—N-(4-(3,5-dimethyl-1H-pyrazol-4-yl)phenyl)-2,3-dihydrobenzo[b][1,4]dioxine-2-carboxamide

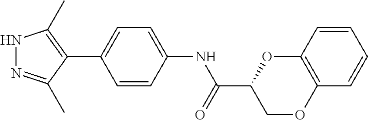

Procedures in Scheme 1 were used to prepare the title compound. Single peak in analytical HPLC; LC-MS: C₂₀H₁₉N₃O₃(M⁺+1) 350.

### Example 10

### Synthesis of (R)—N-(4-(2-methylpyridin-4-yl)phenyl)-2,3-dihydrobenzo[b][1,4]dioxine-2-carboxamide

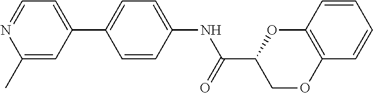

Procedures in Scheme 1 were used to prepare the title compound. Single peak in analytical HPLC; LC-MS: C₂₁H₁₈N₂O₃(M⁺+1) 347.

### Example 11

### Synthesis of (R)—N-(4-(3-fluoropyridin-4-yl)phenyl)-2,3-dihydrobenzo[b][1,4]dioxine-2-carboxamide

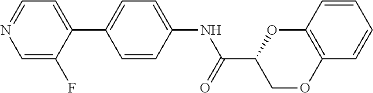

Procedures in Scheme 1 were used to prepare the title compound. Single peak in analytical HPLC; LC-MS: C₂₀H₁₅FN₂O₃(M⁺+1) 351.

### Example 12

### Synthesis of (R)—N-(4-(3-chloropyridin-4-yl)phenyl)-2,3-dihydrobenzo[b][1,4]dioxine-2-carboxamide

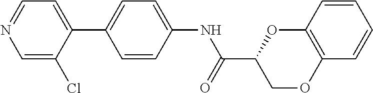

Procedures in Scheme 1 were used to prepare the title compound. ¹H NMR (DMSO, 400 MHz) δ 4.35-4.40 (dd, J=6.0, 12.0 Hz, 1H), 4.47 (dd, J=2.4, 12.0 Hz, 1H), 5.03 (dd, J=2.8, 5.6 Hz, 1H), 6.86-6.91 (m, 3H), 7.03-7.07 (m, 1H), 7.47-7.59 (m, 3H), 7.60-7.70 (m, 2H), 7.78-7.85 (m, 2H), 8.56 (m, 1H), 10.11 (s, 1H); Single peak in analytical HPLC; LC-MS: C₂₀H₁₅ClN₂O₃(M⁺+1) 367.

### Example 13

### Synthesis of (R)—N-(4-(2-(4-methylpiperazin-1-yl)pyridin-4-yl)phenyl)-2,3-dihydrobenzo[b][1,4]dioxine-2-carboxamide

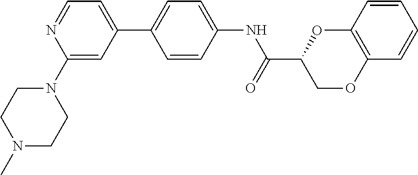

Procedures in Scheme 1 were used to prepare the title compound. Single peak in analytical HPLC; LC-MS: C₂₅H₂₆N₄O₃(M⁺+1) 431.

### Example 14

### Synthesis of (R)—N-(2-fluoro-4-(pyridin-4-yl)phenyl)-2,3-dihydrobenzo[b][1,4]dioxine-2-carboxamide

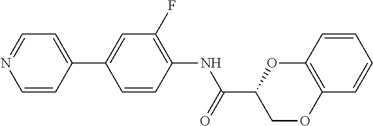

Procedures in Scheme 1 were used to prepare the title compound. ¹H NMR (DMSO, 400 MHz) δ 4.39 (dd, J=5.6, 12.0 Hz, 1H), 4.46 (dd, J=2.8, 11.6 Hz, 1H), 5.16 (dd, J=2.8, 5.2 Hz, 1H), 6.84-6.92 (m, 3H), 7.01-7.04 (m, 1H), 7.83 (dd, J=2.0, 8.4 Hz, 1H), 7.98 (dd, J=2.0, 10.0 Hz, 1H), 8.06 (t, J=8.0 Hz, 1H), 8.12 (dd, J=1.6, 5.2 Hz, 2H), 8.82 (dd, J=1.2, 4.8 Hz, 2H), 10.18 (s, 1H); Single peak in analytical HPLC; LC-MS: C₂₀H₁₅FN₂O₃(M⁺+1) 351.

### Example 15

### Synthesis of (R)—N-(2-fluoro-4-(1H-pyrazol-4-yl)phenyl)-2,3-dihydrobenzo[b][1,4]dioxine-2-carboxamide

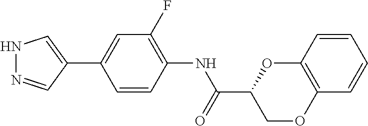

Procedures in Scheme 1 were used to prepare the title compound. ¹H NMR (DMSO, 400 MHz) δ 4.34 (dd, J=4.8, 11.6 Hz, 1H), 4.45 (dd, J=2.8, 11.6 Hz, 1H), 5.06 (dd, J=3.2, 6.0 Hz, 1H), 6.84-6.92 (m, 3H), 7.01-7.04 (m, 1H), 7.43 (dd, J=2.0, 8.4 Hz, 1H), 7.52-7.63 (m, 1H), 7.69 (t, J=8.0 Hz, 1H), 8.10 (b, 2H), 9.87 (s, 1H); Single peak in analytical HPLC; LC-MS: C₁₈H₁₄FN₃O₃ (M⁺+1) 340.

### Example 16

### Synthesis of (R)—N-(3-fluoro-4-(pyridin-4-yl)phenyl)-2,3-dihydrobenzo[b][1,4]dioxine-2-carboxamide

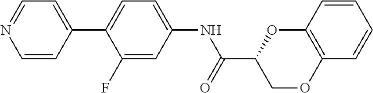

Procedures in Scheme 1 were used to prepare the title compound. Single peak in analytical HPLC; LC-MS: C₂₀H₁₅FN₂O₃(M⁺+1) 351.

### Example 17

### Synthesis of (R)—N-(3-fluoro-4-(2-(4-methylpiperazin-1-yl)pyridin-4-yl)phenyl)-2,3-dihydrobenzo[b][1,4]dioxine-2-carboxamide

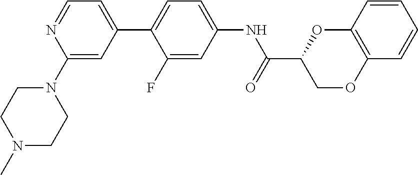

Procedures in Scheme 1 were used to prepare the title compound. Single peak in analytical HPLC; LC-MS: C₂₅H₂₅FN₄O₃(M⁺+1) 449.

### Example 18

### Synthesis of (R)—N-(3-fluoro-4-(1H-pyrazol-4-yl)phenyl)-2,3-dihydrobenzo[b][1,4]dioxine-2-carboxamide

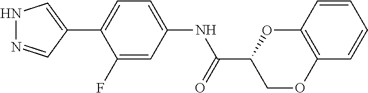

Procedures in Scheme 1 were used to prepare the title compound. ¹H NMR (DMSO, 400 MHz) δ 4.36 (dd, J=6.0, 11.6 Hz, 1H), 4.46 (dd, J=2.8, 11.6 Hz, 1H), 4.99 (dd, J=2.8, 5.6 Hz, 1H), 6.86-6.93 (m, 3H), 7.03-7.06 (m, 1H), 7.42 (dd, J=2.0, 8.8 Hz, 1H), 7.50-7.73 (m, 4H), 8.00 (b, 1H), 10.31 (b, 1H); Single peak in analytical HPLC; LC-MS: C₁₈H₁₄FN₃O₃ (M⁺+1) 340.

### Example 19

### Synthesis of (R)—N-(3-chloro-4-(pyridin-4-yl)phenyl)-2,3-dihydrobenzo[b][1,4]dioxine-2-carboxamide

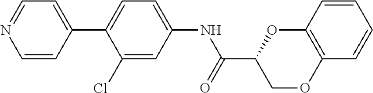

Procedures in Scheme 1 were used to prepare the title compound. Single peak in analytical HPLC; LC-MS: C₂₀H₁₅ClN₂O₃(M⁺+1) 367.

### Example 20

### Synthesis of (R)—N-(3-chloro-4-(1H-pyrazol-4-yl)phenyl)-2,3-dihydrobenzo[b][1,4]dioxine-2-carboxamide

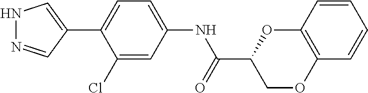

Procedures in Scheme 1 were used to prepare the title compound. Single peak in analytical HPLC; LC-MS: C₁₈H₁₄ClN₃O₃(M⁺+1) 356.

### Example 21

### Synthesis of (2R)—N-(3-chloro-4-(3,5-dimethyl-1H-pyrazol-4-yl)phenyl)-2,3-dihydrobenzo[b][1,4]dioxine-2-carboxamide

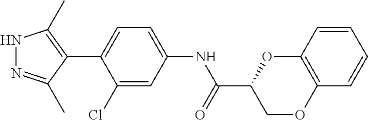

Procedures in Scheme 1 were used to prepare the title compound. Single peak in analytical HPLC; LC-MS: C₂₀H₁₈ClN₃O₃(M⁺+1) 384.

### Example 22

### Synthesis of (R)—N-(4-(2-aminopyrimidin-4-yl)-2-methoxyphenyl)-2,3-dihydrobenzo[b][1,4]dioxine-2-carboxamide

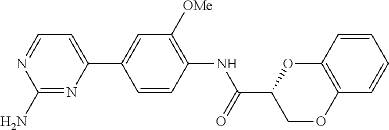

Procedures in Scheme 1 were used to prepare the title compound. Single peak in analytical HPLC; LC-MS: C₂₀H₁₈N₄O₄(M⁺+1) 379.

### Example 23

### Synthesis of (R)—N-(2-methoxy-4-(7H-pyrrolo[2,3-d]pyrimidin-4-yl)phenyl)-2,3-dihydrobenzo[b][1,4]dioxine-2-carboxamide

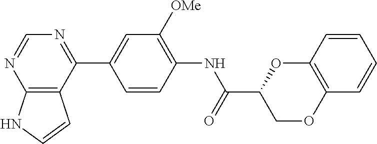

Procedures in Scheme 1 were used to prepare the title compound. Single peak in analytical HPLC; LC-MS: C₂₂H₁₈N₄O₄(M⁺+1) 403.

### Example 24

### Synthesis of (R)—N-(2-methoxy-4-(pyridin-4-yl)phenyl)-2,3-dihydrobenzo[b][1,4]dioxine-2-carboxamide

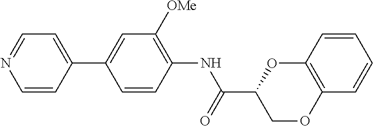

Procedures in Scheme 1 were used to prepare the title compound. ¹H NMR (DMSO, 400 MHz) δ 3.98 (s, 3H), 4.43 (dd, J=5.6, 11.6 Hz, 1H), 5.21 (dd, J=3.2, 4.4 Hz, 1H), 6.88-6.96 (m, 3H), 7.07-7.11 (m, 1H), 7.59 (m, 2H), 8.18 (d, J=6.0 Hz, 2H), 8.28 (d, J=8.8 Hz, 1H), 8.82 (d, J=6.0 Hz, 2H), 9.38 (s, 1H); Single peak in analytical HPLC; LC-MS: C₂₁H₁₈N₂O₄ (M⁺+1) 363.

### Example 25

### Synthesis of N-(3-methyl-4-(pyridin-4-yl)phenyl)-2,3-dihydrobenzo[b][1,4]dioxine-2-carboxamide

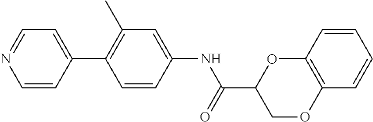

Procedures in Scheme 1 were used to prepare the title compound. ¹H NMR (DMSO, 400 MHz) δ 2.30 (s, 3H), 4.37 (dd, J=5.6, 11.6 Hz, 1H), 4.45 (dd, J=2.8, 11.6 Hz, 1H), 5.01 (dd, J=2.8, 5.6 Hz, 1H), 6.83-6.93 (m, 3H), 7.00-7.06 (m, 1H), 7.31 (d, J=8.0 Hz, 1H), 7.63 (m, 2H), 7.70 (d, J=6.0 Hz, 2H), 8.77 (d, J=7.6 Hz, 2H), 10.26 (s, 1H); Single peak in analytical HPLC; LC-MS: C₂₁H₁₈N₂O₃(M⁺+1) 347.

### Example 26

### Synthesis of N-(4-(6-amino-2-methylpyrimidin-4-yl)-3-methylphenyl)-2,3-dihydrobenzo[b][1,4]dioxine-2-carboxamide

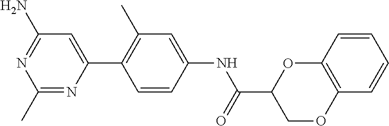

Procedures in Scheme 1 were used to prepare the title compound. Single peak in analytical HPLC; LC-MS: C₂₁H₂₀N₄O₃ (M⁺+1) 377.

### Example 27

### Synthesis of N-(3-methyl-4-(1,8-naphthyridin-3-yl)phenyl)-2,3-dihydrobenzo[b][1,4]dioxine-2-carboxamide

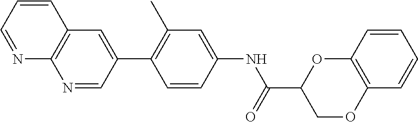

Procedures in Scheme 1 were used to prepare the title compound. Single peak in analytical HPLC; LC-MS: C₂₄H₁₉N₃O₃(M⁺+1) 398.

### Example 28

### Synthesis of N-(4-(3-fluoropyridin-4-yl)-2-methylphenyl)-2,3-dihydrobenzo[b][1,4]dioxine-2-carboxamide

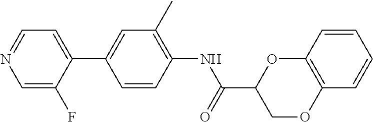

Procedures in Scheme 1 were used to prepare the title compound. Single peak in analytical HPLC; LC-MS: LC-MS: C₂₁H₁₇FN₂O₃(M⁺+1) 365.

### Example 29

### Synthesis of N-(2-methyl-4-(pyridin-4-yl)phenyl)-2,3-dihydrobenzo[b][1,4]dioxine-2-carboxamide

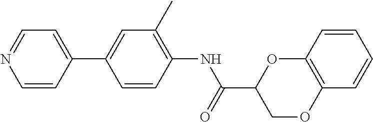

Procedures in Scheme 1 were used to prepare the title compound. ¹H NMR (DMSO, 400 MHz) δ 2.20 (s, 3H), 4.44 (m, 1H), 5.14 (dd, J=3.2, 4.4 Hz, 1H), 6.87-6.94 (m, 3H), 7.07 (dd, J=1.6, 4.8 Hz, 1H), 7.68 (d, J=8.4 Hz, 1H), 7.79 (d, J=2.0 Hz, 1H), 7.85 (d, J=1.6 Hz, 1H), 8.11 (d, J=6.8 Hz, 2H), 8.80 (d, J=5.2 Hz, 2H), 9.55 (s, 1H); Single peak in analytical HPLC; LC-MS: C₂₁H₁₈N₂O₃(M⁺+1) 347.

### Example 30

### Synthesis of N-(2-methyl-4-(1H-pyrazol-4-yl)phenyl)-2,3-dihydrobenzo[b][1,4]dioxine-2-carboxamide

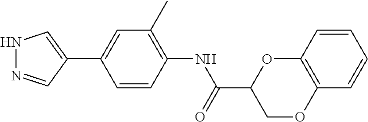

Procedures in Scheme 1 were used to prepare the title compound. Single peak in analytical HPLC; LC-MS: LC-MS: C₁₉H₁₇N₃O₃(M⁺+1) 336.

### Example 31

### Synthesis of N-(2-ethyl-4-(pyridin-4-yl)phenyl)-2,3-dihydrobenzo[b][1,4]dioxine-2-carboxamide

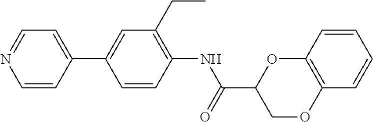

Procedures in Scheme 1 were used to prepare the title compound. ¹H NMR (DMSO, 400 MHz) δ 1.04 (t, J=7.6 Hz, 3H), 2.55 (dd, J=7.6, 11.2 Hz, 2H), 4.40 (dd, J=2.8, 11.6 Hz, 1H), 4.49 (dd, J=4.8, 11.6 Hz, 1H), 5.16 (dd, J=2.8, 4.4 Hz, 1H), 6.87-6.94 (m, 3H), 7.05-7.08 (m, 1H), 7.66 (d, J=8.4 Hz, 1H), 7.80 (d, J=8.0 Hz, 2H), 8.13 (dd, J=1.6, 5.6 Hz, 2H), 8.81 (d, J=5.2 Hz, 2H), 9.62 (s, 1H); Single peak in analytical HPLC; LC-MS: C₂₂H₂₀N₂O₃ (M⁺+1) 361.

### Example 32

### Synthesis of N-(2-ethyl-4-(3-fluoropyridin-4-yl)phenyl)-2,3-dihydrobenzo[b][1,4]dioxine-2-carboxamide

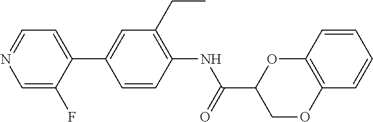

Procedures in Scheme 1 were used to prepare the title compound. Single peak in analytical HPLC; LC-MS: LC-MS: C₂₂H₁₉FN₂O₃(M⁺+1) 379.

### Example 33

### Synthesis of N-(4-(3-cyanopyridin-4-O-2-ethylphenyl)-2,3-dihydrobenzo[b][1,4]dioxine-2-carboxamide

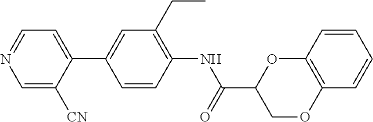

Procedures in Scheme 1 were used to prepare the title compound. Single peak in analytical HPLC; LC-MS: LC-MS: C₂₃H₁₉N₃O₃(M⁺+1) 387.

### Example 34

### Synthesis of N-(2-ethyl-4-(1H-pyrazol-4-yl)phenyl)-2,3-dihydrobenzo[b][1,4]dioxine-2-carboxamide

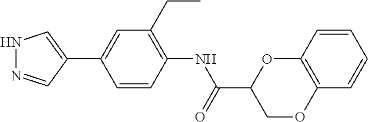

Procedures in Scheme 1 were used to prepare the title compound. Single peak in analytical HPLC; LC-MS: LC-MS: C₂₀H₁₉N₃O₃(M⁺+1) 350.

### Example 35

### Synthesis of N-(4-(pyridin-4-yl)-2-(trifluoromethyl)phenyl)-2,3-dihydrobenzo[b][1,4]dioxine-2-carboxamide

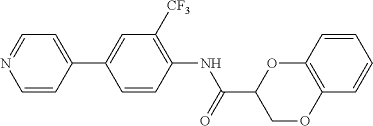

Procedures in Scheme 1 were used to prepare the title compound. Single peak in analytical HPLC; LC-MS: LC-MS: C₂₁H₁₅F₃N₂O₃(M⁺+1) 401.

### Example 36

### Synthesis of N-(4-(1H-pyrazol-4-yl)-2-(trifluoromethyl)phenyl)-2,3-dihydrobenzo[b][1,4]dioxine-2-carboxamide

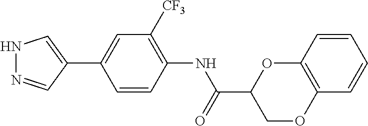

Procedures in Scheme 1 were used to prepare the title compound. ¹H NMR (DMSO, 400 MHz) δ 4.29-4.44 (m, 2H), 5.00-5.11 (m, 1H), 6.85-6.93 (m, 3H), 7.00-7.03 (m, 1H), 7.56-7.63 (m, 1H), 7.91-7.93 (m, 2H), 8.20 (s, 2H), 9.67 (s, 1H), 12.5 (br s, 1H); Single peak in analytical HPLC; LC-MS: C₁₉H₁₄F₃N₃O₃(M⁺+1) 390.

### Example 37

### Synthesis of N-(4-(pyridin-4-yl)-3-(trifluoromethyl)phenyl)-2,3-dihydrobenzo[b][1,4]dioxine-2-carboxamide

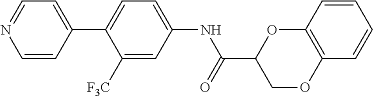

Procedures in Scheme 1 were used to prepare the title compound. Single peak in analytical HPLC; LC-MS: C₂₁H₁₅F₃N₂O₃(M⁺+1) 401.

### Example 38

### Synthesis of N-(4-(1H-pyrazol-4-yl)-3-(trifluoromethyl)phenyl)-2,3-dihydrobenzo[b][1,4]dioxine-2-carboxamide

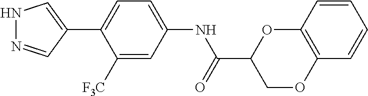

Procedures in Scheme 1 were used to prepare the title compound. Single peak in analytical HPLC; LC-MS: C₁₉H₁₄F₃N₃O₃ (M⁺+1) 390.

### Example 39

### Synthesis of N-(3,4′-bipyridin-6-yl)-2,3-dihydrobenzo[b][1,4]dioxine-2-carboxamide

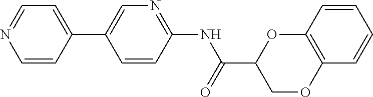

Procedures in Scheme 1 were used to prepare the title compound. Single peak in analytical HPLC; LC-MS: C₁₉H₁₅N₃O₃(M⁺+1) 334.

### Example 40

### Synthesis of N-(5-(1H-pyrazol-4-yl)pyridin-2-yl)-2,3-dihydrobenzo[b][1,4]dioxine-2-carboxamide

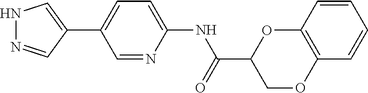

Procedures in Scheme 1 were used to prepare the title compound. ¹H NMR (DMSO, 400 MHz) δ 4.26 (dd, J=2.8, 11.6 Hz, 1H), 4.41 (dd, J=3.6, 11.6 Hz, 1H), 5.03 (dd, J=2.8, 3.2 Hz, 1H), 6.79-6.84 (m, 3H), 6.90-6.92 (m, 1H), 7.30-8.70 (multibroad peaks, 5H), 10.52 (s, 1H), 13.0 (br s, 1H); Single peak in analytical HPLC; LC-MS: C₁₇H₁₄N₄O₃(M⁺+1) 323.

### Example 41

### Synthesis of N-(2,6-dimethyl-4-(pyridin-4-yl)phenyl)-2,3-dihydrobenzo[b][1,4]dioxine-2-carboxamide

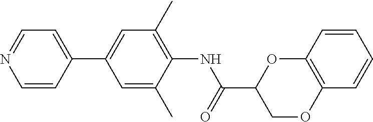

Procedures in Scheme 1 were used to prepare the title compound. ¹H NMR (DMSO, 400 MHz) δ 2.13 (s, 6H), 4.38 (dd, J=2.4, 11.2 Hz, 1H), 4.51 (dd, J=4.4, 11.6 Hz, 1H), 5.15 (dd, J=2.8, 4.8 Hz, 1H), 6.87-6.93 (m, 3H), 7.04-7.07 (m, 1H), 7.67 (s, 2H), 8.11 (d, J=6.8 Hz, 2H), 8.81 (dd, J=1.2, 5.2 Hz, 2H), 9.68 (s, 1H); Single peak in analytical HPLC; LC-MS: C₂₂H₂₀N₂O₃ (M⁺+1) 361.

### Example 42

### Synthesis of N-(2,6-dimethyl-4-(1H-pyrazol-4-yl)phenyl)-2,3-dihydrobenzo[b][1,4]dioxine-2-carboxamide

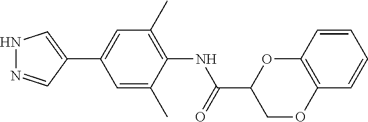

Procedures in Scheme 1 were used to prepare the title compound. Single peak in analytical HPLC; LC-MS: C₂₀H₁₉N₃O₃(M⁺+1) 350.

### Example 43

### Synthesis of chroman-2-carboxylic acid [4-(1H-pyrazol-4-yl)-phenyl]-amide

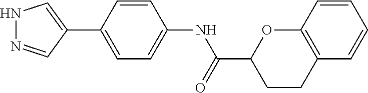

The title compound was prepared according to the procedure described in Scheme 1 (42 mg, 28% over three steps). ¹H NMR (DMSO-d₆, 400 MHz) δ 9.94 (s, 1H), 8.13 (br s, 1H), 7.89 (br s, 1H), 7.66 (d, J=8.4 Hz, 2H), 7.56 (d, J=8.8 Hz, 2H), 7.14-7.09 (m, 2H), 6.93 (dd, J=8.0, 0.8 Hz, 1H), 6.86 (td, J=8.0, 1.2 Hz, 1H), 4.71 (dd, J=8.8, 2.8 Hz, 1H), 2.91-2.83 (m, 1H), 2.75 (dt, J=16.4, 5.2 Hz, 1H), 2.26-2.21 (m, 1H), 2.20-2.09 (m, 1H). LC-MS: single peak at 254 nm, MH⁺ calcd. for C₁₉H₁₈N₃O₂: 320, obtained: 320.

### Example 44

### Synthesis of chroman-3-carboxylic acid [4-(1H-pyrazol-4-yl)-phenyl]-amide

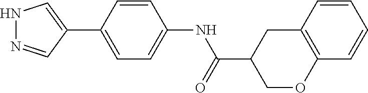

The title compound was prepared from according to the procedure described in Scheme 1 except the crude product was purified by filtration and washing with water, ethanol, and ethyl acetate (52 mg, 37% over three steps). ¹H NMR (DMSO-d₆, 400 MHz) δ 10.18 (br s, 1H), 8.13 (br s, 1H), 7.87 (br s, 1H), 7.59 (d, J=8.8 Hz, 2H), 7.57 (d, J=8.8 Hz, 2H), 7.15 (d, J=7.6, 1.2 Hz, 1H), 7.09 (t, J=7.6 Hz, 1H), 6.86 (td, J=7.6, 1.2 Hz, 1H), 6.79 (dd, J=8.0, 1.2 Hz, 1H), 4.45 (d, J=10.8 Hz, 1H), 3.99 (m, 1H), 3.05-2.95 (m, 3H). LC-MS: single peak at 254 nm, MH⁺ calcd. for C₁₉H₁₈N₃O₂: 320, obtained: 320.

### Example 45

### Synthesis of 2-Methyl-2,3-dihydro-benzo[1,4]dioxine-2-carboxylic acid [4-(1H-pyrazol-4-yl)-phenyl]-amide

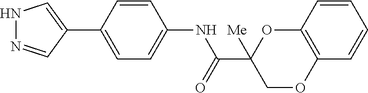

### Step A. 2-Methyl-2,3-dihydro-benzo[1,4]dioxine-2-carboxylic acid

The title compound was prepared according to the procedure of Salimbeni and Manghisi J. Heterocyclic Chem. 1980, 17, 489-493.

### Step B. 2-Methyl-2,3-dihydro-benzo[1,4]dioxine-2-carboxylic acid (4-bromo-phenyl)-amide

The title compound was prepared according to the procedure described in Scheme 1 (10 mg, 18% over three steps). ¹H NMR (DMSO-d₆, 400 MHz) δ 9.75 (s, 1H), 7.99 (br s, 2H), 7.60 (d, J=8.8 Hz, 2H), 7.52 (d, J=8.8 Hz, 2H), 7.09 (dd, J=7.6, 1.2 Hz, 1H), 6.93-6.85 (m, 3H), 4.59 (d, J=11.2 Hz, 1H), 3.99 (d, J=11.2 Hz, 1H), 1.56 (s, 3H). LC-MS: single peak at 254 nm, MH⁺ calcd. for C₁₉H₁₈N₃O₃: 336, obtained: 336.

### Example 46

### Synthesis of chroman-3-carboxylic acid [3-methoxy-4-(1H-pyrazol-4-yl)-phenyl]-amide

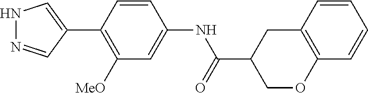

The title compound was prepared according to the procedure described in Scheme 1 (58 mg, 24% over three steps). ¹H NMR (DMSO-d₆, 400 MHz) δ 10.21 (s, 1H), 8.01 (br s, 2H), 7.55 (d, J=8.4 Hz, 1H), 7.49 (d, J=2.0 Hz, 1H), 7.16 (td, J=8.0, 2.0 Hz, 1H), 6.86 (t, J=7.6 Hz, 1H), 6.79 (d, J=8.8 Hz, 1H), 4.45 (d, J=10.4 Hz, 1H), 4.00 (m, 1H), 3.84 (s, 3H), 3.06-2.94 (m, 3H). LC-MS: single peak at 254 nm, MH⁺ calcd. for C₂₀H₂₀N₃O₃: 350, obtained: 350.

### Example 47

### Synthesis of chroman-3-carboxylic acid [2-methoxy-4-(1H-pyrazol-4-yl)-phenyl]-amide

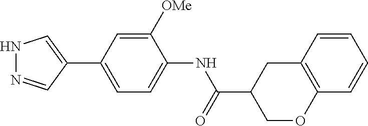

The title compound was prepared according to the procedure described in Scheme 1 (21 mg, 26% over three steps). ¹H NMR (DMSO-d₆, 400 MHz) δ 9.40 (s, 1H), 8.15 (br s, 2H), 7.89 (d, J=8.0 Hz, 1H), 7.26 (d, J=2.0 Hz, 1H), 7.15 (d, J=8.4 Hz, 1H), 7.09 (t, J=8.4 Hz, 1H), 6.85 (t, J=7.6 Hz, 1H), 6.78 (d, J=8.0 Hz, 1H), 4.41 (m, 1H), 3.98 (t, J=10.0 Hz, 1H), 3.90 (s, 3H), 3.05-2.90 (m, 3H). LC-MS: single peak at 254 nm, MH⁺ calcd. for C₂₀H₂₀N₃O₃: 350, obtained: 350.

### Example 48

### Chroman-3-carboxylic acid [2-fluoro-4-(1H-pyrazol-4-yl)-phenyl]-amide

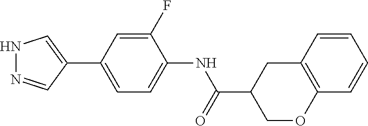

The title compound was prepared according to the procedure described in Scheme 1 (18 mg, 28% over three steps). ¹H NMR (DMSO-d₆, 400 MHz) δ 9.96 (s, 1H), 8.23 (br s, 1H), 7.95 (br s, 1H), 7.81 (t, J=8.4 Hz, 1H), 7.55 (dd, J=12.4, 2.0 Hz, 1H), 7.42 (dd, J=8.4, 1.6 Hz, 1H), 7.15 (d, J=7.6 Hz, 1H), 7.07 (d, J=7.2 Hz, 1H), 6.86 (td, J=7.6, 0.8 Hz, 1H), 6.79 (d, J=8.0 Hz, 1H), 4.44 (ddd, J=11.2, 3.2, 1.6 Hz, 1H), 3.99 (t, J=10.4 Hz, 1H), 3.06-2.93 (m, 3 H). LC-MS: single peak at 254 nm, MH⁺ calcd. for C₁₉H₁₇FN₃O₂: 338, obtained: 338.

### Example 49

### Synthesis of chroman-3-carboxylic acid [3,5-difluoro-4-(1H-pyrazol-4-yl)-phenyl]-amide

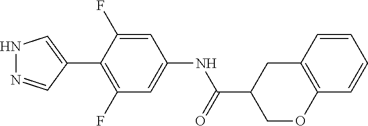

The title compound was prepared according to the procedure described in Scheme 1 (23 mg, 10% over three steps). ¹H NMR (DMSO-d₆, 400 MHz) δ 10.55 (s, 1H), 7.97 (br s, 2H), 7.43 (d, J=10.8 Hz, 2H), 7.15 (dd, J=7.6, 1.2 Hz, 1H), 7.09 (td, J=7.6, 1.6 Hz, 1H), 6.87 (td, J=7.6, 1.2 Hz, 1H), 6.79 (dd, J=8.0, 0.8 Hz, 1H), 4.46 (d, J=11.2 Hz, 1H), 4.02 (m, 1H), 3.06-2.94 (m, 3H). LC-MS: single peak at 254 nm, MH⁺ calcd. for C₁₉H₁₆F₂N₃O₂: 356, obtained: 356.

### Example 50

### Synthesis of chroman-3-carboxylic acid [2-(2-dimethylamino-ethoxy)-4-bromo-phenyl]-amide

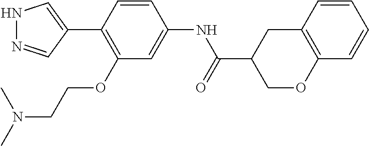

The title compound was prepared according to the procedure described in Scheme 1 (31 mg, 25% over three steps). ¹H NMR (DMSO-d₆, 400 MHz) δ 10.27 (s, 1H), 9.59 (br s, 1H), 8.04 (br s, 2H), 7.66 (d, J=1.6 Hz, 1H), 7.57 (d, J=8.4 Hz, 1H), 7.11-7.07 (m, 3H), 6.87 (td, J=8.0, 1.2 Hz, 1H), 6.79 (d, J=8.0 Hz, 1H), 4.44 (dd, J=8.8, 2.8 Hz, 1H), 4.35 (t, J=4.8 Hz, 1H), 4.02 (t, J=10.0 Hz, 1H), 3.66 (m, 2H), 3.08-2.91 (m, 3H), 2.88 (s, 3H), 2.87 (s, 3H). LC-MS: single peak at 254 nm, MH⁺ calcd. for C₂₃H₂₇N₄O₃: 407, obtained: 407.

### Example 51

### Synthesis of 2H-chromene-3-carboxylic acid [4-(1H-pyrazol-phenyl]-amide

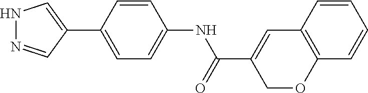

The title compound was prepared according to the procedure described in Scheme 1 except the crude product was purified by filtration and washing with water, ethanol, and ethyl acetate (45 mg, 21% over three steps). ¹H NMR (DMSO-d₆, 400 MHz) δ 10.04 (s, 1H), 8.13 (br s, 1H), 7.89 (br s, 1H), 7.69 (d, J=8.8 Hz, 2H), 7.57 (d, J=8.4 Hz, 2H), 7.47 (s, 1H), 7.30-7.25 (m, 2H), 6.99 (td, J=7.6, 1.2 Hz, 1H), 6.88 (d, J=8.0 Hz, 1H), 4.99 (d, J=1.2 Hz, 2H). LC-MS: single peak at 254 nm, MH⁺ calcd. for C₁₉H₁₆N₃O₂: 318, obtained: 318.

### Example 52

### Synthesis of N-(2-chloro-4-(pyridin-4-yl)phenyl)-2,3-dihydrobenzo[b][1,4]dioxine-2-carboxamide

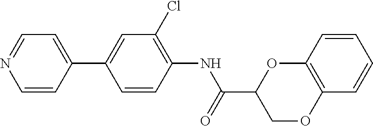

Procedures in Scheme 1 were used to prepare the title compound. Single peak in analytical HPLC; LC-MS: C₂₀H₁₅ClN₂O₃(M⁺+1) 367.

### Example 53

### Synthesis of N-(2-chloro-4-(1H-pyrazol-4-yl)phenyl)-2,3-dihydrobenzo[b][1,4]dioxine-2-carboxamide

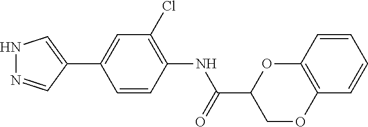

Procedures in Scheme 1 were used to prepare the title compound. Single peak in analytical HPLC; LC-MS: C₁₈H₁₄ClN₃O₃ (M⁺+1) 356.

### Example 54

### Synthesis of N-(4-(pyridin-4-O-2-(trifluoromethoxy)phenyl)-2,3-dihydrobenzo[b][1,4]dioxine-2-carboxamide

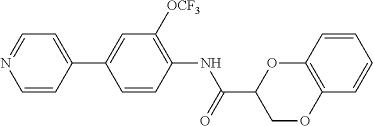

Procedures in Scheme 1 were used to prepare the title compound. Single peak in analytical HPLC; LC-MS: C₂₁H₁₅F₃N₂O₄(M⁺+1) 417.

### Example 55

### Synthesis of N-(4-(1H-pyrazol-4-O-2-(trifluoromethoxy)phenyl)-2,3-dihydrobenzo[b][1,4]dioxine-2-carboxamide

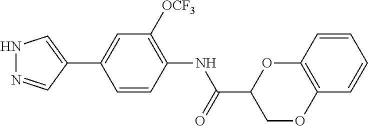

Procedures in Scheme 1 were used to prepare the title compound. ¹H NMR (DMSO, 400 MHz) δ 4.34-4.43 (m, 2H), 5.10 (dd, J=3.2, 4.8 Hz, 1H), 6.84-6.93 (m, 3H), 6.99-7.02 (m, 1H), 7.65 (m, 2H), 7.80 (d, J=8.8 Hz, 1H), 8.14 (s, 2H), 9.77 (s, 1H), 12.5 (br s, 1H); Single peak in analytical HPLC; LC-MS: C₁₉H₁₄F₃N₃O₄(M⁺+1) 406.

### Example 56

### Synthesis of N-(3-methoxy-4-(pyridin-4-yl)phenyl)-2,3-dihydrobenzo[b][1,4]dioxine-2-carboxamide

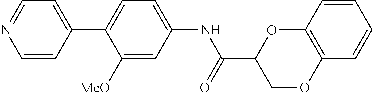

Procedures in Scheme 1 were used to prepare the title compound. Single peak in analytical HPLC; LC-MS: C₂₁H₁₈N₂O₄ (M⁺+1) 363.

### Example 57

### Synthesis of N-(3-methoxy-4-(1H-pyrazol-4-yl)phenyl)-2,3-dihydrobenzo[b][1,4]dioxine-2-carboxamide

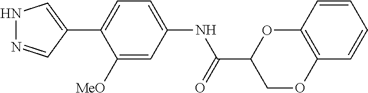

Procedures in Scheme 1 were used to prepare the title compound. Single peak in analytical HPLC; LC-MS: C₁₉H₁₇N₃O₄(M⁺+1) 352.

### Example 58

### Synthesis of N-(3,5-difluoro-4-(pyridin-4-yl)phenyl)-2,3-dihydrobenzo[b][1,4]dioxine-2-carboxamide

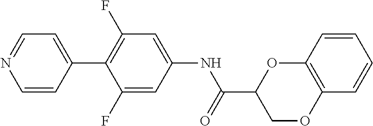

Procedures in Scheme 1 were used to prepare the title compound. ¹H NMR (DMSO, 400 MHz) δ 4.39-4.60 (m, 2H), 5.08 (dd, J=2.8, 4.8 Hz, 1H), 6.84-6.93 (m, 3H), 7.03-7.07 (m, 1H), 7.56-7.66 (m, 4H), 8.76 (dd, J=1.6, 4.8 Hz, 2H), 10.67 (s, 1H); Single peak in analytical HPLC; LC-MS: C₂₀H₁₄F₂N₂O₃(M⁺+1) 369.

### Example 59

### Synthesis of N-(3,5-difluoro-4-(1H-pyrazol-4-yl)phenyl)-2,3-dihydrobenzo[b][1,4]dioxine-2-carboxamide

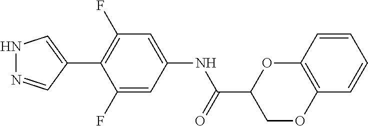

Procedures in Scheme 1 were used to prepare the title compound. Single peak in analytical HPLC; LC-MS: C₁₈H₁₃F₂N₃O₃ (M⁺+1) 358.

### Example 60

### Synthesis of N-(5-fluoro-2-methyl-4-(pyridin-4-yl)phenyl)-2,3-dihydrobenzo[b][1,4]dioxine-2-carboxamide

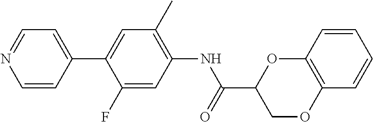

Procedures in Scheme 1 were used to prepare the title compound. Single peak in analytical HPLC; LC-MS: C₂₁H₁₇FN₂O₃(M⁺+1) 365.

### Example 61

### Synthesis of N-(5-fluoro-2-methyl-4-(1H-pyrazol-4-yl)phenyl)-2,3-dihydrobenzo[b][1,4]dioxine-2-carboxamide

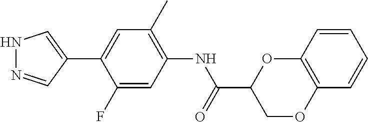

Procedures in Scheme 1 were used to prepare the title compound. ¹H NMR (DMSO, 400 MHz) δ 2.09 (s, 3H), 4.42 (m, 2H), 5.10 (m, 1H), 6.85-6.93 (m, 3H), 7.03-7.06 (m, 1H), 7.37-7.65 (m, 2H), 8.01 (d, J=1.2 Hz, 2H), 9.51 (s, 1H); Single peak in analytical HPLC; LC-MS: C₁₉H₁₆FN₃O₃(M⁺+1) 354.

### Example 62

### Synthesis of N-(6-(pyridin-4-yl)pyridazin-3-yl)-2,3-dihydrobenzo[b][1,4]dioxine-2-carboxamide

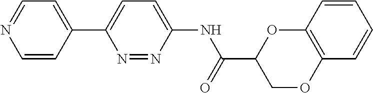

Procedures in Scheme 1 were used to prepare the title compound. Single peak in analytical HPLC; LC-MS: C₁₈H₁₄N₄O₃(M⁺+1) 335.

### Example 63

### Synthesis of N-(6-(1H-pyrazol-4-yl)pyridazin-3-yl)-2,3-dihydrobenzo[b][1,4]dioxine-2-carboxamide

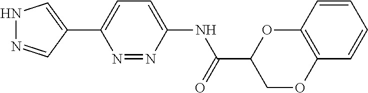

Procedures in Scheme 1 were used to prepare the title compound. ¹H NMR (DMSO, 400 MHz) δ 4.39-4.49 (m, 2H), 5.14 (m, 1H), 6.81-6.93 (m, 3H), 7.00-7.03 (m, 1H), 8.01 (d, J=9.2 Hz, 1H), 8.23 (d, J=9.2 Hz, 1H), 8.29 (s, 2H), 11.26 (s, 1H); Single peak in analytical HPLC; LC-MS: C₁₆H₁₃N₅O₃(M⁺+1) 324.

### Example 64

### Synthesis of N-(2,4′-bipyridin-5-yl)-2,3-dihydrobenzo[b][1,4]dioxine-2-carboxamide

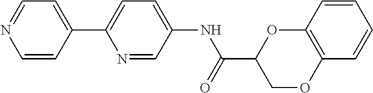

Procedures in Scheme 1 were used to prepare the title compound. Single peak in analytical HPLC; LC-MS: C₁₉H₁₅N₃O₃(M⁺+1) 334.

### Example 65

### Synthesis of N-(6-(1H-pyrazol-4-yl)pyridin-3-yl)-2,3-dihydrobenzo[b][1,4]dioxine-2-carboxamide

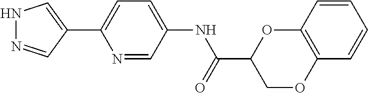

Procedures in Scheme 1 were used to prepare the title compound. ¹H NMR (DMSO, 400 MHz) δ 4.39 (dd, J=2.0, 12.0 Hz, 1H), 4.47 (dd, J=2.8, 11.6 Hz, 1H), 5.05 (dd, J=2.8, 5.6 Hz, 1H), 6.85-6.93 (m, 3H), 7.04-7.07 (m, 1H), 7.83 (d, J=8.8 Hz, 1H), 8.17 (dd, J=2.4, 8.8 Hz, 1H), 8.23 (s, 2H), 8.81 (d, J=2.4 Hz, 1H), 10.51 (s, 1H); Single peak in analytical HPLC; LC-MS: C₁₇H₁₄N₄O₃(M⁺+1) 323.

### Example 66

### Synthesis of N-(2-fluoro-4-(pyridin-4-yl)-5-(trifluoromethyl)phenyl)-2,3-dihydrobenzo[b][1,4]dioxine-2-carboxamide

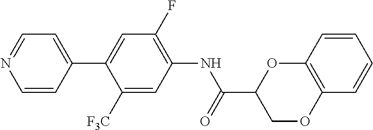

Procedures in Scheme 1 were used to prepare the title compound. ¹H NMR (DMSO, 400 MHz) δ 4.41 (dd, J=4.8, 11.6 Hz, 1H), 4.46 (dd, J=3.2, 11.6 Hz, 1H), 5.17 (dd, J=2.8, 4.8 Hz, 1H), 6.88-6.94 (m, 3H), 7.01-7.04 (m, 1H), 7.55 (m, 3H), 8.40 (d, J=7.2 Hz, 1H), 8.76 (dd, J=1.6, 4.8 Hz, 2H), 10.41 (s, 1H); Single peak in analytical HPLC; LC-MS: C₂₁H₁₄F₂N₂O₃ (M⁺+1) 419.

### Example 67

### Synthesis of N-(2-fluoro-4-(1H-pyrazol-4-yl)-5-(trifluoromethyl)phenyl)-2,3-dihydrobenzo[b][1,4]dioxine-2-carboxamide

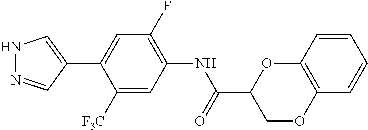

Procedures in Scheme 1 were used to prepare the title compound. Single peak in analytical HPLC; LC-MS: C₁₉H₁₃F₄N₃O₃(M⁺+1) 408.

### Example 68

### Synthesis of N-(2-methoxy-4-(1H-pyrazol-4-yl)phenyl)-2,3-dihydrobenzo[b][1,4]dioxine-2-carboxamide

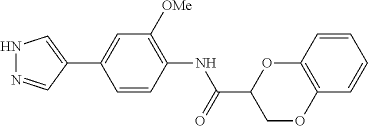

Procedures in Scheme 1 were used to prepare the title compound. ¹H NMR (DMSO, 400 MHz) δ 3.88 (s, 3H), 4.41 (d, J=4.0 Hz, 2H), 5.12 (t, J=4.0 Hz, 1H), 6.86-6.95 (m, 3H), 7.06 (m, 1H), 7.52-7.65 (m, 2H), 8.01 (d, J=8.4 Hz, 1H), 8.09 (b, 2H), 9.15 (s, 1H); Single peak in analytical HPLC; LC-MS: C₁₉H₁₇N₃O₄(M⁺+1) 352.

### Example 69

### Synthesis of (S)—N-(2-methoxy-4-(1H-pyrazol-4-yl)phenyl)-2,3-dihydrobenzo[b][1,4]dioxine-2-carboxamide

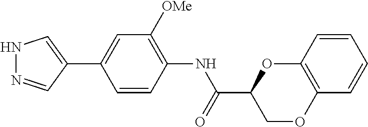

Procedures in Scheme 1 were used to prepare the title compound. Single peak in analytical HPLC; LC-MS: C₁₉H₁₇N₃O₄(M⁺+1) 352.

### Example 70

### Synthesis of (S)—N-(2-methoxy-4-(5-methyl-1H-pyrazol-4-yl)phenyl)-2,3-dihydrobenzo[b][1,4]dioxine-2-carboxamide

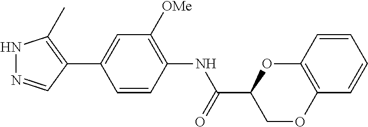

Procedures in Scheme 1 were used to prepare the title compound. Single peak in analytical HPLC; LC-MS: C₂₀H₁₉N₃O₄(M⁺+1) 366.

### Example 71

### Synthesis of (S)—N-(2-fluoro-5-methoxy-4-(1H-pyrazol-4-yl)phenyl)-2,3-dihydrobenzo[b][1,4]dioxine-2-carboxamide

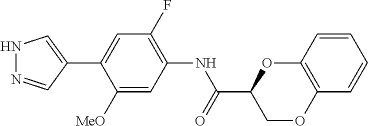

Procedures in Scheme 1 were used to prepare the title compound Single peak in analytical HPLC; LC-MS: C₁₉H₁₆FN₃O₄(M⁺+1) 370.

### Example 72

### Synthesis of N-(2-fluoro-4-(1H-pyrazol-4-yl)phenyl)-2,3-dihydrobenzo[b][1,4]dioxine-2-carboxamide

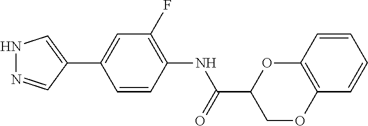

Procedures in Scheme 1 were used to prepare the title compound. ¹H-NMR (DMSO-d₆, 400 MHz), δ: 9.87 (s, 1H), 8.1 (bs, 2H), 7.72 (t, J=8.5 Hz, 1H), 7.63 (m, 1H), 7.43 (dd, J=1.6 Hz, J=8.4 Hz, 1H), 7.02 (m, 1H), 6.90 (m, 3H), 5.07 (dd, J=2.8 Hz, J=5.6 Hz, 1H), 4.45 (dd, J=2.8 Hz, J=11.6 Hz, 1H), 4.38 (dd, J=5.6 Hz, J=11.6 Hz, 1H). Single peak in analytical HPLC; LC-MS: calcd. for C₁₈H₁₄FN₃O₃ (M+H): 340, obsd: 340.

### Example 73

### Synthesis of N-(2,3-dimethyl-4-(pyridin-4-yl)phenyl)-2,3-dihydrobenzo[b][1,4]dioxine-2-carboxamide

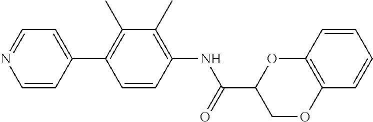

Procedures in Scheme 1 were used to prepare the title compound. Single peak in analytical HPLC; LC-MS: C₂₂H₂₀N₂O₃ (M⁺+1) 361.

### Example 74

### Synthesis of N-(2,3-dimethyl-4-(1H-pyrazol-4-yl)phenyl)-2,3-dihydrobenzo[b][1, 4]dioxine-2-carboxamide

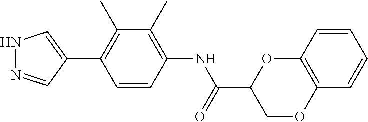

Procedures in Scheme 1 were used to prepare the title compound. Single peak in analytical HPLC; LC-MS: C₂₀H₁₉N₃O₃(M⁺+1) 350.

### Example 75

### Synthesis of N-(2-methyl-4-(pyridin-4-yl)phenyl)-2,3-dihydrobenzo[b][1,4]dioxine-2-carboxamide

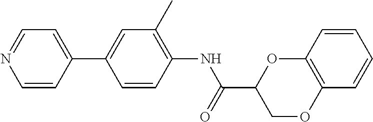

Procedures in Scheme 1 were used to prepare the title compound. Single peak in analytical HPLC; LC-MS: C₂₁H₁₈N₂O₃(M⁺+1) 347.

### Example 76

### Synthesis of N-(2-cyano-4-(pyridin-4-yl)phenyl)-2,3-dihydrobenzo[b][1,4]dioxine-2-carboxamide

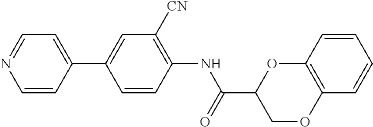

Procedures in Scheme 1 were used to prepare the title compound. Single peak in analytical HPLC; LC-MS: C₂₁H₁₅N₃O₃(M⁺+1) 358.

### Example 77

### Synthesis of 6-chloro-N-(3,5-difluoro-4-(1H-pyrazol-4-yl)phenyl)chroman-3-carboxamide

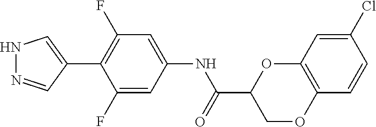

Procedures in Scheme 1 were used to prepare the title compound. ¹H-NMR (DMSO-d₆, 400 MHz), δ: 13.20 (b, 1H), 10.56 (s, 1H), 7.96 (bs, 2H), 7.43 (s, 1H), 7.40 (s, 1H), 7.24 (d, J=2.8 Hz, 1H), 7.12 (dd, J=2.8 Hz, J=12.8 Hz, 1H), 6.81 (d, J=8.4 Hz, 1H), 4.45 (dd, J=2.8 Hz, J=12.0 Hz, 1H), 4.05 (m, 1H), 3.01 (m, 3H). LC-MS: single peak at 254 nm, MH⁺ calcd. for C₁₉H₁₄F₂ClN₃O₂: 390, obtained: 390.

### Example 78

### Synthesis of 6-chloro-N-(3,5-difluoro-4-(3-methyl-1H-pyrazol-4-yl)phenyl)chroman-3-carboxamide

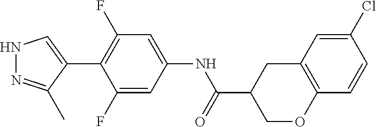

Procedures in Scheme 1 were used to prepare the title compound. LC-MS: single peak at 254 nm, MH⁺ calcd. for C₂₀H₁₆F₂ClN₃O₂: 354, obtained: 354.

### Example 79

### Synthesis of N-(3,5-difluoro-4-(1H-pyrazol-4-yl)phenyl)-7-methoxychroman-3-carboxamide

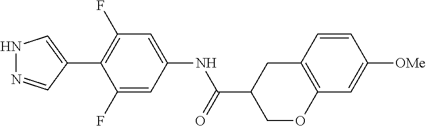

Procedures in Scheme 1 were used to prepare the title compound. ¹H-NMR (DMSO-d₆, 400 MHz), δ: 13.10 (b, 1H), 10.52 (s, 1H), 7.95 (b, 2H), 7.41 (s, 1H), 7.40 (s, 1H), 7.04 (d, J=8.4 Hz, 1H), 6.47 (dd, J=2.4 Hz, J=8.4 Hz, 1H), 6.37 (d, J=2.8 Hz, 1H), 4.43 (m, 1H), 3.98 (t, J=10.0 Hz, 1H), 3.69 (s, 3H), 2.96 (m, 3H). LC-MS: single peak at 254 nm, MH⁺ calcd. for C₂₀H₁₇F₂N₃O₃: 386, obtained: 386.

### Example 80

### Synthesis of 3-(4-(1H-pyrazol-4-yl)phenylcarbamoyl)chroman-6-carboxylic acid

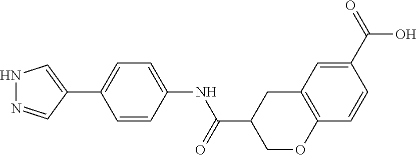

The title compound was prepared from Example 81 by hydrolysis. LC-MS: single peak at 254 nm, MH⁺ calcd. for C₂₀H₁₇N₃O₄: 364, obtained: 364.

### Example 81

### Synthesis of methyl 3-(4-(1H-pyrazol-4-yl)phenylcarbamoyl)chroman-6-carboxylate

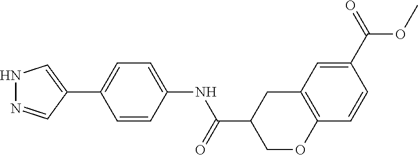

Procedures in Scheme 1 were used to prepare the title compound. LC-MS: single peak at 254 nm, MH⁺ calcd. for C₂₁H₁₉N₃O₄: 378, obtained: 378.

### Example 82

### Synthesis of N3-(4-(1H-pyrazol-4-yl)phenyl)-N6,N6-dimethylchroman-3,6-dicarboxamide

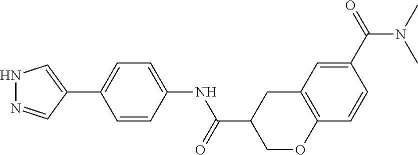

The title compound was prepared from Example 80 by a one-step amide formation. ¹H-NMR (DMSO-d₆, 400 MHz), δ: 10.18 (s, 1H), 8.01 (bs, 2H), 7.59 (d, J=8.8 Hz, 2H), 7.55 (d, J=8.8 Hz, 2H), 7.24 (d, J=2.0 Hz, 1H), 7.16 (dd, J=2.0 Hz, J=10.4 Hz, 1H), 6.82 (d, J=8.4 Hz, 1H), 4.50 (m, 1H), 4.05 (m, 1H), 3.04 (m, 3H), 2.95 (s, 6H). LC-MS: single peak at 254 nm, MH⁺ calcd. for C₂₂H₂₂N₄O₃: 390, obtained: 390.

### Example 83

### Synthesis of N-(2-fluoro-4-(1H-pyrazol-4-yl)phenyl)-2,3-dihydrobenzo[b][1,4]dioxine-2-carboxamide

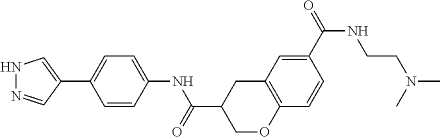

The title compound was prepared from Example 80 by a one-step amide formation. ¹H-NMR (DMSO-d₆, 400 MHz), δ: 10.21 (s, 1H), 9.42 (b, 1H), 8.53 (t, J=8.5 Hz, 1H), 8.00 (s, 2H), 7.70 (d, J=2.0 Hz, 1H), 7.58 (m, 5H), 6.88 (d, J=8.4 Hz, 1H), 4.52 (m, 1H), 4.11 (m, 1H), 3.58 (dd, J=5.6 Hz, J=11.6 Hz, 2H), 3.25 (dd, J=5.6 Hz, J=11.6 Hz, 1H), 3.08 (m, 3H), 2.85 (s, 3H), 2.84 (s, 3H). LC-MS: single peak at 254 nm, MH⁺ calcd. for C₂₄H₂₇N₅O₃: 434, obtained: 434.

### Example 84

### Synthesis of N-(2-fluoro-4-(1H-pyrazol-4-yl)phenyl)-2,3-dihydrobenzo[b][1,4]dioxine-2-carboxamide

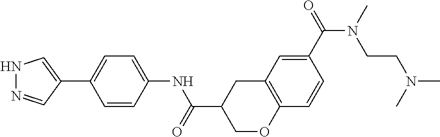

The title compound was prepared from Example 80 by a one-step amide formation. LC-MS: single peak at 254 nm, MH⁺ calcd. for C₂₅H₂₉N₅O₃: 448, obtained: 448.

### Example 85

### Synthesis of N-(4-(1H-pyrazol-4-yl)phenyl)-6-(4-methylpiperazine-1-carbonyl)chroman-3-carboxamide

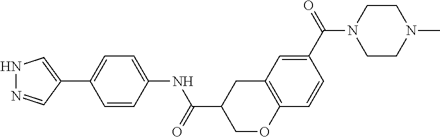

The title compound was prepared from Example 80 by a one-step amide formation. LC-MS: single peak at 254 nm, MH⁺ calcd. for C₂₅H₂₇N₅O₃: 446, obtained: 446.

### Example 86

### Synthesis of N3-(3,5-difluoro-4-(1H-pyrazol-4-yl)phenyl)-N6-(2-(dimethylamino)ethyl)-N6-methylchroman-3,6-dicarboxamide

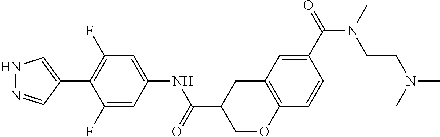

Procedures in Scheme 1 were used to prepare the title compound. LC-MS: single peak at 254 nm, MH⁺ calcd. for C₂₅H₂₈FN₅O₃: 466, obtained: 466.

### Example 87

### Synthesis of (S)—N-(2-(dimethylcarbamoyl)-4-(1H-pyrazol-4-yl)phenyl)-2,3-dihydrobenzo[b][1,4]dioxine-2-carboxamide

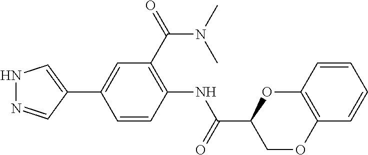

Procedures in Scheme 1 were used to prepare the title compound. ¹H NMR (DMSO, 400 MHz) δ 2.83 (s, 3H), 2.96 (s, 3H), 4.35 (m, 2H), 5.05 (m, 1H), 6.89 (m, 3H), 7.02 (dd, J=1.2 Hz, 8.0 Hz, 1H), 7.57 (d, J=2.0 Hz, 1H), 7.67 (dd, J=2.0 Hz, 8.4 Hz, 1H), 7.89 (d, J=8.8 Hz, 1H), 8.09 (s, 2H), 9.82 (s, 1H), 13.05 (b, 1H); LC-MS: single peak at 254 nm, C₂₁H₂₀N₄O₄ (M⁺+1) 393.19.

### Example 88

### Synthesis of N-(2-(dimethylcarbamoyl)-4-(1H-pyrazol-4-yl)phenyl)-6-methoxychroman-3-carboxamide

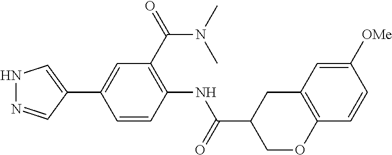

Procedures in Scheme 1 were used to prepare the title compound. ¹H NMR (DMSO, 400 MHz) δ 2.83 (s, 3H), 2.98 (s, 3H), 3.69 (s, 3H), 3.50-3.80 (m, 2H), 3.90 (m, 2H), 4.32 (m, 1H), 6.70 (m, 3H), 7.47 (d, J=8.4 Hz, 1H), 7.51 (d, J=2.4 Hz, 1H), 7.63 (dd, J=2.0 Hz, 8.0 Hz, 1H), 8.09 (s, 2H), 9.77 (s, 1H), 13.05 (b, 1H); LC-MS: single peak at 254 nm, C₂₃H₂₄N₄O₃(M⁺+1) 421.19.

### Example 89

### Synthesis of 8-methoxy-chroman-3-carboxylic acid [4-(1H-pyrazol-4-yl)-phenyl]-amide

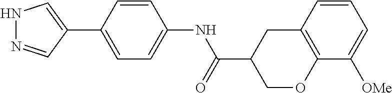

The title compound was prepared according to the procedure described in Scheme 1 (90 mg, 39% over three steps). ¹H NMR (DMSO-d₆, 400 MHz) δ 10.16 (s, 1H), 7.99 (br s, 2H), 7.60 (d, J=8.8 Hz, 2H), 7.55 (d, J=8.8 Hz, 2H), 6.82-6.77 (m, 2H), 6.75-6.70 (m, 1H), 4.47 (dd, J=10.8, 3.2 Hz, 1H), 3.95 (m, 1H), 3.73 (s, 3H), 3.06-2.91 (m, 3H). LC-MS: single peak at 254 nm, MH⁺ calcd. for C₂₀H₂₀N₃O₃: 350, obtained: 350.

### Example 90

### Synthesis of 8-methoxy-chroman-3-carboxylic acid [3-methoxy-4-(1H-pyrazol-4-yl)-phenyl]-amide

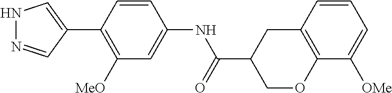

The title compound was prepared according to the procedure described in Scheme 1 (11 mg, 14% over three steps). LC-MS: single peak at 254 nm, MH⁺ calcd. for C₂₁H₂₂N₃O₄: 380, obtained: 380.

### Example 91

### Synthesis of 6-methoxy-chroman-3-carboxylic acid [3-methoxy-4-(1H-pyrazol-4-yl)phenyl]-amide

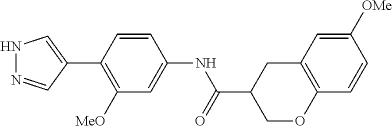

The title compound was prepared according to the procedure described in Scheme 1 (11 mg, 5% over three steps). ¹H NMR (DMSO-d₆, 400 MHz) δ 10.20 (s, 1H), 8.06 (br s. 1H), 7.91 (br s, 1H), 7.54 (d, J=8.4 Hz, 1H), 7.49 (d, J=2.0 Hz, 1H), 7.17 (dd, J=8.4, 2.0 Hz, 1H), 6.74-6.69 (m, 3H), 4.39 (d, J=10.8 Hz, 1H), 3.94 (t, J=10.0 Hz, 1H), 3.84 (s, 3H), 3.69 (s, 3H), 3.02-2.90 (m, 3H). LC-MS: single peak at 254 nm, MH⁺ calcd. for C₂₁H₂₂N³O₄: 380, obtained: 380.

### Example 92

### Synthesis of 6-methoxy-chroman-3-carboxylic acid [4-(1H-pyrazol-4-yl)-phenyl]-amide

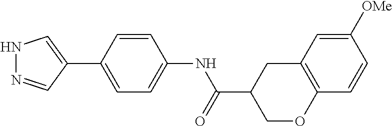

The title compound was prepared according to the procedure described in Scheme 1 except the crude product was purified by filtration and washing with water, ethanol, and ethyl acetate (20 mg, 36% over two steps). ¹H NMR (DMSO-d₆, 400 MHz) δ 10.09 (s, 1H), 8.12 (br s, 1 H), 7.87 (br s, 1H), 7.60 (d, J=8.8 Hz, 2H), 7.55 (d, J=8.8 Hz, 2H), 6.74-6.67 (m, 3H), 4.39 (d, J=10.8 Hz, 1H), 3.93 (m, 1H), 3.09-2.89 (m, 3H). LC-MS: single peak at 254 nm, MH⁺ calcd. for C₂₀H₂₀N₃O₃: 350, obtained: 350.

### Example 93

### Synthesis of 6-methoxy-chroman-3-carboxylic acid [2-methoxy-4-(1H-pyrazol-4-yl)-phenyl]-amide

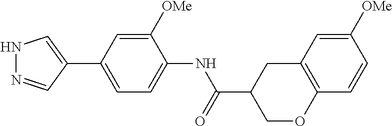

The title compound was prepared according to the procedure described in Scheme 1 (29 mg, 36% over three steps). ¹H NMR (DMSO-d₆, 400 MHz) δ 9.38 (s, 1H), 8.05 (br s, 2H), 7.89 (d, J=8.0 Hz, 1H), 7.26 (d, J=2.0 Hz, 1H), 7.15 (d, J=8.4 Hz, 1H), 6.74-6.66 (m, 3H), 4.35 (br d, J=10.4 Hz, 1H), 3.92 (m, 1H), 3.89 (s, 3H), 3.68 (s, 3H), 3.23-3.16 (m, 1H), 2.98 (dd, J=16.4, 9.8 Hz, 1H), 2.90 (dd, J=16.0, 4.4 Hz, 1H). LC-MS: single peak at 254 nm, MH⁺ calcd. for C₂₁H₂₂N₃O₄: 380, obtained: 380.

### Example 94

### Synthesis of 6-methoxy-chroman-3-carboxylic acid [3,5-difluoro-4-(1H-pyrazol-4-yl)-phenyl]-amide

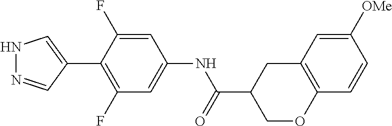

The title compound was prepared according to the procedure described in Scheme 1 (29 mg, 21% over three steps). ¹H NMR (DMSO-d₆, 400 MHz) δ 10.54 (s, 1H), 8.08 (br s, 1H), 7.84 (br s, 1H), 7.43 (d, J=10.8 Hz, 2H), 6.74-6.67 (m, 3H), 4.40 (d, J=10.8 Hz, 1H), 3.95 (t, J=10.0 Hz, 1H), 3.69 (s, 3H), 3.04-2.91 (m, 3H). LC-MS: single peak at 254 nm, MH⁺ calcd. for C₂₀H₁₈F₂N₃O₃: 386, obtained: 386.

### Example 95

### Synthesis of 6-methoxy-chroman-3-carboxylic acid [3-(2-dimethylamino-ethoxy)-4-(1H-pyrazol-4-yl)-phenyl]-amide

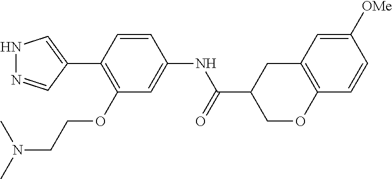

The title compound was prepared according to the procedure described in Scheme 1 (28 mg, 22% over three steps). ¹H NMR (DMSO-d₆, 400 MHz) δ 10.26 (s, 1H), 9.53 (br s, 1H), 8.04 (br s, 2H), 7.66 (d, J=1.6 Hz, 1H), 7.57 (d, J=8.4 Hz, 1H), 7.11 (dd, J=8.0, 1.6 Hz, 1H), 6.74-6.67 (m, 3H), 4.38 (dd, J=10.8, 2.8 Hz, 1H), 4.35 (t, J=4.8 Hz, 2H), 3.96 (t, J=10.0 Hz, 1H), 3.69 (s, 3H), 3.67 (m, 2H), 3.07-2.90 (m, 3H), 2.88 (s, 3H), 2.87 (s, 3H). LC-MS: single peak at 254 nm, MH⁺ calcd. for C₂₄H₂₉N₄O₄: 437, obtained: 437.

### Example 96

### Synthesis of 6-chloro-chroman-3-carboxylic acid [3-methoxy-4-(1H-pyrazol-4-yl)phenyl]-amide

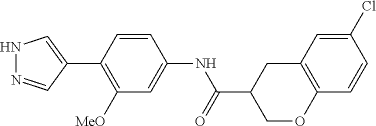

The title compound was prepared according to the procedure described in Scheme 1 (22 mg, 23% over three steps). ¹H NMR (DMSO-d₆, 400 MHz) δ 10.23 (s, 1H), 7.99 (br s, 2H), 7.55 (d, J=8.4 Hz, 1H), 7.46 (d, J=2.0 Hz, 1H), 7.25 (d, J=2.8 Hz, 1H), 7.16 (dd, J=8.4, 2.0 Hz, 1H), 7.13 (dd, J=8.8, 2.8 Hz, 1H), 6.82 (d, J=8.8 Hz, 1H), 4.45 (dd, J=10.8, 3.2 Hz, 1H), 4.04 (m, 1H), 3.84 (s, 3H), 3.06-2.94 (m, 3H). LC-MS: single peak at 254 nm, MH⁺ calcd. for C₂₀H₁₉ClN₃O₃: 384, obtained: 384.

### Example 97

### Synthesis of 6-chloro-chroman-3-carboxylic acid [4-(1H-pyrazol-4-yl)-phenyl]-amide

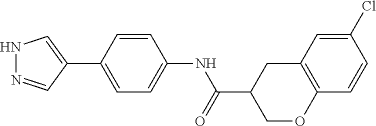

The title compound was prepared according to the procedure described in Scheme 1 except the crude product was purified by filtration and washing with water, ethanol, and ethyl acetate (25 mg, 40% over three steps). ¹H NMR (DMSO-d₆, 400 MHz) δ 10.18 (s, 1H), 8.12 (br s, 1 H), 7.87 (br s, 1H), 7.59 (d, J=8.8 Hz, 2H), 7.55 (d, J=8.8 Hz, 2H), 7.24 (d, J=2.4 Hz, 1H), 7.12 (dd, J=8.8, 2.4 Hz, 1H), 6.82 (d, J=8.8 Hz, 1H), 4.46 (d, J=10.8 Hz, 1H), 4.02 (m, 1H), 3.05-2.94 (m, 3H). LC-MS: single peak at 254 nm, MH⁺ calcd. for C₁₉H₁₇ClN₃O₂: 354, obtained: 354.

### Example 98

### Synthesis of 6-chloro-chroman-3-carboxylic acid [2-methoxy-4-(1H-pyrazol-4-4-yl)-phenyl]-amide

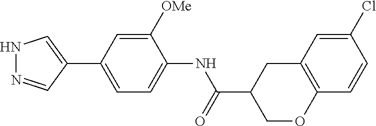

The title compound was prepared according to the procedure described in Scheme 1 (16 mg, 24% over three steps). ¹H NMR (DMSO-d₆, 400 MHz) δ 9.42 (s, 1H), 8.18 (br s, 1H), 7.93 (br s, 1H), 7.88 (d, J=8.0 Hz, 1H), 7.25 (dd, J=8.4, 2.0 Hz, 2H), 7.13 (dd, J=8.4, 2.8 Hz, 2 H), 6.81 (d, J=8.8 Hz, 1H), 4.42 (dd, J=10.8, 2.0 Hz, 1H), 4.01 (t, J=10.0 Hz, 1H), 3.90 (s, 3 H), 3.25-3.21 (m, 1H), 3.04-2.92 (m, 2H). LC-MS: single peak at 254 nm, MH⁺ calcd. for C₂₀H₁₉ClN₃O₃: 384, obtained: 384.

### Example 99

### Synthesis of 4-oxo-1,2,3,4-tetrahydro-naphthalene-2-carboxylic acid [4-(1H-pyrazol-4-yl)-phenyl]-amide

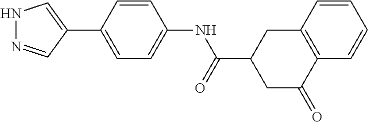

### Step A. 4-Oxo-1,2,3,4-tetrahydro-naphthalene-2-carboxylic acid

The title compound was prepared according to the procedure of Okubo et al. Bioorg. Med. Chem. 2004, 12, 356-3580.

### Step B. 4-Oxo-1,2,3,4-tetrahydro-naphthalene-2-carboxylic acid [4-(1H-pyrazol-4-yl)-phenyl]-amide

The title compound was prepared according to the procedure described in Scheme 1 except the crude product was purified by filtration and washing with water, ethanol, and ethyl acetate (22 mg, 19% over three steps). ¹H NMR (DMSO-d₆, 400 MHz) δ 10.11 (s, 1H), 8.11 (br s, 1 H), 7.88 (dd, J=8.0, 1.2 Hz, 1H), 7.87 (br s, 1H), 7.59-7.52 (m, 5H), 7.38 (m, 2H), 3.29-3.22 (m, 3H), 2.83 (m, 2H). LC-MS: single peak at 254 nm, MH⁺ calcd. for C₂₀H₁₈N₃O₂: 332, obtained: 332.

### Example 100

### Synthesis of 4-oxo-1,2,3,4-tetrahydro-naphthalene-2-carboxylic acid [3-methoxy-4-(1H-pyrazol-4-yl)-phenyl]-amide

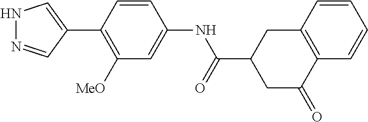

The title compound was prepared according to the procedure described in Scheme 1 (21 mg, 19%). ¹H NMR (DMSO-d₆, 400 MHz) δ 10.11 (s, 1H), 7.98 (br s, 2H), 7.88 (dd, J=8.0, 1.2 Hz, 1H), 7.57 (td, J=7.2, 1.6 Hz, 1H), 7.53 (d, J=8.4 Hz, 1H), 7.46 (d, J=2.0 Hz, 1H), 7.40 (d, J=7.6 Hz, 1H), 7.38 (t, J=7.2 Hz, 1H), 7.14 (dd, J=8.0, 1.6 Hz, 1H), 3.83 (s, 3H), 3.33-3.17 (m, 2H), 2.89-2.77 (m, 2H). LC-MS: single peak at 254 nm, MH⁺ calcd. for C₂₁H₂₀N₃O₃: 362, obtained: 362.

### Example 101

### Synthesis of 2,3-Dihydro-benzofuran-2-carboxylic acid [4-(1H-pyrazol-4-yl)-phenyl]-amide

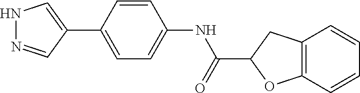

The title compound was prepared according to the procedure described in Scheme 1 except the crude product was purified by filtration and washing with water, ethanol, and ethyl acetate (24 mg, 51% over three steps). ¹H NMR (DMSO-d₆, 400 MHz) δ 10.13 (s, 1H), 8.00 (br s, 1 H), 7.65 (d, J=8.4 Hz, 2H), 7.55 (d, J=8.8 Hz, 2H), 7.25 (dd, J=7.6, 1.2 Hz, 1H), 7.15 (td, J=7.6, 1.2 Hz, 1H), 6.88 (m, 2H), 5.31 (dd, J=10.0, 6.8 Hz, 1H), 3.52 (dd, J=16.0, 10.0 Hz, 1H), 3.38 (dd, J=16.0, 6.8 Hz, 1H). LC-MS: single peak at 254 nm, MH⁺ calcd. for C₁₈H₁₆N₃O₂: 306, obtained: 306.

### Example 102

### Synthesis of 2,3-Dihydro-benzofuran-2-carboxylic acid [3-methoxy-4-(1H-pyrazol-4-yl)-phenyl]-amide

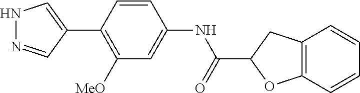

The title compound was prepared according to the procedure described in Scheme 1 (18 mg, 34% over three steps). ¹H NMR (DMSO-d₆, 400 MHz) δ 10.14 (s, 1H), 7.99 (s, 2H), 7.55 (d, J=8.4 Hz, 1H), 7.50 (d, J=2.0 Hz, 1H), 7.29 (dd, J=8.4, 2.0 Hz, 1H), 7.25 (dd, J=8.0, 1.2 Hz, 1H), 7.15 (t, J=8.0 Hz, 1H), 6.89 (m, 2H), 5.31 (dd, J=10.0, 6.8 Hz, 1H), 3.83 (s, 3H), 3.53 (dd, J=16.0, 10.4 Hz, 1H), 3.39 (dd, J=16.0, 6.4 Hz, 1H). LC-MS: single peak at 254 nm, MH⁺ calcd. for C₁₉H₁₈N₃O₃: 336, obtained: 336.

### Example 103

### Synthesis of 2,3-Dihydro-benzofuran-2-carboxylic acid [2-methoxy-4-(1H-pyrazol-4-yl)-phenyl]-amide

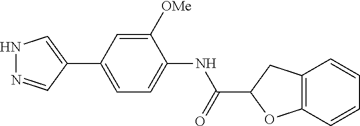

The title compound was prepared according to the procedure described in Scheme 1 (25 mg, 43% over three steps). ¹H NMR (DMSO-d₆, 400 MHz) δ 9.17 (s, 1H), 8.06 (br s, 2H), 8.03 (d, J=8.4 Hz, 1H), 7.27 (m, 2H), 7.17 (m, 2H), 6.96 (d, J=7.6 Hz, 1H), 6.91 (td, J=7.6, 1.2 Hz, 1H), 5.43 (dd, J=10.8, 6.0 Hz, 1H), 3.91 (s, 3H), 3.58 (dd, J=16.0, 10.4 Hz, 1H), 3.36 (dd, J=16.0, 10.0 Hz, 1H). LC-MS: single peak at 254 nm, MH⁺ calcd. for C₁₉H₁₈N₃O₃: 336, obtained: 336.

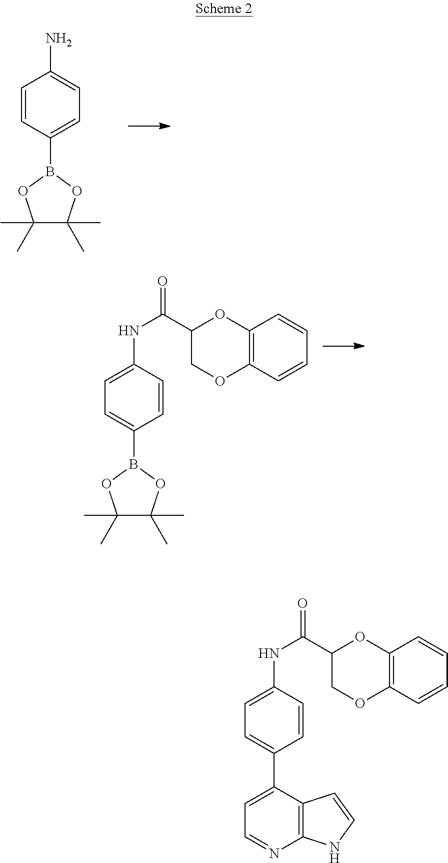

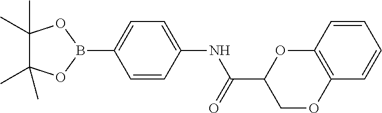

### Synthesis of N-(4-(4,4,5,5-tetramethyl-1,3,2-dioxaborolan-2-yl)phenyl)-2,3-dihydrobenzo[b][1,4]dioxine-2-carboxamide

A mixture of amine (1.0 equiv.), acid (1.0 equiv.), HATU (1.5 equiv.), and DIPEA (1.5 equiv.) in DMF was allowed to stir at room temperature for 20 h. The reaction mixture was partitioned between water and EtOAc, the organic layer was separated, dried, and concentrated. The residue was purified by silica chromatography to afford the title compound. ¹H NMR (CDCl₃, 400 MHz) δ 4.29-4.30 (m, 1H), 4.61-4.64 (m, 1H), 4.80-4.82 (m, 1H), 6.94-6.95 (m, 3H), 7.08-7.12 (m, 1H), 7.60-7.62 (m, 1H), 7.80-7.82 (m, 1H), 8.37 (br s, 1H).

### Example 104

### Synthesis of N-(4-(1H-Pyrrolo[2,3-b]pyridine-4-yl)phenyl)-2,3-dihydrobenzo[b][1,4]dioxine-2-carboxamide

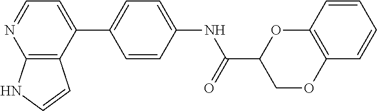

A mixture of above pinnacol boronic ester (1.0 equiv.), 4-chloro-1H-pyrrolo[2,3-b]pyridine (1.0 equiv.), Pd(PPh₃)₄ (0.03 equiv.), and Na₂CO₃ (2 M aqueous solution, 3.0 equiv.) in THF was heated to 120° C. for 30 min in microwave tube. The reaction mixture was partitioned between water and EtOAc, the organic layer was separated, dried, and concentrated. The residue was purified by HPLC to afford the title compound. ¹H NMR (DMSO, 400 MHz) δ 4.30-4.35 (dd, J=5.6, 11.6 Hz, 1H), 4.40-4.43 (dd, J=2.8, 11.6 Hz, 1H), 4.96-4.98 (dd, J=2.8, 5.6 Hz, 1H), 6.65-6.67 (m, 1H), 6.78-6.86 (m, 3H), 6.97-7.01 (m, 1H), 7.23-7.24 (m, 1H), 7.52-7.56 (m, 1H), 7.73-7.80 (m, 4H), 8.26-8.27 (d, J=5.6 Hz, 1H), 10.3 (s, 1H), 12.0 (s, 1H); LC-MS: C₂₂H₁₈N₃O₃ (M⁺+1) 372.19.

### Example 105

### Synthesis of N-(4-(7H-Pyrrolo[2,3-b]pyrimidin-4-yl)phenyl)-2,3-dihydrobenzo[b][1,4]dioxine-2-carboxamide

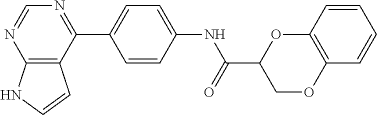

Procedures in Scheme 2 were used to prepare the title compound. ¹H NMR (DMSO, 400 MHz) δ 4.31-4.35 (dd, J=5.6, 11.6 Hz, 1H), 4.40-4.43 (dd, J=2.8, 11.6 Hz, 1H), 4.97-4.99 (dd, J=2.8, 5.6 Hz, 1H), 6.68-6.86 (m, 3H), 6.93-6.94 (m, 1H), 6.97-7.00 (m, 1H), 7.68 (s, 1H), 7.82-7.84 (m, 2H), 8.10-8.12 (m, 2H), 8.81 (s, 1H), 10.4 (s, 1H), 12.5 (br s, 1H); LC-MS:

C₂₁H₁₇N₄O₃(M⁺+1) 373.19.

### Example 106

### Synthesis of N-(4-(1,8-Naphthyridin-4-yl)phenyl)-2,3-dihydrobenzo[b][1,4]dioxine-2-carboxamide

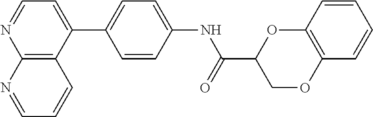

Procedures in Scheme 2 were used to prepare the title compound. ¹H NMR (DMSO, 400 MHz) δ 4.32-4.37 (dd, J=5.6, 11.6 Hz, 1H), 4.40-4.43 (dd, J=2.8, 11.6 Hz, 1H), 4.99-5.01 (dd, J=2.8, 5.6 Hz, 1H), 6.78-6.87 (m, 3H), 6.98-6.99 (m, 1H), 7.53-7.56 (m, 2H), 7.69-7.73 (m, 2H), 7.82-7.86 (m, 2H), 8.45-8.48 (dd, J=1.6, 8.4 Hz, 1H), 9.14-9.15 (m, 2H), 10.4 (s, 1H); LC-MS: C₂₃H₁₈N₃O₃(M⁺+1) 384.21.

### Example 107

### Synthesis of N-(4-(Pyrido[2,3-d]pyrimidin-4-yl)phenyl)-2,3-dihydrobenzo[b][1,4]dioxine-2-carboxamide

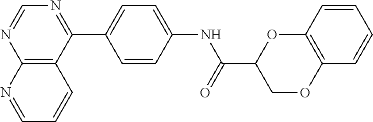

Procedures in Scheme 2 were used to prepare the title compound. ¹H NMR (DMSO, 400 MHz) δ 4.32-4.37 (dd, J=5.6, 11.6 Hz, 1H), 4.40-4.44 (dd, J=2.8, 11.6 Hz, 1H), 4.99-5.01 (dd, J=2.8, 5.6 Hz, 1H), 6.78-6.87 (m, 4H), 6.99-7.01 (m, 1H), 7.69-7.73 (m, 1H), 7.77-7.79 (m, 2H), 7.84-7.87 (m, 2H), 8.51-8.54 (dd, J=2.0, 8.4 Hz, 1H), 9.23-9.24 (m, 1H), 9.42 (m, 1H), 10.4 (s, 1H); LC-MS: C₂₂H₁₇N₄O₃(M⁺+1) 385.13.

### Example 108

### Synthesis of N-(4-(Pyrimidin-4-yl)phenyl)-2,3-dihydrobenzo[b][1,4]dioxine-2-carboxamide

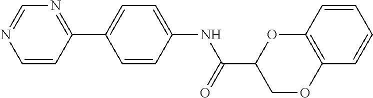

Procedures in Scheme 2 were used to prepare the title compound. ¹H NMR (DMSO, 400 MHz) δ 4.30-4.34 (dd, J=5.6, 11.6 Hz, 1H), 4.38-4.42 (dd, J=2.8, 11.6 Hz, 1H), 4.96-4.97 (dd, J=2.8, 5.6 Hz, 1H), 6.81-6.86 (m, 3H), 6.97-6.99 (m, 1H), 7.76-7.78 (m, 2H), 7.98-8.00 (m, 1H), 8.14-8.16 (m, 2H), 8.75-8.76 (m, 1H), 9.14 (m, 1H), 10.4 (s, 1H); LC-MS: C₁₉H₁₆N₃O₃ (M⁺+1) 334.15.

### Example 109

### Synthesis of N-(4-(1-Methyl-1H-pyrazol-4-yl)phenyl)-2,3-dihydrobenzo[b][1,4]dioxine-2-carboxamide

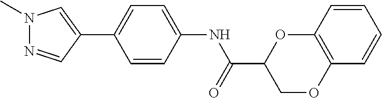

Procedures in Scheme 2 were used to prepare the title compound. ¹H NMR (DMSO, 400 MHz) δ 3.75 (s, 3H), 4.24-4.28 (dd, J=5.6, 11.6 Hz, 1H), 4.37-4.40 (dd, J=2.8, 11.6 Hz, 1H), 4.88-4.93 (dd, J=2.8, 5.6 Hz, 1H), 6.74-6.85 (m, 3H), 6.96-6.98 (m, 1H), 7.35-7.43 (m, 2H), 7.54-7.57 (m, 2H), 7.74 (s, 1H), 7.95 (s, 1H), 10.1 (s, 1H); LC-MS: C₁₉H₁₈N₃O₃(M⁺+1) 336.17.

### Example 110

### Synthesis of N-(3-Fluoro-4-(7H-pyrrolo[2,3-b]pyrimidin-4-yl)phenyl)-2,3-dihydrobenzo[b][1,4]dioxine-2-carboxamide

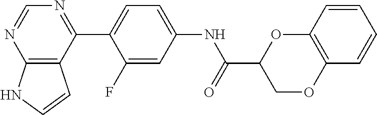

Procedures in Scheme 2 were used to prepare the title compound. ¹H NMR (DMSO, 400 MHz) δ 4.39-4.41 (m, 2H), 5.07-5.09 (m, 1H), 6.56 (m, 1H), 6.80-6.94 (m, 3H), 7.06-7.08 (m, 1H), 7.37-7.67 (complex, 6H), 7.80-7.96 (m, 2H), 8.88 (s, 1H), 10.60 (s, 1H), 12.36 (s, 1H); LC-MS: C₂₁H₁₆FN₄O₃(M⁺+1) 391.20.

### Example 111

### Synthesis of N-(3-Fluoro-4-(pyrimidin-4-yl)phenyl)-2,3-dihydrobenzo[b][1,4]dioxine-2-carboxamide

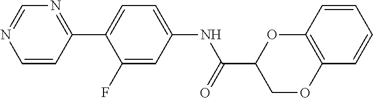

Procedures in Scheme 2 were used to prepare the title compound. ¹H NMR (DMSO, 400 MHz) δ 4.31-4.35 (dd, J=5.6, 11.6 Hz, 1H), 4.38-4.42 (dd, J=2.8, 11.6 Hz, 1H), 4.98-5.00 (dd, J=2.8, 5.6 Hz, 1H), 6.78-6.86 (m, 3H), 6.97-6.99 (m, 1H), 7.52-7.55 (m, 1H), 7.72-7.76 (dd, J=2.0, 14.4 Hz, 1H), 7.80-7.82 (m, 1H), 8.05-8.09 (m, 1H), 8.79-8.80 (m, 1H), 9.20-9.21 (d, J=1.2 Hz, 1H), 10.54 (s, 1H); LC-MS: C₁₉H₁₅FN₃O₃(M⁺+1) 352.16.

### Example 112

### Synthesis of N-(3-Fluoro-4-(pyrido[2,3-d]pyrimidin-4-yl)phenyl)-2,3-dihydrobenzo[b][1,4]dioxine-2-carboxamide

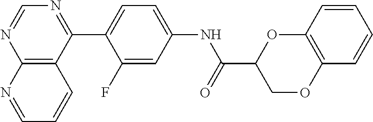

Procedures in Scheme 2 were used to prepare the title compound. ¹H NMR (DMSO, 400 MHz) δ 4.28-4.32 (dd, J=5.6, 11.6 Hz, 1H), 4.33-4.37 (dd, J=2.8, 11.6 Hz, 1H), 4.95-4.97 (dd, J=2.8, 5.6 Hz, 1H), 6.74-6.80 (m, 3H), 6.92-6.95 (m, 1H), 7.39-7.66 (complex, 3H), 7.74-7.78 (dd, J=2.0, 14.4 Hz, 1H), 8.18-8.21 (m, 1H), 9.18-9.20 (m, 1H), 9.42 (s, 1H), 10.54 (s, 1H); LC-MS: C₂₂H₁₆FN₄O₃ (M⁺+1) 403.19.

### Example 113

### Synthesis of N-(3-chloro-4-(pyrido[2,3-d]pyrimidin-4-yl)phenyl)-2,3-dihydrobenzo[b][1,4]dioxine-2-carboxamide

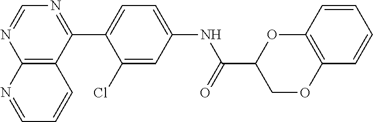

Procedures in Scheme 2 were used to prepare the title compound. Thus, to a solution of the amine (1 equiv) and acid (1 equiv) in DMF was added HATU (1 equiv) and DIEA (3 equiv) sequentially. The resulting mixture was stirred at room temperature for 1 hour. The solution was diluted with EtOAc and washed with saturated NaHCO₃ solution. The organic layer was dried over sodium sulfate and concentrated in vacuo. To a solution of the bromide and the bispinacolatoboronic ester in dioxane was added PdCl₂(dppf) and KOAc. The resulting mixture was stirred at 80° C. overnight. Water was added and extracted with EtOAc. The organic layers were combined, dried over sodium sulfate and concentrated in vacuo. Thus the chloride (1 equiv) and the boronic ester (1.5 equiv) were dissolved in THF in a sealed tube. Pd(PPh₃)₄ (0.03 equiv) and 2M solution of Na₂CO₃ (3 equiv) were added sequentially. The resulting mixture was heated to 100° C. for one hour in microwave reactor. After cooling to room temperature, the mixture was diluted with water and extracted with ethyl acetate. The organic layers were combined, dried over sodium sulfate and concentrated in vacuo. The residue thus produced was purified by preparative HPLC to give the product as solid (5% yield). LC-MS: single peak at 254 nm, MH⁺ calcd. for C₂₁H₁₅ClN₄O₃: 407, obtained: 407. ¹H-NMR (DMSO-d₆, 400 MHz), δ 10.51 (s, 1H), 8.85 (s, 1H), 8.04 (d, J=2.0 Hz, 1H), 7.76 (dd, J=8.5, 2.0 Hz, 1H), 7.59 (d, J=8.5 Hz, 1H), 7.77-7.39 (m, 3H), 7.07 (d, J=7.8 Hz 1H), 6.91 (m, 2H), 6.38 (d, J=1.5 Hz, 1H), 5.07 (dd, J=5.4, 2.9 Hz, 1H), 4.48 (dd, J=11.7, 2.9 Hz, 1H), 4.42 (dd, J=11.7, 5.4 Hz, 1H).

### Example 114

### Synthesis of N-(3-chloro-4-(1-methyl-1H-pyrazol-4-yl)phenyl)-2,3-dihydrobenzo[b][1,4]dioxine-2-carboxamide

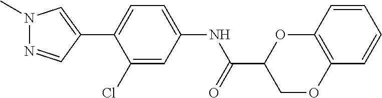

Procedures in Scheme 2 were used to prepare the title compound. Preparative HPLC gave 5 mg of the title compound (8%) from 28 mg starting material (bromide). LC-MS: single peak at 254 nm, MH⁺ calcd. for C₁₉H₁₆ClN₃O₃: 370, obtained: 370. ¹H-NMR (DMSO-d₆, 400 MHz), δ 10.25 (s, 1H), 8.06 (s, 1H), 7.83 (d, J=1.8 Hz, 1H), 7.73 (d, J=0.7 Hz, 1H), 7.50 (m, 3H), 6.97 (m, 1H), 6.82 (m, 2H), 4.93 (dd, J=5.7, 2.8 Hz, 1H), 4.38 (dd, J=11.6, 2.8 Hz, 1H), 4.29 (dd, J=11.6, 5.7 Hz, 1H), 3.81 (s, 3H).

### Example 115

### Synthesis of N-(3-methyl-4-(pyridin-4-yl)phenyl)-2,3-dihydrobenzo[b][1,4]dioxine-2-carboxamide

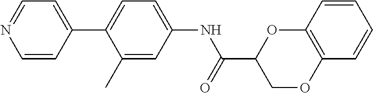

Procedures in Scheme 2 were used to prepare the title compound. Preparative HPLC gave 5 mg of the title compound (8%) from 28 mg starting material (bromide). LC-MS: single peak at 254 nm, MH⁺ calcd. for C₂₁H₁₈N₂O₃: 347, obtained: 347. ¹H-NMR (DMSO-d₆, 400 MHz), δ 10.25 (s, 1H), 8.06 (s, 1H), 7.83 (d, J=1.8 Hz, 1H), 7.73 (d, J=0.7 Hz, 1H), 7.50 (m, 3H), 6.97 (m, 1H), 6.82 (m, 2H), 4.93 (dd, J=5.7, 2.8 Hz, 1H), 4.38 (dd, J=11.6, 2.8 Hz, 1H), 4.29 (dd, J=11.6, 5.7 Hz, 1H), 3.81 (s, 3H).

### Example 116

### Synthesis of N-(4-(6-amino-2-methylpyrimidin-4-yl)-3-methylphenyl)-2,3-dihydrobenzo[b][1,4]dioxine-2-carboxamide

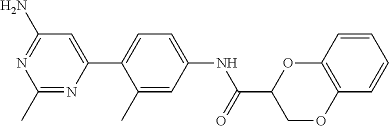

Procedures in Scheme 2 were used to prepare the title compound. Preparative HPLC gave 5 mg of the title compound (8%) from 28 mg starting material (bromide). LC-MS: single peak at 254 nm, MH⁺ calcd. for C₂₁H₂₀N₄O₃: 377, obtained: 377. ¹H-NMR (DMSO-d₆, 400 MHz), δ 10.25 (s, 1H), 8.06 (s, 1H), 7.83 (d, J=1.8 Hz, 1H), 7.73 (d, J=0.7 Hz, 1H), 7.50 (m, 3H), 6.97 (m, 1H), 6.82 (m, 2H), 4.93 (dd, J=5.7, 2.8 Hz, 1H), 4.38 (dd, J=11.6, 2.8 Hz, 1H), 4.29 (dd, J=11.6, 5.7 Hz, 1H), 3.81 (s, 3H).

### Example 117

### Synthesis of N-(3-methyl-4-(1,8-naphthyridin-4-yl)phenyl)-2,3-dihydrobenzo[b][1,4]dioxine-2-carboxamide

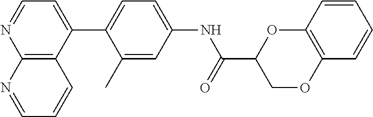

Procedures in Scheme 2 were used to prepare the title compound. Preparative HPLC gave 5 mg of the title compound (8%) from 28 mg starting material (bromide). LC-MS: single peak at 254 nm, MH⁺ calcd. for C₂₄H₁₉N₃O₃: 398, obtained: 398. ¹H-NMR (DMSO-d₆, 400 MHz), δ 10.25 (s, 1H), 8.06 (s, 1H), 7.83 (d, J=1.8 Hz, 1H), 7.73 (d, J=0.7 Hz, 1H), 7.50 (m, 3H), 6.97 (m, 1H), 6.82 (m, 2H), 4.93 (dd, J=5.7, 2.8 Hz, 1H), 4.38 (dd, J=11.6, 2.8 Hz, 1H), 4.29 (dd, J=11.6, 5.7 Hz, 1H), 3.81 (s, 3H).

### Example 118

### Synthesis of N-(2-fluoro-4-(1H-pyrazol-4-yl)phenyl)-2,3-dihydrobenzo[b][1,4]dioxine-2-carboxamide

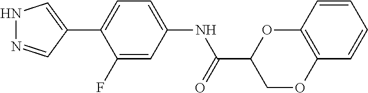

Procedures in Scheme 2 were used to prepare the title compound. ¹H-NMR (DMSO-d₆, 400 MHz), S: 10.33 (s, 1H), 8.01 (d, J=1.6 Hz, 2H), 7.72-7.53 (m, 2H), 7.43 (dd, J=2.0 Hz, J=8.4 Hz, 1H), 7.06-7.03 (m, 1H), 6.92-6.84 (m, 3H), 5.00 (dd, J=2.8 Hz, J=5.6 Hz, 1H), 4.46 (dd, J=2.8 Hz, J=11.6 Hz, 1H), 4.36 (dd, J=6.0 Hz, J=11.6 Hz, 1H); LC-MS: single peak at 254 nm, MH⁺ calcd. for C₁₈H₁₄FN₃O₃: 340, obtained: 340.

### General Procedures:

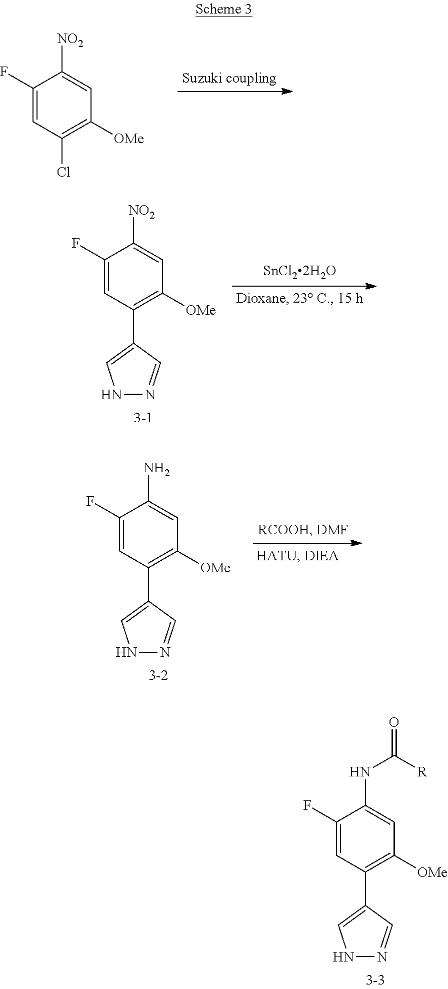

Pd[(Ph)₃]₄ (15%) was added to a degassed (using argon) solution of the 4-nitrochlorobenzene derivative (1 equiv), 4-pyrazoleboronic acid (2 equiv), and K₂CO₃ (5 equiv) in dioxane/H₂O (4:1 by volume). After the solution was sealed in a high-pressure reactor, the suspension was stirred at 100° C. for 40 h, at which the Suzuki coupling was complete based on LC-MS analysis. After a standard extraction process, the crude product was subjected to flash chromatography (Combi-Flash machine, a gradient MeOH in DCM was applied) to obtain the pure 3-1.

The nitro group was reduced using the SnCl₂ method. Hydrated SnCl₂ (6 equiv) was added to a solution of 3-1 in dioxane, and the resulting suspension was stirred at 23° C. overnight. The solvent was removed under reduced pressure, and a lot excess KOH (10 equiv relative to SnCl₂) in water was added to the residue. The suspension was then extracted 4× by DCM. The organic phase was then washed by brine (2×), dried over Na₂SO₄, and evaporated to give the aniline 3-2. This crude aniline was used directly in the next amide formation step without further purification. HATU coupling method was applied to prepare the final amide product 3-3. HATU (1.2 equiv) was added to a solution of 3-2 (1.0 equiv), an acid RCOOH (1.1 equiv), and DIEA (3 equiv) in DMF. After gently stirring the solution at 23° C. for 2 h, the mixture was directly subjected to preparative HPLC to obtain the final pure 3-3.

### Example 119

### Synthesis of N-(2-fluoro-5-methoxy-4-(1H-pyrazol-4-yl)phenyl)chroman-3-carboxamide

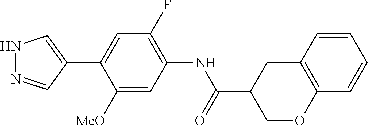

Procedures in Scheme 3 were used to prepare the title compound. LC-MS: single peak at 254 nm, MH⁺ calcd. for C₂₀H₁₈FN₃O₃: 368, obtained: 368.

### Example 120

### Synthesis of N-(2-fluoro-5-methoxy-4-(1H-pyrazol-4-yl)phenyl)chroman-2-carboxamide

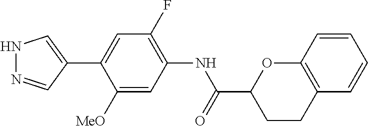

Procedures in Scheme 3 were used to prepare the title compound. LC-MS: single peak at 254 nm, MH⁺ calcd. for C₂₀H₁₈FN₃O₃: 368, obtained: 368.

### General Procedures:

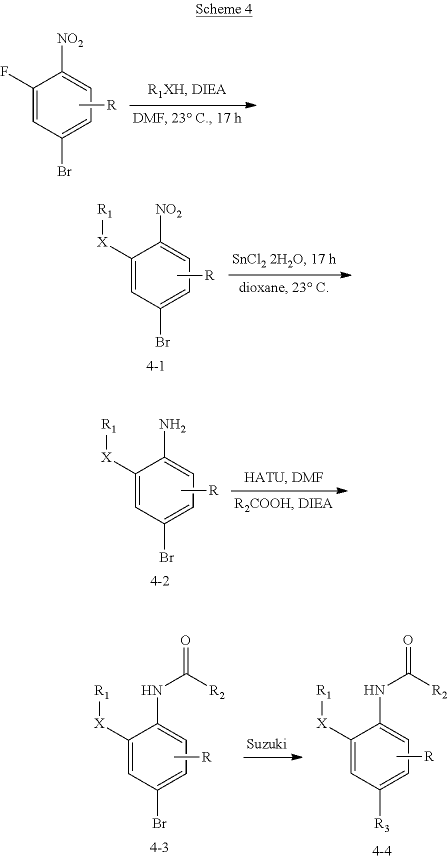

A SNAr reaction was used to prepare compound 4-1. Thus, an amine or an alcohol (1.1 equiv) was added to a solution of the 2-fluoronitrobenzene compound (1.0 equiv), and DIEA or KO^{t}BU (1.5 equiv) in DMF or THF. The suspension was gently stirred at 23° C. overnight. The solvents were then removed under reduced pressure, and the resulting residue was subjected to SnCl₂ reduction without further purification. Hydrated SnCl₂ (6 equiv) was therefore added to a solution of 4-1 in dioxane, and the suspension was stirred at 23° C. overnight. The solvent was removed under reduced pressure, and a lot excess KOH (10 equiv relative to SnCl₂) in water was added to the residue. The suspension was then extracted 4× by DCM. The organic phase was washed by brine (2×), dried over NaSO₄, and evaporated to give the crude aniline 4-2. This crude product was subjected to flash chromatography (Combi-Flash machine, a gradient MeOH in DCM was applied) to obtain the pure 4-2.

A HATU coupling method was applied to prepare the amide 4-3. Thus, HATU (1.2 equiv) was added to a solution of 4-2 (1.0 equiv), an acid RCOOH (1.1 equiv), and DIEA (3 equiv) in DMF. After gently stirring the solution at 23° C. for 2 h, the solvents were evaporated in a rotovapor. The residue was suspended in EtOAc, washed with brine (2×), saturated NaHCO₃ (2×), brine (2×), dried over Na₂SO₄, and evaporated to the crude 4-3, which was used directly in the next step without further purification. Thus, Pd[(Ph)₃]₄ (15%) was added to a degassed (using argon) solution of 4-3 (1 equiv), a boronic acid or ester (2 equiv), and K₂CO₃ (5 equiv) in dioxane/H₂O (4:1 by volume). After the solution was sealed in a high-pressure reactor, the suspension was stirred at 100° C. for 10 h, at which the Suzuki coupling was complete based on LC-MS analysis. After removing the solvents by evaporation, the resulting residue was subjected to preparative HPLC to give the final pure product 4-4.

### Example 121

### Synthesis of N-(2-(2-(dimethylamino)ethoxy)-4-(1H-pyrazol-4-yl)phenyl)-2,3-dihydrobenzo[b][1,4]dioxine-2-carboxamide

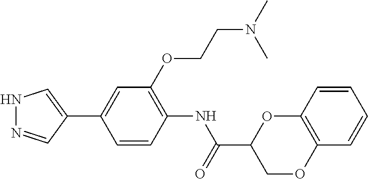

Procedures in Scheme 4 were used to prepare the title compound. ¹H-NMR (DMSO-d₆, 400 MHz), δ: 9.73 (b, 1H), 9.23 (s, 1H), 8.07 (s, 2H), 7.88 (d, J=8.4 Hz, 1H), 7.34 (d, J=1.6 Hz, 1H), 7.25 (dd, J=1.6 Hz, J=4.4 Hz, 1H), 7.09 (m, 1H), 6.93 (m, 3H), 5.09 (dd, J=2.8 Hz, J=5.6 Hz, 1H), 4.46 (dd, J=2.8 Hz, J=10.6 Hz, 1H), 4.37 (dd, J=6.0 Hz, J=11.6 Hz, 1H), 3.55 (m, 2H), 2.92 (m, 2H), 2.89 (s, 6H). LC-MS: single peak at 254 nm, MH⁺ calcd. for C₂₂H₂₄N₄O₄: 409, obtained: 409.

### Example 122

### Synthesis of (S)—N-(2-(2-(dimethylamino)ethylamino)-4-(1H-pyrazol-4-yl)phenyl)-2,3-dihydrobenzo[b][1,4]dioxine-2-carboxamide

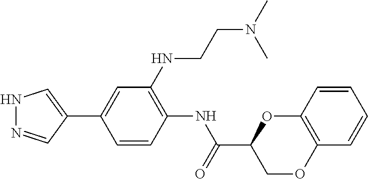

Procedures in Scheme 4 were used to prepare the title compound. LC-MS: single peak at 254 nm, MH⁺ calcd. for C₂₂H₂₅N₅O₃: 408, obtained: 408.

### Example 123

### Synthesis of N-(2-(1-methylpiperidin-4-yloxy)-4-(1H-pyrazol-4-yl)phenyl)-2,3-dihydrobenzo[b][1,4]dioxine-2-carboxamide

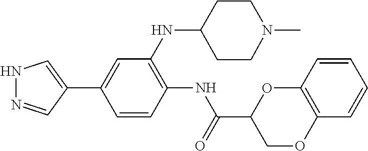

Procedures in Scheme 4 were used to prepare the title compound. ¹H-NMR (DMSO-d₆, 400 MHz), δ: 9.80 (b, 1H), 9.24 ((s, 1H), 8.05 (bs, 2H), 7.77 (m, 1H), 7.30 (m, 1H), 7.23 (m, 1H), 7.04 (m, 1H), 6.93 (m, 3H), 5.17 (m, 1H), 4.70 (m, 1H), 4.43 (m, 2H), 3.55-3.00 (m, 4H), 2.80 (m, 3H), 2.40-1.65 (m, 4H). LC-MS: single peak at 254 nm, MH⁺ calcd. for C₂₄H₂₆N₄O₄: 435, obtained: 435.

### Example 124

### Synthesis of N-(2-isopropoxy-4-(1H-pyrazol-4-yl)phenyl)-2,3-dihydrobenzo[b][1,4]dioxine-2-carboxamide

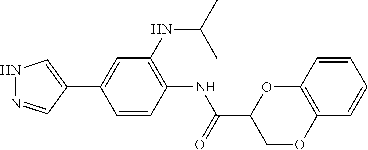

Procedures in Scheme 4 were used to prepare the title compound. LC-MS: single peak at 254 nm, MH⁺ calcd. for C₂₁H₂₁N₃O₄: 380, obtained: 380.

### Example 125

### Synthesis of N-(2-(2-methoxyethoxy)-4-(1H-pyrazol-4-yl)phenyl)-2,3-dihydrobenzo[b][1,4]dioxine-2-carboxamide

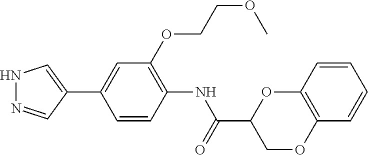

Procedures in Scheme 4 were used to prepare the title compound. ¹H-NMR (DMSO-d₆, 400 MHz), δ: 9.10 (s, 1H), 8.11 (s, 1H), 8.06 (bs, 2H), 7.60 (m, 1H), 7.31 (d, J=2.0 Hz, 1H), 7.20 (d, J=2.0 Hz, 1H), 7.20 (dd, J=1.6 Hz, J=8.4 Hz, 1H), 7.06 (dd, J=1.6 Hz, J=8.4 Hz, 1H), 6.96 (m, 1H), 6.91 (m, 2H), 5.13 (m, 2H), 4.50-4.15 (m, 5H), 3.70 (t, J=4.4 Hz, 2H), 3.37 (s, 3H). LC-MS: single peak at 254 nm, MH⁺ calcd. for C₂₁H₂₁N₃O₅: 396, obtained: 396.

### Example 126

### Synthesis of N-(2-ethoxy-4-(1H-pyrazol-4-yl)phenyl)-2,3-dihydrobenzo[b][1,4]dioxine-2-carboxamide

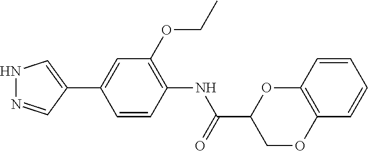

Procedures in Scheme 4 were used to prepare the title compound. ¹H-NMR (DMSO-d₆, 400 MHz), δ: 9.03 (s, 1H), 8.08 (d, J=4.0 Hz, 1H), 8.04 (bs, 2H), 7.25 (m, 1H), 7.17 (d, J=8.0 Hz, 1H), 7.06 (m, 1H), 6.93 (m, 3H), 5.15 (b, 1H), 4.46 (m, 1H), 4.32 (m, 1H), 3.91 (m, 2H), 1.33 (t, J=7.2 Hz, 3H). LC-MS: single peak at 254 nm, MH⁺ calcd. for C₂₀H₁₉N₃O₄: 366, obtained: 366.

### Example 127

### Synthesis of N-(5-fluoro-2-methoxy-4-(1H-pyrazol-4-yl)phenyl)chroman-3-carboxamide

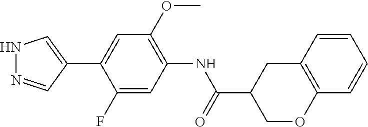

Procedures in Scheme 4 were used to prepare the title compound. ¹H-NMR (DMSO-d₆, 400 MHz), δ: 9.59 (s, 1H), 8.06 (bs, 2H), 7.97 (d, J=12.8 Hz, 1H), 7.32 (d, J=7.2 Hz, 1H), 7.10 (m, 2H), 6.86 (dd, J=1.2 Hz, J=7.6 Hz, 1H), 6.78 (dd, J=1.2 Hz, J=8.4 Hz, 1H), 4.41 (m, 1H), 3.97 (m, 1H), 3.27 (m, 1H), 2.98 (m, 2H). LC-MS: single peak at 254 nm, MH⁺ calcd. for C₂₀H₁₈FN₃O₃: 368, obtained: 368.

### Example 128

### Synthesis of (S)—N-(2-(2-(dimethylamino)ethoxy)-5-fluoro-4-(1H-pyrazol-4-yl)phenyl)-2,3-dihydrobenzo[b][1,4]dioxine-2-carboxamide

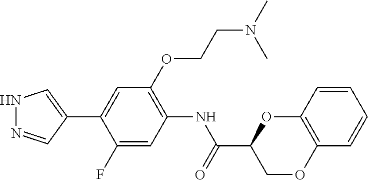

Procedures in Scheme 4 were used to prepare the title compound. ¹H-NMR (DMSO-d₆, 400 MHz), δ: 9.40 (b, 1H), 8.88 (s, 1H), 7.65 (bs, 2H), 7.51 (s, 1H), 7.01 (d, J=6.8 Hz, 1H), 6.67 (m, 1H), 6.51 (m, 3H), 4.71 (dd, J=2.8 Hz, J=5.6 Hz, 1H), 4.05 (m, 4H), 3.12 (m, 2H), 2.09 (s, 6H). LC-MS: single peak at 254 nm, MH⁺ calcd. for C₂₂H₂₃FN₄O₄: 427, obtained: 427.

### Example 129

### Synthesis of N-(2-(2-(dimethylamino)ethoxy)-5-fluoro-4-(1H-pyrazol-4-yl)phenyl)chroman-3-carboxamide

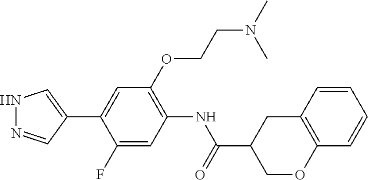

Procedures in Scheme 4 were used to prepare the title compound. ¹H-NMR (DMSO-d₆, 400 MHz), δ: 9.79 (b, 1H), 9.39 (s, 1H), 8.07 (bs, 2H), 7.93 (d, J=12.8 Hz, 1H), 7.42 (d, J=6.8 Hz, 1H), 7.15 (d, J=7.6 Hz, 1H), 7.08 (m, 1H), 6.85 (m, 2H), 4.46 (m, 3H), 4.01 (t, J=10.0 Hz, 1H), 3.60 (m, 2H), 3.22 (m, 1H), 3.06 (m, 1H), 2.97 (m, 1H), 2.94 (s, 3H), 2.93 (s, 3H). LC-MS: single peak at 254 nm, MH⁺ calcd. for C₂₃H₂₅FN₄O₃: 425, obtained: 425.

### Example 130

### Synthesis of 6-chloro-N-(2-(2-(dimethylamino)ethoxy)-5-fluoro-4-(1H-pyrazol-4-yl)phenyl)chroman-3-carboxamide

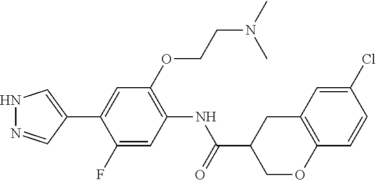

Procedures in Scheme 4 were used to prepare the title compound. LC-MS: single peak at 254 nm, MH⁺ calcd. for C₂₃H₂₄FClN₄O₃: 459, obtained: 459.

### Example 131

### Synthesis of 6-chloro-N-(2-(3-(dimethylamino)propoxy)-5-fluoro-4-(1H-pyrazol-4-yl)phenyl)chroman-3-carboxamide

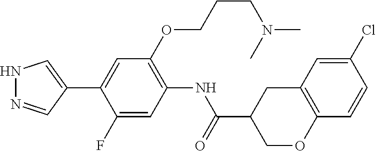

Procedures in Scheme 4 were used to prepare the title compound. LC-MS: single peak at 254 nm, MH⁺ calcd. for C₂₄H₂₆FClN₄O₃: 473, obtained: 473.

### Example 132

### Synthesis of N-(2-(2-(dimethylamino)ethoxy)-5-fluoro-4-(1H-pyrazol-4-yl)phenyl)-6-methylchroman-3-carboxamide

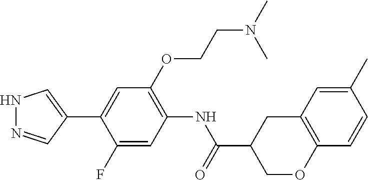

Procedures in Scheme 4 were used to prepare the title compound. ¹H-NMR (DMSO-d₆, 400 MHz), δ: 9.65 (b, 1H), 9.37 (s, 1H), 8.07 (bs, 2H), 7.93 (d, J=12.8 Hz, 1H), 7.42 (d, J=7.2 Hz, 1H), 6.95 (b, 1H), 6.90 (dd, J=1.6 Hz, J=8.0 Hz, 1H), 6.69 (d, J=8.0 Hz, 1H), 4.44 (m, 3H), 3.96 (t, J=10.4 Hz, 1H), 3.59 (m, 2H), 3.20 (m, 1H), 3.02 (m, 1H), 2.93 (s, 3H), 2.92 (s, 3H), 2.88 (m, 1H), 2.22 (s, 3H). LC-MS: single peak at 254 nm, MH⁺ calcd. for C₂₄H₂₇FN₄O₃: 439, obtained: 439.

### Example 133

### Synthesis of N-(2-(2-(dimethylamino)ethoxy)-5-fluoro-4-(1H-pyrazol-4-yl)phenyl)-7-methoxychroman-3-carboxamide

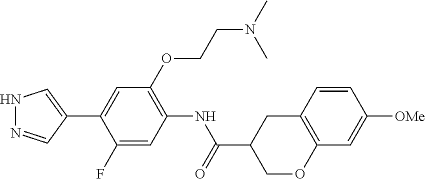

Procedures in Scheme 4 were used to prepare the title compound. LC-MS: single peak at 254 nm, MH⁺ calcd. for C₂₄H₂₇FN₄O₄: 455, obtained: 455.

### Example 134

### Synthesis of N-(5-fluoro-2-morpholino-4-(1H-pyrazol-4-yl)phenyl)-7-methoxychroman-3-carboxamide

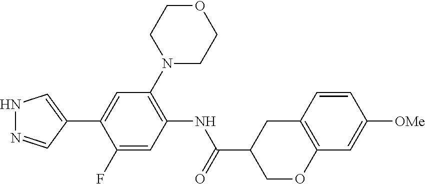

Procedures in Scheme 4 were used to prepare the title compound. ¹H-NMR (DMSO-d₆, 400 MHz), δ: 9.31 (s, 1H), 8.06 (bs, 2H), 7.93 (d, J=14.2 Hz, 1H), 7.54 (m, 1H), 7.05 (d, J=8.4 Hz, 1H), 6.48 (dd, J=2.4 Hz, J=8.4 Hz, 1H), 6.36 (d, J=2.4 Hz, 1H), 4.39 (d, J=3.2 Hz, 1H), 4.08 (dd, J=0.8 Hz, J=6.8 Hz, 1H), 3.83 (m, 4H), 3.69 (s, 3H), 3.23 (m, 1H), 2.96 (m, 2H), 2.86 (m, 4H). LC-MS: single peak at 254 nm, MH⁺ calcd. for C₂₄H₂₅FN₄O₄: 453, obtained: 453.

### Example 135

### Synthesis of 6-chloro-N-(5-fluoro-2-morpholino-4-(1H-pyrazol-4-yl)phenyl)chroman-3-carboxamide

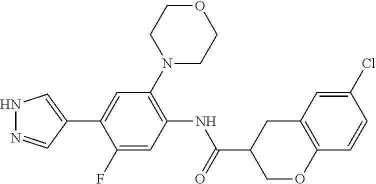

Procedures in Scheme 4 were used to prepare the title compound. ¹H-NMR (DMSO-d₆, 400 MHz), δ: 9.87 (s, 1H), 7.95 (bs, 2H), 7.77 (m, 3H), 6.76 (dd, J=6.0 Hz, J=12.4 Hz, 1H), 6.40 (dd, J=7.2 Hz, J=14.8 Hz, 1H), 4.30 (m, 2H), 3.80-3.30 (m, 5H), 3.20-3.00 (m, 4H). LC-MS: single peak at 254 nm, MH⁺ calcd. for C₂₃H₂₂FClN₄O₃: 457, obtained: 457.

### Example 136

### Synthesis of (S)—N-(5-fluoro-2-morpholino-4-(1H-pyrazol-4-yl)phenyl)-2,3-dihydrobenzo[b][1,4]dioxine-2-carboxamide

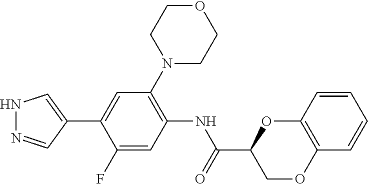

Procedures in Scheme 4 were used to prepare the title compound. LC-MS: single peak at 254 nm, MH⁺ calcd. for C₂₂H₂₁FN₄O₄: 425, obtained: 425.

### Example 137

### Synthesis of (S)—N-(5-fluoro-2-(4-hydroxypiperidin-1-yl)-4-(1H-pyrazol-4-yl)phenyl)-2,3-dihydrobenzo[b][1,4]dioxine-2-carboxamide

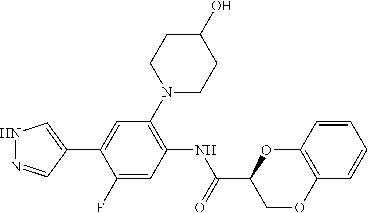

Procedures in Scheme 4 were used to prepare the title compound. ¹H-NMR (DMSO-d₆, 400 MHz), δ: 9.70 (s, 1H), 8.12 (d, J=13.2 Hz, 1H), 8.05 (bs, 2H), 7.60 (m, 1H), 7.12 (m, 1H), 6.94 (m, 3H), 5.15 (m, 1H), 4.42 (m, 2H), 3.68 (m, 1H), 3.00-2.60 (m, 4H), 2.00-1.50 (m, 4H). LC-MS: single peak at 254 nm, MH⁺ calcd. for C₂₃H₂₃FN₄O₄: 439, obtained: 439.

### Example 138

### Synthesis of N-(5-fluoro-2-(4-hydroxypiperidin-1-yl)-4-(1H-pyrazol-4-yl)phenyl)-6-methoxychroman-3-carboxamide

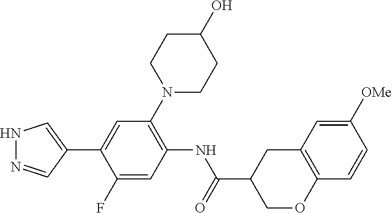

Procedures in Scheme 4 were used to prepare the title compound. LC-MS: single peak at 254 nm, MH⁺ calcd. for C₂₅H₂₇FN₄O₄: 467, obtained: 467.

### Example 139

### Synthesis of chroman-3-carboxylic acid [2-(2-dimethylamino-ethoxy)-4-(1H-pyrazol-4-yl)-phenyl)]-amide

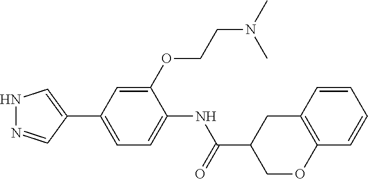

### Step A. 2-(5-bromo-2-nitrophenoxy)-N,N-dimethylethanamine

To a solution of 2-(dimethylamino)ethanol (0.610 mL, 6.06 mmol) in THF (25 mL) at 0° C. was added NaH (60% oil dispersion, 0.360 g, 9 mmol). After stirring for 15 min, 4-bromo-2-fluoro-1-nitrobenzene (1.32 g, 6.00 mmol) was added and the resulting mixture was allowed to warm to room temperature and stirred for 7 h. The solvent was removed by rotary evaporation and the residue was treated with diethyl ether (50 mL) and an aqueous 0.5 N HCl solution (100 mL). The layers were separated and the aqueous layer was basified with an aqueous saturated NaCO₃ solution and extracted with ethyl acetate (3×50 mL). The combined organic layers were washed with brine (50 mL), dried over Na₂SO₄, filtered, and concentrated to give the title compound (1.59 g, 92%). ¹H NMR (CDCl₃, 400 MHz) δ 7.74 (d, J=8.8 Hz, 1H), 7.25 (d, J=2.0 Hz, 1H), 7.17 (dd, J=8.8, 2.0 Hz, 1H), 4.19 (t, J=5.6 Hz, 2H), 2.80 (t, J=5.6 Hz, 2H), 2.35 (s, 6H). LC-MS: single peak at 254 nm, MH⁺ calcd. for C₁₀H₁₄BrN₂O₃: 289, obtained: 289.

### Step B. 4-bromo-2-(2-(dimethylamino)ethoxy)aniline

To a solution of 2-(5-bromo-2-nitrophenoxy)-N,N-dimethylethanamine (Step A, 1.59 g, 5.50 mmol) in ethanol (50 mL) was added stannous chloride (6.2 g, 27.5 mmol) and the mixture was heated to 70° C. for 2 h. The reaction was mixture was diluted with ice water and concentrated by rotary evaporation. An aqueous Na₂CO₃ solution was added and the aqueous phase was extracted with ethyl acetate (3×60 mL). The combined organic layers were washed with brine (50 mL), dried over Na₂SO₄, filtered, and concentrated to dryness to afford the title compound (1.30 g, 91%). ¹H NMR (CDCl₃, 400 MHz) δ 6.90 (m, 2H), 6.57 (d, J=8.8 Hz, 1H), 4.07 (t, J=5.6 Hz, 2H), 3.92 (br s, 2H), 2.74 (t, J=5.6 Hz, 2H), 2.33 (s, 6H). LC-MS: single peak at 254 nm, MH⁺ calcd. for C₁₀H₁₆BrN₂O: 259, obtained: 259.

### Step C. Chroman-3-carboxylic acid [2-(2-dimethylamino-ethoxy)-4-bromo-phenyl]-amide

The title compound was prepared from chroman-3-carboxylic acid and 4-bromo-2-(2-dimethylamino-ethoxy)aniline according to the procedure described in Scheme 4 (76 mg, 33% over three steps). ¹H NMR (DMSO-d₆, 400 MHz) δ 9.57 (br s, 1H), 9.32 (s, 1H), 8.07 (br s, 2H), 7.83 (d, J=8.4 Hz, 1H), 7.34 (d, J=1.6 Hz, 1H), 7.23 (dd, J=8.4, 1.6 Hz, 1H), 7.15 (d, J=7.2 Hz, 1H), 7.10 (t, J=8.0 Hz, 1H), 6.87 (td, J=7.6, 1.2 Hz, 1H), 6.80 (d, J=8.0 Hz, 1H), 4.46 (m, 3H), 4.01 (t, J=10.2 Hz, 1H), 3.59 (m, 2H), 3.22-3.15 (m, 1H), 3.06 (dd, J=16.0, 10.4 Hz, 1H), 2.97-2.92 (m, 7H). LC-MS: single peak at 254 nm, MH⁺ calcd. for C₂₃H₂₇N₄O₃: 407, obtained: 407.

### Example 140

### Synthesis of 6-methoxy-chroman-3-carboxylic acid [2-(2-dimethylamino-ethoxy)-4-(1H-pyrazol-4-yl)-phenyl]-amide

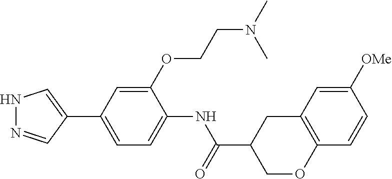

The title compound was prepared from 6-methoxy-chroman-3-carboxylic acid and 4-bromo-2-(2-dimethylamino-ethoxy)aniline (Example 139, Step B) according to the procedure described in Scheme 4 (52 mg, 26% over two steps). ¹H NMR (DMSO-d₆, 400 MHz) δ 9.67 (br s, 1H), 9.33 (s, 1H), 8.07 (br s, 2H), 7.83 (d, J=8.0 Hz, 1H), 7.34 (d, J=1.6 Hz, 1H), 7.23 (dd, J=8.4, 1.6 Hz, 1H), 6.74-6.68 (m, 3H), 4.45 (t, J=4.8 Hz, 1H), 4.37 (d, J=15.6 Hz, 1H), 3.95 (t, J=10.4 Hz, 1H), 3.69 (s, 3H), 3.59 (m, 2H), 3.18 (m, 1H), 3.06 (dd, J=16.4, 10.4 Hz, 1H), 2.93-2.89 (m, 7H). LC-MS: single peak at 254 nm, MH⁺ calcd. for C₂₄H₂₉N₄O₄: 437, obtained: 437.

### Example 141

### Synthesis of 6-methoxy-chroman-3-carboxylic acid [2-(2-dimethylamino-ethoxy)-5-fluoro-4-(1H-pyrazol-4-yl)-phenyl]-amide

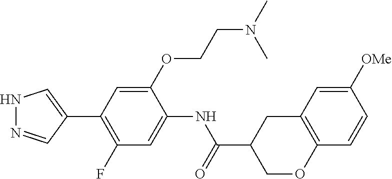

The title compound was prepared from 6-methoxy-chroman-3-carboxylic acid and 4-bromo-2-(2-dimethylamino-ethoxy)-5-fluoroaniline according to the procedure described in Scheme 4 (18 mg, 14% over three steps). ¹H NMR (DMSO-d₆, 400 MHz) δ 9.60 (br s, 1H), 9.38 (s, 1H), 8.07 (br s, 2H), 7.93 (d, J=12.8 Hz, 1H), 7.42 (d, J=7.2 Hz, 1H), 6.75-6.69 (m, 3H), 4.45 (t, J=5.0 Hz, 2H), 4.41 (d, J=10.8 Hz, 1H), 3.96 (t, J=10.2 Hz, 1H), 3.59 (m, 2H), 3.22 (m, 1H), 3.05 (dd, J=16.2, 10.2 Hz, 1H), 2.93 (s, 3H), 2.92 (s, 3H), 2.93-2.89 (m, 1H). LC-MS: single peak at 254 nm, MH⁺ calcd. for C₂₄H₂₈FN₄O₄: 455, obtained: 455.

### Example 142

### Synthesis of 6-methoxy-chroman-3-carboxylic acid [2-(2-dimethylamino-ethoxy)-5-fluoro-4-(3-methyl-1H-pyrazol-4-yl)-phenyl]-amide

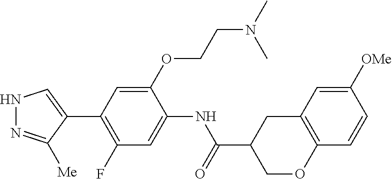

The title compound was prepared from 6-methoxy-chroman-3-carboxylic acid and 4-bromo-2-(2-dimethylamino-ethoxy)-5-fluoroaniline according to the procedure described in Scheme 4 (21 mg, 17% over three steps). ¹H NMR (DMSO-d₆, 400 MHz) δ 9.59 (br s, 1H), 9.43 (s, 1H), 7.92 (d, J=12.4 Hz, 1H), 7.67 (br s, 1H), 7.10 (d, J=6.8 Hz, 1H), 6.75-6.68 (m, 3H), 4.41 (m, 3H), 3.96 (t, J=10.2 Hz, 1H), 3.69 (s, 3H), 3.57 (m, 2H), 3.26-3.20 (m, 1H), 3.05 (dd, J=16.2, 10.2 Hz, 1H), 2.92 (s, 3H), 2.91 (s, 3H), 2.95-2.90 (m, 1H). LC-MS: single peak at 254 nm, MH⁺ calcd. for C₂₅H₃₀FN₄O₄: 469, obtained: 469.

### Example 143

### Synthesis of 6-chloro-chroman-3-carboxylic acid [2-(2-dimethylamino-ethoxy)-4-(1H-pyrazol-4-yl)-phenyl]-amide

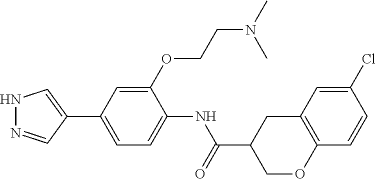

The title compound was prepared from 6-chloro-chroman-3-carboxylic acid and 4-bromo-2-(2-dimethylamino-ethoxy)aniline according to the procedure described in Scheme 4 (52 mg, 26% over three steps). ¹H NMR (DMSO-d₆, 400 MHz) δ 9.54 (br s, 1H), 9.33 (s, 1H), 8.13 (br s, 2H), 7.81 (d, J=8.4 Hz, 1H), 7.34 (d, J=2.0 Hz, 1H), 7.24 (m, 2H), 7.14 (dd, J=8.8, 2.8 Hz, 1H), 6.83 (d, J=8.8 Hz, 1H), 4.46 (m, 3H), 4.04 (t, J=10.0 Hz, 1H), 3.59 (m, 2H), 3.17 (m, 1H), 3.08-3.02 (m, 1H), 2.97 (m, 1H), 2.94 (s, 3H), 2.93 (s, 3H). LC-MS: single peak at 254 nm, MH⁺ calcd. for C₂₃H₂₆ClN₄O₃: 441, obtained: 441.

### Example 144

### Synthesis of 6-methyl-chroman-3-carboxylic acid [2-(2-dimethylamino-ethoxy)-4-(1H-pyrazol-4-yl)-phenyl]-amide

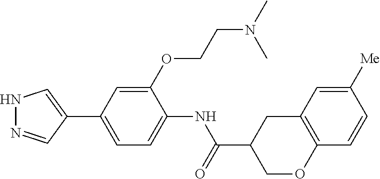

The title compound was prepared from 6-methyl-chroman-3-carboxylic acid and 4-bromo-2-(2-dimethylamino-ethoxy)aniline according to the procedure described in Scheme 4 (28 mg, 32% over three steps). ¹H NMR (DMSO-d₆, 400 MHz) δ 9.52 (br s, 1H), 9.29 (s, 1H), 8.07 (br s, 2H), 7.83 (d, J=8.4 Hz, 1H), 7.34 (d, J=2.0 Hz, 1H), 7.23 (dd, J=8.0, 1.6 Hz, 1H), 6.95 (s, 1H), 6.90 (d, J=8.0 Hz, 1H), 4.44 (m, 3H), 3.96 (t, J=10.2 Hz, 1H), 3.59 (m, 2H), 3.17-3.11 (m, 1H), 3.02 (dd, J=16.0, 10.8 Hz, 1H), 2.94-2.86 (m, 7H), 2.21 (s, 3H). LC-MS: single peak at 254 nm, MH⁺ calcd. for C₂₄H₂₉N₄O₃: 421, obtained: 421.

### Example 145

### Synthesis of 6-fluoro-chroman-3-carboxylic acid [2-(2-dimethylamino-ethoxy)-4-(1H-pyrazol-4-yl)-phenyl]-amide

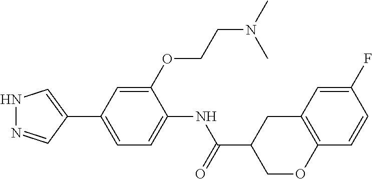

The title compound was prepared from 6-fluoro-chroman-3-carboxylic acid and 4-bromo-2-(2-dimethylamino-ethoxy)aniline according to the procedure described in Scheme 4 (22 mg, 29% over three steps). ¹H NMR (DMSO-d₆, 400 MHz) δ 9.59 (br s, 1H), 9.33 (s, 1H), 8.07 (s, 2H), 7.82 (d, J=8.4 Hz, 1H), 7.34 (d, J=1.6 Hz, 1H), 7.23 (dd, J=8.4, 2.0 Hz, 1H), 7.03 (dd, J=8.8, 2.8 Hz, 1H), 2H), 6.94 (td, J=8.8, 3.2 Hz, 1H), 6.81 (dd, J=8.8, 5.2 Hz, 1H), 4.46 (m, 3H), 4.10 (t, J=10.4 Hz, 1H), 3.59 (dd, J=9.6, 5.2 Hz, 2H), 3.21-3.14 (m, 1H), 3.06 (dd, J=16.8, 10.0 Hz, 1H), 2.97-2.92 (m, 7H). LC-MS: single peak at 254 nm, MH⁺ calcd. for C₂₃H₂₆FN₄O₃: 425, obtained: 425.

### Example 146

### Synthesis of 6-chloro-3-methyl-chroman-3-carboxylic acid [2-(2-dimethylamino-ethoxy)-4-(1H-pyrazol-4-yl)-phenyl]-amide

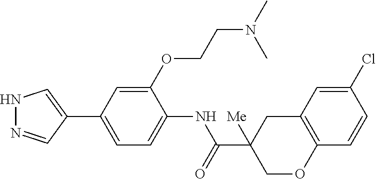

The title compound was prepared from 6-chloro-3-methyl-chroman-3-carboxylic acid and 4-bromo-2-(2-dimethylamino-ethoxy)aniline according to the procedure described in Scheme 4 (21 mg, 24% over three steps). ¹H NMR (DMSO-d₆, 400 MHz) δ 9.61 (br s, 1H), 8.95 (s, 1H), 8.07 (s, 2H), 7.52 (d, J=8.4 Hz, 1H), 7.32 (d, J=1.6 Hz, 1H), 7.22 (m, 2H), 7.13 (dd, J=8.8, 2.8 Hz, 1H), 6.85 (d, J=8.4 Hz, 1H), 4.43 (m, 3H), 4.09 (t, J=10.8 Hz, 1H), 3.53 (m, 2H), 3.28 (d, J=16.8 Hz, 1H), 2.92 (t, J=5.2 Hz, 6H), 2.79 (d, J=16.4 Hz, 1H), 1.31 (s, 3H). LC-MS: single peak at 254 nm, MH⁺ calcd. for C₂₃H₂₆ClN₄O₃: 441, obtained: 441.

### General Procedures for the Synthesis of Compound 5-3:

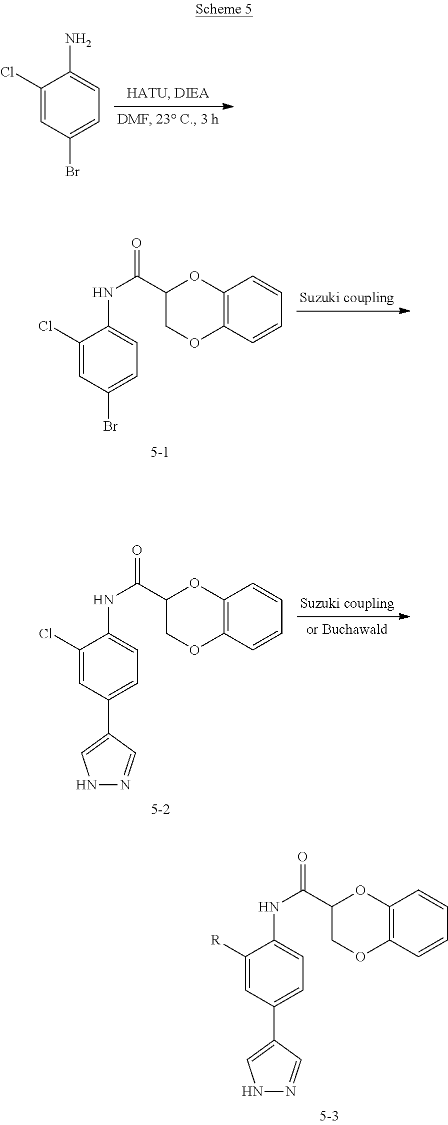

The HATU coupling method was used to prepare compound 5-1. Thus, HATU (1.3 equiv) was added to a solution of 4-bromo-5-chloroaniline (1.0 equiv), the benzodioxane-3-carboxylic acid (1.2 equiv), DIEA (4 equiv) in DMF. After gently stirring at 23° C. for 10 h, the solvents were removed under reduced pressure. The residue was suspended in EtOAc, standard washing applied (brine, NaHCO₃, brine, 1N HCl, and brine), dried over Na₂SO₄, and evaporated to give the crude product 5-1. This crude material was used directly in the next step without further purification.

A general Suzuki coupling method was utilized to make compound 5-2. The reaction was run in high pressure reactor or a Microwave reactor (Biotage). Pd[P(Ph)₃]₄ (15%) was therefore added to a degassed (with argon) solution of 5-1 (1.0 equiv), 4-pyrazoleboronic acid (2.0 equiv), and K₂CO₃ (5 equiv) in dioxane/H₂O (4:1 by volume). After the reactor was sealed, the solution was heated at 90-100° C. for 10 h on thermal conditions or at 110° C. for 2 h in a Microwave setup (monitored by LC-MS). The solvents were evaporated, and the residue was subjected to flash chromatography (Combi-Flash, a gradient MeOH in DCM was applied) to give compound 5-2. This compound 5-2 was used as the starting material in a second Suzuki coupling reaction to give compound 5-3. Thus, a similar procedure was applied as above in the second Suzuki coupling, and the crude product was subjected to preparative HPLC to give the final product 5-3 as a TFA salt.

### Example 147

### Synthesis of N-(4′-(N,N-dimethylsulfamoyl)-5-(1H-pyrazol-4-yl)biphenyl-2-yl)-2,3-dihydrobenzo[b][1,4]dioxine-2-carboxamide

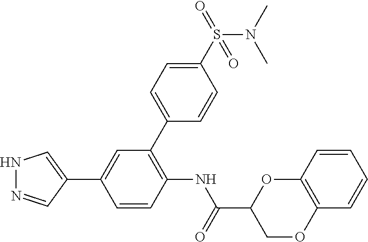

Procedures in Scheme 5 were used to prepare the title compound. LC-MS: single peak at 254 nm, MH⁺ calcd. for C₂₆H₂₄N₄O₅S: 505, obtained: 505.

### Example 148

### Synthesis of N-(5-(1H-pyrazol-4-yl)-3′-sulfamoylbiphenyl-2-yl)-2,3-dihydrobenzo[b][1,4]dioxine-2-carboxamide

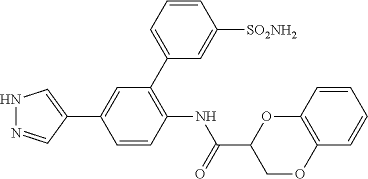

Procedures in Scheme 5 were used to prepare the title compound. LC-MS: single peak at 254 nm, MH⁺ calcd. for C₂₄H₂₀N₄O₅S: 477, obtained: 477.

### Example 149

### Synthesis of N-(5-(1H-pyrazol-4-yl)-4′-sulfamoylbiphenyl-2-yl)-2,3-dihydrobenzo[b][1,4]dioxine-2-carboxamide

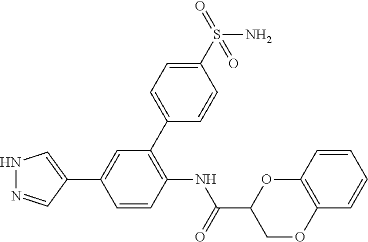

Procedures in Scheme 5 were used to prepare the title compound. LC-MS: single peak at 254 nm, MH⁺ calcd. for C₂₄H₂₀N₄O₅S: 477, obtained: 477.

### Example 150

### Synthesis of N-(4′-(N-methylsulfamoyl)-5-(1H-pyrazol-4-yl)biphenyl-2-yl)-2,3-dihydrobenzo[b][1,4]dioxine-2-carboxamide

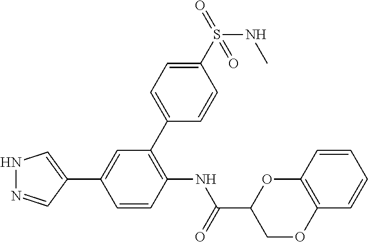

Procedures in Scheme 5 were used to prepare the title compound. LC-MS: single peak at 254 nm, MH⁺ calcd. for C₂₅H₂₂N₄O₅S: 491, obtained: 491.

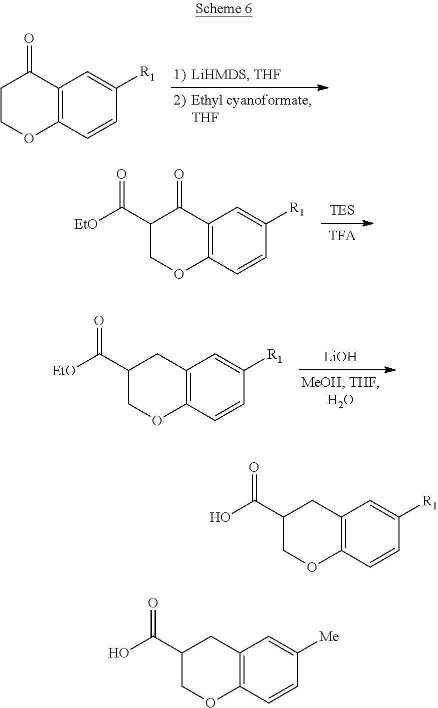

### Synthesis of 6-methyl-chroman-3-carboxylic acid

To a solution of 6-methyl-4-chromanone (2.5 g, 15.6 mmol) in THF (40 mL) at −78° C. was added lithium hexamethyldisilazide (1 M in THF, 17.1 mL, 17.1 mmol). After stirring at −78° C. for 30 min, a solution of ethyl cyanoformate (1.9 g, 19.0 mmol) in THF (5 mL) was added and the resulting mixture was stirred for an additional 30 min at −78° C. The reaction was warmed to room temperature, diluted with ether (150 mL), washed with a saturated aqueous NH₄Cl solution (100 mL) and brine (50 mL), dried over Na₂SO₄, filtered, and concentrated to dryness. The residue was purified by flash column chromatography (1-5% ethyl acetate in hexanes) to give ethyl 6-methyl-4-oxochromane-3-carboxylate as a mixture of tautomers (1.7 g, 47%).

To a solution of this β-ketoester in TFA (1 mL) was added triethylsilane (0.6 mL) and the mixture was stirred overnight at room temperature. The solvent was removed by rotary evaporation, and the residue was taken up in ethyl acetate (10 mL) and an aqueous saturated Na₂CO₃ solution (10 mL). The layers were separated and the aqueous phase was extracted with two portions of ethyl acetate (10 mL). The combined organic layers were washed with brine, dried over Na₂SO₄, filtered, and concentrated to dryness to afford the title compound. Without further purification, the crude material was dissolved in methanol (1 mL) and THF (1 mL). To this solution was added LiOH (37 mg, 1.6 mmol) in water (1 mL) and the reaction was stirred overnight at room temperature. The solution was concentrated, acidified with an aqueous 1 N HCl solution to pH 1, and extracted with ethyl acetate (3×5 mL). The combined organic layers were washed with brine, dried over Na₂SO₄, filtered, and concentrated to dryness to give the title compound (0.19 g, 84% over two steps). ¹H NMR (CDCl₃, 400 MHz) δ 6.91 (m, 2H), 6.73 (d, J=8.0 Hz, 1H), 4.42 (m, 1H), 4.18-4.13 (m, 1 H), 3.08-2.99 (m, 3H), 2.26 (s, 3H).

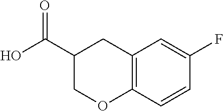

### Synthesis of 6-fluoro-chroman-3-carboxylic acid

The title compound was prepared from 6-fluoro-4-oxochromanone according to the procedure described in Scheme 6 (0.16 g, 44% over three steps). ¹H NMR (CDCl₃, 400 MHz) δ 7.00-6.75 (m, 3H), 4.40 (m, 1H), 4.19 (m, 1H), 3.13-3.01 (m, 3H).

### Example 151

### Synthesis of 6-methyl-chroman-3-carboxylic acid [4-(1H-pyrazol-4-yl)-phenyl]-amide

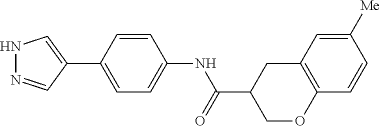

The title compound was prepared according to the procedure described in Scheme 1 (27 mg, 46% over three steps). ¹H NMR (DMSO-d₆, 400 MHz) δ 10.16 (s, 1H), 8.09 (br s, 1H), 7.89 (br s, 1H), 7.59 (d, J=8.8 Hz, 2H), 7.55 (d, J=8.8 Hz, 2H), 6.94 (s, 1H), 6.89 (d, J=8.4 Hz, 1H), 6.68 (d, J=8.0 Hz, 1H), 4.41 (d, J=11.2 Hz, 1H), 3.94 (d, J=10.2 Hz, 1H), 2.89 (t, J=11.2 Hz, 1H), 2.21 (s, 3H). LC-MS: single peak at 254 nm, MH⁺ calcd. for C₂₀H₂₀N₃O₂: 334, obtained: 334.

### Example 152

### Synthesis of 6-methyl-chroman-3-carboxylic acid [3-methoxy-4-(1H-pyrazol-4-yl)-phenyl]-amide

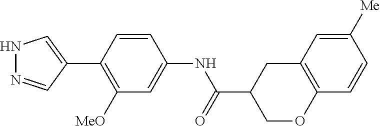

The title compound was prepared according to the procedure described in Scheme 1 (23 mg, 29% over three steps). ¹H NMR (DMSO-d₆, 400 MHz) δ 10.20 (s, 1H), 7.98 (br s, 2H), 7.54 (d, J=8.4 Hz, 1H), 7.49 (d, J=2.0 Hz, 1H), 7.16 (dd, J=8.2, 2.0 Hz, 1H), 6.94 (s, 1H), 6.89 (dd, J=10.2, 2.0 Hz, 1H), 6.68 (d, J=8.0 Hz, 1H), 4.41 (d, J=11.6 Hz, 1H), 3.95 (t, J=10.2 Hz, 1H), 3.84 (s, 3H), 3.05-2.97 (m, 2H), 2.90 (t, J=10.4 Hz, 1H), 2.21 (s, 3H). LC-MS: single peak at 254 nm, MH⁺ calcd. for C₂₁H₂₂N₃O₃: 364, obtained: 364.

### Example 153

### Synthesis of 6-fluoro-chroman-3-carboxylic acid [4-(1H-pyrazol-4-yl)-phenyl]-amide

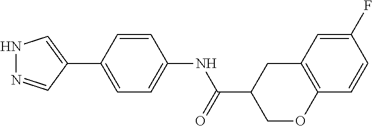

The title compound was prepared according to the procedure described in Scheme 1 (23 mg, 29% over three steps). LC-MS: single peak at 254 nm, MH⁺ calcd. for C₁₉H₁₇FN₃O₂: 338, obtained: 338. ¹H NMR (DMSO-d₆, 400 MHz) δ 10.17 (s, 1H), 8.09 (br s, 1H), 7.89 (br s, 1H), 7.59 (d, J=8.8 Hz, 2H), 7.55 (d, J=8.8 Hz, 2H), 7.03 (dd, J=9.2, 2.8 Hz, 1H), 6.93 (td, J=8.8, 3.2 Hz, 1H), 6.80 (dd, J=9.2, 4.8 Hz, 1H), 4.43 (dd, J=10.4, 3.2 Hz, 1H), 3.99 (m, 1 H), 3.08-2.93 (m, 3H).

### Example 154

### Synthesis of 6-Fluoro-chroman-3-carboxylic acid [3-methoxy-4-(1H-pyrazol-4-yl)-phenyl]-amide

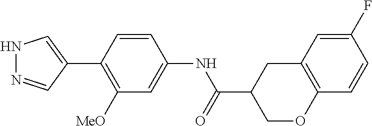

### Step A. 6-Fluoro-chroman-3-carboxylic acid [3-methoxy-4-(1H-pyrazol-4-yl)-phenyl]-amide

The title compound was prepared according to the procedure described in Scheme 1 (16 mg, 22% over three steps). LC-MS: single peak at 254 nm, MH⁺ calcd. for C₂₀H₁₉FN₃O₃: 368, obtained: 368. ¹H NMR (DMSO-d₆, 400 MHz) δ 10.22 (s, 1H), 7.99 (br s, 2H), 7.55 (d, J=8.4 Hz, 1H), 7.47 (d, J=2.0 Hz, 1H), 7.16 (dd, J=8.2, 1.8 Hz, 1H), 7.03 (dd, J=9.4, 3.0 Hz, 1H), 6.93 (td, J=8.8, 3.2 Hz, 1H), 6.80 (dd, J=8.8, 5.0 Hz, 1H), 4.43 (dd, J=10.8, 3.2 Hz, 1H), 4.01 (t, J=10.0 Hz, 1H), 3.84 (s, 3H), 3.06-2.94 (m, 3H).

### Example 155

### Synthesis of 6-Fluoro-chroman-3-carboxylic acid [2-methoxy-4-(1H-pyrazol-4-yl)-phenyl]-amide

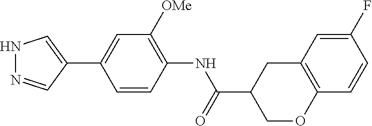

The title compound was prepared according to the procedure described in Scheme 1 (16 mg, 34% over three steps). LC-MS: single peak at 254 nm, MH⁺ calcd. for C₂₀H₁₉FN₃O₃: 368, obtained: 368. ¹H NMR (DMSO-d₆, 400 MHz) δ 9.41 (s, 1H), 8.06 (s, 2H), 7.88 (d, J=8.0 Hz, 1H), 7.26 (d, J=2.0 Hz, 1H), 7.15 (dd, J=8.0, 2.0 Hz, 1H), 7.02 (dd, J=9.2, 2.8 Hz, 1H), 6.92 (td, J=8.8, 2.8 Hz, 1H), 6.80 (dd, J=8.8, 4.8 Hz, 1H), 4.39 (m, 1H), 3.98 (t, J=10.2 Hz, 1H), 3.90 (s, 3H), 3.25-3.15 (m, 1H), 3.05-2.91 (m, 2H).

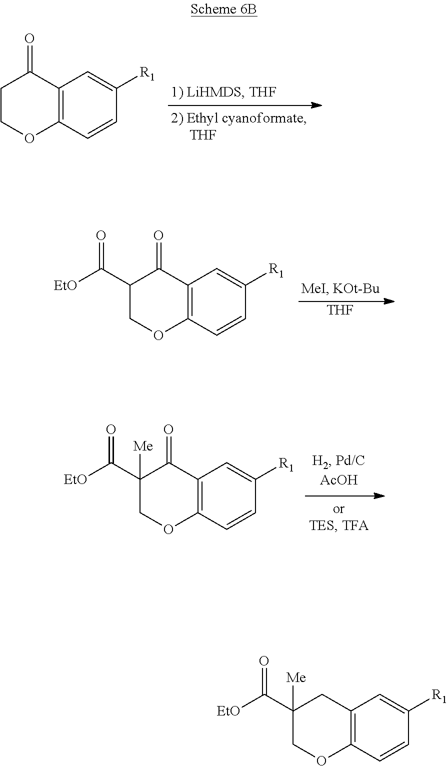

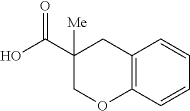

### Synthesis of 3-methyl-chroman-3-carboxylic acid

To a solution of chroman-4-one (2.0 g, 13.5 mmol) in THF (40 mL) at −78° C. was added lithium hexamethyldisilazide (1 M in THF, 14.9 mL, 14.9 mmol). After stirring at −78° C. for 30 min, a solution of ethyl cyanoformate (1.6 g, 16.0 mmol) in THF (5 mL) was added and the resulting mixture was stirred for an additional 30 min at −78° C. The reaction was warmed to room temperature, diluted with ether (150 mL), washed with a saturated aqueous NH₄Cl solution (100 mL) and brine (50 mL), dried over Na₂SO₄, filtered, and concentrated to dryness. The residue was purified by flash column chromatography (1-5% ethyl acetate in hexanes) to give ethyl 6-methyl-4-oxochromane-3-carboxylate as a mixture of tautomers. ¹H NMR (CDCl₃, 400 MHz) δ 12.03 (br s, 0.3H), 7.93 (dd, J=8.0, 1.6 Hz, 0.7H), 7.66 (dd, J=8.0, 1.6 Hz, 0.3H), 7.50 (m, 0.7H), 7.32 (m, 0.3H), 7.04 (m, 1H), 6.99 (d, J=8.0 Hz, 0.7H), 6.87 (dd, J=8.0, 1.2 Hz, 0.3H), 4.96 (s, 0.7H), 4.80 (dd, J=10.8, 8.4 Hz, 0.7H), 4.63 (dd, J=12.0, 4.4 Hz, 0.7H), 4.32-4.22 (m, 2H), 3.74 (dd, J=8.4, 4.8 Hz, 0.7H), 1.34 (t, J=7.2 Hz, 1H), 1.28 (t, J=7.2 Hz, 2H).

To a solution of ethyl 4-oxochromane-3-carboxylate (0.19 g, 0.92 mmol) in THF (5 mL) at 0° C. was added potassium t-butoxide (0.15 g, 1.38 mmol) and methyl iodide (0.14 mL, 0.46 mmol). The reaction was allowed to warm to room temperature overnight and diluted with ethyl acetate (20 mL) and a saturated aqueous NH₄Cl solution (10 mL). The layers were separated and the organic phase was washed with brine (10 mL), dried over Na₂SO₄, filtered, and concentrated to dryness. The residue was purified by flash column chromatography (2-5% ethyl acetate in hexanes) to give the α-methyl ester. ¹H NMR (CDCl₃, 400 MHz) δ 7.94 (dd, J=8.0, 1.6 Hz, 1H), 7.48 (ddd, J=8.4, 7.2, 1.6 Hz, 1H), 7.05 (ddd, J=8.8, 7.2, 0.8 Hz, 1H), 6.97 (dd, J=8.4, 0.8 Hz, 1H), 4.81 (d, J=11:6 Hz, 1H), 4.21-4.14 (m, 3H), 1.46 (s, 3H), 1.17 (t, J=7.2 Hz, 3H).

To a solution of ethyl 3-methyl-4-oxochromane-3-carboxylate in TFA (0.5 mL) was added triethylsilane (0.25 mL) and the reaction was stirred overnight at room temperature. After the solvent was removed, the residue was taken up in ethyl acetate (10 mL) and an aqueous saturated Na₂CO₃ solution (10 mL). The layers were separated and the aqueous phase was extracted with two portions of ethyl acetate (10 mL). The combined organic layers were washed with brine, dried over Na₂SO₄, filtered, and concentrated to dryness to give ethyl 3-methyl-chromane-3-carboxylate which was taken directly to the next reaction without further purification.

The crude ester was dissolved in ethanol (1.5 mL) and KOH (2 M aqueous solution, 3 equiv) was added. After stirring overnight, the reaction mixture was concentrated, treated with an aqueous 1 M HCl solution to pH 2, and extracted with ethyl acetate (3×5 mL). The combined organic layers were washed with brine (5 mL), dried over Na₂SO₄, filtered, and concentrated to dryness to give the title compound (57 mg, 37% over four steps). ¹H NMR (CDCl₃, 400 MHz) δ 7.11 (t, J=8.0 Hz, 1H), 7.07 (d, J=7.6 Hz, 1H), 6.88 (td, J=7.6, 1.2 Hz, 1H), 6.83 (dd, J=8.0, 1.2 Hz, 1H), 4.32 (dd, J=10.8, 1.2 Hz, 1H), 3.95 (dd, J=10.8, 1.2 Hz, 1H), 3.28 (d, J=16.0 Hz, 1H), 2.71 (d, J=16.4 Hz, 1H), 1.29 (s, 3H).

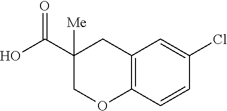

### Synthesis of 6-chloro-3-methyl-chroman-3-carboxylic acid

The title compound was prepared from 6-chloro-4-oxochromanone according to the procedure described above (0.22 g, 51% over four steps). ¹H NMR (CDCl₃, 400 MHz) δ 7.06 (m, 2H), 6.76 (d, J=8.8 Hz, 1H), 4.31 (dd, J=10.8, 1.6 Hz, 1H), 3.92 (dd, J=10.8, 0.8 Hz, 1H), 3.23 (d, J=16.4 Hz, 1H), 2.66 (d, J=16.4 Hz, 1H), 1.33 (s, 3H).

### Example 156

### Synthesis of 3-methyl-chroman-3-carboxylic acid [4-(1H-pyrazol-4-yl)-phenyl]-amide

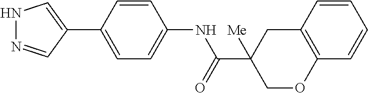

The title compound was prepared according to the procedure described in Scheme 1 (34 mg, 26% over three steps). ¹H NMR (DMSO-d₆, 400 MHz) δ 9.52 (s, 1H), 7.99 (br s, 2H), 7.60 (d, J=8.8 Hz, 2H), 7.53 (d, J=8.0 Hz, 2H), 7.11 (d, J=7.6 Hz, 1H), 7.07 (t, J=7.6 Hz, 1H), 6.86 (t, J=7.6 Hz, 1H), 6.77 (d, J=8.0 Hz, 1H), 4.39 (d, J=11.2 Hz, 1H), 4.06 (d, J=10.8 Hz, 1H), 3.29 (d, J=16.4 Hz, 1H), 2.78 (d, J=16.4 Hz, 1H), 1.30 (s, 3H). LC-MS: single peak at 254 nm, MH⁺ calcd. for C₂₀H₂₀N₃O₂: 334, obtained: 334.

### General Procedures:

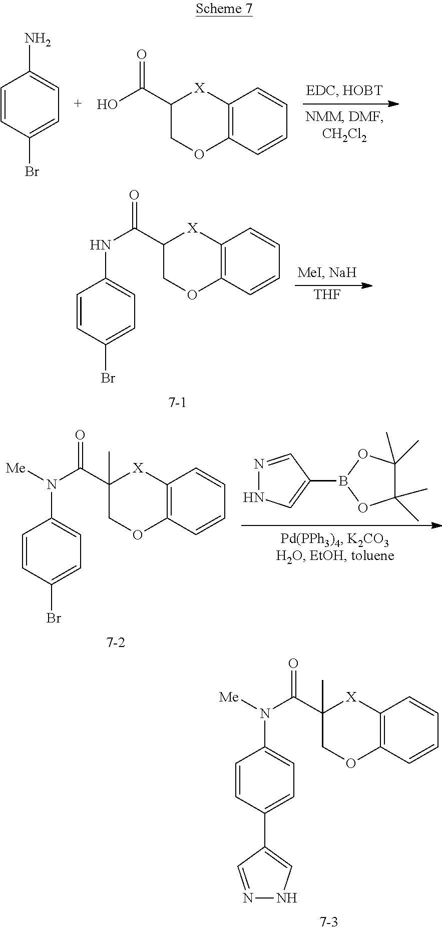

To a solution of the carboxylic acid (1 equiv) and 4-bromoaniline (1 equiv) in methylene chloride (1 mL) was added HOBT (1.1 equiv), EDC (1.1 equiv), and N-methylmorpholine (2.2 equiv). The resulting mixture was stirred at room temperature overnight. After removal of solvent by rotary evaporation, the residue was purified by flash column chromatography (2-25% ethyl acetate in hexanes) to give amide 7-1.

To a solution of this amide (118 mg, 0.352 mmol) in THF (5 mL) at 0° C. was added NaH (60% oil dispersion, 42 mg, 1.1 mmol). After stirring for 15 min, methyl iodide (0.1 mL) was added and the resulting mixture was stirred for 2 h. The reaction was diluted with a saturated aqueous NH₄Cl solution (10 mL) and extracted with ethyl acetate (3×10 mL). The combined organic layers were washed with brine (15 mL), dried over Na₃SO₄, filtered, and concentrated to dryness. The residue was purified by flash column chromatography (5-16% ethyl acetate in hexanes) to afford methyl amide 7-2.

To a solution of this methyl amide in a 3:2 mixture of ethanol:toluene was added 4-(4,4,5,5,-tetramethyl-1,3,2-dioxaborolan-2-yl)-1H-pyrazole (1.5 equiv), K₂CO₃ (2 M aqueous solution, 3 equiv), and tetrakis(triphenylphosphine)palladium (0) (0.05 equiv). The reaction mixture was degassed, purged with argon, and heated to 140° C. for 45 min by microwave irradiation. After cooling to room temperature, the mixture was treated with an aqueous 10% solution of TFA (1 mL) and the solvent was removed by rotary evaporation. The residue was purified by preparative HPLC to afford the desired final product.

### Example 157

### Synthesis of 2,3-dihydro-benzo[1,4]dioxine-2-carboxylic acid methyl-[4-(1H-pyrazol-4-yl)-phenyl]-amide

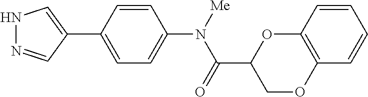

The title compound was prepared according to the procedure described in Scheme 7 (23 mg, 10% over three steps). ¹H NMR (DMSO-d₆, 400 MHz) δ 8.10 (s, 2H), 7.70 (d, J=8.8 Hz, 2 H), 7.45 (d, J=7.2 Hz, 2H), 6.79 (br s, 4H), 4.66 (m, 1H), 4.31 (d, J=12.0 Hz, 1H), 4.09 (m, 1H), 3.21 (s, 3H). LC-MS: single peak at 254 nm, MH⁺ calcd. for C₁₉H₁₈N₃O₃: 336, obtained: 336.

### Example 158

### Synthesis of chroman-3-carboxylic acid methyl-[4-(1H-pyrazol-4-yl)-phenyl]-amide

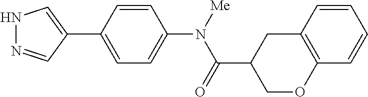

The title compound was prepared according to the procedure described in Scheme 7 (21 mg, 19% over three steps). LC-MS: single peak at 254 nm, MH⁺ calcd. for C₂₀H₂₀N₃O₂: 334, obtained: 334. ¹H NMR (DMSO-d₆, 400 MHz) δ 8.07 (br s, 2H), 7.68 (d, J=8.4 Hz, 2H), 7.43 (d, J=8.0 Hz, 2H), 7.01 (t, J=8.0 Hz, 2H), 6.77 (t, J=7.6 hz, 1H), 6.65 (t, J=7.6 Hz, 1H), 4.28 (d, J=10.4 Hz, 1H), 3.80 (t, J=10.4 Hz, 1H), 3.20 (s, 3H), 2.98 (dd, J=14.8, 10.4 Hz, 1H), 2.77 (m, 1H), 2.70-2.65 (m, 1H).

### Example 159

### Synthesis of 2-methyl-2,3-dihydro-benzo[1,4]dioxine-2-carboxylic acid methyl-[4-(1H-pyrazol-4-yl)-phenyl]-amide

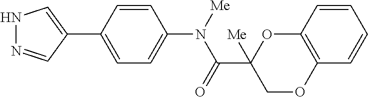

The title compound was prepared according to the procedure described in Scheme 7 except the methylation step was performed at room temperature (17 mg, 15% over three steps). LC-MS: single peak at 254 nm, MH⁺ calcd. for C₂₀H₂₀N₃O₃: 350, obtained: 350. NMR (CDCl₃, 400 MHz) δ 7.87 (s, 2H), 7.50 (d, J=8.8 Hz, 2H), 7.24 (d, J=8.8 Hz, 2H), 7.03 (td, J=7.2, 1.6 Hz, 1H), 6.84-6.74 (m, 2H), 6.47 (m, 1H), 4.99 (d, J=2.4 Hz, 1H), 4.34 (d, J=2.4 Hz, 1H), 3.67 (s, 3H), 3.38 (s, 3H).

### General Procedures:

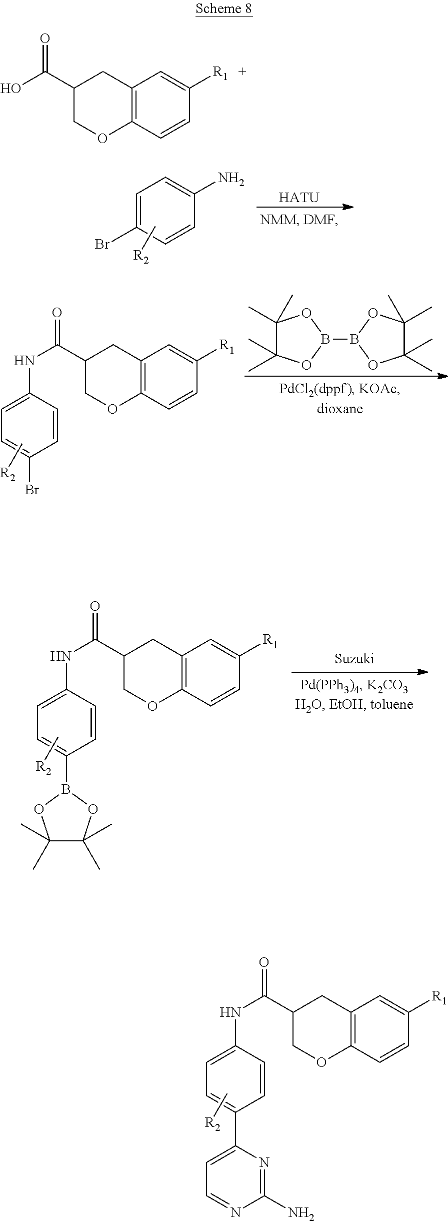

To a solution of the carboxylic acid (1 equiv) and the bromoaniline (1 equiv) in DMF (0.3 mL) was added HATU (1.2 equiv) and N-methylmorpholine (2 equiv). The resulting mixture was stirred at room temperature overnight. After removal of solvent by rotary evaporation, the residue was dissolved in ethyl acetate (15 mL) and washed with an aqueous 1 N HCl solution (2×10 mL), a saturated aqueous Na₂CO₃ solution (2×10 mL), and brine (20 mL), dried over Na₂SO₄, and filtered. The solvent was removed in vacuo and the crude amide product was taken to the next reaction without further purification.

The residue was dissolved in dioxane and to this solution was added bis(pinacolato)diboron (2.5 equiv), potassium acetate (5 equiv), and PdCl₂(dppf) (0.1 equiv). The reaction mixture was degassed, purged with argon, and heated to 100° C. for 75 min by microwave irradiation. After the reaction was determined to be complete by LC-MS, the mixture was diluted with ethyl acetate (20 mL) and washed with brine (10 mL). The organic layer was dried over Na₂SO₄, filtered, and concentrated in vacuo to yield the crude aryl boronic ester. The residue was dissolved in a 3:2 mixture of ethanol:toluene and to this mixture was added 2-amino-4-chloro-pyrimidine (1.2 equiv), K₂CO₃ (2 M aqueous solution, 3 equiv), and tetrakis(triphenylphosphine)palladium (0) (0.05 equiv). The reaction mixture was degassed, purged with argon, and heated to 140° C. for 45 min by microwave irradiation.

After cooling to room temperature, the mixture was treated with an aqueous 10% solution of TFA (1 mL) and the solvent was removed by rotary evaporation. The residue was purified by preparative HPLC to afford the desired final product.

### Example 160

### Synthesis of 6-methoxy-chroman-3-carboxylic acid [4-(2-amino-pyrimidin-4-yl)-3,5-difluoro-phenyl]-amide

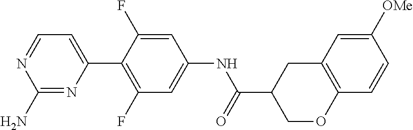

The title compound was prepared according to the procedure described in Scheme 8 (8 mg, 12% over three steps). ¹H NMR (DMSO-d₆, 400 MHz) δ 10.61 (s, 1H), 8.26 (d, J=4.8 Hz, 1H), 7.37 (d, J=10.4 Hz, 2H), 6.72 (br s, 2H), 6.68-6.60 (m, 4H), 4.33 (d, J=12.0 Hz, 1H), 3.90 (t, J=10.0 Hz, 1H), 3.62 (s, 3H), 3.00-2.86 (m, 3H). LC-MS: single peak at 254 nm, MH⁺ calcd. for C₂₁H₀F₂N₄O₃: 413, obtained: 413.

### Example 161

### Synthesis of 6-methoxy-chroman-3-carboxylic acid [4-(2-amino-pyrimidin-4-yl)-3-methoxy phenyl]-amide

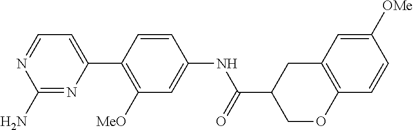

The title compound was prepared according to the procedure described in Scheme 8 (12 mg, 15% over three steps). ¹H NMR (DMSO-d₆, 400 MHz) δ 10.48 (s, 1H), 8.26 (d, J=6.0 Hz, 1H), 7.93 (d, J=8.8 Hz, 1H), 7.60 (d, J=2.0 Hz, 1H), 7.31 (dd, J=8.8, 2.0 Hz, 2H), 7.40-7.25 (br s, 2H), 6.74-6.67 (m, 3H), 4.40 (d, J=10.8 Hz, 1H), 3.98 (t, J=10.4 Hz, 1H), 3.87 (s, 3H), 3.69 (s, 3H), 3.06-2.92 (m, 3H). LC-MS: single peak at 254 nm, MH⁺ calcd. for C₂₂H₂₃N₄O₄: 407, obtained: 407.

### Example 162

### Synthesis of 6-methoxy-chroman-3-carboxylic acid [4-2-amino-pyrimidin-4-yl)-2-(2-dimethylamino-ethoxy)-phenyl]-amide

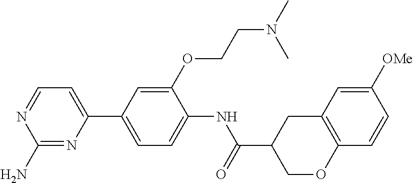

The title compound was prepared according to the procedure described in Scheme 8 (24 mg, 56% over three steps). ¹H NMR (DMSO-d₆, 400 MHz) δ 9.73 (br s, 1H), 9.44 (s, 1H), 8.36 (d, J=5.6 Hz, 1H), 8.16 (d, J=9.2 Hz, 1H), 7.80 (m, 2H), 7.30 (d, J=5.6 Hz, 1H), 7.11 (br s, 2H), 6.75-6.68 (m, 3H), 4.50 (t, J=4.8 Hz, 2H), 4.42 (ddd, J=12.4, 3.2, 2.0 Hz, 1H), 3.98 (t, J=10.2 Hz, 1H), 3.69 (s, 3H), 3.63 (m, 2H), 3.28-3.22 (m, 1H), 3.06 (dd, J=16.0, 10.0 Hz, 1H), 2.93 (m, 7H). LC-MS: single peak at 254 nm, MH⁺ calcd. for C₂₅H₃₀N₅O₄: 464, obtained: 464.

### Example 163

### Synthesis of 6-methyl-chroman-3-carboxylic acid [4-(2-amino-pyrimidin-4-yl)-2-(2-dimethylamino-ethoxy)-phenyl]-amide

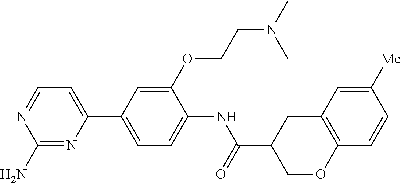

The title compound was prepared according to the procedure described in Scheme 8 (18 mg, 18% over three steps). ¹H NMR (DMSO-d₆, 400 MHz) δ 9.55 (br s, 1H), 9.41 (s, 1H), 8.33 (d, J=5.2 Hz, 1H), 8.13 (d, J=8.8 Hz, 1H), 7.77 (m, 2H), 7.21 (d, J=5.2 Hz, 1H), 6.96 (s, 1H), 6.91 (d, J=8.8, 1H), 6.79 (br s, 2H), 6.69 (d, J=8.4 Hz, 1H), 4.49 (t, J=4.8 Hz, 1H), 4.40 (m, 1H), 3.99 (t, J=10.4 Hz, 1H), 3.62 (m, 2H), 3.03 (dd, J=16.8, 10.4 Hz, 1H), 2.94 (m, 7H), 2.21 (s, 1H). LC-MS: single peak at 254 nm, MH⁺ calcd. for C₂₅H₃₀N₅O₃: 448, obtained: 448.

### Example 164

### Synthesis of 6-fluoro-chroman-3-carboxylic acid [4-(2-amino-pyrimidin-4-yl)-2-(2-dimethylamino-ethoxy)-phenyl]-amide

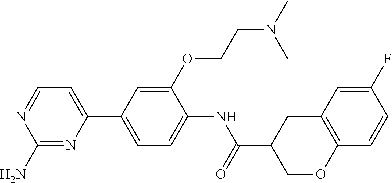

The title compound was prepared according to the procedure described in Scheme 8 (33 mg, 26% over three steps). ¹H NMR (DMSO-d₆, 400 MHz) δ 9.56 (s, 1H), 9.42 (s, 1H), 8.34 (d, J=5.2 Hz, 1H), 8.12 (d, J=8.8 Hz, 1H), 7.79 (m, 2H), 7.24 (d, J=5.6 Hz, 1H), 7.04 (dd, J=8.8, 2.8 Hz, 1H), 6.95 (td, J=8.4, 2.8, 1H), 6.89 (br s, 2H), 6.82 (dd, J=8.8, 4.8 Hz, 1H), 4.51-4.45 (m, 3H), 4.04 (t, J=10.0 Hz, 1H), 3.62 (m, 2H), 3.26-3.23 (m, 1H), 3.08 (dd, J=16.8, 10.0 Hz, 1H), 2.99 (d, J=5.2 Hz, 1H), 2.95 (s, 3H), 2.94 (s, 3H). LC-MS: single peak at 254 nm, MH⁺ calcd. for C₂₄H₂₇FN₅O₃: 452, obtained: 452.

### Example 165

### Synthesis of chroman-3-carboxylic acid [4-(2-amino-pyrimidin-4-yl)-2-(2-dimethylamino-ethoxy)-phenyl]-amide

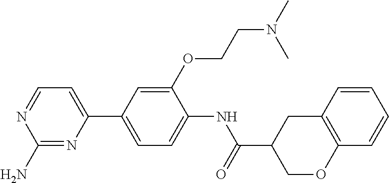

The title compound was prepared according to the procedure described in Scheme 8 (6 mg, 5% over three steps). ¹H NMR (DMSO-d₆, 400 MHz) δ 9.54 (br s, 1H), 9.40 (s, 1H), 8.32 (d, J=5.2 Hz, 1H), 8.12 (d, J=9.2 Hz, 1H), 7.76 (d, J=8.8 Hz, 1H), 7.19 (d, J=5.2 Hz, 1H), 7.16 (d, J=8.4 Hz, 1H), 7.11 (t, J=7.6 Hz, 1H), 6.88 (td, J=7.2, 1.2, 1H), 6.81 (d, J=8.0 Hz, 1H), 6.70 (br s, 2H), 4.51-4.46 (m, 3H), 4.04 (t, J=10.0 Hz, 1H), 3.62 (m, 2H), 3.28-3.23 (m, 1 H), 3.07 (dd, J=15.6, 10.0 Hz, 1H), 2.99-2.94 (m, 1H), 2.95 (s, 3H), 2.94 (s, 3H). LC-MS: single peak at 254 nm, MH⁺ calcd. for C₂₄H₂₈N₅O₃: 434, obtained: 434.

### Example 166

### Synthesis of 6-chloro-chloro-chroman-3-carboxylic acid [4-(2-amino-pyrimidin-4-yl)-2-(2-dimethylamino-ethoxy)-phenyl]-amide

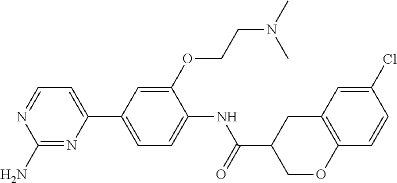

The title compound was prepared according to the procedure described in Scheme 8 (20 mg, 17% over three steps). NMR (DMSO-d₆, 400 MHz) δ 9.53 (br s, 1H), 9.42 (s, 1H), 8.33 (d, J=5.2 Hz, 1H), 8.11 (d, J=8.8 Hz, 1H), 7.76 (m, 2H), 7.26 (d, J=2.8 Hz, 1H), 7.21 (d, J=5.6 Hz, 1H), 7.15 (dd, J=8.8, 2.8 Hz, 1H), 6.83 (d, J=8.4, 1H), 6.78 (br s, 2H), 4.50-4.47 (m, 3H), 4.06 (t, J=10.4 Hz, 1H), 3.62 (m, 2H), 3.28-3.23 (m, 1H), 3.09-2.98 (m, 2H), 2.95 (s, 3H), 2.94 (s, 3H). LC-MS: single peak at 254 nm, MH⁺ calcd. for C₂₄H₂₇ClN₅O₃: 469, obtained: 469.

### Example 167

### Synthesis of (S)-2,3-dihydro-benzo[1,4]dioxine-2-carboxylic acid [4-(2-amino-pyrimidin-4-yl)-2-(2-dimethylamino-ethoxy)-phenyl]-amide

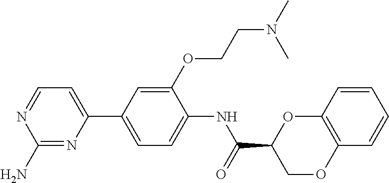

The title compound was prepared according to the procedure described in Scheme 8 (0.097 g, 18% over three steps). ¹H NMR (DMSO-d₆, 400 MHz) δ 9.71 (br s, 1H), 9.35 (s, 1H), 8.36 (d, J=5.6 Hz, 1H), 8.16 (d, J=8.0 Hz, 1H), 7.81-7.79 (m, 2H), 7.25 (d, J=5.6 Hz, 1H), 7.10 (dd, J=8.0, 1.6 Hz, 1H), 6.97-6.88 (m, 4H), 5.16 (dd, J=5.6, 2.8 Hz, 1H), 4.56-4.46 (m, 3H), 4.41 (dd, J=11.6, 5.6 Hz, 1H), 3.58 (m, 2H), 2.93 (t, J=4.8 Hz, 6H). LC-MS: single peak at 254 nm, MH⁺ calcd. for C₂₃H₂₆N₅O₄: 436, obtained: 436.

### Example 168

### The synthesis of N-(4-(2-aminopyrimidin-4-yl)-2-2-dimethylamino)ethoxy)-5-fluorophenyl)-2,3-dihydrobenzo[b][1,4]dioxine-2-carboxamide

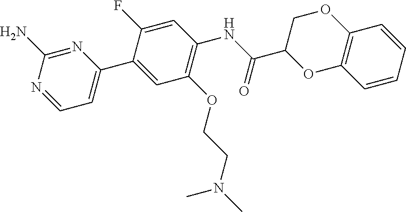

The title compound was prepared according to the procedure described in Scheme 8. ¹H-NMR (DMSO-d₆, 400 MHz), δ: 10.17 (br, 1H), 9.55 (s, 1H), 8.44-8.41 (m, 1H), 8.15-8.12 (m, 1H), 7.73-7.71 (m, 1H), 7.16-6.96 (m, 6H), 5.27-5.25 (m, 1H), 4.54-4.48 (m, 4H), 3.71-3.59 (m, 2H), 3.02 (s, 6H); LC/MS: C₂₃H₂₄FN₅O₄ (M+1) 454.13.

### Example 169

### N-(2-(2-(dimethylamino)ethoxy)-5-fluoro-4-(1H-pyrrolo[2,3-b]pyridin-4-yl)phenyl)-2,3-dihydrobenzo[b][1,4]dioxine-2-carboxamide

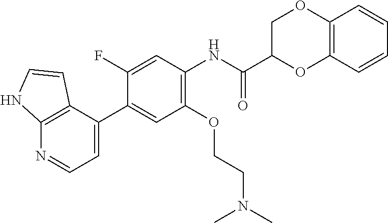

Similar procedures as in Scheme 8 were utilized to synthesize this compound. ¹H-NMR (DMSO-d₆, 400 MHz), δ: 11.90 (br, 1H), 10.01 (br, 1H), 9.55 (s, 1H), 8.33-8.32 (m, 1H), 8.12-8.09 (m, 1H), 7.57-7.55 (m, 1H), 7.36-7.35 (m, 1H), 7.19-7.17 (m, 2H), 6.97-6.92 (m, 3H), 5.27-5.25 (m, 1H), 3.58-3.52 (m, 2H), 2.95 (s, 6H); LC/MS: C₂₆H₂₅FN₄O₄ (M+1) 477.14.

### Example 170

### N-(2-(2-(dimethylamino)ethoxy)-5-fluoro-4-(3H-imidazo[4,5-b]pyridin-7-yl)phenyl)-2,3-dihydrobenzo[b][1,4]dioxine-2-carboxamide

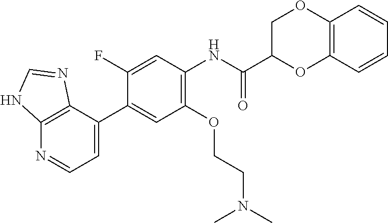

Similar procedures as in Scheme 8 were utilized to synthesize this compound. ¹H-NMR (DMSO-d₆, 400 MHz), δ: 9.89 (br, 1H), 9.45 (s, 1H), 8.82 (s, 1H), 8.17-8.06 (m, 2H), 7.77-7.69 (m, 2H), 7.13-6.92 (m, 4H), 5.22-5.20 (m, 1H), 4.51-4.44 (m, 4H), 3.58-3.53 (m, 2H), 2.94 (s, 6H); LC/MS: C₂₅H₂₄FN₅O₄ (M+1) 478.12.

### Example 171

### N-(2-(2-(dimethylamino)ethoxy)-5-fluoro-4-(1H-imidazol-4-yl)phenyl)-2,3-dihydrobenzo[b][1,4]dioxine-2-carboxamide

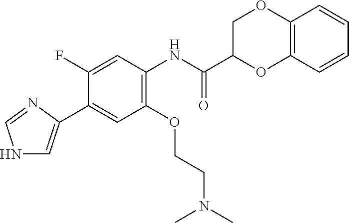

Similar procedures as in Scheme 8 were utilized to synthesize this compound. ¹H-NMR

(DMSO-d₆, 400 MHz), δ: 10.03 (br, 1H), 9.53 (m, 1H), 8.70 (br, 1H), 8.06-8.04 (m, 1H), 7.80-7.67 (m, 2H), 7.09-6.90 (m, 4H), 5.25-5.23 (m, 1H), 4.49-4.39 (m, 4H), 2.92 (s, 6H); LC/MS:

C₂₂H₂₃FN₄O₄ (M+1) 427.10.

### Example 172

### N-(2-(2-(dimethylamino)ethoxy)-5-fluoro-4-(2-methyl-1H-imidazol-4-yl)phenyl)-2,3-dihydrobenzo[b][1,4]dioxine-2-carboxamide

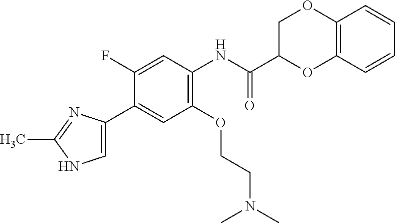

Similar procedures as in Scheme 8 were utilized to synthesize this compound. ¹H-NMR (DMSO-d₆, 400 MHz), δ: 10.07 (br, 1H), 9.46 (s, 1H), 8.10-8.07 (m, 1H), 7.84 (s, 1H), 7.60-7.58 (m, 1H), 7.10-6.91 (m, 4H), 5.20-5.18 (m, 1H), 4.49-4.40 (m, 4H), 3.64-3.52 (m, 2H), 2.90 (s, 6H), 2.63 (s, 3H); LC/MS: C₂₃H₂₅FN₄O₄ (M+1) 441.12.

### Example 173

### N-(2-(2-(dimethylamino)ethoxy)-5-fluoro-4-(7H-pyrrolo[2,3-d]pyrimidin-4-yl)phenyl)-2,3-dihydrobenzo[b][1,4]dioxine-2-carboxamide

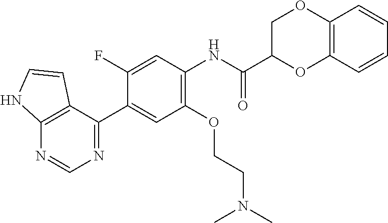

Similar procedures as in Scheme 8 were utilized to synthesize this compound. ¹H-NMR (DMSO-d₆, 400 MHz), δ: 12.23 (br, 1H), 9.77 (br, 1H), 9.49 (s, 1H), 8.89 (s, 1H), 8.17-8.14 (m, 1H), 7.66-7.53 (m, 2H), 7.13-6.92 (m, 4H), 5.24-5.22 (m, 1H), 4.51-4.45 (m, 4H), 3.55 (m, 2H), 2.85 (s, 6H); LC/MS: C₂₅H₂₄FN₅O₄ (M+1) 478.16.

### Example 174

### (+)—N-(5-methoxy-6-(1H-pyrazol-4-yl)pyridin-3-yl)chroman-3-carboxamide

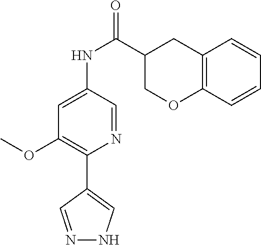

Following the procedures in Scheme 8, this product was obtained using 6-chloro-5-methoxypyridine-3-amine and chroman-3-carboxylic acid in the amide coupling step and 4-(4,4,5,5-tetramethyl-1,3,2-dioxaborolan-2-yl)-1H-pyrazole as the boronic ester coupling partner in the Suzuki heteroarylation step. Data for this compound: MS: MH⁺ calcd. for C₁₉H₁₉N₄O₃⁺: 351.1, obtained: 351.1.

### Example 175

### (+)—N-(5-methoxy-6-(1H-pyrazol-4-yl)pyridin-3-yl)-2,3-dihydrobenzo[b][1,4]dioxine-2-carboxamide

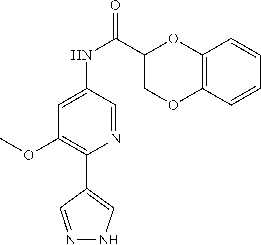

Following the procedures in Scheme 8, this product was obtained using 6-chloro-5-methoxypyridine-3-amine and 2,3-dihydrobenzo[b][1,4]dioxine-2-carboxylic acid in the amide coupling step and 4-(4,4,5,5-tetramethyl-1,3,2-dioxaborolan-2-yl)-1H-pyrazole as the boronic ester coupling partner in the Suzuki heteroarylation step. Data for this compound: MS: MH⁺ calcd. for C₁₈H₁₇N₄O₄⁺: 353.1, obtained: 353.1.

### General Procedures:

Examples 176-180 were made following the procedures in Scheme 9.

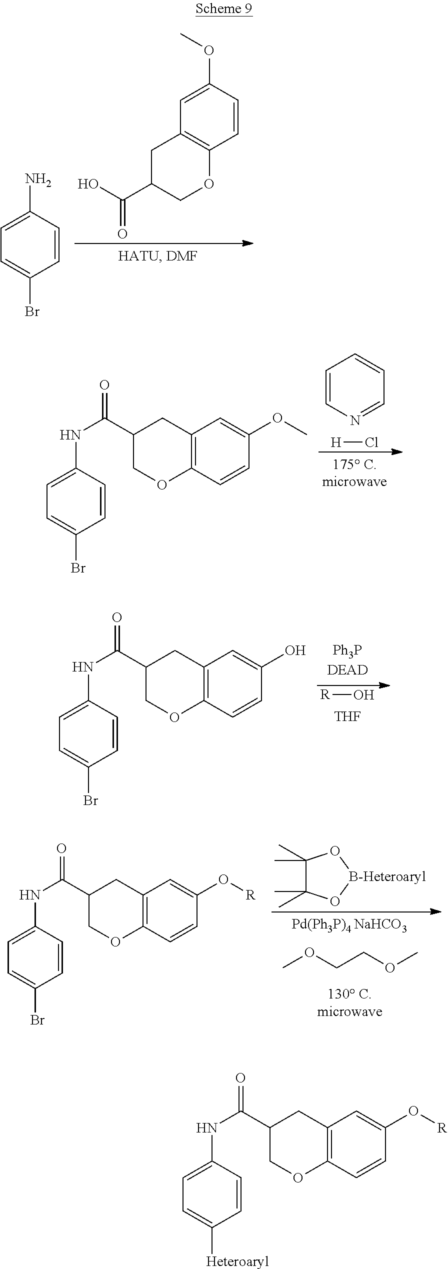

A mixture of 6-methoxychroman-3-carboxylic acid (700 mg, 3.36 mmol), 4-bromoaniline (694 mg, 1.2 equiv), HATU (1.92 g, 1.5 equiv.), diisopropylethylamine (1.76 mL, 3 equiv) in 5 mL of DMF was maintained at 25° C. overnight. Extractive workup with water/ethyl acetate following by drying of the organic layer over sodium sulfate gave the crude amide. This amide was purified on silica gel using a gradient elution of 0 to 50% ethyl acetate in hexane to obtain the pure product (956 mg, 78% yield).

### Procedure for the Demethylation:

The powdered amide (724 mg, 2.0 mmol) was placed in a 5 mL microwave tube equipped with a stir bar. Solid pyridinium hydrochloride (1.16 g, 5 equiv.) was added. This neat mixture of solids was heated in a microwave reactor to 175 degrees for 20 minutes using 300 W of microwave energy. The cooled melt was dissolved in a minimal amount of chloroform. The organic solution was washed with water, dried over sodium sulfate, concentrated to an oil, and was purified on silica gel using a gradient elution of 0 to 50% ethyl acetate in hexane to obtain the pure product (415 mg, 60% yield).

### General Procedure for the Misunobu Etherifications:

The phenol (0.3-0.4 mmol) was dissolved in dry THF. Added to the solution, in succession, was 1.5 equivalents of diethylazodicarboxylate (40 wt % solution in toluene), 1.5 equivalents of triphenylphosphine, and 1.5 equivalents of an appropriate alcohol. For alcohols with boiling point below 100° C., 10 equivalents were used. The solution was maintained under Argon at room temperature for 48 hrs., concentrated in vacuo, and purified on silica gel using a gradient elution of 0 to 100% ethyl acetate in hexane followed by elution with a 9:1:0.1 mixture of dichloromethane: methanol: ammonia to obtain the product. The product was further purified a second silica gel column using an isocratic elution of an appropriate solvent mixture. The products were obtained in 25-65% yield.

### General Procedure for the Suzuki Heteroarylation:

A mixture of the bromide (1 equiv), heteroaryl boronic ester (1.2 equiv), tetrakistriphenylphosphine palladium (0.03 equiv), and sodium bicarbonate (3.4 equiv) was suspended in a 2:1 mixture of dimethoxyethane and water (3 mL total, when 0.2 mmol of bromide was used) in a microwave pressure vessel. The sealed vessel was heated for 20 minutes at 130° C. using 300 W of microwave energy. The solution was cooled, poured into water, and extracted with chloroform. The organic extracts were died, filtered, concentrated in vacuo, then the residue was dissolved in a minimal amount of chloroform and this solution was purified on silica gel using a gradient elution of dichloromethane to 9:1:0.1 mixture of dichloromethane:methanol:ammonia to obtain the product in 35-70% yield. The product in each case was characterized by LC-MS spectroscopy.

### General LC-MS Analytical Method for Examples 177-181:

Analytical LC-MS was obtained using an Agilent 1200 HPLC with a 6140 quadrupole MS. The HPLC column used was an Agilent XDB-C18 column, 50×4.6 mm with 1.8 micron packing. The general method was a gradient from 3% acetonitrile in water to 100% acetonitrile over 3.5 minutes using a flow rate of 1.5 ml/min. Mobile phases were acidified with 0.1% formic acid, and ions were scanned in positive mode using chemical ionization. Retention times are given in minutes, and the method will be described as Agilent LC-MS general method 1.

### Example 176

### (+)—N-(4-(1H-pyrazol-4-yl)phenyl)-6-(2-(dimethylamino)ethoxy)chroman-3-carboxamide

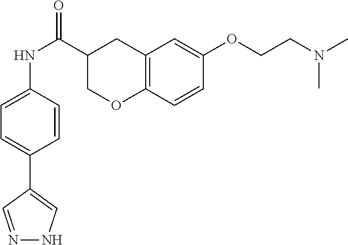

Following the procedures in Scheme 9, this product was obtained using 2-(dimethylamino)ethanol as the alcohol in the Mitsunobu etherification step and 4-(4,4,5,5-tetramethyl-1,3,2-dioxaborolan-2-yl)-1H-pyrazole as the boronic ester coupling partner in the Suzuki heteroarylation step. Data for this compound: LC-MS: single peak at 254 nm, retention time 1.91 minutes using Agilent LC-MS general method 1, MH⁺ calcd. for C₂₃H₂₇N₄O₃⁺: 407.2, obtained: 407.2.

### Example 177

### (+)—N-(4-(1H-pyrazol-4-yl)phenyl)-6-(3-(dimethylamino)propoxy)chroman-3-carboxamide

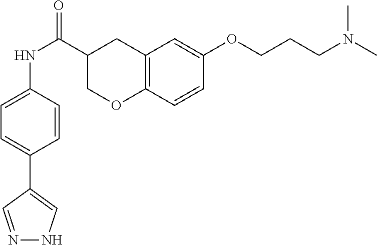

Following the procedures in Scheme 9, this product was obtained using 3-(dimethylamino)propanol as the alcohol in the Mitsunobu etherification step and 4-(4,4,5,5-tetramethyl-1,3,2-dioxaborolan-2-yl)-1H-pyrazole as the boronic ester coupling partner in the Suzuki heteroarylation step. Data for this compound: LC-MS: single peak at 254 nm, retention time 1.97 minutes using Agilent LC-MS general method 1, MH⁺ calcd. for C₂₄H₂₉N₄O₃⁺: 421.2, obtained: 421.2.

### Example 178

### (+)—N-(4-(1H-pyrazol-4-yl)phenyl)-6-isopropoxychroman-3-carboxamide

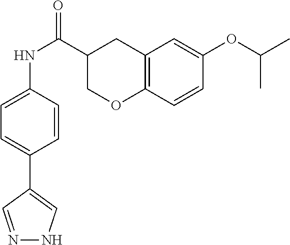

Following the procedures in Scheme 9, this product was obtained using isopropanol as the alcohol in the Mitsunobu etherification step and 4-(4,4,5,5-tetramethyl-1,3,2-dioxaborolan-2-yl)-1H-pyrazole as the boronic ester coupling partner in the Suzuki heteroarylation step. Data for this compound: LC-MS: single peak at 254 nm, retention time 2.96 minutes using Agilent LC-MS general method 1, MH⁺ calcd. for C₂₂H₂₄N₃O₃⁺: 378.2, obtained: 378.2.

### Example 179

### (+)—N-(4-(1H-pyrazol-4-yl)phenyl)-6-(1-methylpiperidin-4-yloxy)chroman-3-carboxamide

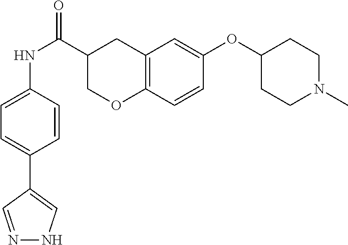

Following the procedures in Scheme 9, this product was obtained using N-methyl-4-hydroxypiperidine as the alcohol in the Mitsunobu etherification step and 4-(4,4,5,5-tetramethyl-1,3,2-dioxaborolan-2-yl)-1H-pyrazole as the boronic ester coupling partner in the Suzuki heteroarylation step. Data for this compound: LC-MS: single peak at 254 nm, retention time 1.98 minutes using Agilent LC-MS general method 1, MH⁺ calcd. for C₂₅H₂₉N₄O₃⁺: 433.2, obtained: 433.2.

### Example 180

### (+)—N-(4-(1H-pyrazol-4-yl)phenyl)-6-ethoxychroman-3-carboxamide

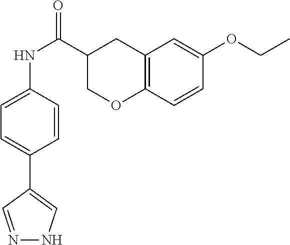

Following the procedures in Scheme 9, this product was obtained using ethanol as the alcohol in the Mitsunobu etherification step and 4-(4,4,5,5-tetramethyl-1,3,2-dioxaborolan-2-yl)-1H-pyrazole as the boronic ester coupling partner in the Suzuki heteroarylation step. Data for this compound: LC-MS: single peak at 254 nm, retention time 2.94 minutes using Agilent LC-MS general method 1, MH⁺ calcd. for C₂₁H₂₂N₃O₃⁺: 364.17, obtained: 364.1.

### Example 181

### N-(4-(5-amino-1H-pyrazol-4-yl)phenyl)chroman-3-carboxamide

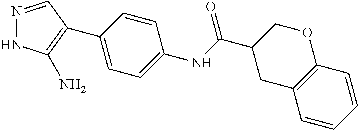

### Synthesis of 4-(4-nitrophenyl)-1H-pyrazol-5-amine

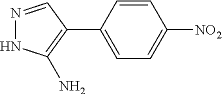

In a sealed microwave tube, 4-nitrobenzacetonitrile (2.00 g, 12.3 mmol) and N,N-dimethylformamide/dimethylacetamide (DMF/DMA, 1.80 mL, 1.1 eq) were heated in the microwave reactor for 30 minutes at 90 C. The reaction was dissolved in ethanol and 1 mL of hydrazine was added. The reaction was refluxed overnight. The solvent was removed in vacuo and the residue was taken up in 10% aqueous TFA and acetonitrile. Preparative HPLC (0.1% aqueous TFA: acetonitrile gradient) was used to produce the product as a yellow solid (932 mg, 37% yield). Single peak by HPLC. LCMS (found 205.1 MH+ calculated for C₉H₈N₄O₂: 205.1). ¹H-NMR (DMSO-d₆, 400 MHz) δ 7.73-7.79 (m, 2H), 8.04 (s, 1H), 8.15-8.20 (m, 2H). ¹³C-NMR (DMSO-d₆, 400 MHz) δ 105.7, 125.0, 126.6, 134.2, 141.6, 144.9, 149.0. Single peak by HPLC.

### Synthesis of tert-butyl 5-amino-4-(4-nitrophenyl)-1H-pyrazole-1-carboxylate

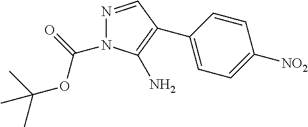

The above aminopyrazole (362 mg, 1.77 mmol) was dissolved in acetonitrile. To this solution was added Boc anhydride (852 mg, 2.2 eq), DIEA (676 ml, 2.2 eq), and a catalytic amount of 4-N,N-dimethylaminopyridine. The reaction was stirred overnight and the solvent was removed. The residue was taken up in DCM and purified by silica gel chromatography (12 g column, DCM:MeOH gradient) to give 175 mg (95% yield) of the desired product. ¹H-NMR (CD₃OD-d₄, 400 MHz) δ 1.64 (s, 9H), 7.78-7.83 (m, 2H), 8.26-8.30 (m, 3H). Single peak by HPLC.

### Synthesis of tert-butyl 5-amino-4-(4-aminophenyl)-1H-pyrazole-1-carboxylate

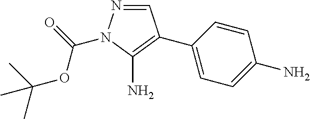

In a Parr bottle the above nitrobenzene was dissolved in 5 mL methanol along with a catalytic amount of 10% Pd/C. The vessel was placed under a 45-55 psi hydrogen atmosphere and shaken vigorously for 4 hours. HPLC indicated a complete reaction so the reaction was filtered through a syringe filter and concentrated to give 150 mg of the desired product (95% yield). ¹H-NMR (CD₃OD-d₄, 400 MHz) δ 1.60 (s, 9H), 6.74-6.79 (m, 2H), 7.21-7.26 (m, 2H), 7.85 (s, 1H). Single peak by HPLC.

The above aniline (56 mg, 0.204 mmol) was dissolved in 1.5 mL DMF along with 3-chromancarboxylic acid (36 mg, 1.0 eq), HATU (85 mg, 1.1 eq), and DIEA (39 uL, 1.1 eq). The reaction was stirred for 2 hours before it was dumped into 5 mL water. A white precipitate formed that was filtered off and washed with saturated sodium bicarbonate and distilled water. The precipitate was 97 mg (>100% yield due to presence of water) of the desired Boc-protected compound. ¹H-NMR (CDCl₃-d, 400 MHz) δ 1.65 (s, 9H), 2.90-3.10 (m, 2H), 3.22 (dd, 9.2 Hz, 16.4 Hz, 1H), 4.24 (dd, 8.4 Hz, 11.2 Hz, 1H), 4.40-4.46 (m, 1H), 6.83-6.92 (m, 2H), 7.06-7.16 (m, 2H), 7.40 (d, 8.8 Hz, 2H), 7.51 (d, 8.8 Hz, 2H), 7.68 (s, 1H), 7.88 (s, 1H). Single peak by HPLC. A portion of this compound was dissolved in 1 mL methanol and 1 mL 4M HCl in dioxane was added to remove the Boc protecting group. The reaction was monitored by HPLC. Upon completion (6 hours) the solvent was removed in vacuo. The residue was taken up in ethanol which was removed in vacuo (repeated twice) to eliminate any traces of acid or dioxane. The desired product was obtained as an off-white solid (32 mg). LCMS (found 335.2 MH+ calculated for C₁₉H₁₉N₄O₂: 335.2). Single peak by HPLC.

### Example 182

### N-(4-(5-amino-1H-pyrazol-4-yl)phenyl)-2,3-dihydrobenzo[b][1,4]dioxine-2-carboxamide

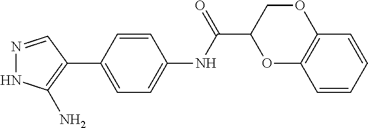

The above aniline (37.5 mg, 0.137 mmol) was dissolved in 1 mL DMF along with 2-benzodioxanecarboxylic acid (25 mg, 1.0 eq), HATU (57 mg, 1.1 eq), and DIEA (26 uL, 1.1 eq). The reaction was stirred for 2 hours before it was dumped into 5 mL water. A white precipitate formed that was filtered off and washed with saturated sodium bicarbonate and distilled water. Single peak by HPLC. A portion of this precipitate was dissolved in 1 mL methanol and 1 mL 4M HCl in dioxane was added to remove the Boc protecting group. The reaction was monitored by HPLC. Upon completion (6 hours) the solvent was removed in vacuo. The residue was taken up in ethanol which was removed in vacuo (repeated twice) to eliminate any traces of acid or dioxane. The desired product was obtained as the hydrochloride salt (14 mg). LCMS (found 337.2 MH+ calculated for C₁₈H₁₇N₄O₃: 337.1). Single peak by HPLC.

### Example 183

### N-(4-(5-amino-1H-pyrazol-4-yl)phenyl)-2,3-dihydrobenzo[b][1,4]dioxine-2-carboxamide

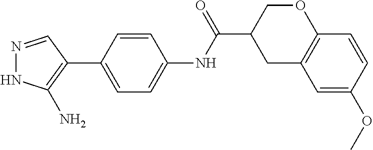

The above aniline (37.5 mg, 0.137 mmol) was dissolved in 1 mL DMF along with 6-methoxy-3-chromancarboxylic acid (25 mg, 1.0 eq), HATU (57 mg, 1.1 eq), and DIEA (26 uL, 1.1 eq). The reaction was stirred for 2 hours before it was dumped into 5 mL water. A white precipitate formed that was filtered off and washed with saturated sodium bicarbonate and distilled water. Single peak by HPLC. A portion of this precipitate was dissolved in 1 mL methanol and 1 mL 4M HCl in dioxane was added to remove the Boc protecting group. The reaction was monitored by HPLC. Upon completion (6 hours) the solvent was removed in vacuo. The residue was taken up in ethanol which was removed in vacuo (repeated twice) to eliminate any traces of acid or dioxane. The desired product was obtained as the hydrochloride salt (11 mg). LCMS (found 365.1 MH+ calculated for C₂₀H₂₁N₄O₃: 365.2). ¹H-NMR (DMSO-d₆, 400 MHz) δ 2.88-3.05 (m, 3H), 3.68 (s, 3H), 3.88-3.96 (m, 1H), 4.35-4.41 (m, 1H), 6.64-6.75 (m, 3H), 7.50 (dd, 2.0 Hz, 6.8 Hz, 2H), 7.62 (dd, 2.0 Hz, 6.8 Hz, 2H), 8.12 (s, 1H), 10.2 (s, 1H). Single peak by HPLC.

### Example 184

### Synthesis of 6-methyl-chroman-3-carboxylic acid [2-methoxy-4-(1H-pyrazol-4-yl)-phenyl]-amide

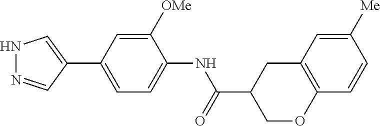

The title compound was prepared according to the procedure described in Scheme 1 (16 mg, 24% over two steps). ¹H NMR (DMSO-d₆, 400 MHz) δ 9.38 (s, 1H), 8.06 (br s, 2H), 7.89 (d, J=8.4 Hz, 1H), 7.26 (d, J=2.0 Hz, 2H), 7.15 (dd, J=8.4, 2.0 Hz, 1H), 6.94 (s, 1H), 6.89 (d, J=8.0 Hz, 1H), 6.67 (d, J=8.0 Hz, 1H), 4.37 (d, J=10.4 Hz, 1H), 3.94 (t, J=10.0 Hz, 1H), 3.89 (s, 3H), 3.22-3.16 (m, 1H), 3.01-2.80 (m, 2H), 2.21 (s, 3H). LC-MS: single peak at 254 nm, MH⁺ calcd. for C₂₁H₂₂N₃O₃: 364, obtained: 364.

### Example 185

### Synthesis of 6-fluoro-chroman-3-carboxylic acid [2-(2-dimethylamino-ethoxy)-5-fluoro-4-(1H-pyrazol-4-yl)-phenyl]-amide

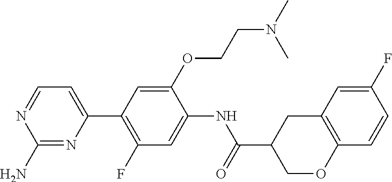

The title compound was prepared from 6-fluoro-chroman-3-carboxylic acid and 4-bromo-2-(2-dimethylamino-ethoxy)-5-fluoroaniline according to the procedure described in Scheme 8 (5 mg, 4% over three steps). ¹H NMR (DMSO-d₆, 400 MHz) δ 9.65 (br s, 1H), 9.56 (s, 1H), 8.33 (d, J=5.2 Hz, 1H), 7.66 (d, J=7.2 Hz, 1H), 7.04 (dd, J=9.2, 4.8 Hz, 1H), 6.98 (dd, J=5.2, 2.0 Hz, 1H), 6.94 (dd, J=8.8, 3.6 Hz, 1H), 6.82 (dd, J=8.8, 4.8 Hz, 1H), 6.71 (br s, 2 H), 4.47-4.42 (m, 3H), 4.04 (dd, J=10.4, 9.6 Hz, 1H), 3.61 (m, 2H), 3.30 (m, 1H), 3.07 (dd, J=16.4, 9.6 Hz, 1H), 2.98 (dd, J=16.0, 4.8 Hz, 1H), 2.93 (d, J=4.8 Hz, 6H). LC-MS: single peak at 254 nm, MH⁺ calcd. for C₂₄H₂₆F₂N₅O₃: 470, obtained: 470.

### Example 186

### Synthesis of 6-methoxy-chroman-3-carboxylic acid [2-(2-dimethylamino-ethoxy)-5-fluoro-4-(1H-pyrazol-4-yl)-phenyl]-amide

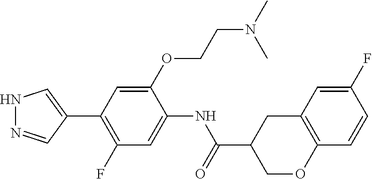

The title compound was prepared from 6-fluoro-chroman-3-carboxylic acid and 4-bromo-2-(2-dimethylamino-ethoxy)-5-fluoroaniline according to the procedure described in Scheme 4 (12 mg, 16% over two steps). ¹H NMR (DMSO-d₆, 400 MHz) δ 9.55 (br s, 1H), 9.40 (s, 1H), 8.10-7.99 (m, 2H), 7.92 (d, J=12.8 Hz, 1H), 7.42 (d, J=6.8 Hz, 1H), 7.04 (dd, J=8.8, 3.2 Hz, 1H), 6.95 (td, J=8.8, 3.2 Hz, 1H), 6.82 (dd, J=8.8, 4.8 Hz, 1H), 4.45 (m, 3H), 4.02 (dd, J=10.4, 9.6 Hz, 1H), 3.59 (m, 2H), 3.24-3.20 (m, 1H), 3.06 (dd, J=16.8, 11.2 Hz, 1H), 2.98-2.92 (m, 1H), 2.93 (d, J=4.8 Hz, 6H). LC-MS: single peak at 254 nm, MH⁺ calcd. for C₂₃H₂₅F₂N₄O₃: 443, obtained: 443.

### Example 187

### Synthesis of (R)-2,3-dihydro-benzo[1,4]dioxine-2-carboxylic acid [4-(2-amino-pyrimidin-4-yl)-2-(2-dimethylamino-ethoxy)-phenyl]-amide

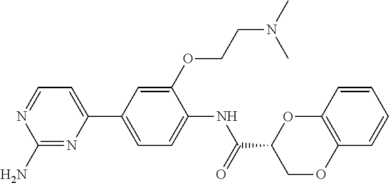

The title compound was prepared according to the procedure described in Scheme 8 (0.041 g, 22% over two steps). ¹H NMR (DMSO-d₆, 400 MHz) δ 9.66 (br s, 1H), 9.34 (s, 1H), 8.34 (d, J=5.2 Hz, 1H), 8.15 (d, J=8.0 Hz, 1H), 7.81-7.78 (m, 2H), 7.23 (d, J=5.6 Hz, 1H), 7.10 (dd, J=8.0, 1.6 Hz, 1H), 6.98-6.91 (m, 3H), 6.85 (br s, 1H), 5.16 (dd, J=5.6, 2.8 Hz, 1H), 4.56-4.46 (m, 3H), 4.40 (dd, J=11.6, 5.6 Hz, 1H), 3.59 (m, 2H), 2.93 (t, J=4.8 Hz, 6H). LC-MS: single peak at 254 nm, MH⁺ calcd. for C₂₃H₂₆N₅O₄: 436, obtained: 436.

### Example 188

### Synthesis of chroman-3-carboxylic acid [2-(3-methyl-butoxy)-4-(1H-pyrazol-4-yl)-phenyl]-amide

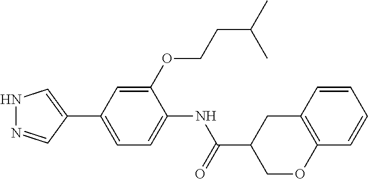

The title compound was prepared from 4-bromo-2-isopentyloxy(aniline) and chroman-3-carboxylic acid according to the procedure described in Scheme 4 (0.032 g, 32% over two steps). ¹H NMR (DMSO-d₆, 400 MHz) δ 9.18 (s, 1H), 8.06 (br s, 2H), 7.78 (d, J=8.0 Hz, 1H), 7.27 (d, J=1.6 Hz, 1H), 7.15-7.06 (m, 3H), 6.84 (t, J=6.8 Hz, 1H), 6.77 (d, J=8.4 Hz, 1H), 4.41 (d, J=11.6 Hz, 1H), 4.11 (t, J=6.8 Hz, 2H), 4.01 (dd, J=10.4, 9.6 Hz, 1H), 3.22-3.16 (m, 1H), 3.10-2.85 (m, 2H), 1.86 (m, 1H), 1.69 (q, J=6.4 Hz, 2H), 0.97 (d, J=6.4 Hz, 6H). LC-MS: single peak at 254 nm, MH⁺ calcd. for C₂₄H₂₈N₃O₃: 406, obtained: 406.

### Example 189

### Synthesis of 6-methoxy-chroman-3-carboxylic acid [2-(3-methyl-butoxy)-4-(1H-pyrazol-4-yl)-phenyl]-amide

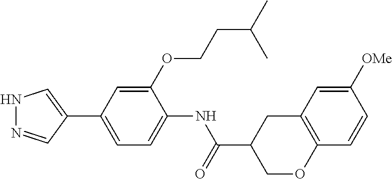

The title compound was prepared from 4-bromo-2-isopentyloxy(aniline) and 6-methoxy-chroman-3-carboxylic acid according to the procedure described in Scheme 4 (0.014 g, 14% over two steps). ¹H NMR (DMSO-d₆, 400 MHz) δ 9.17 (s, 1H), 8.06 (br s, 2H), 7.78 (d, J=8.4 Hz, 1H), 7.26 (d, J=2.0 Hz, 1H), 7.14 (dd, J=8.4, 2.0 Hz, 1H), 6.73-6.67 (m, 3H), 4.35 (d, J=10.8 Hz, 1H), 4.11 (t, J=6.4 Hz, 2H), 3.95 (t, J=10.4 Hz, 1H), 3.69 (s, 3H), 3.20-3.15 (m, 1H), 3.01 (dd, J=16.4, 9.6 Hz, 1H), 2.91 (dd, J=16.4, 6.0 Hz, 1H), 1.86 (m, 1H), 1.69 (q, J=6.8 Hz, 2H), 0.97 (d, J=6.8 Hz, 6H). LC-MS: single peak at 254 nm, MH⁺ calcd. for C₂₅H₃₀N₃O₄: 436, obtained: 436.

### Example 190

### Synthesis of 6-fluoro-chroman-3-carboxylic acid [2-3-methyl-butoxy)-4-(1H-pyrazol-4-yl)-phenyl]-amide

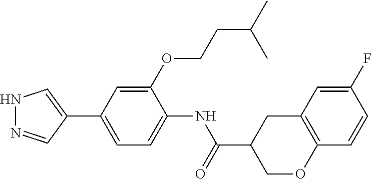

The title compound was prepared from 4-bromo-2-isopentyloxy(aniline) and chroman-3-carboxylic acid according to the procedure described in Scheme 4 (0.032 g, 30% over two steps). ¹H NMR (DMSO-d₆, 400 MHz) δ 9.19 (s, 1H), 8.06 (br s, 2H), 7.77 (d, J=8.4 Hz, 1H), 7.27 (d, J=1.2 Hz, 1H), 7.14 (dd, J=8.4, 1.2 Hz, 1H), 7.00 (dd, J=9.2, 2.8 Hz, 1H), 6.93 (td, J=8.4, 2.8 Hz, 1H), 6.80 (dd, J=9.2, 4.8 Hz, 1H), 4.39 (d, J=10.8 Hz, 1H), 4.11 (t, J=6.4 Hz, 2H), 4.01 (dd, J=10.4, 9.6 Hz, 1H), 3.22-3.15 (m, 1H), 3.03 (dd, J=16.4, 9.2 Hz, 1H), 2.94 (dd, J=16.4, 4.8 Hz, 1H), 1.86 (m, 1H), 1.69 (q, J=6.8 Hz, 2H), 0.97 (d, J=6.4 Hz, 6H). LC-MS: single peak at 254 nm, MH⁺ calcd. for C₂₄H₂₇FN₃O₃: 424, obtained: 424.

### Example 191

### Synthesis of chroman-3-carboxylic acid [2-dimethylcarbamoylmethoxy-4-(1H-pyrazol-4-yl)-phenyl]-amide

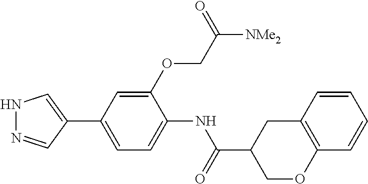

### Synthesis of 2-Hydroxy-N,N-dimethylacetamide

The title compound was prepared according to the procedure of Haberman and Gin. Org. Lett. 2003, 5, 2539-2541.

### Synthesis of 2-(5-bromo-2-nitrophenoxy)-N,N-dimethylacetamide

To a solution of 2-hydroxy-N,N-dimethylacetamide (0.950 g, 9.21 mmol) in THF (25 mL) at 0° C. was added NaH (60% oil dispersion, 0.550 g, 13.8 mmol). After stirring for 10 min, 4-bromo-2-fluoro-1-nitrobenzene (2.03 g, 9.21 mmol) was added and the resulting mixture was allowed to warm to room temperature and stirred for 7 h. The solvent was removed by rotary evaporation and the residue was treated with ethyl acetate (100 mL) and an aqueous solution of saturated NH₄Cl (100 mL). The layers were separated and the organic layer was washed with brine, dried over Na₂SO₄, filtered, and concentrated in vacuo. The residue was purified by flash column chromatography (10-75% ethyl acetate in hexanes) to give the title compound (2.27 g, 81%). ¹H NMR (CDCl₃, 400 MHz) δ 7.76 (d, J=8.8 Hz, 1H), 7.32 (d, J=1.6 Hz, 1H), 7.22 (dd, J=8.8, 2.0 Hz, 1H), 4.85 (s, 2H), 3.12 (s, 3H), 3.00 (s, 3H).

### Synthesis of 2-(2-amino-5-bromophenoxy)-N,N-dimethylacetamide

To a solution of 2-(5-bromo-2-nitrophenoxy)-N,N-dimethylacetamide (Step B, 1.45 g, 4.78 mmol) in ethanol (50 mL) was added stannous chloride (5.4 g, 24.0 mmol) and the mixture was heated to 70° C. for 2 h. The reaction was mixture was diluted with ice water and concentrated by rotary evaporation. An aqueous Na₂CO₃ solution was added and the aqueous phase was extracted with ethyl acetate (3×50 mL). The combined organic layers were washed with brine (50 mL), dried over Na₂SO₄, filtered, and concentrated to dryness to afford the title compound (0.96 g, 74%).

### Synthesis of chroman-3-carboxylic acid [2-dimethylcarbamoylmethoxy-4-(1H-pyrazol-4-yl)-phenyl]-amide

The title compound was prepared from 2-(2-amino-5-bromophenoxy)-N,N-dimethylacetamide and chroman-3-carboxylic acid according to the procedure described in Scheme 4 (0.029 g, 21% over two steps). ¹H NMR (DMSO-d₆, 400 MHz) δ 10.01 (s, 1H), 8.01 (br s, 2H), 7.96 (d, J=8.0 Hz, 1H), 7.31 (d, J=1.6 Hz, 1H), 7.21 (dd, J=8.4, 2.0 Hz, 1H), 7.14 (d, J=7.6 Hz, 1H), 7.09 (t, J=7.6 Hz, 1H), 6.86 (td, J=7.2, 1.2 Hz, 1H), 6.78 (d, J=8.0 Hz, 1H), 4.97 (s, 2H), 4.47 (dd, J=8.8, 1.6 Hz, 1H), 4.05 (dd, J=10.8, 9.6 Hz, 1H), 3.19-3.12 (m, 1 H), 3.07 (dd, J=16.0, 9.2 Hz, 1H), 3.01-2.95 (m, 1H), 2.99 (s, 3H), 2.87 (s, 3H). LC-MS: single peak at 254 nm, MH⁺ calcd. for C₂₃H₂₅N₄O₄: 421, obtained: 421.

### Example 192

### Synthesis of 6-methoxy-chroman-3-carboxylic acid [2-dimethylcarbamoylmethoxy-4-(1H-pyrazol-4-yl)-phenyl]-amide

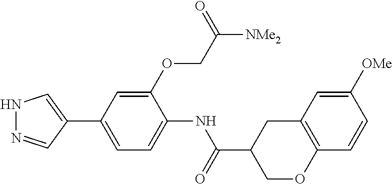

The title compound was prepared from 2-(2-amino-5-bromophenoxy)-N,N-dimethylacetamide and 6-methoxy-chroman-3-carboxylic acid according to the procedure described in Scheme 4 (0.027 g, 25% over two steps). ¹H NMR (DMSO-d₆, 400 MHz) δ 10.00 (s, 1H), 8.01 (br s, 2 H), 7.96 (d, J=8.4 Hz, 1H), 7.31 (d, J=1.6 Hz, 1H), 7.22 (dd, J=8.4, 2.0 Hz, 1H), 6.73 (m, 4H), 4.97 (s, 2H), 4.40 (d, J=9.2 Hz, 1H), 4.00 (dd, J=10.4, 9.2 Hz, 1H), 3.68 (s, 3 H), 3.17-3.10 (m, 1H), 3.05 (dd, J=16.0, 9.2 Hz, 1H), 2.99-2.93 (m, 1H), 2.99 (s, 3H), 2.88 (s, 3H). LC-MS: single peak at 254 nm, MH⁺ calcd. for C₂₄H₂₇N₄O₅: 451, obtained: 451.

### Example 193

### Synthesis of 6-fluoro-chroman-3-carboxylic acid [2-dimethylcarbamoylmethoxy-4-(1H-pyrazol-4-yl)-phenyl]-amide

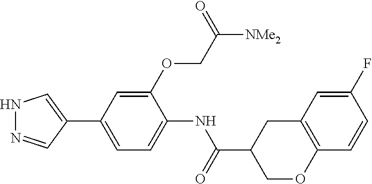

The title compound was prepared from 2-(2-amino-5-bromophenoxy)-N,N-dimethylacetamide and 6-fluoro-chroman-3-carboxylic acid according to the procedure described in Scheme 4 (0.029 g, 21% over two steps). ¹H NMR (DMSO-d₆, 400 MHz) δ 10.02 (s, 1H), 8.01 (br s, 2H), 7.95 (d, J=8.0 Hz, 1H), 7.31 (d, J=1.6 Hz, 1H), 7.22 (dd, J=8.4, 1.6 Hz, 1H), 7.01 (dd, J=9.2, 2.8 Hz, 1H), 6.93 (td, J=8.8, 2.8 Hz, 1H), 6.80 (dd, J=8.8, 4.8 Hz, 1H), 4.97 (s, 2H), 4.45 (ddd, J=10.4, 3.2, 1.6 Hz, 1H), 4.06 (dd, J=10.8, 8.8 Hz, 1H), 3.19-3.12 (m, 1H), 3.08 (dd, J=16.0, 9.2 Hz, 1H), 3.01-2.94 (m, 1H), 2.99 (s, 3H), 2.88 (s, 3H). LC-MS: single peak at 254 nm, MH⁺ calcd. for C₂₃H₂₄FN₄O₄: 439, obtained: 439.

### Example 194

### N6-(2-(dimethylamino)ethyl)-N6-methyl-N3-(4-(5-methyl-1H-pyrazol-4-yl)phenyl)chroman-3,6-dicarboxamide

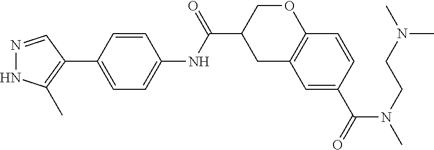

The titled compound was synthesized based on procedures in Scheme 1. LCMS (found 462 MH+ calculated for C₂₆H₃₁N₅O₃: 462). Single peak observed on the analytical HPLC trace.

### Example 195

### Synthesis of 6-fluoro-chroman-3-carboxylic acid [4-(3-amino-1H-pyrazol-4-yl)-phenyl]-amide

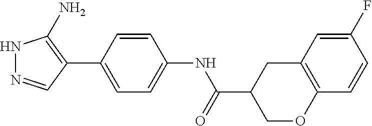

To a suspension of tert-butyl 5-amino-4-(4-aminophenyl)-1H-pyrazole-1-carboxylate (75 mg, 0.274 mmol) and N-Boc-4-phenylglycine (54 mg, 0.274 mmol) in DMF (0.3 mL) was added HATU (1.2 equiv) and N-methylmorpholine (2 equiv) and resulting mixture was stirred overnight at room temperature. After the solvent was removed by rotary evaporation, the residue was dissolved in ethyl acetate (10 mL) and washed twice with a saturated aqueous solution of NaHCO₃ (5 mL) and brine (5 mL). The organic layer was dried over Na₂SO₄, filtered, and concentrated to dryness. The residue was purified by flash column chromatography (20 to 45% ethyl acetate in hexanes). The resulting colorless oil was dissolved in a solution of 50% TFA in methylene chloride and stirred for 1 h. After determining the reaction to be complete by HPLC, the solvent was removed by rotary evaporation and the residue was purified by preparative HPLC to give the title compound (28 mg, 22% over two steps). ¹H NMR (DMSO-d₆, 400 MHz) δ 10.19 (s, 1H), 7.86 (s, 1H), 7.60 (d, J=8.8 Hz, 2H), 7.45 (d, J=8.8 Hz, 2H), 7.03 (dd, J=9.2 Hz, 2.8 Hz, 1H), 6.93 (td, J=8.8 Hz, 4.8 Hz, 1H), 4.43 (dd, J=10.4, 3.2 Hz, 1H), 4.00 (m, 1H), 3.11-2.93 (m, 3H). LC-MS: single peak at 254 nm, MH⁺ calcd. for C₁₉H_{B}FN₄O₂: 353, obtained: 353.

### Example 196

### Synthesis of 6-methoxy-chroman-3-carboxylic acid [2-[2-(benzyl-methyl-amino)-ethoxy]-4-(1H-pyrazol-4-yl)-phenyl]-amide

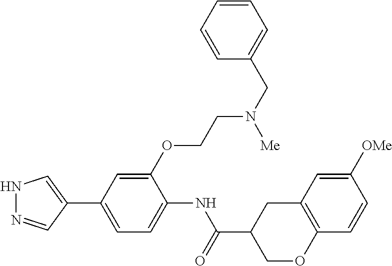

### Step A. Benzyl-[2-(5-bromo-2-nitro-phenoxy)-ethyl]-methyl-amine

To a solution of 2-(benzyl(methyl)amino)ethanol (1.75 g, 10.6 mmol) in THF (40 mL) at 0° C. was added NaH (60% oil dispersion, 1.00 g, 15.0 mmol). After stirring for 10 min, 4-bromo-2-fluoro-1-nitrobenzene (2.33 g, 10.6 mmol) was added and the resulting mixture was allowed to warm to room temperature and stirred for 7 h. The solvent was removed by rotary evaporation and the residue was treated with ethyl acetate (100 mL) and an aqueous solution of saturated NH₄Cl (100 mL). The layers were separated and the organic layer was washed with brine, dried over Na₂SO₄, filtered, and concentrated in vacuo to afford the title compound (3.37 g, 87%). LC-MS: single peak at 254 nm, MH⁺ calcd. for C₁₆H₁₈BrN₂O₃: 366, obtained: 366.

### Step B. 2-(2-(Benzyl(methyl)amino)ethoxy)-4-bromoaniline

2-(benzyl(methyl)amino)ethanol (2.00 g, 5.48 mmol) was dissolved in ethanol (50 mL) and treated with stannous chloride (6.19 g, 27.4 mmol) and the mixture was heated to 70° C. for 2 h. The reaction was mixture was diluted with ice water and concentrated by rotary evaporation. An aqueous Na₂CO₃ solution was added and the aqueous phase was extracted with ethyl acetate (3×50 mL). The combined organic layers were washed with brine (50 mL), dried over Na₂SO₄, filtered, and concentrated to dryness to afford the title compound (1.63 g, 89%). LC-MS: single peak at 254 nm, MH⁺ calcd. for C₁₆H₂₀BrN₂O: 335, obtained: 335.

### Step C. 6-Methoxy-chroman-3-carboxylic acid [2-[2-(benzyl-methyl-amino)-ethoxy]-4-(1H-pyrazol-4-yl)-phenyl]-amide

The title compound was prepared from 6-methoxy-chroman-3-carboxylic acid and 2-(2-(benzyl(methyl)amino)ethoxy)-4-bromoaniline according to the procedure described in Scheme 4 (0.145 g, 54% over two steps). LC-MS: single peak at 254 nm, MH⁺ calcd. for C₃₀H₃₃N₄O₄: 513, obtained: 513.

### Example 197

### Synthesis of chroman-3-carboxylic acid [2-[2-(benzyl-methyl-amino)-ethoxy]-4-(1H-pyrazol-4-yl)-phenyl]-amide

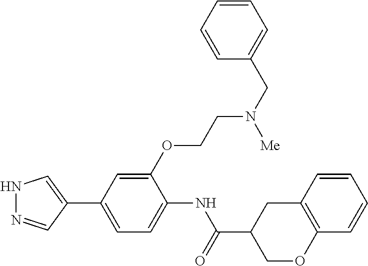

The title compound was prepared from chroman-3-carboxylic acid and 2-(2-(benzyl(methyl)amino)ethoxy)-4-bromoaniline according to the procedure described in Scheme 4 (0.098 g, 39% over two steps). ¹H NMR (DMSO-d₆, 400 MHz) δ 9.72 (br s, 1H), 9.38 (d, J=8.4 Hz, 1H), 8.06 (br s, 2H), 7.77 (dd, J=8.0, 2.4 Hz, 1H), 7.55 (m, 2H), 7.45 (m, 3H), 7.31 (d, J=2.0 Hz, 1H), 7.23 (dd, J=8.4, 2.0 Hz, 1H), 7.10 (m, 2H), 6.87 (t, J=7.6 Hz, 1H), 6.80 (d, J=8.8 Hz, 1H), 4.56-4.46 (m, 3H), 4.45-4.37 (m, 2H), 3.98 (dd, J=11.2, 10.4 Hz, 1H), 3.61 (m, 2H), 3.18-3.11 (m, 1H), 3.06-2.98 (m, 1H), 2.93-2.87 (m, 1H), 2.86 (t, J=4.8 Hz, 3H). LC-MS: single peak at 254 nm, MH⁺ calcd. for C₂₉H₃₁N₄O₃: 483, obtained: 483.

### Example 198

### Synthesis of 6-fluoro-chroman-3-carboxylic acid [2-[2-(benzyl-methyl-amino)-ethoxy]-4-(1H-pyrazol-4-yl)-phenyl]-amide

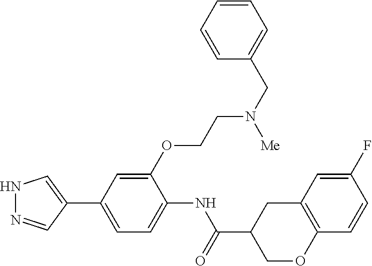

The title compound was prepared from 6-fluoro-chroman-3-carboxylic acid and 2-(2-(benzyl(methyl)amino)ethoxy)-4-bromoaniline according to the procedure described in Scheme 4 (0.081 g, 35% over two steps). ¹H NMR (DMSO-d₆, 400 MHz) δ 9.80 (br s, 1H), 9.42 (d, J=10.0 Hz, 1H), 8.06 (br s, 2H), 7.76 (dd, J=8.0, 2.8 Hz, 1H), 7.55 (m, 2H), 7.46 (m, 3H), 7.31 (s, 1H), 7.25 (dd, J=8.4, 2.0 Hz, 1H), 6.95 (m, 2H), 6.81 (dd, J=8.0, 3.2 Hz, 1H), 4.55-4.46 (m, 3H), 4.43-4.36 (m, 2H), 3.98 (dd, J=10.8, 9.2 Hz, 1H), 3.61 (m, 2H), 3.19-3.11 (m, 1H), 3.05-2.98 (m, 1H), 2.94-2.90 (m, 1H), 2.86 (t, J=4.8 Hz, 3H). LC-MS: single peak at 254 nm, MH⁺ calcd. for C₂₉H₃₀FN₄O₃: 501, obtained: 501.

### Example 199

### Synthesis of 6-methoxy-chroman-3-carboxylic acid [2-(2-methylamino-ethoxy)-4-(1H-pyrazol-4-yl)-phenyl]-amide

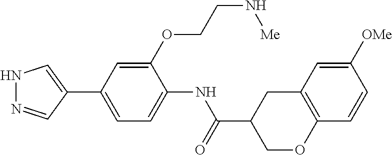

To a solution of 6-methoxy-chroman-3-carboxylic acid [2-[2-(benzyl-methyl-amino)-ethoxy]-4-(1H-pyrazol-4-yl)-phenyl]-amide (0.125 g, 0.169 mmol) in ethyl acetate (1 mL) and methanol (4 mL) was added 10% palladium on carbon (20 mg) and the reaction was held under a hydrogen atmosphere using a balloon for 30 h at room temperature. After removal of catalyst by filtration through a pad of Celite, the filtrate was concentrated by rotary evaporation. The residue was purified by preparative HPLC to afford the title compound (0.057 g, 52%). ¹H NMR (DMSO-d₆, 400 MHz) δ 9.27 (s, 1H), 8.61 (br s, 2H), 8.07 (br s, 2H), 7.90 (d, J=8.4 Hz, 1H), 7.32 (d, J=1.6 Hz, 1H), 7.23 (dd, J=8.4, 1.6 Hz, 1H), 6.75-6.68 (m, 3H), 4.42 (m, 1H), 4.36 (t, J=4.8 Hz, 2H), 3.95 (t, J=10.4 Hz, 1H), 3.69 (s, 3H), 3.43 (m, 2H), 3.21-3.14 (m, 1H), 3.06 (dd, J=16.4, 10.4 Hz, 1H), 2.92 (dd, J=16.8, 6.4 Hz, 1H), 2.72 (t, J=5.6 Hz, 3H). LC-MS: single peak at 254 nm, MH⁺ calcd. for C₂₃H₂₇N₄O₄: 423, obtained: 423.

### Example 200

### Synthesis of chroman-3-carboxylic acid [2-(2-methylamino-ethoxy)-4-(1H-pyrazol-4-yl)-phenyl]-amide

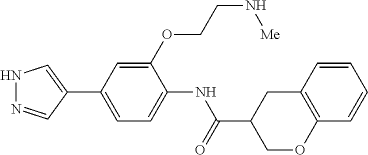

The title compound was prepared from chroman-3-carboxylic acid [2-[2-(benzyl-methyl-amino)-ethoxy]-4-(1H-pyrazol-4-yl)-phenyl]-amide according to the procedure described in Example 199 (0.038 g, 57%). ¹H NMR (DMSO-d₆, 400 MHz) δ 9.27 (s, 1H), 8.61 (br s, 2H), 8.07 (br s, 2H), 7.90 (d, J=8.4 Hz, 1H), 7.32 (d, J=1.6 Hz, 1H), 7.23 (dd, J=8.4, 1.6 Hz, 1H), 6.75-6.68 (m, 3H), 4.42 (m, 1H), 4.36 (t, J=4.8 Hz, 2H), 3.95 (t, J=10.4 Hz, 1H), 3.69 (s, 3H), 3.43 (m, 2H), 3.21-3.14 (m, 1H), 3.06 (dd, J=16.4, 10.4 Hz, 1H), 2.92 (dd, J=16.8, 6.4 Hz, 1H), 2.72 (t, J=5.6 Hz, 3H). LC-MS: single peak at 254 nm, MH⁺ calcd. for C₂₂H₂₅N₄O₃: 393, obtained: 393.

### Example 201

### 6-fluoro-chroman-3-carboxylic acid [2-(2-methylamino-ethoxy)-4-(1H-pyrazol-4-yl)-phenyl]-amide

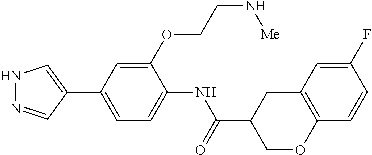

The title compound was prepared from 6-fluoro-chroman-3-carboxylic acid [2-[2-(benzyl-methyl-amino)-ethoxy]-4-(1H-pyrazol-4-yl)-phenyl]-amide according to the procedure described in Example 199 (0.048 g, 89%). ¹H NMR (DMSO-d₆, 400 MHz) δ 9.26 (s, 1H), 8.57 (br s, 2H), 7.88 (d, J=8.0 Hz, 1H), 7.32 (d, J=1.6 Hz, 1H), 7.23 (dd, J=8.4, 1.6 Hz, 1H), 7.04 (dd, J=9.2, 3.2 Hz, 1H), 6.95 (td, J=8.8, 2.8 Hz, 1H), 6.82 (dd, J=9.2, 4.8 Hz, 1H), 4.46 (m, 1H), 4.36 (t, J=4.8 Hz, 2H), 4.02 (t, J=10.0 Hz, 1H), 3.43 (m, 2H), 3.21-3.16 (m, 1H), 3.08 (dd, J=16.4, 10.0 Hz, 1H), 2.96 (dd, J=16.8, 4.0 Hz, 1H), 2.72 (t, J=5.2 Hz, 3H). LC-MS: single peak at 254 nm, MH⁺ calcd. for C₂₂H₂₄FN₄O₃: 411, obtained: 411.

### Example 202

### Synthesis of chroman-3-carboxylic acid [2-[(2-dimethylamino-ethyl)-methyl-carbamoyl]-4-(1H-pyrazol-4-yl)-phenyl]-amide

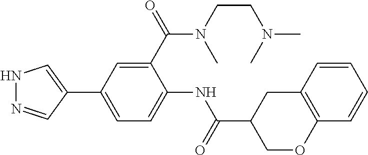

### Step A. 5-bromo-N-(2-(dimethylamino)ethyl)-N-methyl-2-nitrobenzamide

To a suspension of 2-amino-5-bromobenzoic acid (1.23 g, 5.00 mmol) in toluene (10 mL) was added thionyl chloride (0.75 mL) and the resulting mixture was heated to reflux for 2 h. After cooling, the solvent was removed by rotary evaporation and the residue was dissolved in methylene chloride (20 mL). To this solution was added N,N,N′-trimethyl-ethane-1,2-diamine (0.56 g, 5.50 mmol) dissolved in methylene chloride (3 mL) at 0° C. and the mixture was stirred for 0.5 h. The solvent was removed and the residue was purified by flash column chromatography (1-10% methanol in methylene chloride) to afford the title compound (1.39 g, 84%). LC-MS: single peak at 254 nm, MH⁺ calcd. for C₁₂H₁₇BrN₃O₃: 331, obtained: 331.

### Step B. 5-bromo-N-(2-(dimethylamino)ethyl)-N-methyl-2-nitrobenzamide

5-Bromo-N-(2-(dimethylamino)ethyl)-N-methyl-2-nitrobenzamide (1.39 g, 4.20 mmol) was dissolved in ethanol (40 mL) and treated with stannous chloride (4.75 g, 21.0 mmol) and the mixture was heated to 80° C. for 2 h. The reaction was mixture was diluted with ice water and concentrated by rotary evaporation. An aqueous Na₂CO₃ solution was added and the aqueous phase was extracted with ethyl acetate (3×50 mL). The combined organic layers were washed with brine (50 mL), dried over Na₂SO₄, filtered, and concentrated to dryness to afford the title compound (1.63 g, 89%). LC-MS: single peak at 254 nm, MH⁺ calcd. for C₁₂H₁₉BrN₃O: 301, obtained: 301.

### Step C. Chroman-3-carboxylic acid [2-[(2-dimethylamino-ethyl)-methyl-carbamoyl]-4-(1H-pyrazol-4-yl)-phenyl]-amide

The title compound was prepared according to the procedure described in Scheme 4 (0.042 g, 27% over two steps). ¹H NMR (DMSO-d₆, 400 MHz) δ 10.01 (s, 1H), 9.52 (br s, 1H), 8.08 (br s, 2H), 7.67 (dd, J=8.4, 2.0 Hz, 1H), 7.54 (d, J=2.0 Hz, 1H), 7.37 (d, J=8.4 Hz, 1H), 7.15 (d, J=7.6 Hz, 1H), 7.09 (t, J=7.6 Hz, 1H), 6.86 (t, J=7.6 Hz, 1H), 6.79 (d, J=8.0 Hz, 1H), 4.41 (d, J=11.2 Hz, 1H), 3.97 (t, J=10.0 Hz, 1H), 3.74 (m, 2H), 3.34 (m, 2H), 3.03-2.95 (m, 3H), 2.92 (s, 3H), 2.91 (s, 3H), 2.90 (s, 3H). LC-MS: single peak at 254 nm, MH⁺ calcd. for C₂₅H₃₀N₅O₃: 448, obtained: 448.

### Example 203

### Synthesis of 6-fluoro-chroman-3-carboxylic acid [2-[(2-dimethylamino-ethyl)-methyl-carbamoyl]-4-(1H-pyrazol-4-yl)-phenyl]-amide

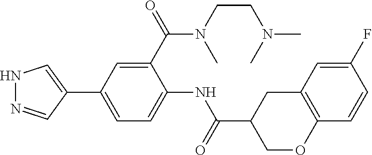

The title compound was prepared from 6-fluoro-chroman-3-carboxylic acid and 5-bromo-N-(2-(dimethylamino)ethyl)-N-methyl-2-nitrobenzamide according to the procedure described in Scheme 4 (0.049 g, 30% over two steps). ¹H NMR (DMSO-d₆, 400 MHz) δ 10.02 (s, 1H), 9.33 (br s, 1H), 8.08 (br s, 2H), 7.67 (dd, J=8.4, 1.6 Hz, 1H), 7.54 (s, 1H), 7.36 (d, J=8.0 Hz, 1H), 7.03 (d, J=9.2 Hz, 1H), 6.94 (td, J=8.8, 3.2 Hz, 1H), 6.81 (dd, J=8.8, 4.8 Hz, 1H), 4.41 (d, J=11.2 Hz, 1H), 3.96 (dd, J=10.0, 8.8 Hz, 1H), 3.73 (m, 2H), 3.33 (m, 2H), 3.03-2.95 (m, 3H), 2.91 (s, 6H), 2.90 (s, 3H). LC-MS: single peak at 254 nm, MH⁺calcd. for C₂₅H₂₉FN₅O₃: 466, obtained: 466.

### Example 204

### N-(4-(2-aminopyrimidin-4-yl)-2-(2-(dimethylamino)ethoxy)-5-fluorophenyl)chroman-3-carboxamide

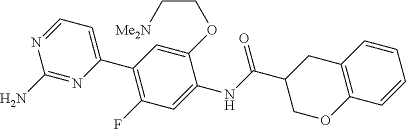

The title compound was prepared according to the procedure described in Scheme 8. ¹H-NMR (DMSO-d₆, 400 MHz), δ: 9.57-9.56 (m, 1H), 9.49 (br, 1H), 8.33-8.32 (m, 1H), 8.01-8.06 (m, 1H), 7.67-7.65 (m, 1H), 7.17-7.09 (m, 2H), 7.00-6.98 (m, 1H), 6.90-6.89 (m, 1H), 6.88-6.86 (m, 1H), 6.84-6.81 (m, 1H), 4.46-4.42 (m, 3H), 4.07-4.02 (m, 1H), 3.62-3.61 (m, 2H), 3.30-3.26 (m, 1H), 3.10-3.04 (m, 1H), 2.99-2.98 (m, 1H), 2.94 (s, 3H), 2.93 (s, 3H); LC-Ms: single peak at 254 nm, MH⁺ calcd. For C₂₄H₂₆FN₅O₃: 452, obtained: 452.

### Example 205

### N-(4-(2-aminopyrimidin-4-yl)-2-(2-(dimethylamino)ethoxy)-5-fluorophenyl)-6-chlorochroman-3-carboxamide

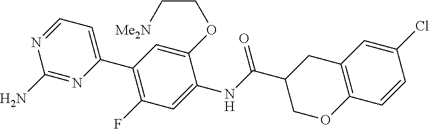

The title compound was prepared according to the procedure described in Scheme 8. ¹H-NMR (DMSO-d₆, 400 MHz), δ: 9.62-9.61 (m, 1H), 9.52 (br, 1H), 8.35-8.34 (m, 1H), 8.19-8.17 (m, 1H), 7.68-7.65 (m, 1H), 7.29-7.27 (m, 1H), 7.16-7.14 (m, 1H), 6.99-6.98 (m, 1H), 6.77-6.76 (m, 1H), 6.75-6.73 (m, 1H), 4.46-4.42 (m, 3H), 4.09-4.04 (m, 1H), 3.62-3.61 (m, 3H), 3.05-3.00 (m, 2H), 2.94 (s, 3H), 2.93 (s, 3H); LC-Ms: single peak at 254 nm, MH⁺ calcd. For C₂₄H₂₅ClFN₅O₃: 486, obtained: 486.

### Example 206

### N-(4-(2-aminopyrimidin-4-yl)-2-(2-(dimethylamino)ethoxy)-5-fluorophenyl)-6-methoxychroman-3-carboxamide

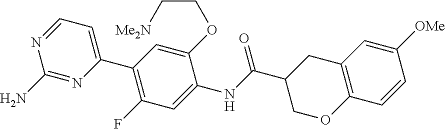

The title compound was prepared according to the procedure described in Scheme 8. ¹H-NMR (DMSO-d₆, 400 MHz), δ: 9.59-9.57 (m, 1H), 9.49 (br, 1H), 8.34-8.33 (m, 1H), 8.10-8.07 (m, 1H), 7.67-7.65 (m, 1H), 7.01-7.00 (m, 1H), 6.83-6.82 (m, 1H), 6.74-6.69 (m, 3H), 4.45-4.40 (m, 4H), 4.00-3.96 (m, 1H), 3.69 (s, 3H), 3.62-3.61 (m, 2H), 3.28-3.25 (m, 1H), 3.09-3.03 (m, 1H), 2.94 (s, 3H), 2.93 (s, 3H); LC-Ms: single peak at 254 nm, MH⁺ calcd. For C₂₅H₂₈FN₅O₄: 482, obtained: 482.

### Example 207

### (S)—N-(4-(2-aminopyrimidin-4-yl)-2-(2-(dimethylamino)ethoxy)-5-fluorophenyl)-2,3-dihydrobenzo[b][1,4]dioxine-2-carboxamide

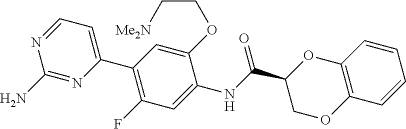

The title compound was prepared according to the procedure described in Scheme 8. ¹H-NMR (DMSO-d₆, 400 MHz), δ: 9.68 (br, 1H), 9.36 (br, 1H), 8.28-8.27 (m, 1H), 8.01-7.98 (m, 1H), 7.60-7.58 (m, 1H), 7.04-7.02 (m, 1H), 6.94-6.92 (m, 1H), 6.90-6.87 (m, 3H), 6.85-6.81 (m, 1H), 5.13-5.11 (m, 1H), 4.42-4.32 (m, 4H), 3.51-3.50 (m, 2H), 2.87 (s, 6H); LC-Ms: single peak at 254 nm, MH⁺ calcd. For C₂₃H₂₄FN₅O₄: 454, obtained: 454.

### Example 208

### N-(4-(2-aminopyrimidin-4-yl)-2-hydroxyphenyl)-6-methoxychroman-3-carboxamide

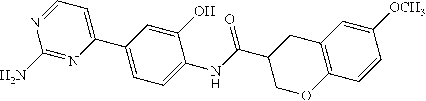

The title compound was prepared according to the procedure described in Scheme 8. LC-Ms: single peak at 254 nm, MH⁺ calcd. For C₂₁H₂₀N₄O₄: 393, obtained: 393.

### Example 209

### N-(2-hydroxy-4-(1H-pyrazol-4-yl)phenyl)-6-methoxychroman-3-carboxamide

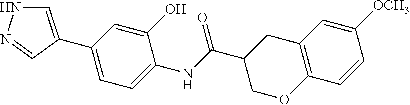

The title compound was prepared according to the procedure described in Scheme 1. LC-Ms: single peak at 254 nm, MH⁺ calcd. For C₂₀H₁₉N₃O₄: 366, obtained: 366.

### Example 210

### N-(2-hydroxy-4-(3-methyl-1H-pyrazol-4-yl)phenyl)-6-methoxychroman-3-carboxamide

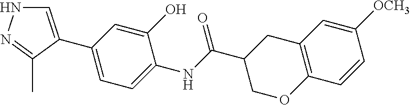

The title compound was prepared according to the procedure described in Scheme 1. LC-Ms: single peak at 254 nm, MH⁺ calcd. For C₂₁H₂₁N₃O₄: 380, obtained: 380.

### Example 211

### N-(2-(dimethylcarbamoyl)-4-(3-methyl-1H-pyrazol-4-yl)phenyl)-6-methoxychroman-3-carboxamide

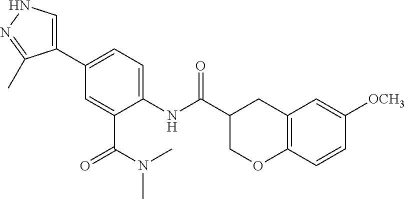

The title compound was prepared according to the procedure described in Scheme 4. LC-Ms: single peak at 254 nm, MH⁺ calcd. For C₂₄H₂₆N₄O₄: 435, obtained: 435.

### Example 212

### N-(4-(2-aminopyrimidin-4-yl)-2-(dimethylcarbamoyl)phenyl)-6-methoxychroman-3-carboxamide

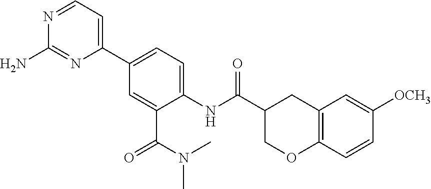

The title compound was prepared according to the procedure described in Scheme 8. LC-Ms: single peak at 254 nm, MH⁺ calcd. For C₂₄H₂₅N₅O₄: 448, obtained: 448.

### Example 213

### N-(4-(2-aminopyridin-4-yl)-2-(dimethylcarbamoyl)phenyl)-6-methoxychroman-3-carboxamide

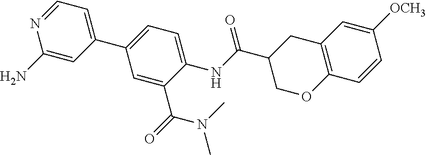

The title compound was prepared according to the procedure described in Scheme 8. LC-Ms: single peak at 254 nm, MH⁺ calcd. For C₂₅H₂₆N₄O₄: 447, obtained: 447.

### Example 214

### N-(2-(dimethylcarbamoyl)-4-(1H-pyrazol-4-yl)phenyl)-6-methylchroman-3-carboxamide

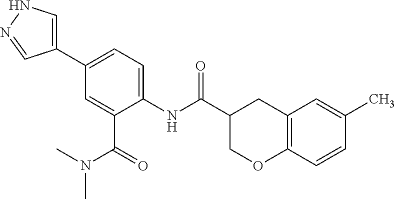

The title compound was prepared according to the procedure described in Scheme 4. LC-Ms: single peak at 254 nm, MH⁺ calcd. For C₂₃H₂₄N₄O₃: 405, obtained: 405.

### Example 215

### N-(2-(dimethylcarbamoyl)-4-(1H-pyrazol-4-yl)phenyl)-6-fluorochroman-3-carboxamide

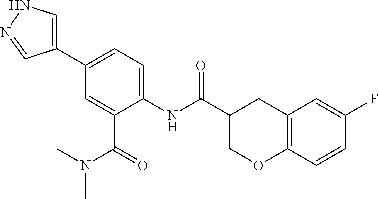

The title compound was prepared according to the procedure described in Scheme 4. LC-Ms: single peak at 254 nm, MH⁺ calcd. For C₂₂H₂₁FN₄O₃: 409, obtained: 409.

### Example 216

### N-(2-(3-(dimethylamino)propoxy)-5-fluoro-4-(1H-pyrazol-4-yl)phenyl)-6-methoxychroman-3-carboxamide

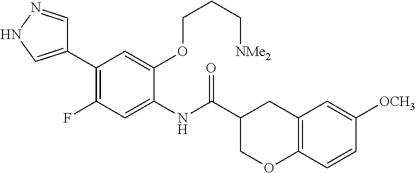

The title compound was prepared according to the procedure described in Scheme 4. LC-Ms: single peak at 254 nm, MH⁺ calcd. For C₂₅H₂₉FN₄O₄: 469, obtained: 469.

### Example 217

### N-(2-(3-(dimethylamino)propoxy)-5-fluoro-4-(1H-pyrazol-4-yl)phenyl)-2,3-dihydrobenzo[b][1,4]dioxine-2-carboxamide

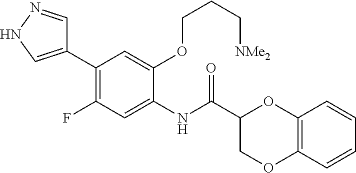

The title compound was prepared according to the procedure described in Scheme 4. LC-Ms: single peak at 254 nm, MH⁺ calcd. For C₂₃H₂₅FN₄O₄: 441, obtained: 441.

### Example 218

### N-(4-(1H-pyrazol-4-yl)-2-(tetrahydro-2H-pyran-4-yloxy)phenyl)-2,3-dihydrobenzo[b][1,4]dioxine-2-carboxamide

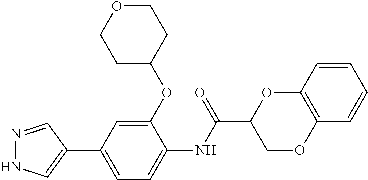

The title compound was prepared according to the procedure described in Scheme 4. LC-Ms: single peak at 254 nm, MH⁺ calcd. For C₂₃H₂₅N₃O₅: 422, obtained: 422.

### Example 219

### methyl 3-(2-(2-(dimethylamino)ethoxy)-4-(1H-pyrazol-4-yl)phenylcarbamoyl)chroman-6-carboxylate

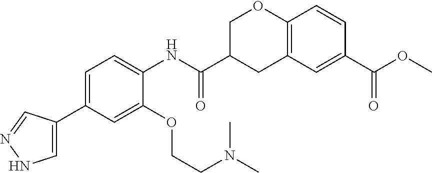

Procedures in Scheme 4 were utilized to synthesize this compound. ¹H-NMR (MeOD-d₄, 400 MHz) δ 8.06 (1H, d, J=8.3 Hz), 7.96 (2H, bs), 7.85 (1H, s), 7.77 (1H, dd, J=2.1 Hz, 8.6 Hz), 7.24 (1H, d, J=1.6 Hz), 7.17 (1H, dd, J=1.7 Hz, 8.4 Hz), 6.88 (1H, d, J=8.6 Hz), 4.56 (1H, m), 4.22 (2H, t, J=5.1 Hz), 4.17 (1H, m), 3.86 (3H, s), 3.19 (3H, m), 2.81 (2H, t, J=5.1 Hz), 2.39 (6H, s). LC/MS: C₂₅H₂₈N₄O₅ (M+1) 465. Single peak at both 215 nm and 254 nm in analytical HPLC traces.

### Example 220

### 3-(2-(2-(dimethylamino)ethoxy)-4-(1H-pyrazol-4-yl)phenylcarbamoyl) chroman-6-carboxylic acid

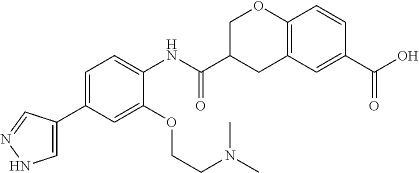

Procedures in Scheme 4 were utilized to synthesize this compound. LC/MS: C₂₄H₂₆N₄O₅ (M+1) 451. Single peak at both 215 nm and 254 nm in analytical HPLC traces.

### Example 221

### N³-(2-(2-(dimethylamino)ethoxy)-4-(1H-pyrazol-4-yl)phenyl)-N⁶-isobutylchroman-3,6-dicarboxamide

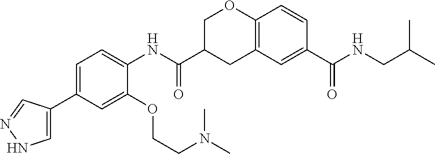

Procedures in Scheme 4 were utilized to synthesize this compound. ¹H-NMR (MeOD-d₄, 400 MHz) δ 8.02 (2H, s), 7.71 (1H, d, J=8.2 Hz), 7.68 (1H, d, J=1.9 Hz), 7.60 (1H, dd, J=2.2 Hz, 8.5 Hz), 7.35 (1H, d, J=1.5 Hz), 7.27 (1H, dd, J=1.6 Hz, 8.2 Hz), 6.88 (1H, d, J=8.5 Hz), 4.55 (3H, m), 4.24 (1H, m), 3.62 (2H, m), 3.21 (3H, m), 3.17 (2H, d, J=7.1 Hz), 2.98 (6H, d, J=10.0 Hz), 1.91 (1H, m), 0.96 (6H, d, J=6.7 Hz). LC/MS: C₂₈H₃₅N₅O₄ (M+1) 506. Single peak at both 215 nm and 254 nm in analytical HPLC traces.

### Example 222

### N⁶-cyclopropyl-N³-(2-(2-(dimethylamino)ethoxy)-4-(1H-pyrazol-4-yl)phenyl)chroman-3,6-dicarboxamide

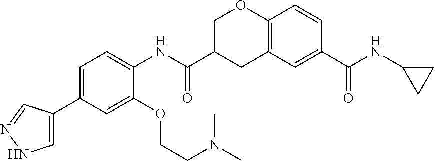

Procedures in Scheme 4 were utilized to synthesize this compound. LC/MS: C₂₇H₃₁N₅O₄ (M+1) 490. Single peak at both 215 nm and 254 nm in analytical HPLC traces.

### Example 223

### N³-(2-(2-(dimethylamino)ethoxy)-4-(1H-pyrazol-4-yl)phenyl)-N⁶-(2-methoxyethyl)chroman-3,6-dicarboxamide

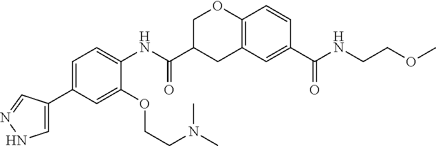

Procedures in Scheme 4 were utilized to synthesize this compound. ¹H-NMR (MeOD-d₄, 400 MHz) δ 8.02 (2H, s), 7.70 (2H, m), 7.61 (1H, dd, J=1.4 Hz, 8.6 Hz), 7.34 (1H, s), 7.27 (1H, d, J=8.3 Hz), 6.87 (1H, d, J=8.6 Hz), 4.54 (3H, m), 4.24 (1H, m), 3.62 (2H, m), 3.54 (4H, m), 3.38 (3H, s), 3.20 (2H, m), 3.10 (1H, m), 2.98 (6H, d, J=8.4 Hz). LC/MS: C₂₇H₃₃N₅O₅ (M+1) 508. Single peak at both 215 nm and 254 nm in analytical HPLC traces.

### Example 224

### N³-(2-(2-(dimethylamino)ethoxy)-4-(1H-pyrazol-4-yl)phenyl)-N⁶-(2-(dimethylamino)ethyl)chroman-3,6-dicarboxamide

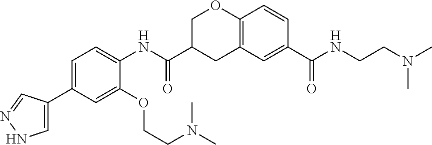

Procedures in Scheme 4 were utilized to synthesize this compound. LC/MS: C₂₈H₃₆N₆O₄ (M+1) 521. Single peak at both 215 nm and 254 nm in analytical HPLC traces.

### Example 225

### N³-(2-(2-(dimethylamino)ethoxy)-4-(1H-pyrazol-4-yl)phenyl)-N⁶-(2-(thiophen-2-yl)ethyl)chroman-3,6-dicarboxamide

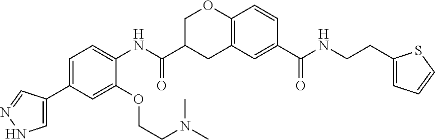

Procedures in Scheme 4 were utilized to synthesize this compound. ¹H-NMR (MeOD-d₄, 400 MHz) δ 8.02 (2H, s), 7.72 (1H, d, J=8.3 Hz), 7.66 (1H, s), 7.58 (1H, d, J=8.6 Hz), 7.35 (1H, s), 7.27 (1H, d, J=8.3 Hz), 7.20 (1H, d, J=5.1 Hz), 6.93 (1H, m), 6.88 (2H, m), 4.54 (3H, m), 4.24 (1H, m), 3.61 (4H, m), 3.14 (5H, m), 2.98 (6H, d, J=7.8 Hz). LC/MS: C₃₀H₃₃N₅O₄S (M+1) 560. Single peak at both 215 nm and 254 nm in analytical HPLC traces.

### Example 226

### N⁶-(2-(1H-imidazol-5-yl)ethyl)-N³-(2-(2-(dimethylamino)-ethoxy)-4-(1H-pyrazol-4-yl)phenyl)chroman-3,6-dicarboxamide

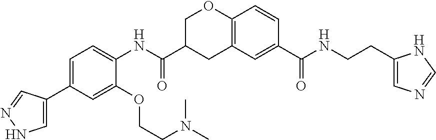

Procedures in Scheme 4 were utilized to synthesize this compound. LC/MS: C₂₉H₃₃N₇O₄ (M+1) 544. Single peak at both 215 nm and 254 nm in analytical HPLC traces.

The following examples were also prepared based on Scheme 4:

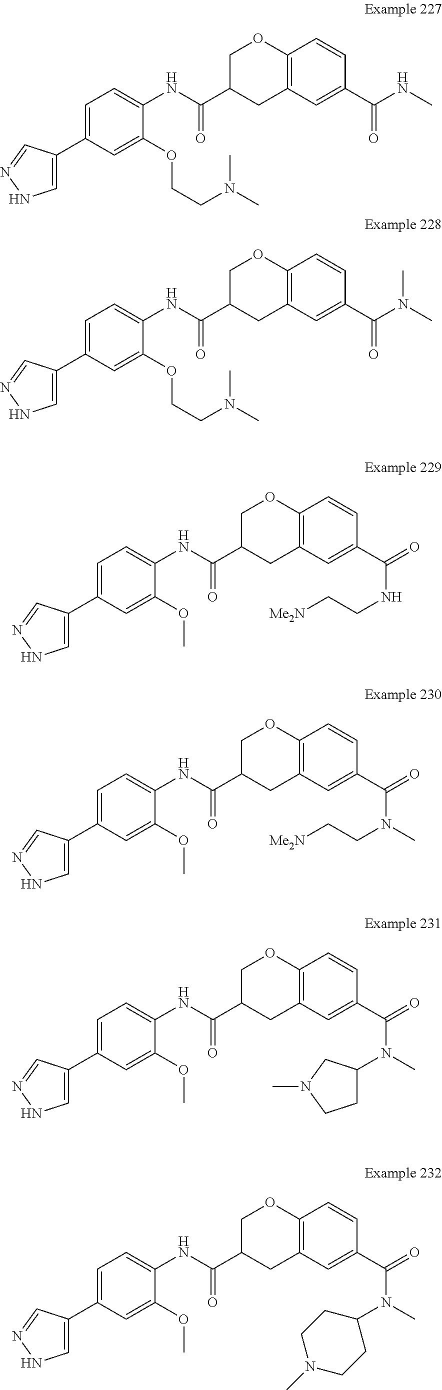

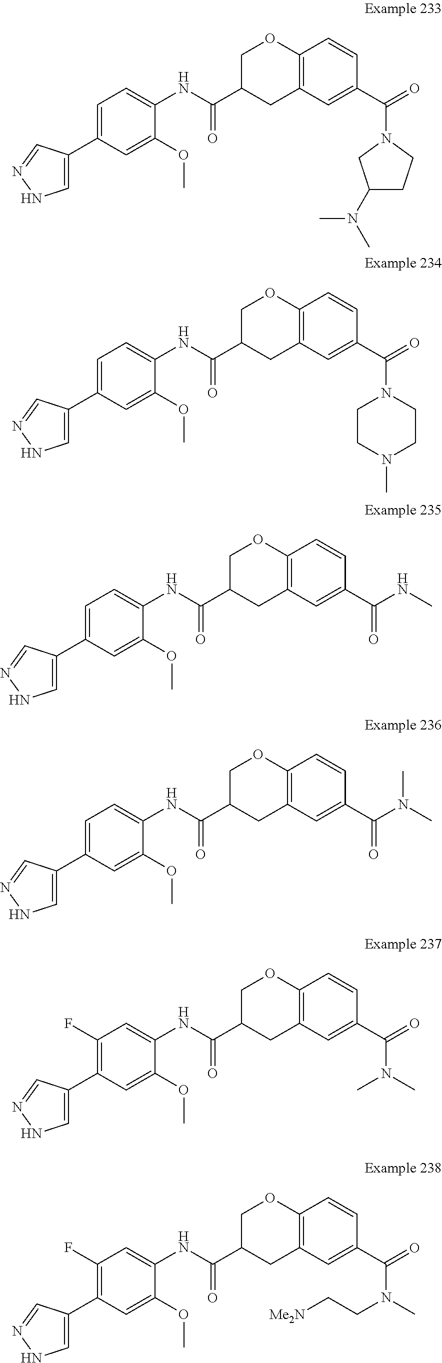

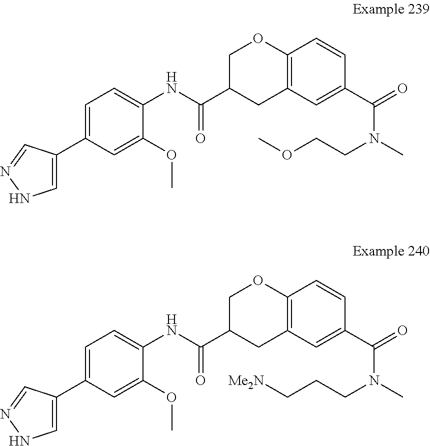

### Example 241

### N-(2-(2-(dimethylamino)ethoxy)-4-(pyridin-4-yl)phenyl)-8-fluorochroman-3-carboxamide

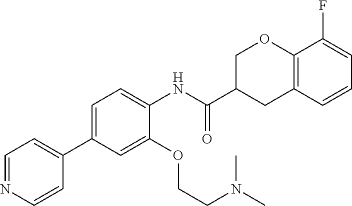

Procedures in Scheme 4 were utilized to synthesize this compound. LC/MS: C₂₅H₂₆FN₃O₃ (M+1) 436. Single peak at both 215 nm and 254 nm in analytical HPLC traces.

### Example 242

### N-(2-(2-(dimethylamino)ethoxy)-4-(1H-pyrazol-4-yl)phenyl)-8-fluorochroman-3-carboxamide

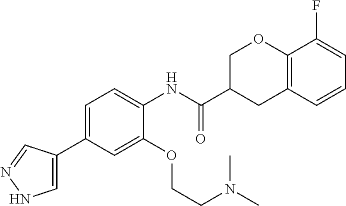

Procedures in Scheme 4 were utilized to synthesize this compound. ¹H-NMR (MeOD-d₄, 400 MHz) δ 8.02 (2H, s), 7.71 (1H, d, J=7.9 Hz), 7.34 (1H, s), 7.26 (1H, d, J=8.2 Hz), 6.94 (2H, m), 6.83 (1H, m), 4.54 (3H, m), 4.26 (1H, m), 3.63 (2H, m), 3.20 (2H, m), 3.09 (1H, m), 2.99 (6H, s). LC/MS: C₂₃H₂₅FN₄O₃ (M+1) 425. Single peak at 254 nm in analytical HPLC trace.

### Example 243

### N-(2-(2-(dimethylamino)ethoxy)-4-(3-methyl-1H-pyrazol-4-yl)phenyl)-8-fluorochroman-3-carboxamide

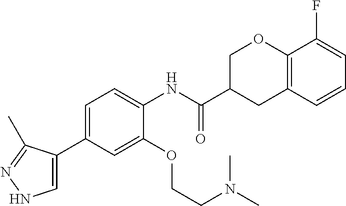

Procedures in Scheme 4 were utilized to synthesize this compound. LC/MS: C₂₄H₂₇FN₄O₃ (M+1) 439. Single peak at both 215 nm and 254 nm in analytical HPLC traces.

### Example 244

### N-(4-(2-aminopyrimidin-4-yl)-2-(2-(dimethylamino)ethoxy-phenyl)-8-fluorochroman-3-carboxamide

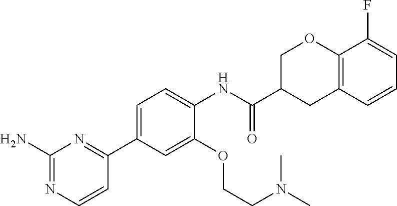

Procedures in Scheme 8 were utilized to synthesize this compound. LC/MS: C₂₄H₂₆FN₅O₃ (M+1) 452. Single peak at both 215 nm and 254 nm in analytical HPLC traces.

### Example 245

### N-(4-(2-aminopyridin-4-yl)-2-(2-(dimethylamino)ethoxy)-phenyl)-8-fluorochroman-3-carboxamide

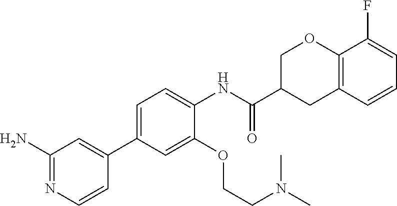

Procedures in Scheme 8 were utilized to synthesize this compound. ¹H-NMR (MeOD-d₄, 400 MHz) δ 8.18 (1H, m), 7.89 (1H, m), 7.49 (2H, m), 7.26 (2H, m) 6.95 (2H, m), 6.84 (1H, m), 4.57 (3H, m), 4.25 (1H, m), 3.70 (2H, bs), 3.16 (3H, m), 3.02 (6H, s). LC/MS: C₂₅H₂₇FN₄O₃ (M+1) 451. Single peak at both 215 nm and 254 nm in analytical HPLC traces.

### Example 246

### N-(2-(2-(dimethylamino)ethoxy)-4-(1H-pyrazol-4-yl)phenyl)-5-methoxychroman-3-carboxamide

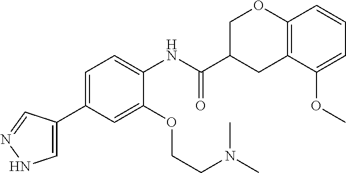

Procedures in Scheme 4 were utilized to synthesize this compound. ¹H-NMR (MeOD-d₄, 400 MHz) δ 8.01 (2H, s), 7.67 (1H, d, J=8.2 Hz), 7.34 (1H, d, J=1.3 Hz), 7.27 (1H, dd, J=1.4 Hz, 8.2 Hz), 7.06 (1H, t, J=8.2 Hz), 6.53 (1H, d, J=8.2 Hz), 6.46 (1H, d, J=8.3 Hz), 4.55 (2H, m), 4.42 (1H, m), 4.11 (1H, dd, J=9.2 Hz, 10.2 Hz), 3.83 (3H, s), 3.61 (2H, m), 3.12 (1H, m), 2.95 (8H, m). LC/MS: C₂₄H₂₈N₄O₄ (M+1) 437. Single peak at both 215 nm and 254 nm in analytical HPLC traces.

### Example 247

### N-(2-(2-(dimethylamino)ethoxy)-4-(3-methyl-1H-pyrazol-4-yl)phenyl)-5-methoxychroman-3-carboxamide

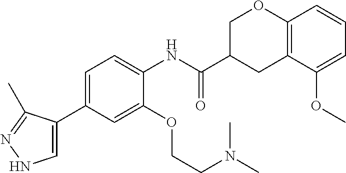

Procedures in Scheme 4 were utilized to synthesize this compound. LC/MS: C₂₅H₃₀N₄O₄ (M+1) 451. Single peak at both 215 nm and 254 nm in analytical HPLC traces.

### Example 248

### N³-(4-(1H-pyrazol-4-yl)-2-(2(pyrrolidin-1-yl)ethylthio)phenyl)-N⁶-isobutylchroman-3,6-dicarboxamide

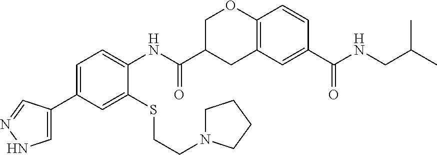

Procedures in Scheme 4 were utilized to synthesize this compound. LC/MS: C₃₀H₃₇N₅O₃S (M+1) 548. Single peak at both 215 nm and 254 nm in analytical HPLC traces.

### Example 249

### N-(4-(1H-pyrazol-4-yl)-2-(2-(pyrrolidin-1-yl)ethylthio)phenyl)-8-fluorochroman-3-carboxamide

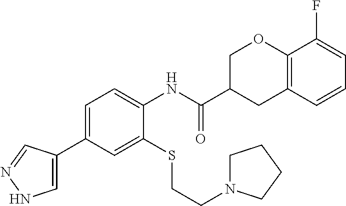

Procedures in Scheme 4 were utilized to synthesize this compound. ¹H-NMR (MeOD-d₄, 400 MHz) δ 8.05 (2H, s), 7.82 (1H, d, J=1.6 Hz), 7.59 (1H, dd, J=1.8 Hz, 8.2 Hz), 7.42 (1H, d, J=8.3 Hz), 6.94 (2H, m), 6.85 (1H, m), 4.51 (1H, dd, J=2.5 Hz, 10.7 Hz), 4.41 (1H, dd, J=6.5 Hz, 10.8 Hz), 3.47 (2H, m), 3.36 (2H, m), 3.31 (2H, m under MeOH), 3.19 (3H, m), 2.97 (2H, m), 2.05 (2H, m), 1.90 (2H, m). LC/MS: C₂₅H₂₇FN₄O₂S (M+1) 467. Single peak at both 215 nm and 254 nm in analytical HPLC traces.

### Example 250

### 8-fluoro-N-(4-(3-methyl-1H-pyrazol-4-yl)-2-(2-(pyrrolidin-1-yl)ethylthio)phenyl)chroman-3-carboxamide

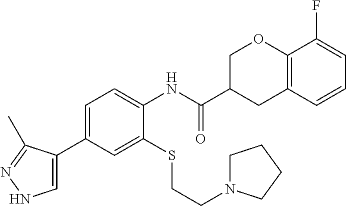

Procedures in Scheme 4 were utilized to synthesize this compound. LC/MS: C₂₆H₂₉FN₄O₂S (M+1) 481. Single peak at both 215 nm and 254 nm in analytical HPLC traces.

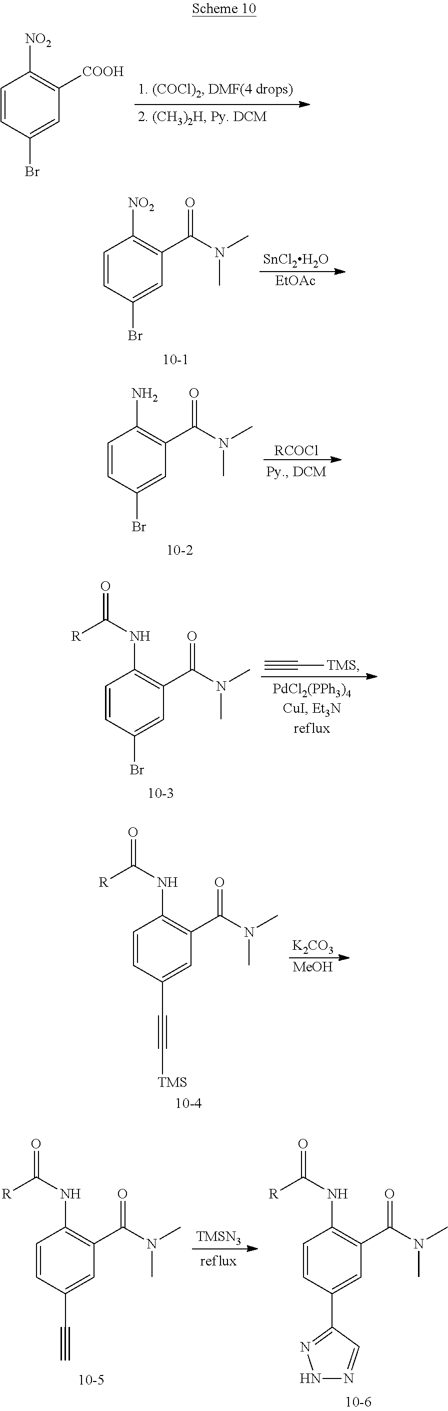

Dry DMF (a few drops) was added to a mixture of 5-bromo-2-nitrobenzoic acid (1 equiv) and Oxalyl dichloride (5 equiv) in dry DCM. After stirred 4 h, the solvent was removed under reduced pressure to give a solid residue. The residue was resolved in dry DCM and was added to a mixture of dimethyl amine (1 equiv) and dry pyridine (3 equiv) in dry DCM at 0° C. After stirring 0.5 h, the reaction was quenched with water and purified by flash chromatography to give 10-1 in 92% yield.

Tin chloride (3 equiv) was added to a mixture of 10-1 (1 equiv) in EtOAC. After the reaction was completed (detected by LC-MS), it was quenched by saturated NaOH and extracted with EtOAC. The combined organic extraction was dried over sodium sulfate and concentrated in vacuo to give crude 10-2 which was then purified by flash chromatography (75% yield).

Acid chloride (1.2 equiv) was added to a mixture of 10-2 (1 equiv) and dry pyridine. (3 equiv) in dry DCM at 0° C. After stirred at room temperature for 0.5 h, the reaction was quenched with water and purified with flash chromatography to give pure 10-3 in 78% yield.

Under Argon, ethynyltrimethylsilane (5 equiv) was added to a mixture of 10-3 (1 equiv), PhCl₂(PPh₃)₄ (0.1 equiv), and CuI (0.2 equiv) in Et₃N. After refluxing 6 h, the reaction solution was cooled to room temperature, filtered, and concentrated under reduced pressure to give crude ethynyl amide 10-4 in 95% yield.

10-4 was added to a suspension of 10 equiv of K₂CO₃ in MeOH. After stirred 4 h, the solvent was removed under reduced pressure to give a residue. The residue was washed with water and extracted with EtOAC. The combined organic extraction was washed with saturated NaCl, dried over sodium sulfate and concentrated in vacuo to give crude 10-5 as a yellow solid in 88% yield.

10-5 (1 equiv) and azidotrimethylsilane (1.5 equiv) was refluxing for 6 h, then the mixture was subjected to preparative HPLC to give the white solid 10-6 in 76% yield (two steps).

### Example 251

### N-(2-(dimethylcarbamoyl)-4-(2H-1,2,3-triazol-4-yl)phenyl)-6-methoxychroman-3-carboxamide

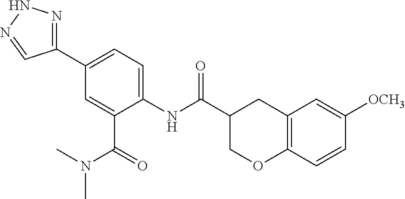

The title compound was prepared according to the procedure described in Scheme 10. LC-Ms: single peak at 254 nm, MH⁺ calcd. For C₂₂H₂₃N₅O₄: 422, obtained: 422.

### Example 252

### N-(2-(dimethylcarbamoyl)-4-(2H-1,2,3-triazol-4-yl)phenyl)chroman-3-carboxamide

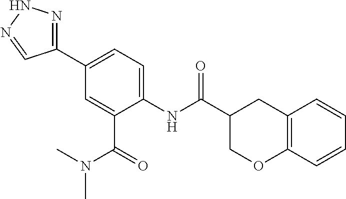

The title compound was prepared according to the procedure described in Scheme 10. LC-Ms: single peak at 254 nm, MH⁺ calcd. For C₂₁H₂₁N₅O₃: 392, obtained: 392.

### Example 253

### N-(2-(dimethylcarbamoyl)-4-(2H-1,2,3-triazol-4-yl)phenyl)-6-methylchroman-3-carboxamide

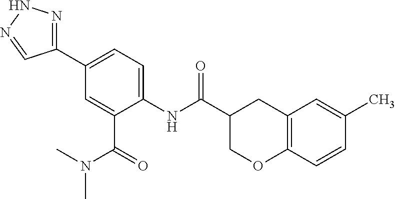

The title compound was prepared according to the procedure described in Scheme 10. LC-Ms: single peak at 254 nm, MH⁺ calcd. For C₂₂H₂₃N₅O₃: 406, obtained: 406.

### Example 254

### 6-Chloro-N-(2-(dimethylcarbamoyl)-4-(2H-1,2,3-triazol-4-yl)phenyl)chroman-3-carboxamide

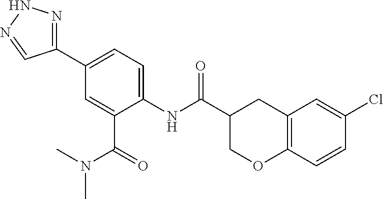

The title compound was prepared according to the procedure described in Scheme 10. LC-Ms: single peak at 254 nm, MH⁺ calcd. For C₂₁H₂₀ClN₅O₃: 426, obtained: 426.

### Example 255

### N-(2-(dimethylcarbamoyl)-4-(2H-1,2,3-triazol-4-yl)phenyl)-6-fluorochroman-3-carboxamide

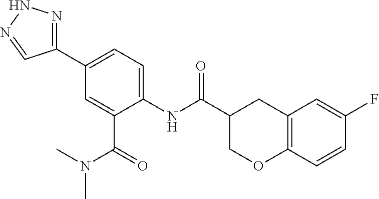

The title compound was prepared according to the procedure described in Scheme 10. LC-Ms: single peak at 254 nm, MH⁺ calcd. For C₂₁H₂₀FN₅O₃: 410, obtained: 410.

### Example 256

### N-(2-(dimethylcarbamoyl)-4-(2H-1,2,3-triazol-4-yl)phenyl)-2,3-dihydrobenzo[b][1,4]dioxine-2-carboxamide

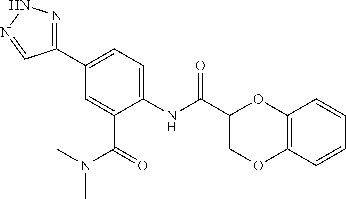

The title compound was prepared according to the procedure described in Scheme 10. LC-Ms: single peak at 254 nm, MH⁺ calcd. For C₂₀H₁₉N₅O₄: 394, obtained: 394.

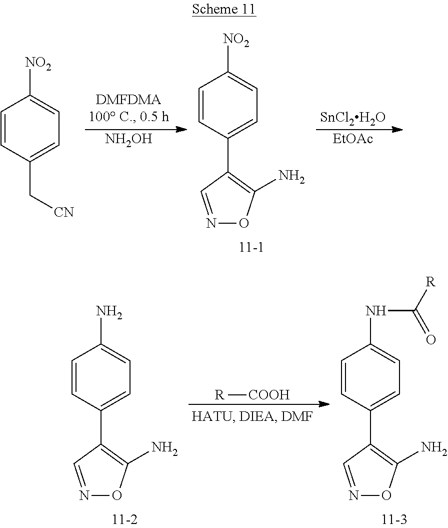

In a sealed tube, 2-(4-nitrophenyl)acetonitrile (2.00 g, 12.3 mmol) and N,N-dimethylformamide/dimethylacetamide (DMF/DMA, 1.8 mL, 1.1 equiv) were heated in the oil bath for 30 minutes at 90° C. After cooled to room temperature, hydroxylamine (3 equiv) was added. The reaction was refluxed overnight. The solvent was removed under reduced pressure to give 11-1 as a green solid in 57% yield.

Tin chloride (3 equiv) was added to a mixture of 11-1 (1 equiv) in EtOAC. After stirred 4 h, the reaction was quenched with saturated NaOH, and extracted with EtOAC. The combined organic extraction was washed with saturated NaCl, dried over sodium sulfate, and concentrated in vacuo to give crude 11-2.

11-2 (1 equiv) was added to a mixture of carbonyl acid (1.1 equiv), HATU (1.1 equiv), and DIEA (3 equiv) in DMF. After the disappearance of 11-2, the reaction was quenched with saturated NaHCO₃ and extracted with EtOAC. The combined organic extraction was washed with saturated NaCl, dried over sodium sulfate, and concentrated to give the amide residue.

The residue was subjected to preparative HPLC to give pure 11-3 as a white solid in 65%-83% yields.

### Example 257

### N-(4-(5-aminoisoxazol-4-yl)phenyl)-6-methoxychroman-3-carboxamide

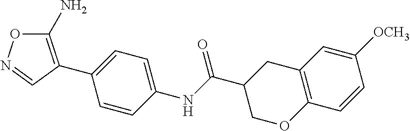

The title compound was prepared according to the procedure described in Scheme 11. LC-Ms: single peak at 254 nm, MH⁺ calcd. For C₂₀H₁₉N₃O₄: 366, obtained: 366.

### Example 258

### N-(4-(5-aminoisoxazol-4-yl)phenyl)-6-methylchroman-3-carboxamide

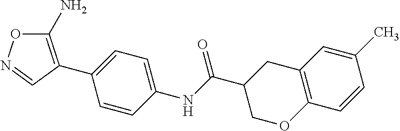

The title compound was prepared according to the procedure described in Scheme 11. LC-Ms: single peak at 254 nm, MH⁺ calcd. For C₂₀H₁₉N₃O₃: 350, obtained: 350.

### Example 259

### N-(4-(5-aminoisoxazol-4-yl)phenyl)chroman-3-carboxamide

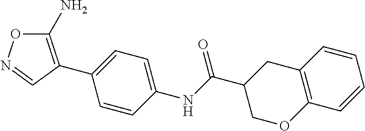

The title compound was prepared according to the procedure described in Scheme 11. LC-Ms: single peak at 254 nm, MH⁺ calcd. For C₁₉H₁₇N₃O₃: 336, obtained: 336.

### Example 260

### N-(4-(5-aminoisoxazol-4-yl)phenyl)-6-fluorochroman-3-carboxamide

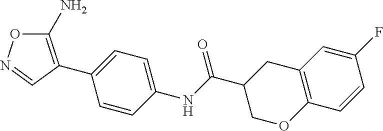

The title compound was prepared according to the procedure described in Scheme 11. LC-Ms: single peak at 254 nm, MH⁺ calcd. For C₁₉H₁₆FN₃O₃: 354, obtained: 354.

### Example 261

### N-(4-(5-aminoisoxazol-4-yl)phenyl)-2,3-dihydrobenzo[b][1,4]dioxine-2-carboxamide

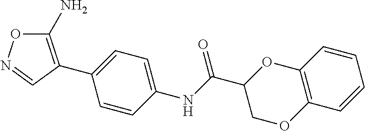

The title compound was prepared according to the procedure described in Scheme 11. LC-Ms: single peak at 254 nm, MH⁺ calcd. For C₁₈H₁₅N₃O₄: 338, obtained: 338.

### Example 262

### N-(2-cyano-4-(1H-pyrazol-4-yl)phenyl)-2,3-dihydrobenzo[b][1,4]dioxine-2-carboxamide

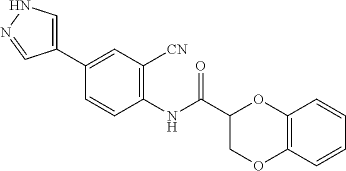

The title compound was prepared according to the procedure described in Scheme 1. LC-Ms: single peak at 254 nm, MH⁺ calcd. For C₁₉H₁₄N₄O₃: 347, obtained: 347.

### Example 263

### N-(2-(3-(dimethylamino)-3-oxopropylthio)-4-(1H-pyrazol-4-yl)phenyl)-6-fluorochroman-3-carboxamide

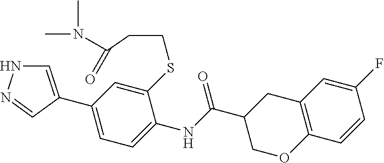

The title compound was prepared according to the procedure described in Scheme 4. ¹H-NMR (DMSO-d₆, 400 MHz), δ: 9.74 (br, 1H), 8.10 (s, 2H), 7.71-7.70 (m, 1H), 7.64-7.55 (m, 2H), 7.52-7.49 (m, 1H), 7.03-7.01 (m, 1H), 6.95-6.90 (m, 1H), 6.82-6.78 (m, 1H), 4.47-4.44 (m, 1H), 3.21-3.19 (m, 1H), 3.12-3.08 (m, 2H), 3.05 (t, J=6.8 Hz, 2H), 2.90 (s, 3H), 2.80 (s, 3H), 2.67-2.66 (m, 1H), 2.60 (t, J=6.8 Hz, 2H); LC-Ms: single peak at 254 nm, MH⁺ calcd. For C₂₄H₂₅FN₄O₃S: 469, obtained: 469.

### Example 264

### N-(2-(3-(dimethylamino)-3-oxopropylthio)-4-(3-methyl-1H-pyrazol-4-yl)phenyl)-6-fluorochroman-3-carboxamide

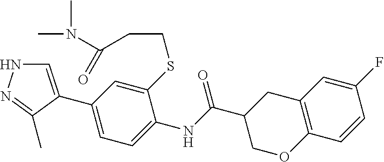

The title compound was prepared according to the procedure described in Scheme 4. LC-Ms: single peak at 254 nm, MH⁺ calcd. For C₂₅H₂₇FN₄O₃S: 483, obtained: 483.

### Example 265

### N-(2-(3-(dimethylamino)-3-oxopropylsulfonyl)-4-(1H-pyrazol-4-yl)phenyl)-6-fluorochroman-3-carboxamide

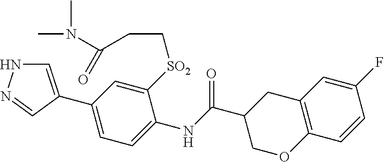

A mixture of N-(2-(3-(dimethylamino)-3-oxopropylthio)-4-(1H-pyrazol-4-yl)phenyl)-6-fluorochroman-3-carboxamide (0.5 equiv), and 30% hydrogen peroxide (1 mL) in formic acid (2 mL) was stirred at room temperature for 0.5 h. The solvent was removed under reduced pressure to give a residue. Then, the residue was subjected to preparative HPLC to give pure sulfone as a white solid in 92% yield. ¹H-NMR (DMSO-d₆, 400 MHz), δ: 9.88 (br, 1H), 8.16 (s, 2H), 8.04-8.03 (m, 1H), 7.99-7.94 (m, 2H), 7.03-7.00 (m, 1H), 6.95-6.90 (m, 1H), 6.81-6.77 (m, 1H), 4.46 (dd, J=10.8, 2.8 Hz, 1H), 4.09 (dd, J=10.8, 8.4 Hz, 1H), 3.61 (t, J=7.6 Hz, 2H), 3.20 (dd, J=8.4, 2.8 Hz, 1H), 3.04 (m, 2H), 2.92 (s, 3H), 2.75 (s, 3H), 2.73 (t, J=7.6 Hz, 2H); LC-Ms: single peak at 254 nm, MH⁺ calcd. For C₂₄H₂₅FN₄O₅S: 501, obtained: 501.

### Example 266

### N-(2-(3-(dimethylamino)-3-oxopropylthio)-4-(1H-pyrazol-4-yl)phenyl)-6-methoxychroman-3-carboxamide

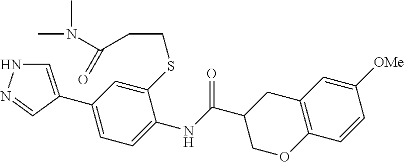

The title compound was prepared according to the procedure described in Scheme 4. LC-Ms: single peak at 254 nm, MH⁺ calcd. For C₂₅H₂₈N₄O₄S: 481, obtained: 481.

### Example 267

### N-(2-(3-(dimethylamino)-3-oxopropylthio)-4-(3-methyl-1H-pyrazol-4-yl)phenyl)-6-methoxychroman-3-carboxamide

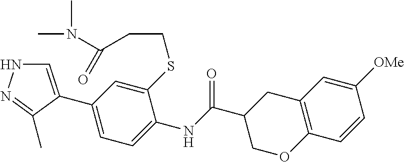

The title compound was prepared according to the procedure described in Scheme 4. LC-Ms: single peak at 254 nm, MH⁺ calcd. For C₂₆H₃₀N₄O₄S: 495, obtained: 495.

### Example 268

### N-(2-(3-(dimethylamino)-3-oxopropylthio)-4-(pyridin-4-yl)phenyl)-6-methoxychroman-3-carboxamide

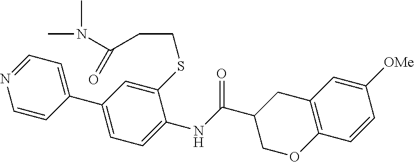

The title compound was prepared according to the procedure described in Scheme 4. LC-Ms: single peak at 254 nm, MH⁺ calcd. For C₂₇H₂₉N₃O₄S: 492, obtained: 492.

### Example 269

### N-(4-(2-aminopyridin-4-yl)-2-methoxyphenyl)chroman-3-carboxamide

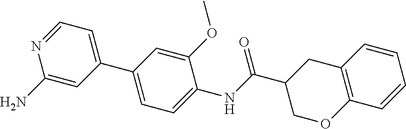

The title compound was prepared according to the procedure described in Scheme 8. LC-Ms: single peak at 254 nm, MH⁺ calcd. For C₂₂H₂₁N₃O₃: 376, obtained: 376.

### Example 270

### 6-Methoxy-N-(2-(4-methoxybenzylthio)-4-(1H-pyrazol-4-yl)phenyl)chroman-3-carboxamide

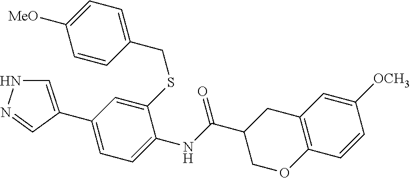

The title compound was prepared according to the procedure described in Scheme 4. LC-Ms: single peak at 254 nm, MH⁺ calcd. For C₂₈H₂₇N₃O₃S: 502, obtained: 502.

### Example 271

### Synthesis of chroman-3-carboxylic acid [2-dimethylcarbamoyl-4-(1H-pyrazol-4-yl)-phenyl]-amide

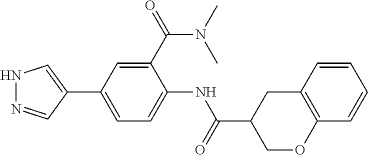

The title compound was prepared from chroman-3-carboxylic acid and 2-Amino-5-bromo-N,N-dimethyl-benzamide according to the procedure described in Scheme 1. ¹H NMR (DMSO-d₆, 400 MHz) δ 9.78 (s, 1H), 8.10 (br s, 2H), 7.64 (dd, J=8.4, 2.0 Hz, 1H), 7.52 (d, J=2.0 Hz, 1H), 7.48 (d, J=8.4 Hz, 1H), 7.14 (d, J=7.6 Hz, 1H), 7.09 (t, J=8.4 Hz, 1H), 6.85 (t, J=7.6 Hz, 1H), 6.78 (d, J=8.0 Hz, 1H), 4.39 (m, 1H), 3.96 (t, J=10.0 Hz, 1H), 3.08-2.97 (m, 2H), 2.98 (s, 3H), 2.89 (m, 1H), 2.82 (s, 3H). LC-MS: single peak at 254 nm, MH⁺ calcd. for C₂₂H₂₃N₄O₃: 391, obtained: 391.

### Example 272

### Synthesis of chroman-3-carboxylic acid (2-dimethylcarbamoyl-4-pyridin-4-yl-phenyl)-amide

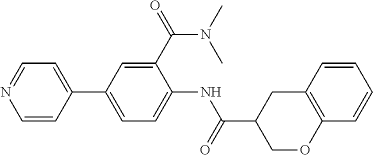

The title compound was prepared from chroman-3-carboxylic acid and 2-Amino-5-bromo-N,N-dimethyl-benzamide according to the procedure described in Scheme 1. ¹H NMR (DMSO-d₆, 400 MHz) δ 10.02 (s, 1H), 8.81 (br s, 2H), 8.08 (br s, 2H), 7.99 (dd, J=8.4, 2.0 Hz, 1H), 7.87 (d, J=2.0 Hz, 1H), 7.79 (d, J=8.4 Hz, 1H), 7.15 (d, J=7.6 Hz, 1H), 7.09 (t, J=8.0 Hz, 1H), 6.86 (td, J=7.6, 1.2 Hz, 1H), 6.79 (dd, J=8.0, 2.0 Hz, 1H), 4.41 (ddd, J=10.4, 3.2, 1.6 Hz, 1H), 3.99 (t, J=10.0 Hz, 1H), 3.16-3.09 (m, 1H), 3.05-2.99 (m, 1H), 3.01 (s, 3H), 2.93 (dd, J=16.0, 5.6 Hz, 1H), 2.86 (s, 3H). LC-MS: single peak at 254 nm, MH⁺ calcd. for C₂₄H₂₄N₄O₃: 402, obtained: 402.

### Example 273

### Synthesis of chroman-3-carboxylic acid [4-(2-amino-pyrimidin-4-yl)-2-dimethylcarbamoylmethoxy-phenyl]-amide

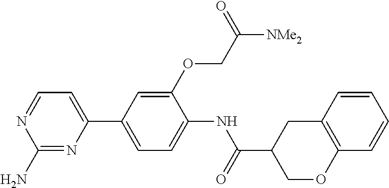

The title compound was prepared according to the procedure described in Scheme 8 (0.024 g, 28% over three steps). ¹H NMR (DMSO-d₆, 400 MHz) δ 10.31 (s, 1H), 8.34 (d, J=5.6 Hz, 1H), 8.25 (d, J=8.4 Hz, 1H), 7.82 (d, J=8.0 Hz, 1H), 7.81 (s, 1H), 7.33-7.21 (br s, 1H), 7.27 (d J=5.6 Hz, 1H), 7.14 (d, J=7.6 Hz, 1H), 7.09 (t, J=8.4 Hz, 1H), 6.86 (td, J=7.6, 1.2 Hz, 1H), 6.79 (dd, J=8.0, 1.2 Hz, 1H), 5.04 (s, 2H), 4.47 (ddd, J=8.4, 3.2, 1.6 Hz, 1H), 4.09 (dd, J=10.4, 9.2 Hz, 1H), 3.28-3.21 (m, 1H), 3.11-3.02 (m, 2H), 2.99 (s, 3H), 2.88 (s, 3H). LC-MS: single peak at 254 nm, MH⁺ calcd. for C₂₄H₂₆N₅O₄: 448, obtained: 448.

### Example 274

### Synthesis of chroman-3-carboxylic acid [2-[2-(dibenzylamino)-ethoxy]-4-(1H-pyrazol-4-yl)-phenyl]-amide

### Step A. 4-Bromo-2-(2-(dibenzylamino)ethoxy)aniline

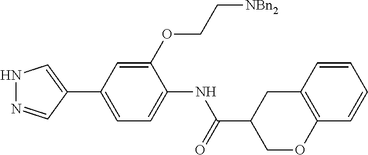

To a solution of 2-(dibenzylamino)ethanol (0.563 g, 2.33 mmol) in THF (40 mL) at rt was added NaH (60% oil dispersion, 0.121 g, 3.03 mmol). After stirring for 10 min, 4-bromo-2-fluoro-1-nitrobenzene (0.513 g, 2.33 mmol) was added and the resulting mixture was stirred for 7 h. The solvent was removed by rotary evaporation and the residue was treated with ethyl acetate (100 mL) and an aqueous solution of saturated NH₄Cl (100 mL). The layers were separated and the organic layer was washed with brine, dried over Na₂SO₄, filtered, and concentrated in vacuo. Without further purification, the residue was dissolved in ethanol (50 mL) and treated with stannous chloride (6.19 g, 27.4 mmol), and the mixture was heated to 70° C. for 2 h. The reaction mixture was diluted with ice water and concentrated by rotary evaporation. An aqueous Na₂CO₃ solution was added and the aqueous phase was extracted with ethyl acetate (3×50 mL). The combined organic layers were washed with brine (50 mL), dried over Na₂SO₄, filtered, and concentrated to dryness to afford the title compound (0.396 g, 41% over two steps). ¹H NMR (CDCl₃, 400 MHz) δ 7.39 (d, J=7.2 Hz, 4H), 7.32 (t, J=7.2 Hz, 4H), 7.24 (t, J=7.6 Hz, 2H), 6.87 (dd, J=8.4, 2.4 Hz, 1H), 6.79 (d, J=2.4 Hz, 1H), 6.55 (d, J=8.4 Hz, 1H), 3.70 (app s, 6H), 4.01 (t, J=6.0 Hz, 2H), 2.92 (t, J=6.0 Hz, 2H), LC-MS: single peak at 254 nm, MH⁺ calcd. for C₂₂H₂₄BrN₂O: 412, obtained: 3412.

### Step B. Chroman-3-carboxylic acid [2-[2-(dibenzylamino)-ethoxy]-4-(1H-pyrazol-4-yl)-phenyl]-amide

The title compound was prepared from chroman-3-carboxylic acid and 4-bromo-2-(2-(dibenzylamino)ethoxy)aniline according to the procedure described in Scheme 4. LC-MS: single peak at 254 nm, MH⁺ calcd. for C₃₅H₃₅N₄O₃: 559, obtained: 559.

### Example 275

### Synthesis of 6-methoxy-chroman-3-carboxylic acid [2-[2-(dibenzylamino)-ethoxy]-4-(1H-pyrazol-4-yl)-phenyl]-amide

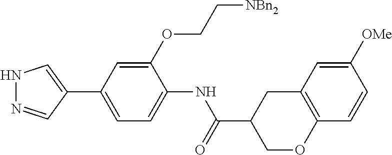

The title compound was prepared from 6-methoxy-chroman-3-carboxylic acid and 4-bromo-2-(2-(dibenzylamino)ethoxy)aniline according to the procedure described in Scheme 4. LC-MS: single peak at 254 nm, MH⁺ calcd. for C₃₆H₃₇N₄O₄: 589, obtained: 589.

### Example 276

### Synthesis of chroman-3-carboxylic acid [2-(2-amino-ethoxy)-4-(1H-pyrazol-4-yl)-phenyl]-amide

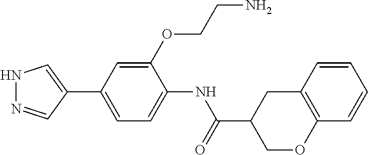

The title compound was prepared from chroman-3-carboxylic acid [2-[2-(dibenzylamino)-ethoxy]-4-(1H-pyrazol-4-yl)-phenyl]-amide according to the procedure described in Example 199. LC-MS: single peak at 254 nm, MH⁺ calcd. for C₂₁H₂₃N₄O₃: 379, obtained: 379.

### Example 277

### Synthesis of 6-methoxy-chroman-3-carboxylic acid [2-(2-amino-ethoxy)-4-(1H-pyrazol-4-yl)-phenyl]-amide

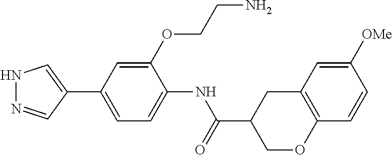

The title compound was prepared from 6-methoxy-chroman-3-carboxylic acid [2-[2-(dibenzylamino)-ethoxy]-4-(1H-pyrazol-4-yl)-phenyl]-amide according to the procedure described in Example 199. LC-MS: single peak at 254 nm, MH⁺ calcd. for C₂₁H₂₃N₄O₄: 409, obtained: 409.

### Example 278

### Synthesis of chroman-3-carboxylic acid [4-(2-amino-pyrimidin-4-yl)-2-dimethylcarbamoylmethoxy-phenyl]-amide

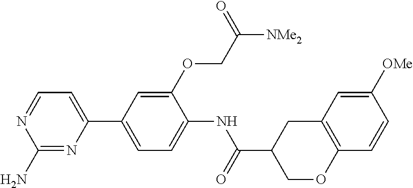

The title compound was prepared according to the procedure described in Scheme 8 (0.022 g, 18% over two steps). ¹H NMR (DMSO-d₆, 400 MHz) δ 10.30 (s, 1H), 8.34 (d, J=5.6 Hz, 1H), 8.25 (d, J=8.4 Hz, 1H), 7.81 (m, 2H), 7.81 (s, 1H), 7.27 (d J=6.0 Hz, 1H), 7.23 (br s, 2H), 6.74-6.69 (m, 3H), 5.04 (s, 2H), 4.41 (m, 1H), 4.04 (dd, J=10.8, 9.2 Hz, 1H), 3.69 (s, 3H), 3.24-3.19 (m, 1H), 3.06 (dd, J=16.4, 9.6 Hz, 1H), 3.00-2.95 (m, 1H), 2.99 (s, 3H), 2.88 (s, 3H). LC-MS: single peak at 254 nm, MH⁺ calcd. for C₂₅H₂₈N₅O₄: 478, obtained: 478.

### Example 279

### Synthesis of 6-chloro-chroman-3-carboxylic acid [2-dimethylcarbamoylmethoxy-4-(1H-pyrazol-4-yl)-phenyl]-amide

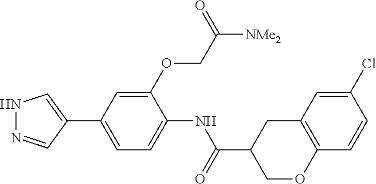

The title compound was prepared from 2-(2-amino-5-bromophenoxy)-N,N-dimethylacetamide and 6-chloro-chroman-3-carboxylic acid according to the procedure described in Scheme 4 (0.028 g, 29% over two steps). ¹H NMR (DMSO-d₆, 400 MHz) δ 10.01 (s, 1H), 8.01 (br s, 2H), 7.94 (d, J=8.4 Hz, 1H), 7.31 (d, J=2.0 Hz, 1H), 7.22 (m, 2H), 7.13 (dd, J=8.8, 2.8 Hz), 6.81 (d, J=8.8 Hz, 1H), 4.97 (s, 2H), 4.46 (dd, J=10.8, 1.6 Hz, 1H), 4.09 (dd, J=10.8, 9.2 Hz, 1H), 3.18-3.13 (m, 1 μl), 3.09-3.00 (m, 2H), 2.96 (s, 3H), 2.88 (s, 3H). LC-MS: single peak at 254 nm, MH⁺ calcd. for C₂₃H₂₄ClN₄O₄: 455, obtained: 455.

### Example 280

### Synthesis of 6-methoxy-chroman-3-carboxylic acid [2-[(2-dimethylamino-ethyl)-methyl-carbamoyl]-4-(1H-pyrazol-4-yl)-phenyl]-amide

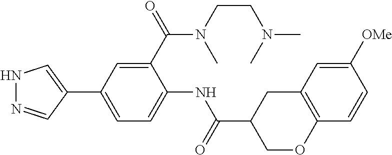

The title compound was prepared from 6-methoxy-chroman-3-carboxylic acid and 2-Amino-5-bromo-N-(2-dimethylamino-ethyl)-N-methyl-benzamide according to the procedure described in Scheme 4 (0.011 g, 24% over two steps). LC-MS: single peak at 254 nm, MH⁺ calcd. for C₂₆H₃₂N₅O₄: 478, obtained: 478.

### Example 281

### Synthesis of 6-chloro-chroman-3-carboxylic acid [2-dimethylcarbamoyl-4-(1H-pyrazol-4-yl)-phenyl]-amide

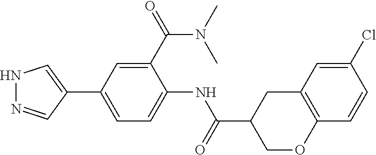

The title compound was prepared from 6-chloro-chroman-3-carboxylic acid and 2-Amino-5-bromo-N,N-dimethyl-benzamide according to the procedure described in Scheme 1 (0.33 mg, 30% over two steps). LC-MS: single peak at 254 nm, MH⁺ calcd. for C₂₂H₂₂ClN₄O₃: 425, obtained: 425.

### Example 282

### Synthesis of 6-methyl-chroman-3-carboxylic acid [2-dimethylcarbamoylmethoxy-4-(1H-pyrazol-4-yl)-phenyl]-amide

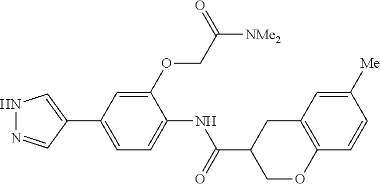

The title compound was prepared from 2-(2-amino-5-bromophenoxy)-N,N-dimethylacetamide and 6-methyl-chroman-3-carboxylic acid according to the procedure described in Scheme 4 (0.036 g, 30% over two steps). LC-MS: single peak at 254 nm, MH⁺ calcd. for C₂₄H₂₇N₄O₄: 435, obtained: 435.

### Example 283

### Synthesis of 6-methoxy-chroman-3-carboxylic acid [4-(2-amino-pyrimidin-4-0-2-dimethylcarbamoyl-phenyl]-amide

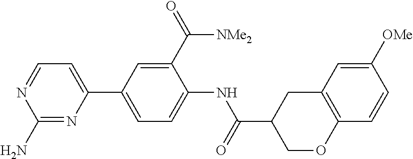

The title compound was prepared according to the procedure described in Scheme 8 (0.038 g, 27% over three steps). LC-MS: single peak at 254 nm, MH⁺ calcd. for C₂₄H₂₆N₅O₄: 448, obtained: 448.

### Example 284

### Synthesis of 6-methoxy-chroman-3-carboxylic acid [4-(2-amino-pyrimidin-4-yl)-2-methoxy-phenyl]-amide

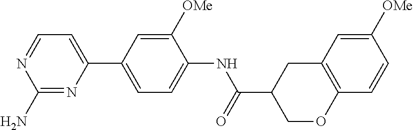

The title compound was prepared from 6-methoxy-chroman-3-carboxylic acid (4-bromo-2-methoxy-phenyl)-amide according to the procedure described in Scheme 8 (0.017 g, 32% over two steps). LC-MS: single peak at 254 nm, MH⁺ calcd. for C₂₂H₂₃N₄O₄: 407, obtained: 407.

### Example 285

### Synthesis of 6-fluoro-chroman-3-carboxylic acid [4-(2-amino-pyrimidin-4-yl)-2-dimethylcarbamoylmethoxy-phenyl]-amide

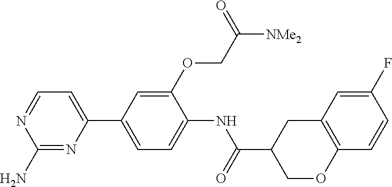

The title compound was prepared according to the procedure described in Scheme 8 (0.041 mg, 32% over three steps). LC-MS: single peak at 254 nm, MH⁺ calcd. for C₂₄H₂₄FN₅O₄: 466, obtained: 466.

### Example 286

### Synthesis of 6-methoxy-chroman-3-carboxylic acid [2-diethylcarbamoylmethoxy-4-(1H-pyrazol-4-yl)-phenyl]-amide

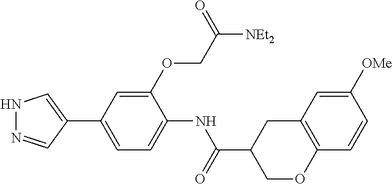

The title compound was prepared according to the procedure described in Example 191. LC-MS: single peak at 254 nm, MH⁺ calcd. for C₂₆H₃₁N₄O₅: 479, obtained: 479.

### Example 287

### Synthesis of 6-methyl-chroman-3-carboxylic acid [2-diethylcarbamoylmethoxy-4-(1H-pyrazol-4-yl)-phenyl]-amide

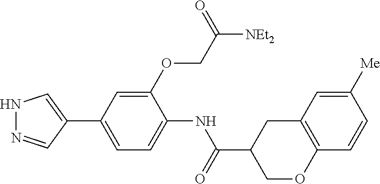

The title compound was prepared according to the procedure described in Example 191. LC-MS: single peak at 254 nm, MH⁺ calcd. for C₂₆H₃₁N₄O₄: 463, obtained: 463.

### Example 288

### Synthesis of 6-methyoxy-chroman-3-carboxylic acid [4-(2-amino-pyrimidin-4-yl)-2-diethylcarbamoylmethoxy-phenyl]-amide

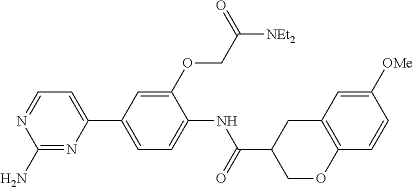

The title compound was prepared according to the procedure described in Scheme 8 (0.041 mg, 32% over three steps). LC-MS: single peak at 254 nm, MH⁺ calcd. for C₂₇H₃₂N₅O₅: 506, obtained: 506.

### Example 289

### Synthesis of 6-methyoxy-chroman-3-carboxylic acid [2-(2-dimethylamino-ethoxy)-4-pyridin-4-yl-phenyl]-amide

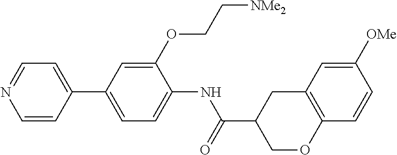

The title compound was prepared according to the procedure described in Scheme 4. LC-MS: single peak at 254 nm, MH⁺ calcd. for C₂₆H₃₀N₃O₄: 448, obtained: 448.

### Example 290

### Synthesis of 6-methyoxy-chroman-3-carboxylic acid [2-(2-dimethylamino-ethylsulfanyl)-4-pyridin-4-yl-phenyl]-amide

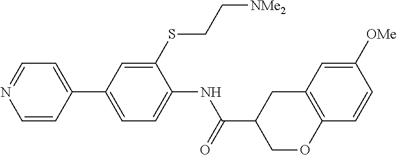

The title compound was prepared according to the procedure described in Scheme 4. LC-MS: single peak at 254 nm, MH⁺ calcd. for C₂₆H₃₀N₃O₃S: 464, obtained: 464.

### Example 291

### Synthesis of 6-methyoxy-chroman-3-carboxylic acid [2-(2-dimethylamino-ethylsulfanyl)-4-(3-methyl-1H-pyrazol-4-yl)-phenyl]-amide

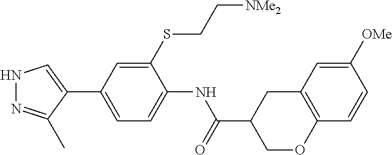

The title compound was prepared according to the procedure described in Scheme 4. LC-MS: single peak at 254 nm, MH⁺ calcd. for C₂₅H₃₁N₄O₃S: 467, obtained: 467.

### Example 292

### Synthesis of 6-methyoxy-chroman-3-carboxylic acid [2-(2-dimethylamino-ethylsulfanyl)-4-(1H-pyrazol-4-yl)-phenyl]-amide

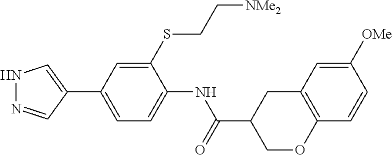

The title compound was prepared according to the procedure described in Scheme 4. LC-MS: single peak at 254 nm, MH⁺ calcd. for C₂₄H₂₉N₄O₃S: 453, obtained: 453.

### Example 293

### Synthesis of 6-methyoxy-chroman-3-carboxylic acid [4-(2-amino-pyrimidin-4-yl)-2-(2-dimethylamino-ethylsulfanyl)-phenyl]-amide

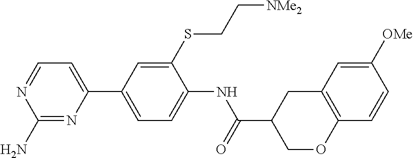

The title compound was prepared according to the procedure described in Scheme 8. LC-MS: single peak at 254 nm, MH⁺ calcd. for C₂₅H₃₀N₅O₃S: 480, obtained: 480.

### Example 294

### Synthesis of 6-fluoro-chroman-3-carboxylic acid [2-(2-dimethylamino-ethoxy)-4-(1H-pyrrolo[2,3-b]pyridin-4-yl)-phenyl]-amide

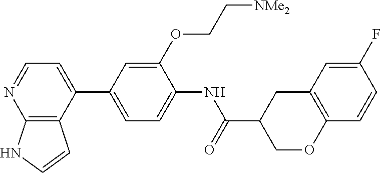

The title compound was prepared according to the procedure described in Scheme 23. LC-MS: single peak at 254 nm, MH⁺ calcd. for C₂₇H₂₈FN₄O₃: 475, obtained: 475.

### Example 295

### Synthesis of 6-fluoro-chroman-3-carboxylic acid [2-(2-dimethylamino-ethoxy)-4-(7H-pyrrolo[2,3-d]pyrimidin-4-yl)-phenyl]-amide

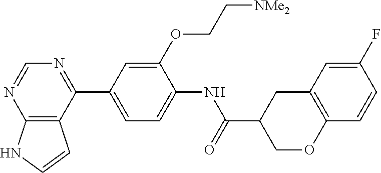

The title compound was prepared according to the procedure described in Scheme 23. LC-MS: single peak at 254 nm, MH⁺ calcd. for C₂₆H₂₇FN₅O₃: 476, obtained: 476.

### Example 296

### Synthesis of 6-fluoro-chroman-3-carboxylic acid [2-(2-dimethylamino-ethoxy)-4-pyridin-4-yl-phenyl]-amide

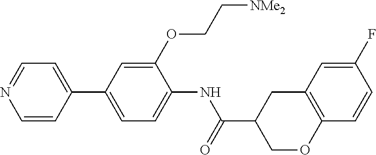

The title compound was prepared according to the procedure described in Scheme 4. LC-MS: single peak at 254 nm, MH⁺ calcd. for C₂₅H₂₇FN₃O₃: 436, obtained: 436.

### Example 297

### Synthesis of 6-methyoxy-chroman-3-carboxylic acid [2-(2-dimethylamino-ethoxy)-4-(3-methyl-1H-pyrazol-4-yl)-phenyl]-amide

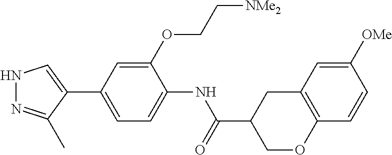

The title compound was prepared according to the procedure described in Scheme 4. LC-MS: single peak at 254 nm, MH⁺ calcd. for C₂₅H₃₁N₄O₄: 451, obtained: 451.

### Example 298

### Synthesis of 6-acetyl-chroman-3-carboxylic acid [2-(2-dimethylamino-ethoxy)-4-(1H-pyrazol-4-yl)-phenyl]-amide

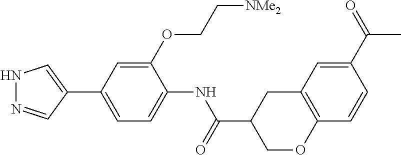

The title compound was prepared according to the procedure described in Scheme 4. LC-MS: single peak at 254 nm, MH⁺ calcd. for C₂₅H₂₉N₄O₄: 449, obtained: 449.

### Example 299

### Synthesis of 6-acetyl-chroman-3-carboxylic acid [4-(2-amino-pyrimidin-4-yl)-2-(2-dimethylamino-ethoxy)-phenyl]-amide

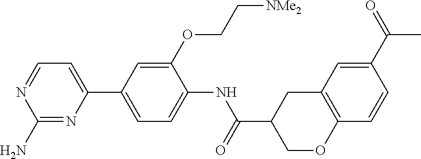

The title compound was prepared according to the procedure described in Scheme 8. LC-MS: single peak at 254 nm, MH⁺ calcd. for C₂₆H₃₀N₅O₄: 476, obtained: 476.

### Example 300

### Synthesis of chroman-3-carboxylic acid [4-(3-methyl-1H-pyrazol-4-yl)-phenyl]-amide

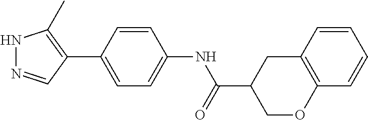

The title compound was prepared according to the procedure described in Scheme 8. LC-MS: single peak at 254 nm, MH⁺ calcd. for C₂₀H₂₀N₃O₂: 334, obtained: 334.

### Example 301

### Synthesis of 6-methoxy-chroman-3-carboxylic acid [2-benzyloxy-4-(1H-pyrazol-4-yl)-phenyl]-amide

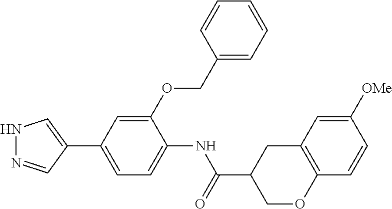

The title compound was prepared according to the procedure described in Scheme 4. LC-MS: single peak at 254 nm, MH⁺ calcd. for C₂₇H₂₆N₃O₄: 456, obtained: 456.

### Example 302

### Synthesis of 6-fluoro-chroman-3-carboxylic acid [2-(2-dimethylamino-ethylsulfanyl)-4-(1H-pyrazol-4-yl)-phenyl]-amide

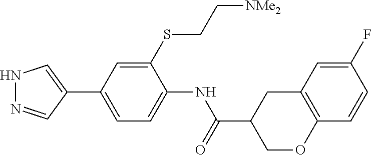

The title compound was prepared according to the procedure described in Scheme 4. LC-MS: single peak at 254 nm, MH⁺ calcd. for C₂₃H₂₅FN₄O₂S: 441, obtained: 441.

### Example 303

### Synthesis of 6-fluoro-chroman-3-carboxylic acid [2-(2-dimethylamino-ethylsulfanyl)-4-(3-methyl-1H-pyrazol-4-yl)-phenyl]-amide

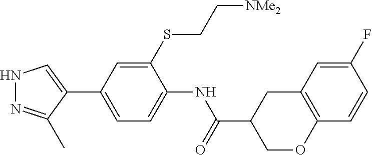

The title compound was prepared according to the procedure described in Scheme 4. LC-MS: single peak at 254 nm, MH⁺ calcd. for C₂₄H₂₇FN₄O₂S: 455, obtained: 455.

### Example 304

### Synthesis of 6-fluoro-chroman-3-carboxylic acid [2-(2-dimethylamino-ethylsulfanyl)-4-pyridin-4-yl-phenyl]-amide

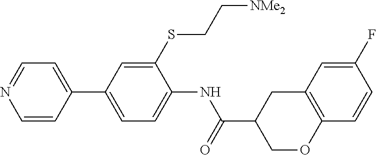

The title compound was prepared according to the procedure described in Scheme 4. LC-MS: single peak at 254 nm, MH⁺ calcd. for C₂₅H₂₇FN₃O₂S: 452, obtained: 452.

### Example 305

### Synthesis of 6-methoxy-chroman-3-carboxylic acid [2-dimethylcarbamoylmethoxy-4-(3-methyl-1H-pyrazol-4-yl)-phenyl]-amide

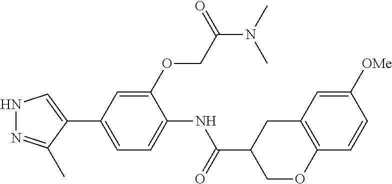

The title compound was prepared from 2-(2-amino-5-bromophenoxy)-N,N-dimethylacetamide and 6-methoxy-chroman-3-carboxylic acid according to the procedure described in Scheme 4. LC-MS: single peak at 254 nm, MH⁺ calcd. for C₂₅H₂₈N₄O₅: 465, obtained: 465.

### Example 306

### Synthesis of 6-fluoro-chroman-3-carboxylic acid [2-(2-dimethylamino-ethoxy)-4-(3-methyl-1H-pyrazol-4-yl)-phenyl]-amide

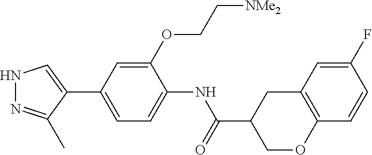

The title compound was prepared according to the procedure described in Scheme 4. LC-MS: single peak at 254 nm, MH⁺ calcd. for C₂₄H₂₈FN₄O₃: 439, obtained: 439.

### Example 307

### Synthesis of 6-methoxy-chroman-3-carboxylic acid [2-(2-morpholin-4-yl-ethoxy)-4-(1H-pyrazol-4-yl)-phenyl]-amide

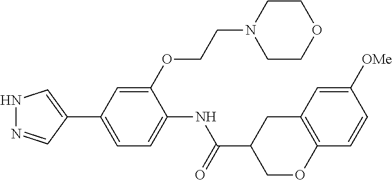

The title compound was prepared according to the procedure described in Scheme 4. LC-MS: single peak at 254 nm, MH⁺ calcd. for C₂₆H₃₁N₄O₅: 479, obtained: 479.

### Example 308

### Synthesis of 6-methoxy-chroman-3-carboxylic acid [2-(2-morpholin-4-yl-ethoxy)-4-(3-methyl-1H-pyrazol-4-yl)-phenyl]-amide

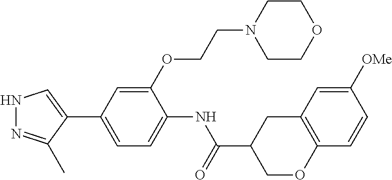

The title compound was prepared according to the procedure described in Scheme 4. LC-MS: single peak at 254 nm, MH⁺ calcd. for C₂₇H₃₃FN₄O₅: 493, obtained: 493.

### Example 309

### Synthesis of 6-methoxy-chroman-3-carboxylic acid [2-(2-morpholin-4-yl-ethoxy)-4-pyridin-4-yl-phenyl]-amide

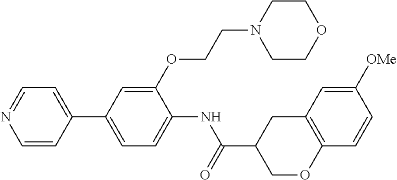

The title compound was prepared according to the procedure described in Scheme 4. LC-MS: single peak at 254 nm, MH⁺ calcd. for C₂₈H₃₂N₃O₅: 490, obtained: 490.

### Example 310

### Synthesis of 6-methoxy-chroman-3-carboxylic acid [2-(2pyrrolidin-1-yl-ethoxy)-4-(1H-pyrazol-4-yl)-phenyl]-amide

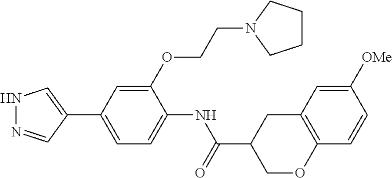

The title compound was prepared according to the procedure described in Scheme 4. LC-MS: single peak at 254 nm, MH⁺ calcd. for C₂₆H₃₁N₄O₄: 463, obtained: 463.

### Example 311

### Synthesis of 6-methoxy-chroman-3-carboxylic acid [2-(2-pyrrolidin-1-yl-ethoxy)-4-(3-methyl-1H-pyrazol-4-yl)-phenyl]-amide

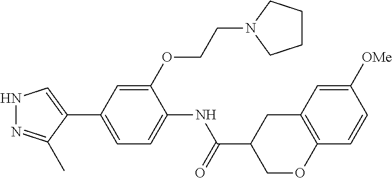

The title compound was prepared according to the procedure described in Scheme 4. LC-MS: single peak at 254 nm, MH⁺ calcd. for C₂₇H₃₃N₄O₄: 477, obtained: 477.

### Example 312

### Synthesis of 6-methoxy-chroman-3-carboxylic acid [2-(2-pyrrolidin-1-yl-ethoxy)-4-pyridin-4-yl-phenyl]-amide

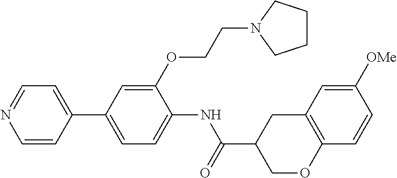

The title compound was prepared according to the procedure described in Scheme 4. LC-MS: single peak at 254 nm, MH⁺ calcd. for C₂₈H₃₂N₃O₄: 474, obtained: 474.

### Example 313

### Synthesis of 6-methyoxy-chroman-3-carboxylic acid [4-(2-amino-pyrimidin-4-yl)-2-(2-morpholin-4-yl-ethoxy)-phenyl]-amide

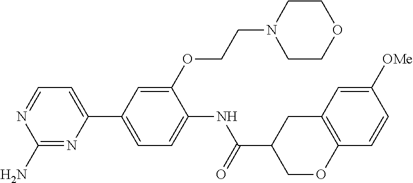

The title compound was prepared according to the procedure described in Scheme 8. LC-MS: single peak at 254 nm, MH⁺ calcd. for C₂₇H₃₂N₅O₅: 506, obtained: 506.

### Example 314

### Synthesis of 6-methyoxy-chroman-3-carboxylic acid [4-(2-amino-pyrimidin-4-yl)-2-(2-pyrrolidin-1-yl-ethoxy)-phenyl]-amide

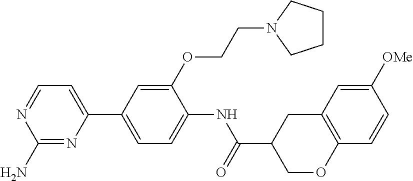

The title compound was prepared according to the procedure described in Scheme 8. LC-MS: single peak at 254 nm, MH⁺ calcd. for C₂₇H₃₂N₅O₄: 490, obtained: 490.

### Example 315

### Synthesis of (R)-2,3-dihydro-benzo[1,4]dioxine-2-carboxylic acid [2-(2-morpholin-4-yl-ethoxy)-4-(1H-pyrazol-4-yl)-phenyl]-amide

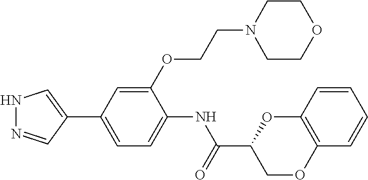

The title compound was prepared according to the procedure described in Scheme 4. LC-MS: single peak at 254 nm, MH⁺ calcd. for C₂₄H₂₇N₄O₅: 451, obtained: 451.

### Example 316

### Synthesis of (R)-2,3-dihydro-benzo[1,4]dioxine-2-carboxylic acid [2-(2-pyrrolidin-1-yl-ethoxy)-4-(1H-pyrazol-4-yl)-phenyl]-amide

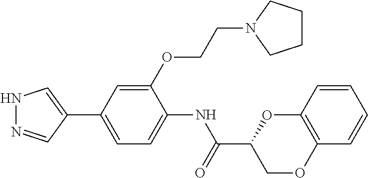

The title compound was prepared according to the procedure described in Scheme 4. LC-MS: single peak at 254 nm, MH⁺ calcd. for C₂₄H₂₇N₄O₄: 435, obtained: 435.

### Example 317

### Synthesis of 6-methoxy-chroman-3-carboxylic acid [2-(2-morpholin-4-yl-ethoxy)-4-(1H-pyrazol-4-yl)-phenyl]-amide

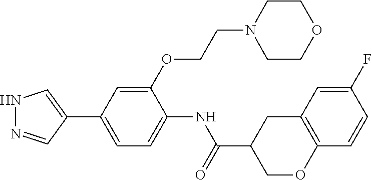

The title compound was prepared according to the procedure described in Scheme 4. LC-MS: single peak at 254 nm, MH⁺ calcd. for C₂₅H₂₈FN₄O₄: 467, obtained: 467.

### Example 318

### Synthesis of 6-fluoro-chroman-3-carboxylic acid [2-(2-pyrrolidin-1-yl-ethoxy)-4-(3-methyl-1H-pyrazol-4-yl)-phenyl]-amide

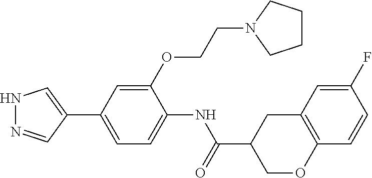

The title compound was prepared according to the procedure described in Scheme 4. LC-MS: single peak at 254 nm, MH⁺ calcd. for C₂₅H₂₈FN₄O₃: 451, obtained: 451.

### Example 319

### Synthesis of 6-methoxy-chroman-3-carboxylic acid [2-(2-dimethylamino-ethoxy)-4-(2-methyl-2H-pyrazol-3-yl)-phenyl]-amide

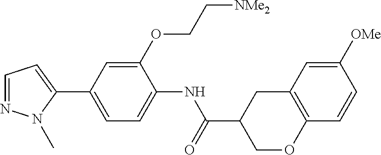

The title compound was prepared according to the procedure described in Scheme 4. LC-MS: single peak at 254 nm, MH⁺ calcd. for C₂₅H₃₁N₄O₄: 451, obtained: 451.

### Example 320

### Synthesis of 6-methoxy-chroman-3-carboxylic acid [2-(2-morpholin-4-yl-ethoxy)-4-(2-methyl-2H-pyrazol-3-yl)-phenyl]-amide

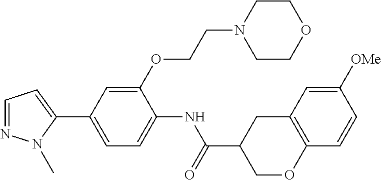

The title compound was prepared according to the procedure described in Scheme 4. LC-MS: single peak at 254 nm, MH⁺ calcd. for C₂₇H₃₃N₄O₅: 493, obtained: 493.

### Example 321

### Synthesis of 6-fluoro-chroman-3-carboxylic acid [2-(2-morpholin-4-yl-ethoxy)-4-(3-methyl-1H-pyrazol-4-yl)-phenyl]-amide

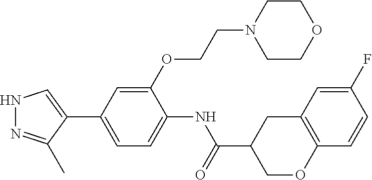

The title compound was prepared according to the procedure described in Scheme 4. LC-MS: single peak at 254 nm, MH⁺ calcd. for C₂₆H₃₀N₄O₄: 481, obtained: 481.

### Example 322

### Synthesis of 6-fluoro-chroman-3-carboxylic acid [2-(2-pyrrolidin-1-yl-ethoxy)-4-(3-methyl-1H-pyrazol-4-yl)-phenyl]-amide

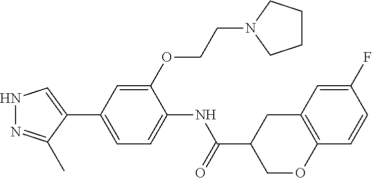

The title compound was prepared according to the procedure described in Scheme 4. LC-MS: single peak at 254 nm, MH⁺ calcd. for C₂₆H₃₀FN₄O₃: 465, obtained: 465.

### Example 323

### Synthesis of 6-methoxy-chroman-3-carboxylic acid [2-[(2-dimethylamino-ethyl)-methyl-amino]-4-(1H-pyrazol-4-yl)-phenyl]-amide

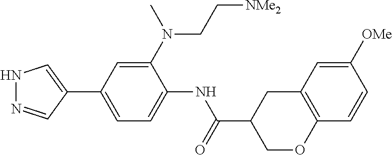

The title compound was prepared according to the procedure described in Scheme 4. LC-MS: single peak at 254 nm, MH⁺ calcd. for C₂₅H₃₂N₅O₃: 450, obtained: 450.

### Example 324

### Synthesis of 6-methoxy-chroman-3-carboxylic acid [2-[(2-dimethylamino-ethyl)-methyl-amino]-4-(3-methyl-1H-pyrazol-4-yl)-phenyl]-amide

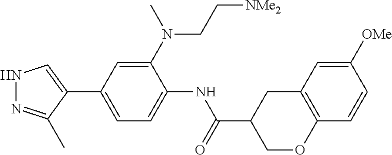

The title compound was prepared according to the procedure described in Scheme 4. LC-MS: single peak at 254 nm, MH⁺ calcd. for C₂₆H₃₄N₅O₃: 464, obtained: 464.

### Example 325

### Synthesis of 6-methoxy-chroman-3-carboxylic acid [2-(3-dimethylamino-propoxy)-4-(1H-pyrazol-4-yl)-phenyl]-amide

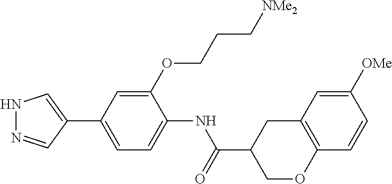

The title compound was prepared according to the procedure described in Scheme 4. LC-MS: single peak at 254 nm, MH⁺ calcd. for C₂₅H₃₁N₄O₄: 451, obtained: 451.

### Example 326

### Synthesis of 6-methoxy-chroman-3-carboxylic acid [2-(3-dimethylamino-propoxy)-4-(3-methyl-1H-pyrazol-4-yl)-phenyl]-amide

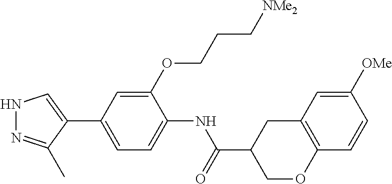

The title compound was prepared according to the procedure described in Scheme 4. LC-MS: single peak at 254 nm, MH⁺ calcd. for C₂₆H₃₃N₄O₄: 465, obtained: 465.

### Example 327

### Synthesis of 6-fluoro-chroman-3-carboxylic acid [2-(2-morpholin-4-yl-ethylsulfanyl)-4-(1H-pyrazol-4-yl)-phenyl]-amide

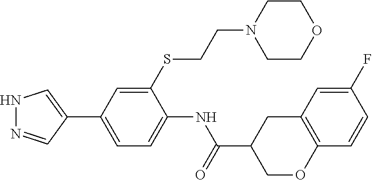

The title compound was prepared according to the procedure described in Scheme 1. LC-MS: single peak at 254 nm, MH⁺ calcd. for C₂₅H₂₈FN₄O₃S: 483, obtained: 483.

### Example 328

### Synthesis of 6-methyl-chroman-3-carboxylic acid [2-(2-morpholin-4-yl-ethylsulfanyl)-4-(1H-pyrazol-4-yl)-phenyl]-amide

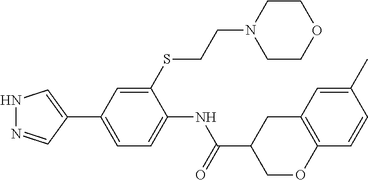

The title compound was prepared according to the procedure described in Scheme 1. LC-MS: single peak at 254 nm, MH⁺ calcd. for C₂₆H₃₁N₄O₃S: 479, obtained: 479.

### Example 329

### Synthesis of 6-fluoro-chroman-3-carboxylic acid [2-(2-morpholin-4-yl-ethylsulfanyl)-4-(3-methyl-1H-pyrazol-4-yl)-phenyl]-amide

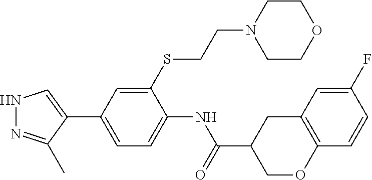

The title compound was prepared according to the procedure described in Scheme 1. LC-MS: single peak at 254 nm, MH⁺ calcd. for C₂₆H₃₀FN₄O₃S: 497, obtained: 497.

### Example 330

### Synthesis of 6-methoxy-chroman-3-carboxylic acid [2-(2-pyrrolidin-1-yl-ethylsulfanyl)-4-(1H-pyrazol-4-yl)-phenyl]-amide

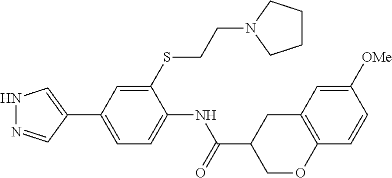

The title compound was prepared according to the procedure described in Scheme 1. LC-MS: single peak at 254 nm, MH⁺ calcd. for C₂₆H₃₁N₄O₃S: 479, obtained: 479.

### Example 331

### Synthesis of 6-methoxy-chroman-3-carboxylic acid [2-(2-pyrrolidin-1-yl-ethylsulfanyl)-4-(3-methyl-1H-pyrazol-4-yl)-phenyl]-amide

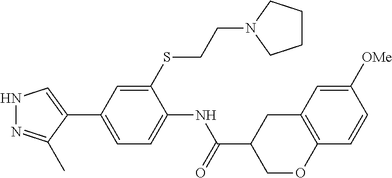

The title compound was prepared according to the procedure described in Scheme 1. LC-MS: single peak at 254 nm, MH⁺ calcd. for C₂₇H₃₃N₄O₃S: 493, obtained: 493.

### Example 332

### Synthesis of 6-methoxy-chroman-3-carboxylic acid [2-(2-morpholin-4-yl-ethylsulfanyl)-4-(1H-pyrazol-4-yl)-phenyl]-amide

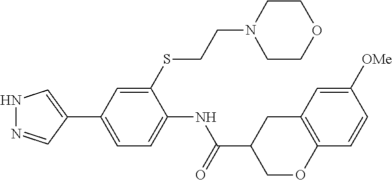

The title compound was prepared according to the procedure described in Scheme 1. LC-MS: single peak at 254 nm, MH⁺ calcd. for C₂₆H₃₁N₄O₄S: 495, obtained: 495.

### Example 333

### Synthesis of 6-methoxy-chroman-3-carboxylic acid [2-(2-morpholin-4-yl-ethylsulfanyl)-4-(3-methyl-1H-pyrazol-4-yl)-phenyl]-amide

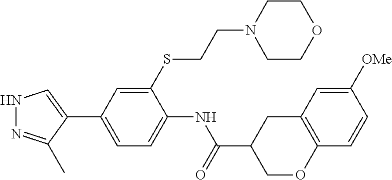

The title compound was prepared according to the procedure described in Scheme 1. LC-MS: single peak at 254 nm, MH⁺ calcd. for C₂₇H₃₃N₄O₄S: 509, obtained: 509.

### Example 334

### Synthesis of 6-fluoro-chroman-3-carboxylic acid [2-(2-pyrrolidin-1-yl-ethylsulfanyl)-4-(1H-pyrazol-4-yl)-phenyl]-amide

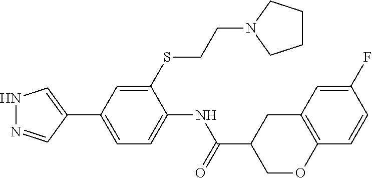

The title compound was prepared according to the procedure described in Scheme 1. LC-MS: single peak at 254 nm, MH⁺ calcd. for C₂₅H₂₈FN₄O₂S: 467, obtained: 467.

### Example 335

### Synthesis of 6-fluoro-chroman-3-carboxylic acid [2-(2-pyrrolidin-1-yl-ethylsulfanyl)-4-(3-methyl-1H-pyrazol-4-yl)-phenyl]-amide

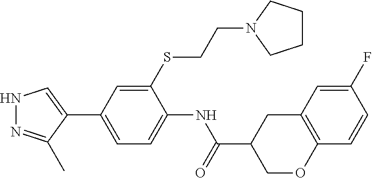

The title compound was prepared according to the procedure described in Scheme 1. LC-MS: single peak at 254 nm, MH⁺ calcd. for C₂₆H₃₀FN₄O₂S: 481, obtained: 481.

### Example 336

### Sunthesis of 2,3-dihydro-naphtho[2,3-b][1,4]dioxine-carboxylic acid [2-(3-dimethylamino-ethoxy)-4-(1H-pyrazol-4-yl)-phenyl]-amide

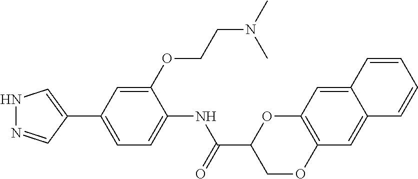

The title compound was prepared according to the procedure described in Scheme 4. LC-MS: single peak at 254 nm, MH⁺ calcd. for C₂₆H₂₇N₄O₄: 459, obtained: 459.

### Example 337

### Synthesis of 6-methoxy-chroman-3-carboxylic acid [2-[methyl-(2-morpholin-4-yl-ethyl)-amino]-4-(1H-pyrazol-4-yl)-phenyl]-amide

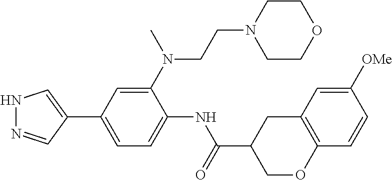

The title compound was prepared according to the procedure described in Scheme 1. LC-MS: single peak at 254 nm, MH⁺ calcd. for C₂₇H₃₄N₅O₄: 492, obtained: 492.

### Example 338

### Synthesis of 6-methoxy-chroman-3-carboxylic acid [2-[methyl-(2-morpholin-4-yl-ethyl)-amino]-4-(3-methyl-1H-pyrazol-4-yl)-phenyl]-amide

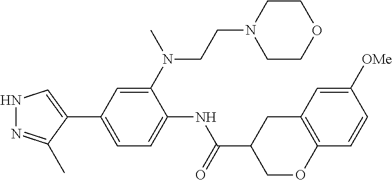

The title compound was prepared according to the procedure described in Scheme 1. LC-MS: single peak at 254 nm, MH⁺ calcd. for C₂₈H₃₆N₅O₄: 506, obtained: 506.

### Example 339

### Synthesis of 2,3-dihydro-benzo[1,4]dioxine-2-carboxylic acid [2-(2-dimethylamino-ethylsulfanyl)-4-(1H-pyrazol-4-yl)-phenyl]-amide

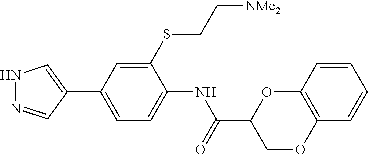

The title compound was prepared according to the procedure described in Scheme 1. LC-MS: single peak at 254 nm, MH⁺ calcd. for C₂₂H₂₅N₄O₃S: 425, obtained: 425.

### Example 340

### Synthesis of 2,3-dihydro-benzo[1,4]dioxine-2-carboxylic acid [2-(2-dimethylamino-ethylsulfanyl)-4-(3-methyl-1H-pyrazol-4-yl)-phenyl]-amide

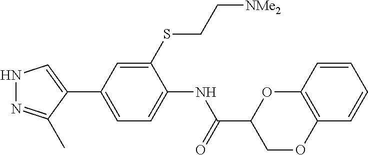

The title compound was prepared according to the procedure described in Scheme 1. LC-MS: single peak at 254 nm, MH⁺ calcd. for C₂₃H₂₇N₄O₃S: 439, obtained: 439.

### Example 341

### Synthesis of 2,3-dihydro-benzo[1,4]dioxine-2-carboxylic acid [2-[(2-dimethylamino-ethyl)-methyl-amino]-4-(1H-pyrazol-4-yl)-phenyl]-amide

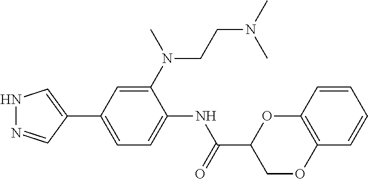

The title compound was prepared according to the procedure described in Scheme 4. LC-MS: single peak at 254 nm, MH⁺ calcd. for C₂₃H₂₈N₅O₃S: 422, obtained: 422.

### Example 342

### Synthesis of 2,3-dihydro-benzo[1,4]dioxine-2-carboxylic acid [2-[(2-dimethylamino-ethyl)-methyl-amino]-4-(3-methyl-1H-pyrazol-4-yl)-phenyl]amide

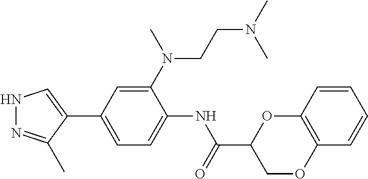

The title compound was prepared according to the procedure described in Scheme 4. LC-MS: single peak at 254 nm, MH⁺ calcd. for C₂₄H₃₀N₅O₃S: 436, obtained 436: 438.

### Example 343

### Synthesis of 6-fluoro-chroman-3-carboxylic acid [2-[(2-dimethylamino-ethyl)-methyl-amino]-4-(1H-pyrazol-4-yl)-phenyl]-amide

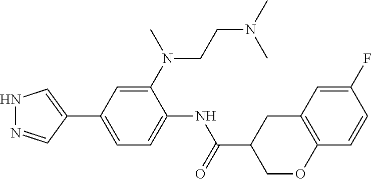

The title compound was prepared according to the procedure described in Scheme 4. LC-MS: single peak at 254 nm, MH⁺ calcd. for C₂₄H₂₉FN₅O₂: 438, obtained: 438.

### Example 344

### Synthesis of 6-fluoro-chroman-3-carboxylic acid [2-[(2-dimethylamino-ethyl)-methyl-amino]-4-(3-methyl-1H-pyrazol-4-yl)-phenyl]-amide

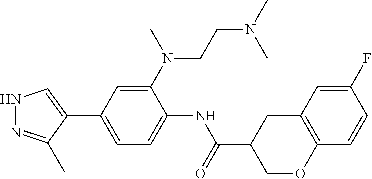

The title compound was prepared according to the procedure described in Scheme 4. LC-MS: single peak at 254 nm, MH⁺ calcd. for C₂₅H₃₁N₅O₂: 452, obtained: 452.

### Example 345

### Synthesis of 6-methoxy-chroman-3-carboxylic acid [2-(2-diethylamino-ethoxy)-4-(1H-pyrazol-4-yl)-phenyl]-amide

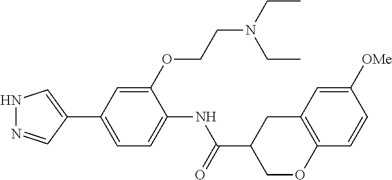

The title compound was prepared according to the procedure described in Scheme 4. LC-MS: single peak at 254 nm, MH⁺ calcd. for C₂₆H₃₃N₄O₄: 465, obtained: 465.

### Example 346

### Synthesis of 6-methoxy-chroman-3-carboxylic acid [4-(1H-pyrazol-4-yl)-2-(2-pyridin-2-yl-ethoxy)-phenyl]-amide

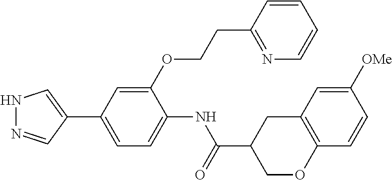

The title compound was prepared according to the procedure described in Scheme 4. LC-MS: single peak at 254 nm, MH⁺ calcd. for C₂₇H₂₇N₄O₄: 471, obtained: 471.

### Example 347

### Synthesis of 6-methoxy-chroman-3-carboxylic acid [2-(2-diethylamino-ethoxy)-4-(3-methyl-1H-pyrazol-4-yl)-phenyl]-amide

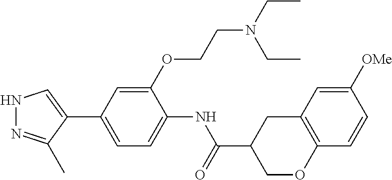

The title compound was prepared according to the procedure described in Scheme 4. LC-MS: single peak at 254 nm, MH⁺ calcd. for C₂₇H₃₅N₄O₄: 479, obtained: 479.

### Example 348

### Synthesis of 6-methoxy-chroman-3-carboxylic acid [4-(1H-pyrazol-4-yl)-2-(pyridin-3-yloxy)-phenyl]-amide

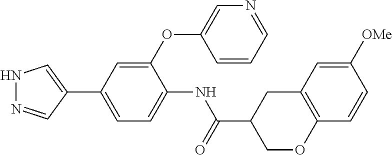

The title compound was prepared according to the procedure described in Scheme 4. LC-MS: single peak at 254 nm, MH⁺ calcd. for C₂₅H₂₃N₄O₄: 443, obtained: 443.

### Example 349

### Synthesis of 6-methoxy-chroman-3-carboxylic acid [2-(2-allyloxy-ethoxy)-4-(1H-pyrazol-4-yl)-phenyl]-amide

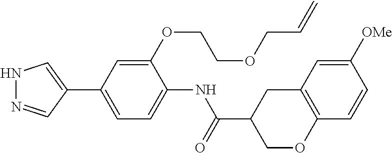

The title compound was prepared according to the procedure described in Scheme 4. LC-MS: single peak at 254 nm, MH⁺ calcd. for C₂₅H₂₈N₃O₅: 450, obtained: 450.

### Example 350

### Synthesis of 6-methoxy-chroman-3-carboxylic acid [2-(2-hydroxy-ethoxy)-4-(3-methyl-1H-pyrazol-4-yl)-phenyl]-amide

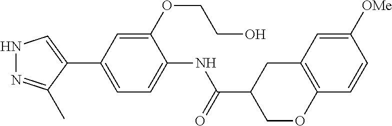

The title compound was prepared according to the procedure described in Scheme 4. LC-MS: single peak at 254 nm, MH⁺ calcd. for C₂₃H₂₆N₃O₅: 424, obtained: 424.

### Example 351

### Synthesis of 6-methoxy-chroman-3-carboxylic acid [2-(2-allyloxy-ethoxy)-4-(3-methyl-1H-pyrazol-4-yl)-phenyl]-amide

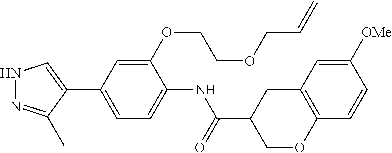

The title compound was prepared according to the procedure described in Scheme 4. LC-MS: single peak at 254 nm, MH⁺ calcd. for C₂₆H₃₀N₃O₅: 464, obtained: 464.

### Example 352

### Synthesis of 6-methoxy-chroman-3-carboxylic acid [4-(1H-pyrazol-4-yl)-2-(pyridin-2-ylmethoxy)-phenyl]-amide

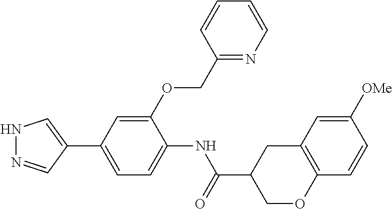

The title compound was prepared according to the procedure described in Scheme 4. LC-MS: single peak at 254 nm, MH⁺ calcd. for C₂₆H₂₅N₄O₄: 457, obtained: 457.

### Example 353

### Synthesis of 6-methoxy-chroman-3-carboxylic acid [2-carbamoylmethoxy-4-(3-methyl-1H-pyrazol-4-yl)-phenyl]-amide

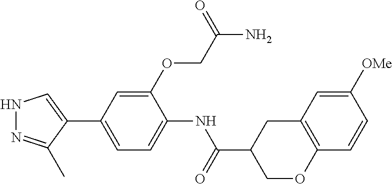

The title compound was prepared according to the procedure described in Scheme 4. LC-MS: single peak at 254 nm, MH⁺ calcd. for C₂₃H₂₅N₄O₅: 437, obtained: 437.

### Example 354

### Synthesis of 6-methoxy-chroman-3-carboxylic acid [2-[methyl-(2-morpholino-4-yl-ethyl)-amino]-4-pyridin-4-yl-phenyl]-amide

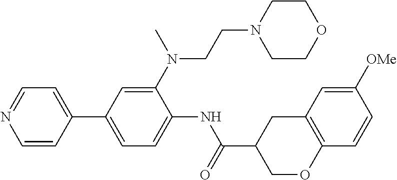

The title compound was prepared according to the procedure described in Scheme 4. LC-MS: single peak at 254 nm, MH⁺ calcd. for C₂₉H₃₅N₄O₄: 503, obtained: 503.

### Example 355

### Synthesis of 6-methoxy-chroman-3-carboxylic acid [2-[(2-dimethylamino-ethyl)-methyl-amino]-4-pyridin-4-yl-phenyl]-amide

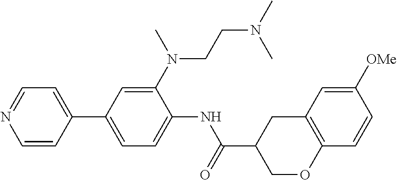

The title compound was prepared according to the procedure described in Scheme 4. LC-MS: single peak at 254 nm, MH⁺ calcd. for C₂₇H₃₃N₄O₃: 461, obtained: 461.

### Example 356

### Synthesis of 6-methoxy-chroman-3-carboxylic acid [2-[methyl-(2-pyrrolidin-1-yl-ethyl)-amino]-4-(1H-pyrazol-4-yl-phenyl]-amide

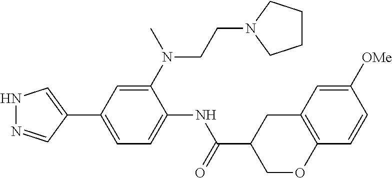

### Step A. N-methyl-2-(pyrrolidin-1-yl)ethanamine

The title compound was prepared according to the procedure of de Costa, et al. J. Med. Chem. 1992, 35, 38-47.

### Step B. Synthesis of 6-methoxy-chroman-3-carboxylic acid [2-[methyl-(2-pyrrolidin-1-yl-ethyl)-amino]-4-(1H-pyrazol-4-yl-phenyl]-amide

The title compound was prepared according to the procedure described in Scheme 4. LC-MS: single peak at 254 nm, MH⁺ calcd. for C₂₇H₃₄N₅O₃: 476, obtained: 476.

### Example 357

### Synthesis of 6-methoxy-chroman-3-carboxylic acid [2-[methyl-(2-pyrrolidin-1-yl-ethyl)-amino]-4-(3-methyl-1H-pyrazol-4-yl-phenyl]-amide

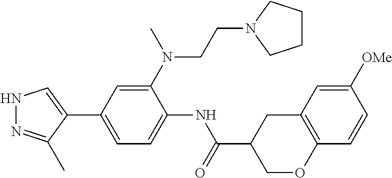

The title compound was prepared according to the procedure described in Scheme 4. LC-MS: single peak at 254 nm, MH⁺ calcd. for C₂₈H₃₆N₅O₃: 490, obtained: 490.

### Example 358

### Synthesis of 6-methoxy-chroman-3-carboxylic acid [4-(1H-pyrazol-4-yl)-2-(pyridin-3-ylmethoxy)-phenyl]-amide

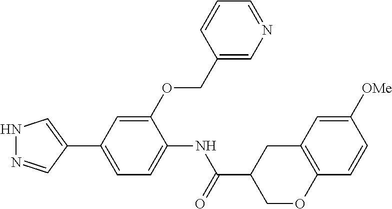

The title compound was prepared according to the procedure described in Scheme 4. LC-MS: single peak at 254 nm, MH⁺ calcd. for C₂₆H₂₅N₄O₄: 457, obtained: 457.

### Example 359

### Synthesis of 6-methoxy-chroman-3-carboxylic acid [4-(3-methyl-1H-pyrazol-4-yl)-2-(pyridin-3-ylmethoxy)-phenyl]-amide

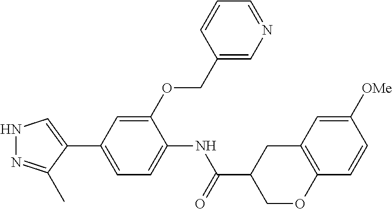

The title compound was prepared according to the procedure described in Scheme 4. LC-MS: single peak at 254 nm, MH⁺ calcd. for C₂₇H₂₇N₄O₄: 471, obtained: 471.

### Example 360

### Synthesis of 6-methoxy-chroman-3-carboxylic acid [24(S)-1-methyl-pyrrolidin-2-ylmethoxy)-4-(1H-pyrazol-4-yl)-phenyl]-amide

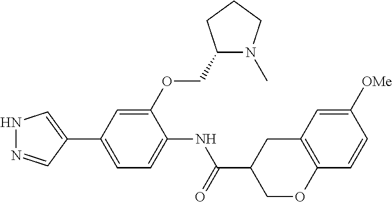

The title compound was prepared according to the procedure described in Scheme 4. LC-MS: single peak at 254 nm, MH⁺ calcd. for C₂₆H₃₁N₄O₄: 463, obtained: 463.

### Example 361

### Synthesis of 6-methoxy-chroman-3-carboxylic acid [24(S)-1-methyl-pyrrolidin-2-ylmethoxy)-4-(3-methyl-1H-pyrazol-4-yl)-phenyl]-amide

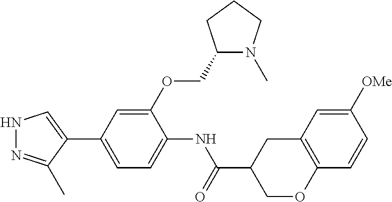

The title compound was prepared according to the procedure described in Scheme 4. LC-MS: single peak at 254 nm, MH⁺ calcd. for C₂₇H₃₃N₄O₄: 477, obtained: 477.

### Example 362

### Synthesis of 6-methoxy-chroman-3-carboxylic acid [2-phenoxy-4-(1H-pyrazol-4-yl)-phenyl]-amide

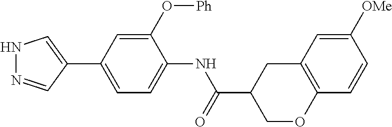

The title compound was prepared according to the procedure described in Scheme 4. LC-MS: single peak at 254 nm, MH⁺ calcd. for C₂₆H₂₄N₃O₄: 442, obtained: 442.

### Example 363

### Synthesis of 6-methoxy-chroman-3-carboxylic acid [2-(1-methyl-piperidin-3-yloxy)-4-(1H-pyrazol-4-yl)-phenyl]-amide

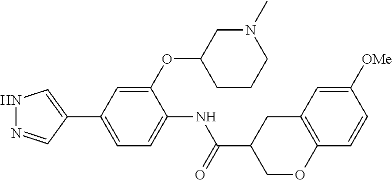

The title compound was prepared according to the procedure described in Scheme 4. LC-MS: single peak at 254 nm, MH⁺ calcd. for C₂₆H₃₁N₄O₄: 463, obtained: 463.

### Example 364

### Synthesis of 6-methoxy-chroman-3-carboxylic acid [2-(1-methyl-piperidin-3-yloxy)-4-(3-methyl-1H-pyrazol-4-yl)-phenyl]-amide

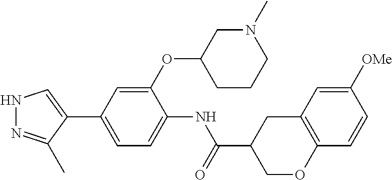

The title compound was prepared according to the procedure described in Scheme 4. LC-MS: single peak at 254 nm, MH⁺ calcd. for C₂₇H₃₃N₄O₄: 477, obtained: 477.

### Example 365

### Synthesis of 6-methoxy-chroman-3-carboxylic acid [2-((S)-1-benzyl-pyrrolidin-2-ylmethoxy)-4-(1H-pyrazol-4-yl)-phenyl]-amide

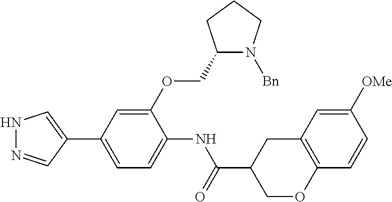

The title compound was prepared according to the procedure described in Scheme 4. LC-MS: single peak at 254 nm, MH⁺ calcd. for C₃₂H₃₅N₄O₄: 539, obtained: 539.

### Example 366

### Synthesis of 6-methoxy-chroman-3-carboxylic acid [2-((S)-1-pyrrolidin-2-ylmethoxy)-4-(1H-pyrazol-4-yl)-phenyl]-amide

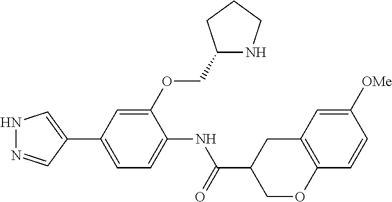

The title compound was prepared from 6-methoxy-chroman-3-carboxylic acid [24(S)-1-benzyl-pyrrolidin-2-ylmethoxy)-4-(1H-pyrazol-4-yl)-phenyl]-amide according to the procedure described in Example 199. LC-MS: single peak at 254 nm, MH⁺ calcd. for C₂₅H₂₉N₄O₄: 449, obtained: 449.

### Example 367

### Synthesis of 6-methoxy-chroman-3-carboxylic acid [2-(S-1-carbamoyl-ethoxy)-4-(1H-pyrazol-4-yl)-phenyl]-amide

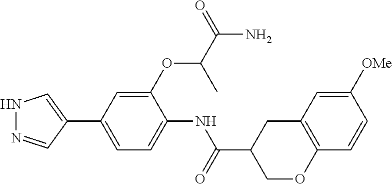

The title compound was prepared according to the procedure described in Scheme 1. LC-MS: single peak at 254 nm, MH⁺ calcd. for C₂₃H₂₅N₄O₅: 437, obtained: 437.

### Example 368

### Synthesis of 6-methoxy-chroman-3-carboxylic acid [2-(S-1-carbamoyl-ethoxy)-4-(3-methyl-1H-pyrazol-4-yl)-phenyl]-amide

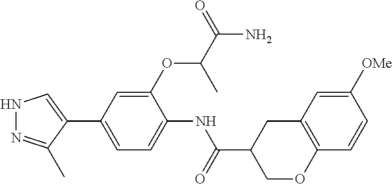

The title compound was prepared according to the procedure described in Scheme 1. LC-MS: single peak at 254 nm, MH⁺ calcd. for C₂₄H₂₇N₄O₅: 451, obtained: 451.

### Example 369

### Synthesis of 6-methoxy-chroman-3-carboxylic acid [4-(3-methyl-1H-pyrazol-4-yl)-2-(pyridin-3-yloxy)-phenyl]-amide

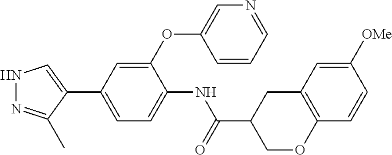

The title compound was prepared according to the procedure described in Scheme 4. LC-MS: single peak at 254 nm, MH⁺ calcd. for C₂₆H₂₅N₄O₄: 457, obtained: 457.

### Example 370

### Synthesis of 6-methoxy-chroman-3-carboxylic acid [4-(3-methyl-1H-pyrazol-4-yl)-2-(pyridin-3-ylmethoxy)-phenyl]-amide

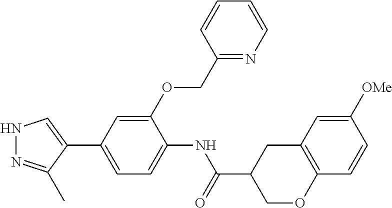

The title compound was prepared according to the procedure described in Scheme 4. LC-MS: single peak at 254 nm, MH⁺ calcd. for C₂₇H₂₇N₄O₄: 471, obtained: 471.

### Example 371

### Synthesis of chroman-3-carboxylic acid [2-(2-pyrrolidin-1-yl-ethoxy)-4-(1H-pyrazol-4-yl)-phenyl]-amide

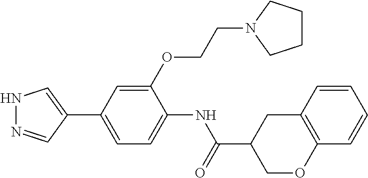

The title compound was prepared according to the procedure described in Scheme 4. LC-MS: single peak at 254 nm, MH⁺ calcd. for C₂₅H₂₉N₄O₃: 433, obtained: 433.

### Example 372

### Synthesis of chroman-3-carboxylic acid [2-(2 pyrrolidin-1-yl-ethoxy)-4-(1H-pyrazol-4-yl)-phenyl]-amide

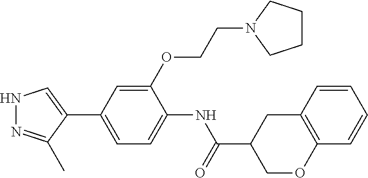

The title compound was prepared according to the procedure described in Scheme 4. LC-MS: single peak at 254 nm, MH⁺ calcd. for C₂₆H₃₁N₄O₃: 446, obtained: 446.

### Example 373

### Synthesis of 2,3-dihydro-benzo[1,4]dioxin-2-carboxylic acid [4-(1H-pyrazol-4-yl)-2-(pyridine-3-yloxy)-phenyl]-amide

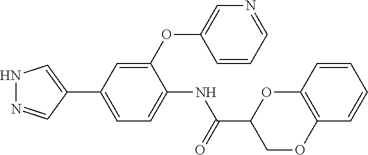

The title compound was prepared according to the procedure described in Scheme 4. LC-MS: single peak at 254 nm, MH⁺ calcd. for C₂₃H₁₉N₄O₄: 415, obtained: 415.

### Example 374

### Synthesis of 6-methoxy-chroman-3-carboxylic acid [2-(1-methyl-pyrrolidin-3-yloxy)-4-(3-methyl-1H-pyrazol-4-yl)-phenyl]-amide

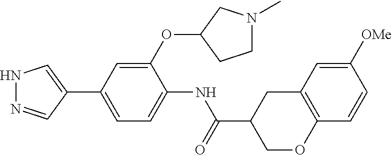

The title compound was prepared according to the procedure described in Scheme 4. LC-MS: single peak at 254 nm, MH⁺ calcd. for C₂₅H₂₉N₄O₄: 449, obtained: 449.

### Example 375

### Synthesis of 6-methoxy-chroman-3-carboxylic acid [2-(1-methyl-pyrrolidin-3-yloxy)-4-(3-methyl-1H-pyrazol-4-yl)-phenyl]-amide

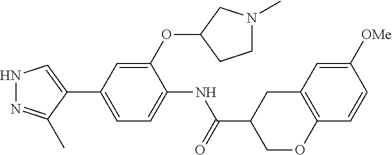

The title compound was prepared according to the procedure described in Scheme 4. LC-MS: single peak at 254 nm, MH⁺ calcd. for C₂₆H₃₁N₄O₄: 463, obtained: 463.

### Example 376

### Synthesis of benzyl 4-(2-(6-methoxychroman-3-carboxamido)-5-(1H-pyrazol-4-yl)phenoxy)piperidine-1-carboxylate

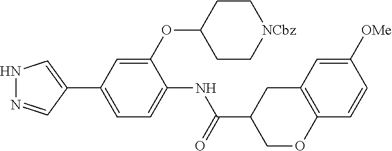

The title compound was prepared according to the procedure described in Scheme 4. LC-MS: single peak at 254 nm, MH⁺ calcd. for C₃₃H₃₅N₄O₆: 583, obtained: 583.

### Example 377

### Synthesis of 6-methoxy-chroman-3-carboxylic acid [2-(piperidin-4-yloxy)-4-(1H-pyrazol-4-yl)-phenyl]-amide

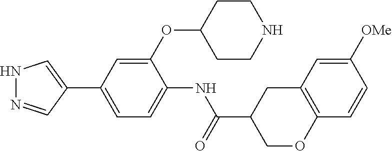

The title compound was prepared from benzyl 4-(2-(6-methoxychroman-3-carboxamido)-5-(1H-pyrazol-4-yl)phenoxy)piperidine-1-carboxylate according to the procedure described in Example 199. LC-MS: single peak at 254 nm, MH⁺ calcd. for C₂₅H₂₉N₄O₄: 449, obtained: 449.

### Example 378

### Synthesis of 6-methoxy-chroman-3-carboxylic acid [4-(1H-pyrazol-4-yl)-2-(pyridin-4-ylmethoxy)-phenyl]-amide

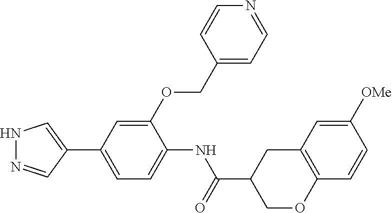

The title compound was prepared according to the procedure described in Scheme 4. LC-MS: single peak at 254 nm, MH⁺ calcd. for C₂₆H₂₅N₄O₄: 457, obtained: 457.

### Example 379

### Synthesis of 6-methoxy-chroman-3-carboxylic acid [2-(2-hydroxy-ethylsulfanyl)-4-(1H-pyrazol-4-yl)-phenyl]-amide

### Step A. 4-Bromo-2-[2-(tert-butyl-dimethyl-silanyloxy)-ethylsulfanyl]-phenylamine

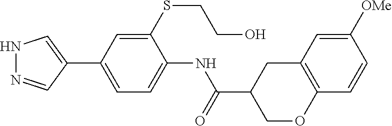

A mixture of 2-mercaptoethanol (0.78 g, 10 mmol), K₂CO₃ (1.52 g, 11 mmol), and 4-bromo-2-fluoro-1-nitrobenzene (2.20 g, 10 mmol) in DMF (20 mL) was stirred overnight at room temperature. After the solvent was removed by rotary evaporation, the residue was treated with aqueous solution of 0.5 N HCl (50 mL) and extracted with ethyl acetate (3×50 mL). The combined organic layer was washed with brine (50 mL), dried over Na₂SO₄, filtered, and concentrated to dryness. The residue was treated with ethanol (80 mL) and stannous chloride (11.28 g, 50 mmol) and the mixture was heated to reflux for 2 h. After cooling, the solvent was removed by rotary evaporation and the residue was treated with an aqueous solution of 1 M NaOH (150 mL) and extracted with methylene chloride (3×100 mL). The combined organic layer was washed with brine (50 mL), dried over Na₂SO₄, filtered, and concentrated. Without further purification, the residue was dissolved in 15 mL DMF and to this solution was added imidazole (1.00 g, 15 mmol), tert-butyldimethylsilylchloride (1.8 g, 12 mmol), and catalytic quantity of DMAP. After the reaction mixture was stirred overnight at room temperature, a 1:1 mixture of ethyl acetate:hexane (200 mL) and water (50 mL) was added and the organic layer was washed with two more portions of water (50 mL), brine (50 mL), dried, and concentrated to dryness. The residue was purified by flash column chromatography (1-15% ethyl acetate in hexane) to afford the title compound (3.06 g, 84%). LC-MS: single peak at 254 nm, MH⁺ calcd. for C₁₄H₂₅BrNOSSi: 362, obtained: 362.

### Step B. 6-methoxy-chroman-3-carboxylic acid [4-bromo-2-(2-hydroxy-ethylsulfanyl)-phenyl]-amide

To a solution of 4-bromo-2-[2-(tert-butyl-dimethyl-silanyloxy)-ethylsulfanyl]-phenylamine (0.14 g, 0.39 mmol) in DMF (0.5 mL) was added 6-methoxy-chroman-3-carboxylic acid (0.08 g, 0.39 mmol), HATU (0.19 g, 0.50 mmol), N-methylmorpholine (0.08 mL, 0.78 mmol) and the mixture was stirred overnight at room temperature. The solvent was removed by rotary evaporation and the residue was treated with ethyl acetate (50 mL) and an aqueous solution of saturated Na₂CO₃. The layers were separated, and the organic layer was washed with an additional portion of saturated Na₂CO₃, brine, dried over Na₂SO₄, filtered, and concentrated to dryness. The residue was treated with a solution of 40% TFA in methylene chloride (5 mL) and stirred for 1 h. The solvent was removed and the residue was purified by flash column chromatography to afford the title compound (0.07 g, 43%). LC-MS: single peak at 254 nm, MH⁺ calcd. for C₁₉H₂₁BrNO₄S: 438, obtained: 438.

### Step C. Synthesis of 6-methoxy-chroman-3-carboxylic acid [2-(3-acetylamino-phenoxy)-4-(1H-pyrazol-4-yl)-phenyl]-amide

The title compound was prepared according to the procedure described in Scheme 4. LC-MS: single peak at 254 nm, MH⁺ calcd. for C₂₂H₂₄N₃O₄S: 426, obtained: 426.

### Example 380

### Synthesis of 6-methoxy-chroman-3-carboxylic acid [2-(2-hydroxy-ethoxy)-4-(-1H-pyrazol-4-yl)-phenyl]-amide

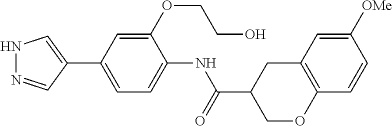

The title compound was prepared according to the procedure described in Scheme 4. LC-MS: single peak at 254 nm, MH⁺ calcd. for C₂₂H₂₄N₃O₅: 410, obtained: 410.

### Example 381

### Synthesis of 2,3-dihydro-benzo[1,4]dioxin-2-carboxylic acid [2-(2-hydroxy-ethoxy)-4-(1H-pyrazol-4-yl)-phenyl]-amide

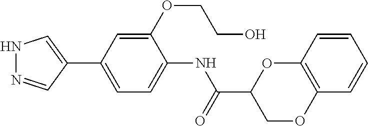

The title compound was prepared according to the procedure described in Scheme 4. LC-MS: single peak at 254 nm, MH⁺ calcd. for C₂₀H₂₀N₃O₅: 382, obtained: 382.

### Example 382

### Synthesis of 6-methoxy-chroman-3-carboxylic acid [2-(3-hydroxy-cyclohexyloxy)-4-(1H-pyrazol-4-yl)-phenyl]-amide

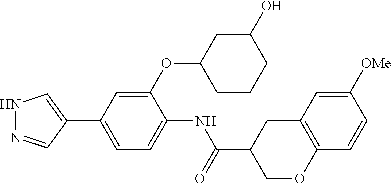

The title compound was prepared according to the procedure described in Scheme 4. LC-MS: single peak at 254 nm, MH⁺ calcd. for C₂₆H₃₀N₃O₅: 464, obtained: 464.

### Example 383

### Synthesis of 6-methoxy-chroman-3-carboxylic acid [2-(3-acetylamino-phenoxy)-4-(1H-pyrazol-4-yl)-phenyl]-amide

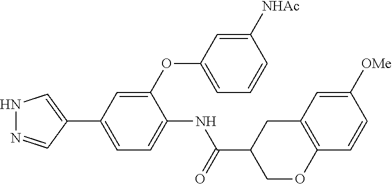

The title compound was prepared according to the procedure described in Scheme 4. LC-MS: single peak at 254 nm, MH⁺ calcd. for C₂₈H₂₇N₄O₅: 499, obtained: 499.

### Example 384

### Synthesis of 2,3-dihydro-benzo[1,4]dioxin-2-carboxylic acid [2-(2-benzyloxy-ethoxy)-4-(1H-pyrazol-4-yl)-phenyl]-amide

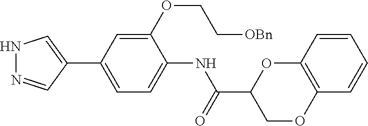

The title compound was prepared according to the procedure described in Scheme 4. LC-MS: single peak at 254 nm, MH⁺ calcd. for C₂₇H₂₆N₃O₅: 472, obtained: 472.

### Example 385

### Synthesis of 6-methoxy-chroman-3-carboxylic acid [2-(2-benzyloxy-ethoxy)-4-(1H-pyrazol-4-yl)-phenyl]-amide

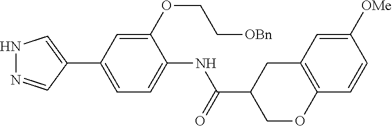

The title compound was prepared according to the procedure described in Scheme 4. LC-MS: single peak at 254 nm, MH⁺ calcd. for C₂₉H₃₀N₃O₅: 500, obtained: 500.

### Example 386

### Synthesis of 6-methoxy-chroman-3-carboxylic acid [2-(2-furan-2-ylmethoxy)-4-(1H-pyrazol-4-yl)-phenyl]-amide

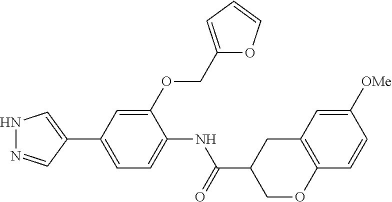

The title compound was prepared according to the procedure described in Scheme 4. LC-MS: single peak at 254 nm, MH⁺ calcd. for C₂₅H₂₄N₃O₅: 446, obtained: 446.

### Example 387

### Synthesis of 2,3-dihydro-benzo[1,4]dioxin-2-carboxylic acid [2-(1-methyl-pyrrolidin-3-yloxy)-4-(1H-pyrazol-4-yl)-phenyl]-amide

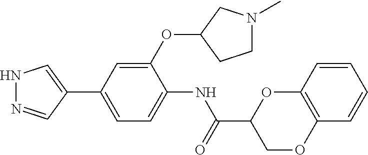

The title compound was prepared according to the procedure described in Scheme 4. LC-MS: single peak at 254 nm, MH⁺ calcd. for C₂₃H₂₅N₄O₄: 421, obtained: 421.

### Example 388

### Synthesis of 6-methoxy-chroman-3-carboxylic acid [2-(4-fluoro-phenoxy)-4-(1H-pyrazol-4-yl)-phenyl]-amide

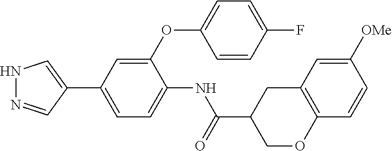

The title compound was prepared according to the procedure described in Scheme 4. LC-MS: single peak at 254 nm, MH⁺ calcd. for C₂₆H₂₃FN₃O₄: 460, obtained: 460.

### Example 389

### Synthesis of 6-fluoro-chroman-3-carboxylic acid [2-(1-methyl-pyrrolidin-3-yloxy)-4-(1H-pyrazol-4-yl)-phenyl]-amide

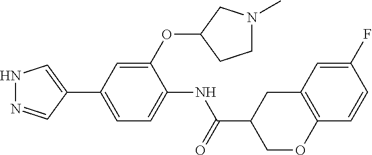

The title compound was prepared according to the procedure described in Scheme 4. LC-MS: single peak at 254 nm, MH⁺ calcd. for C₂₄H₂₆FN₄O₃: 437, obtained: 437.

The following compounds were also prepared using Scheme 4:

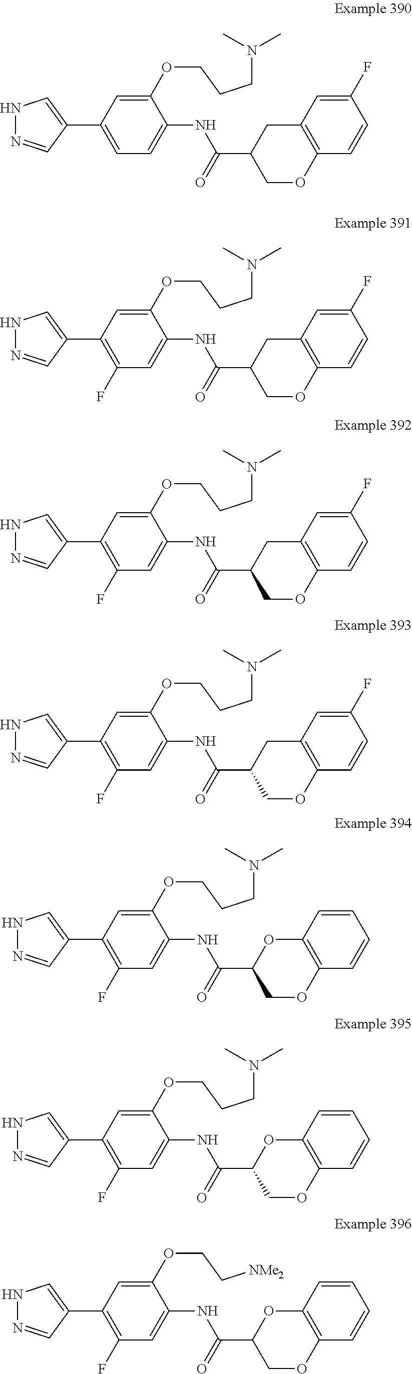

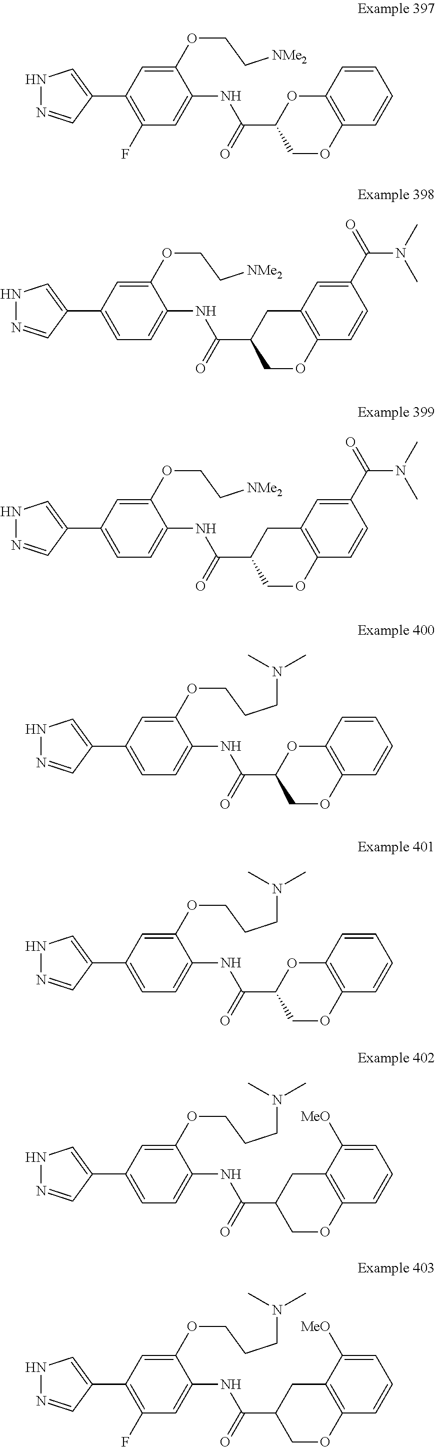

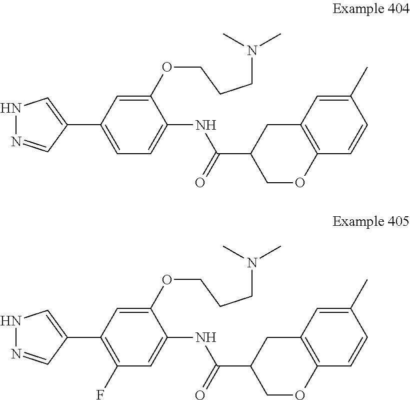

### Example 406

### Synthesis of benzyl 3-(2-(6-methoxychroman-3-carboxamido)-5-(1H-pyrazol-4-yl)phenoxy)piperidine-1-carboxylate

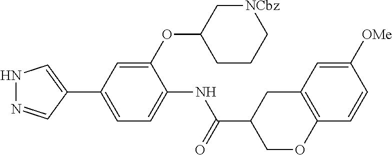

The title compound was prepared according to the procedure described in Scheme 4. LC-MS: single peak at 254 nm, MH⁺ calcd. for C₃₃H₃₅N₄O₆: 583, obtained: 583.

### Example 407

### Synthesis of 6-methoxy-chroman-3-carboxylic acid [2-(piperidin-3-yloxy)-4-(1H-pyrazol-4-yl)-phenyl]-amide

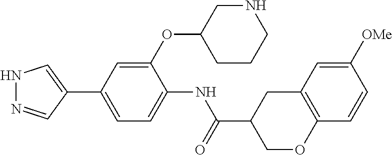

The title compound was prepared from benzyl 3-(2-(6-methoxychroman-3-carboxamido)-5-(1H-pyrazol-4-yl)phenoxy)piperidine-1-carboxylate according to the procedure described in Example 199. LC-MS: single peak at 254 nm, MH⁺ calcd. for C₂₅H₂₉N₄O₄: 449, obtained: 449.

### Example 408

### Synthesis of 6-methoxy-chroman-3-carboxylic acid [4-(1H-pyrazol-4-yl)-2-(tetrahydrofuran-2-ylmethoxy)-phenyl]-amide

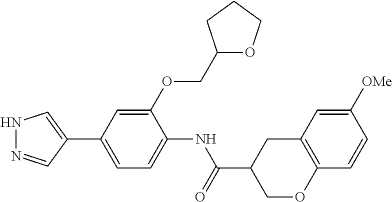

The title compound was prepared according to the procedure described in Scheme 4. LC-MS: single peak at 254 nm, MH⁺ calcd. for C₂₅H₂₈N₃O₅: 450, obtained: 450.

### Example 409

### Synthesis of 6-methoxy-chroman-3-carboxylic acid {4-(1H-pyrazol-4-yl)-2-[(S)-(tetrahydrofuran-2-ylmethoxy)]-phenyl}-amide

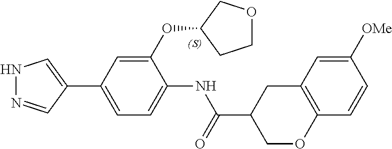

The title compound was prepared according to the procedure described in Scheme 4. LC-MS: single peak at 254 nm, MH⁺ calcd. for C₂₄H₂₆N₃O₅: 436, obtained: 436.

### Example 410

### Synthesis of 6-methoxy-chroman-3-carboxylic acid [4-(1H-pyrazol-4-yl)-2-(tetrahydropyran-4-yloxy)-phenyl]-amide

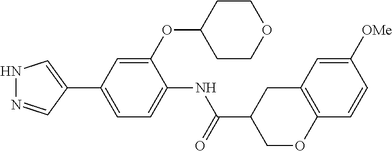

The title compound was prepared according to the procedure described in Scheme 4. LC-MS: single peak at 254 nm, MH⁺ calcd. for C₂₅H₂₈N₃O₅: 450, obtained: 450.

### Example 411

### Synthesis of 6-methoxy-chroman-3-carboxylic acid [2-(3-dimethylamino-phenoxy)-4-(1H-pyrazol-4-yl)-phenyl]-amide

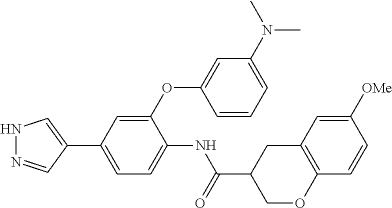

The title compound was prepared according to the procedure described in Scheme 4. LC-MS: single peak at 254 nm, MH⁺ calcd. for C₂₈H₂₈N₄O₄: 485, obtained: 485.

### Example 412

### Synthesis of 6-methoxy-chroman-3-carboxylic acid [2-(2-thiophen-2-ylmethoxy)-4-(1H-pyrazol-4-yl)-phenyl]-amide

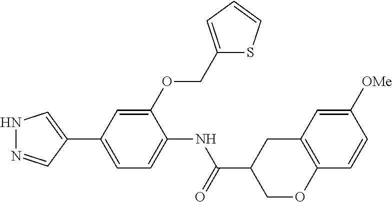

The title compound was prepared according to the procedure described in Scheme 4. LC-MS: single peak at 254 nm, MH⁺ calcd. for C₂₅H₂₄N₃O₄S: 462, obtained: 462.

### Example 413

### Synthesis of 6-methoxy-chroman-3-carboxylic acid [2-(1-methyl-piperidin-2-ylmethoxy)-4-(1H-pyrazol-4-yl)-phenyl]-amide

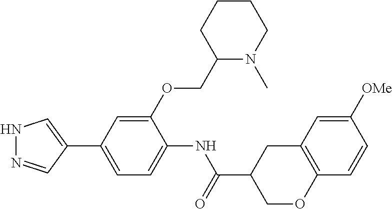

The title compound was prepared according to the procedure described in Scheme 4. LC-MS: single peak at 254 nm, MH⁺ calcd. for C₂₇H₃₃N₄O₄: 477, obtained: 477.

### Example 414

### Synthesis of 6-methoxy-chroman-3-carboxylic acid [2-(4-methy-piperazin-1-yl)-4-(1H-pyrazol-4-yl)-phenyl]-amide

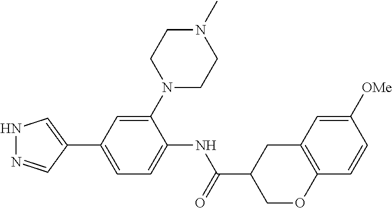

The title compound was prepared according to the procedure described in Scheme 4. LC-MS: single peak at 254 nm, MH⁺ calcd. for C₂₅H₃₀N₅O₃: 448, obtained: 448.

### Example 415

### Synthesis of 6-fluoro-chroman-3-carboxylic acid [2-[methyl-(2-pyrrolidin-1-yl-ethyl)-amino]-4-(1H-pyrazol-4-yl)-phenyl]-amide

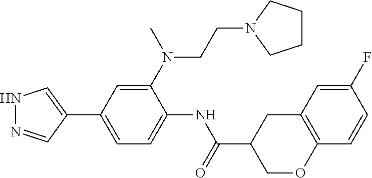

The title compound was prepared according to the procedure described in Scheme 4. LC-MS: single peak at 254 nm, MH⁺ calcd. for C₂₆H₃₁FN₅O₂: 464, obtained: 464.

### Example 416

### Synthesis of 6-fluoro-chroman-3-carboxylic acid [4-(2-amino-pyrimidin-4-yl)-2-(2-pyrrolidin-1-yl-ethoxy)-phenyl]-amide

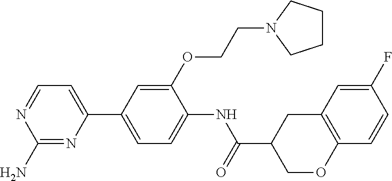

The title compound was prepared according to the procedure described in Scheme 8. LC-MS: single peak at 254 nm, MH⁺ calcd. for C₂₆H₂₉FN₅O₃: 478, obtained: 478.

### Example 417

### Synthesis of 6-methoxy-chroman-3-carboxylic acid [4-(2-amino-pyrimidin-4-yl)-2-(1-methyl-pyrrolidin-3-yloxy)-phenyl]-amide

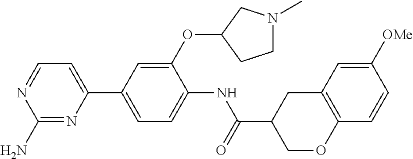

The title compound was prepared according to the procedure described in Scheme 8. LC-MS: single peak at 254 nm, MH⁺ calcd. for C₂₆H₃₀N₅O₄: 476, obtained: 476.

### Example 418

### Synthesis of 6-fluoro-chroman-3-carboxylic acid [4-(2-amino-pyrimidin-4-yl)-2-(1-methyl-pyrrolidin-3-yloxy)-phenyl]-amide

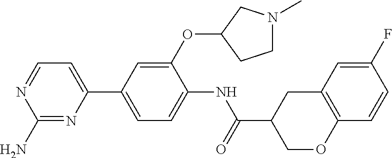

The title compound was prepared according to the procedure described in Scheme 8. LC-MS: single peak at 254 nm, MH⁺ calcd. for C₂₅H₂₆FN₅O₃: 464, obtained: 464.

### Example 419

### Synthesis of 2,3-dihydro-benzo[1,4]dioxine-2-carboxylic acid [4-(2-amino-pyrimidin-4-yl)-2-(1-methyl-pyrrolidin-3-yloxy)-phenyl]-amide

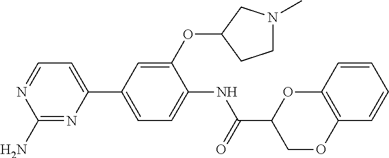

The title compound was prepared according to the procedure described in Scheme 8. LC-MS: single peak at 254 nm, MH⁺ calcd. for C₂₄H₂₆N₅O₄: 448, obtained: 448.

### Example 420

### Synthesis of 6-fluoro-chroman-3-carboxylic acid {4-(2-amino-pyrimidin-4-yl)-2-[methyl-(2-pyrrolidin-1-yl-ethyl)-amino]-phenyl}-amide

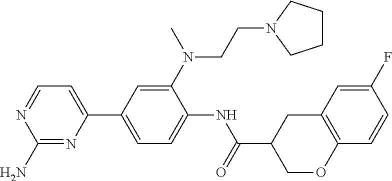

The title compound was prepared according to the procedure described in Scheme 8. LC-MS: single peak at 254 nm, MH⁺ calcd. for C₂₇H₃₂FN₆O₂: 491, obtained: 491.

### Example 421

### Synthesis of 6-methoxy-chroman-3-carboxylic acid [2-(2-methoxy-ethoxy)-4-(1H-pyrazol-4-yl)-phenyl]-amide

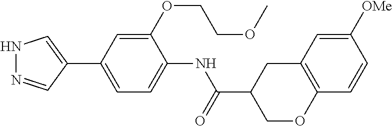

The title compound was prepared according to the procedure described in Scheme 4. LC-MS: single peak at 254 nm, MH⁺ calcd. for C₂₃H₂₆N₃O₅: 424, obtained: 424.

### Example 422

### Synthesis of N-(2-(2-(dimethylamino)ethoxy)-4-(1H-pyrazol-4-yl)phenyl)-1,2,3,4-tetrahydronaphthalene-2-carboxamide

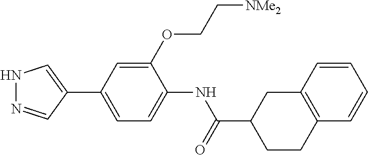

The title compound was prepared according to the procedure described in Scheme 4. LC-MS: single peak at 254 nm, MH⁺ calcd. for C₂₄H₂₈N₄O₂: 405, obtained: 405.

### Example 423

### Synthesis of 1,2,3,4-tetrahydronaphthalene-2-carboxylic acid [4-(1H-pyrazol-4-yl)-phenyl]-amide

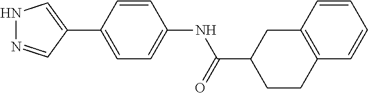

The title compound was prepared according to the procedure described in Scheme 4. LC-MS: single peak at 254 nm, MH⁺ calcd. for C₂₀H₂₀N₃O: 318, obtained: 318.

### Example 424

### Synthesis of N-(2-(2-(dimethylamino)ethoxy)-4-(1H-pyrazol-4-yl)phenyl)-7-methoxy-1,2,3,4-tetrahydronaphthalene-2-carboxamide

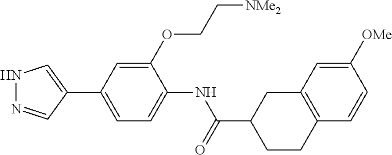

The title compound was prepared according to the procedure described in Scheme 4. LC-MS: single peak at 254 nm, MH⁺ calcd. for C₂₅H₃₀N₄O₃: 435, obtained: 435.

### Example 425

### N-(2-(2-(dimethylamino)ethoxy)-4-(1H-pyrazol-4-yl)phenyl)-7-methoxy-4-oxo-1,2,3,4-tetrahydronaphthalene-2-carboxamide

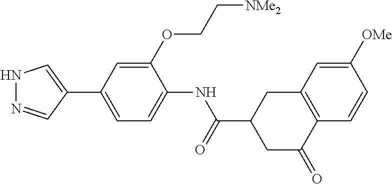

The title compound was prepared according to the procedure described in Scheme 4. LC-MS: single peak at 254 nm, MH⁺ calcd. for C₂₅H₂₈N₄O₄: 449, obtained: 449.

### Example 426

### Synthesis of 6-methoxy-chroman-3-carboxylic acid [2-(4-dimethylamino-piperidin-1-yl)-4-(1H-pyrazol-4-yl)-phenyl]-amide

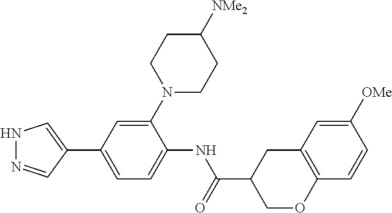

The title compound was prepared according to the procedure described in Scheme 4. LC-MS: single peak at 254 nm, MH⁺ calcd. for C₂₇H₃₄N₅O₃: 476, obtained: 476.

### Example 427

### Synthesis of 6-methoxy-chroman-3-carboxylic acid [2-(3-dimethylamino-pyrrolidin-1-yl)-4-(1H-pyrazol-4-yl)-phenyl]-amide

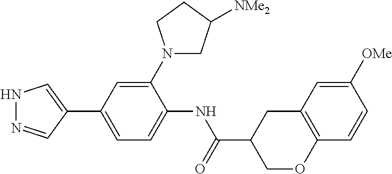

The title compound was prepared according to the procedure described in Scheme 4. LC-MS: single peak at 254 nm, MH⁺ calcd. for C₂₆H₃₂N₅O₃: 462, obtained: 462.

### In Vitro Enzyme Inhibition

The assay is based on ability of Rhok2 to phosphorylate a specific peptide sequence derived from its substrate—ribosomal protein S6 (amino acid residues 229-239). Rhok2 uses ATP as a donor of phosphate for the phosphorylation of the substrate, which leads to the depletion of ATP in the reaction mix. An assay kit (“Kinase-Glo”, Promega) was used to quantify enzyme activity. Using this kit, residual amounts of ATP are measured by a secondary enzymatic reaction, through which luciferase utilizes the remaining ATP to produce luminescence. Luminescent signal is directly proportional to ATP concentration and inversely proportional to Rhok2 activity. This dose response assay was conducted in 1536 well plate format. Each concentration was tested nominally in triplicate.

Protocol Summary: 1.25 microliters of solution containing 20 micromolar ATP and 20 micromolar S6 peptide (substrate) in assay buffer (50 millimolar HEPES pH 7.3, 10 millimolar MgCl₂, 0.1% BSA, 2 millimolar DTT) were dispensed in 1536 microtiter plate. 15 nanoliters of test compound or positive and negative control (2.12 millimolar Y-27632 and DMSO, respectively) were then added to the appropriate wells. Each compound dilution was assayed in triplicate, for a nominal total of 30 data points per dose response curve. The enzymatic reaction was initiated by dispensing 1.25 microliters of 8 nanomolar Rhok2 solution in assay buffer (50 millimolar HEPES pH 7.3, 10 millimolar MgCl₂, 0.1% BSA, 2 millimolar DTT). After 2 hours of incubation at 25 degrees Celsius, 2.5 microliters of Kinase Glo reagent (Promega Corporation, Madison, Wis.) was added to each well. Plates were incubated for 10 minutes and luminescence was read on Perkin-Elmer Viewlux for 60 seconds. Each compound was tested in triplicate. The percent inhibition for each well has been calculated as follows: % inhibition=(test_compound−median_negative_control)/(median_positive_control−median_negative_control)*100 where the positive control is Y-27632 (13 micromolar) and negative control is DMSO only. The IC₅₀ values of most examples were <200 nM, with significant amount of them <50 nM, and many of them <1 nM.

In Vitro Cell-Based ppMLC Assay

A7r5 cells were plated at 5000 cells/well in a 96 well Packard View Plate (Perkin Elmer) in DMEM+10% FBS. After attachment overnight, cells were serum starved for 4 h and treated with inhibitor in 0.25% DMSO final concentration for 1 h at 37° C. Cells were then treated with 10 μM lysophosphatitic acid (LPA) for 10 min. Following treatment, cells were immediately fixed with 4% paraformaldehyde for 30 minutes. After a brief wash in 0.1M glycine, cells were permeablized in 0.2% Triton X for 10 minutes. Cells were then washed once in PBS and blocked in Li-COR blocking buffer (Li-COR Biosciences) for 1 h at 25 C. Cells were probed for either phosphorylated myosin light chain 20 (55 ng/mL), total myosin light chain (525 ng/mL) or anti-bovine α-tubulin (1 μg/mL), and incubated overnight at 4° C. Following three washes, cells were probed with goat-anti-rabbit or goat-anti-mouse IR800 (2 μg/mL in LI-COR block+0.025% Tween) for 1 h at 25° C. Nuclei were stained with TO-PRO-3 iodide (642/661) (1:4000) for 20 min, washed twice in PBS/0.05% Tween and read with an Odyssey Infrared Imaging System (LI-COR Biosciences). The IC₅₀ values of examples tested were between 1 nM to 10 uM, with significant amount of them <50 nM.

### In Vitro Cell-Based Neurite Length (N2a) Assay

N2a cells are maintained in DMEM/FBS at 37 C and 5% CO₂. For the experiment the cells were plated on a poly-D-lysine coated 96-well tissue culture plate. After attachment, cell differentiation was induced for 2 days by addition of 10 uM retinoic acid. Cells were treated for 1 h with a dilution of compounds in 0.3% DMSO final concentration before neurite retraction was induced by 5 uM LPA. Cells were stained for tubulin and nuclei and images were acquired on an INCell 1000 workstation. Images were analyzed using the developer toolbox and neurite length was quantitated. The IC₅₀ values of all examples selected for testing were in the range of 1 nM-10 μM.

Compounds tested in the assays described herein are considered to be active if they exhibit an IC₅₀ of ≦10 μM. Additional examples of activity include IC₅₀'s of ≦1 μM, ≦0.1 μM, ≦0.01 μM, and of ≦0.001 μM. Using the methodology described herein, a number of compounds of the present invention were found to exhibit IC₅₀'s of ≦10 μM, thereby confirming the utility of the compounds of the present invention as effective ROCK inhibitors. Those compounds with IC₅₀ₛ below 150 nM were also tested in the cell assays. The IC₅₀s obtained from cell assays were in the range from 1 nM to 10 uM. For example, the IC50s of Example 92 in enzymatic assay, ppMLC assay, and N2a assay are 2 nM, 38 nM, and 27 nM respectively.

All references cited herein are incorporated by reference. The present invention may be embodied in other specific forms without departing from the spirit or essential attributes thereof and, accordingly, reference should be made to the appended claims, rather than to the foregoing specification, as indicating the scope of the invention.

What is claimed is:

1. A compound of formula (I) 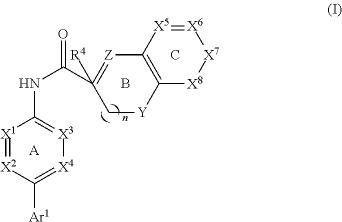
wherein
X¹, X², X³, and X⁴ are each independently N, CH, or CR¹ such that ring A comprises a phenyl, pyridyl, pyrazinyl, pyridazinyl, or pyrimidinyl ring, provided that 0-2 of X¹, X², X³ and X⁴ are CR¹, the remainder being independently N or CH;
R¹ is independently at each occurrence F, Cl, Br, I, CF₃, OCF₃, (C₁₋₆)-alkyl substituted with 0-2 R^{a}, (C₂₋₆)-alkenyl substituted with 0-2 R^{a}, (C₂₋₆)-alkynyl substituted with 0-2 R^{a}, (CH₂)ₚNO₂, (CH₂)ₚCN, (CH₂)ₚOR, (CH₂)ₚNR₂, (CH₂)ₚC(═O)R, (CH₂)ₚOC(═O)R, (CH₂)ₚC(═O)OR, (CH₂)ₚC(═O)NR₂, (CH₂)ₚOC(═O)NR₂, (CH₂)ₚNRC(═O)R, (CH₂)ₚNRC(═O)OR, (CH₂)ₚNRC(═O)NR₂, (CH₂)ₚC(═NH)NH₂, (CH₂)ₚS(O)_{q}R, (CH₂)ₚSO₂NR₂, (CH₂)ₚNRSO₂R, (CH₂)ₚNRSO₂NR₂, (CH₂)ₚ-(3-10 membered)-cycloalkyl substituted with 0-2 R^{a}, or (CH₂)ₚ-(4-10 membered)-heterocyclyl substituted with 0-2 R^{a} comprising 1-4 heteroatoms selected from O, S(O)_{q}, and N; or two adjacent R¹ substituents can form a fused phenyl or a 5-6 membered heteroaryl comprising carbon atoms and 1-2 heteroatoms selected from O, S(O)_{q}, and N, and substituted with 0-3 R^{a}, wherein p is 0-4 and q is 0-2;
R is independently at each occurrence H, (C₁₋₆)-alkyl substituted with 0-2 R^{a}, (C₂₋₆)-alkenyl substituted with 0-2 R^{a}, (C₂₋₆)-alkynyl substituted with 0-2 R^{a}, (3-10 membered)-cycloalkyl substituted with 0-2 R^{a}, or (3-10 membered)-heterocyclyl substituted with 0-2 R^{a} comprising 1-4 heteroatoms selected from O, S(O)_{q}, and N; or, an NR₂ forms a (3-10 membered)-heterocyclyl substituted with 0-2 R^{a} and comprising 0-1 additional ring heteroatoms selected from N, O, and S(O)_{q};
R^{a} is independently at each occurrence oxo, F, Cl, Br, I, CF₃, OCF₃, (C₁₋₆)-alkyl substituted with 0-2 R^{a}, (C₂₋₆)-alkenyl substituted with 0-2 R^{a}, (C₂₋₆)-alkynyl substituted with 0-2 R^{a}, (CH₂)ₚNO₂, (CH₂)ₚCN, (CH₂)ₚOR, (CH₂)ₚNR₂, (CH₂)ₚC(═O)R, (CH₂)ₚOC(═O)R, (CH₂)ₚC(═O)OR, (CH₂)ₚC(═O)NR₂, (CH₂)ₚOC(═O)NR₂, (CH₂)ₚNRC(═O)R, (CH₂)ₚNRC(═O)OR, (CH₂)ₚNRC(═O)NR₂, (CH₂)ₚC(═NH)NH₂, (CH₂)ₚS(O)_{q}R, (CH₂)ₚSO₂NR₂, (CH₂)ₚNRSO₂R, (CH₂)ₚNRSO₂NR₂, (CH₂)ₚ-(3-10 membered)-cycloalkyl substituted with 0-2 R^{a}, or (CH₂)ₚ-(3-10 membered)-heterocyclyl substituted with 0-2 R^{a} comprising 1-4 heteroatoms selected from O, S(O)_{q}, and N;
Ar¹ comprises a 5- or 6-membered heteroaryl comprising at least one nitrogen atom and 0-3 additional heteroatoms selected from O, S(O)_{q}, and N; when Ar¹ is a 5-membered heteroaryl, a nitrogen atom is disposed one atom away from an atom of the heteroaryl bonded to ring A, and when Ar¹ is a 6-membered heteroaryl, a nitrogen atom is disposed two atoms away from an atom of the heteroaryl bonded to ring A; wherein Ar¹ is optionally fused with phenyl or a 5-6 membered heteroaryl comprising 1-2 heteroatoms selected from O, S(O)_{q}, and N, wherein the fused phenyl or 5-6 membered heteroaryl is substituted with 0-3 R^{a};
n is 0 or 1;
wherein a dotted line indicates a single bond or a double bond;
Y is O, CH(R³), S(O)_{q}, N(R³), or C(═O);
Z is O, CH(R³), CR³, S(O)_{q}, N(R³), or C(═O), provided that when the double bond is absent, R⁴ is present, Z is O, CH(R³), or C(═O) and when the double bond is present R⁴ is absent and Z is CR³;
X⁵, X⁶, X⁷ and X⁸ are each independently N, CH, or CR², such that ring C comprises a phenyl, pyridyl, pyrazinyl, pyridazinyl, or pyrimidinyl ring;
R² is independently at each occurrence hydrogen, (C₁-C₆)alkyl, hydroxy(C₁-C₆)alkyl, (C₂-C₆)alkenyl; (C₂-C₆)alkynyl; halogen; —C≡N; —NO₂; —C(═O)R; —C(═O)OR; (C₁-C₆)alkylene-C(═O)OR; —C(═O)NR₂; (C₁-C₆)alkylene-C(═O)NR₂; —C(═NR)NR₂; —OR; (C₁-C₆)alkylene-OR; —OC(═O)(C₁-C₆)alkyl; —OC(═O)O(C₁-C₆)alkyl; —OC(═O)NR₂; —NR₂; —NRC(═O)R; —NRC(═O)O(C₁-C₆)alkyl; —NRC(═O)NR₂; —NR(C₁-C₆)alkylene-NR₂; —NR(C₁-C₆)alkylene-OR; —NR(C₁-C₆)alkylene-Ar²; —NRSO₂R; —SR; —S(O)R; —SO₂R; —OSO₂(C₁-C₆)alkyl; —SO₂NR₂; (C₁-C₃)perfluoroalkyl; —O(C₁-C₃)perfluoroalkyl; pyrazolyl; triazolyl; and tetrazolyl; or two adjacent R² groups taken together form a fused cycloalkyl, heterocyclyl, aryl or heteroaryl ring which is substituted with 0-3 R^{a};
R³ is H, CF₃, OCF₃, OCH₃, (C₁₋₆)-alkyl substituted with 0-3 R^{a}, or NR₂;
R⁴ is H or (C₁₋₆)alkyl substituted with 0-3 R^{a};
or any salt, stereoisomer, tautomer, hydrate, solvate, or prodrug thereof.

2. The compound of claim 1 wherein ring B and ring C together form a chroman ring system.

3. The compound of claim 1 wherein ring B and ring C together form a 2H-chromene ring system.

4. The compound of claim 1 wherein ring B and ring C together form a 2,3-dihydrobenzo[b][1,4]dioxine ring system.

5. The compound of claim 1 wherein ring B and ring C together form a 2,3-dihydrobenzofuran ring system.

6. The compound of claim 1 wherein Y is O and n=1.

7. The compound of claim 6 wherein Z is CH₂.

8. The compound of claim 6 wherein Z is O.

9. The compound of claim 1 wherein Z is O and n=1.

10. The compound of claim 9 wherein Y is CH₂.

11. The compound of claim 1 wherein Z is O and n=0.

12. The compound of claim 1 wherein both Y and Z are CH₂.

13. The compound of claim 1 wherein Ar¹ comprises a pyridinyl, pyrimidinyl, amino-substituted pyridyl, halo-substituted pyridyl, pyridyl substituted with both amino and halo, of amino-substituted pyrimidinyl, and R¹ is other than unsubstituted alkyl.

14. The compound of claim 1 wherein Ar¹ comprises a pyrazolyl, methylpyrazolyl, dimethylpyrazolyl, pyridinyl, methylpyridyl, pyrrolopyridyl, pyrrolopyrimidinyl, naphthyridinyl, pyridopyrimidinyl, imidazolyl, triazolyl, or oxazolyl ring system.

15. The compound of claim 14 wherein Ar¹ comprises a pyrazol-4-yl, 3-methylpyrazol-4-yl, 5-methylpyrazol-4-yl, 3-aminopyridazol-4-yl, 3,5-dimethylpyrazol-4-yl ring system, imidazol-4-yl, 3-methylimidazol-4-yl, 1,2,3-triazol-4-yl, or 5-aminooxazol-4-yl ring system.

16. The compound of claim 14 wherein Ar¹ comprises a pyridin-4-yl, 2-methylpyridin-y-yl ring system, 3-fluoropyridin-4-yl, 3-chloropyridin-4-yl, 3-cyano-pyridin-4-yl, 2-NR₂-substituted-pyridin-4-yl, 1,3-pyrimidin-4-yl, 2-NR₂-substituted-1,3-pyrimidin-4-yl, or 5-NR₂-substituted-1,3-pyrimidin-4-yl ring system.

17. The compound of claim 14 wherein Ar¹ comprises a 1,8-naphthyridin-4-yl, 1,8-naphthyridin-3-yl, pyrido[2,3-d]pyrimidin-4-yl, 1H-pyrrole[2,3-b]pyridin-4-yl, or 7H-pyrrole[2,3-d]pyrimidin-4-yl ring system.

18. The compound of claim 1 wherein ring A comprises phenyl, wherein all R¹ are H or wherein ring A is mono- or independently plurisubstituted with R¹ comprising (C₁₋₆)-alkyl, fluoro, chloro, (C₁₋₆)-alkoxy, carboxamidoalkoxy, aminoalkylcarboxamido, trifluoromethyl, carboxamido, aminoalkoxy, trifluoromethoxy, sulfonamido-substituted phenyl, hydroxy, heterocyclyloxy, heterocyclylalkoxy, aminoalkylthio, aralkoxy, aminoalkylamino, heterocyclylamino, heterocyclylalkylamino, heterocyclylthio, heterocyclylalkylthio, aryloxy, hydroxyalkoxy, alkoxyalkoxy, or alkenyloxyalkoxy, or any combination thereof.

19. The compound of claim 1 wherein ring A comprises pyridyl, pyridazinyl, pyrazinyl, or pyrimidyl.

20. The compound of claim 1 wherein R⁴ is H or methyl.

21. The compound of claim 1 wherein X⁵, X⁶, X⁷ and X⁸ are all CH or CR².

22. The compound of claim 1 wherein X⁵ and X⁶ are both CR² and the CR² together comprise a fused aryl ring, or wherein X⁶ and X⁷ are both CR² and the CR² together comprise a fused aryl ring, or wherein X⁷ and X⁸ are both CR² and the CR² together comprise a fused aryl ring.

23. The compound of claim 1 wherein R² comprises chloro, fluoro, trifluoromethyl, carboxy, (C₁₋₆)-alkoxycarbonyl, carboxamido, aminoalkylcarboxamido, (C₁₋₆)-alkyl, aminoalkoxy, heterocyclyloxy, or heterocyclylalkoxy, or any combination thereof.

24. A compound of claim 1 wherein the compound is any of the following: 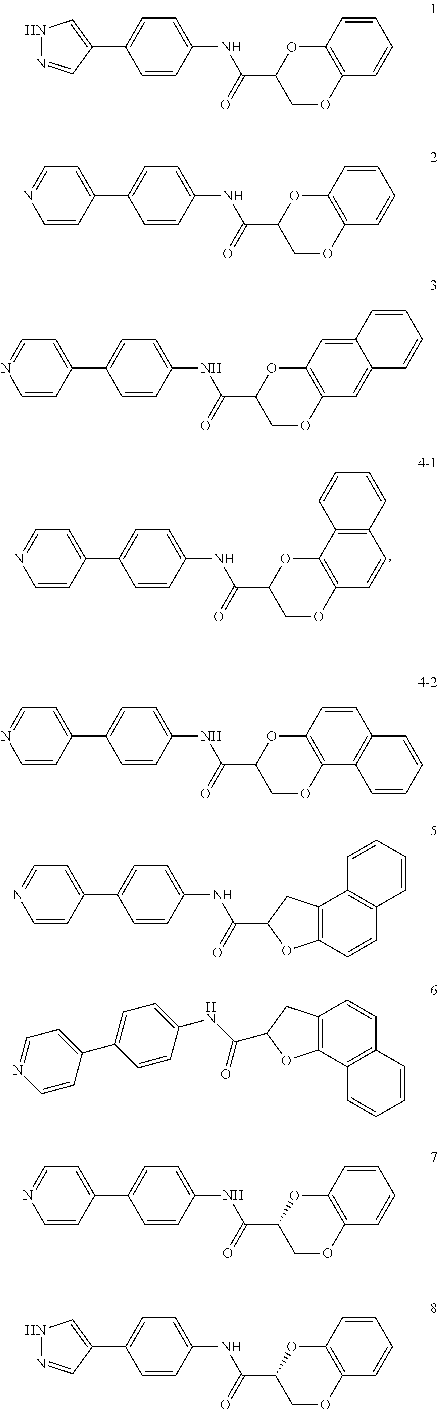 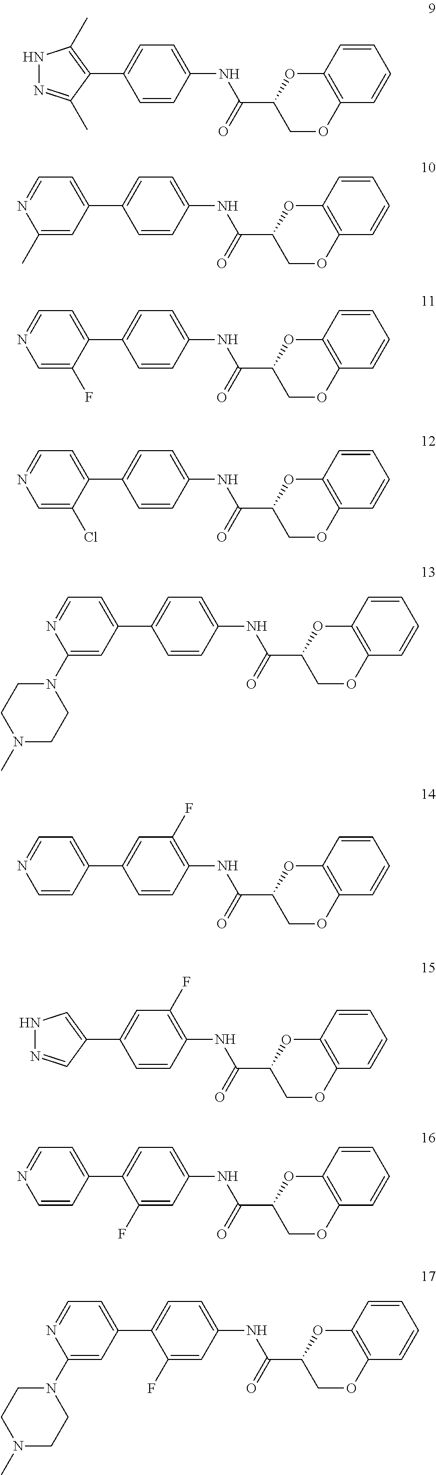 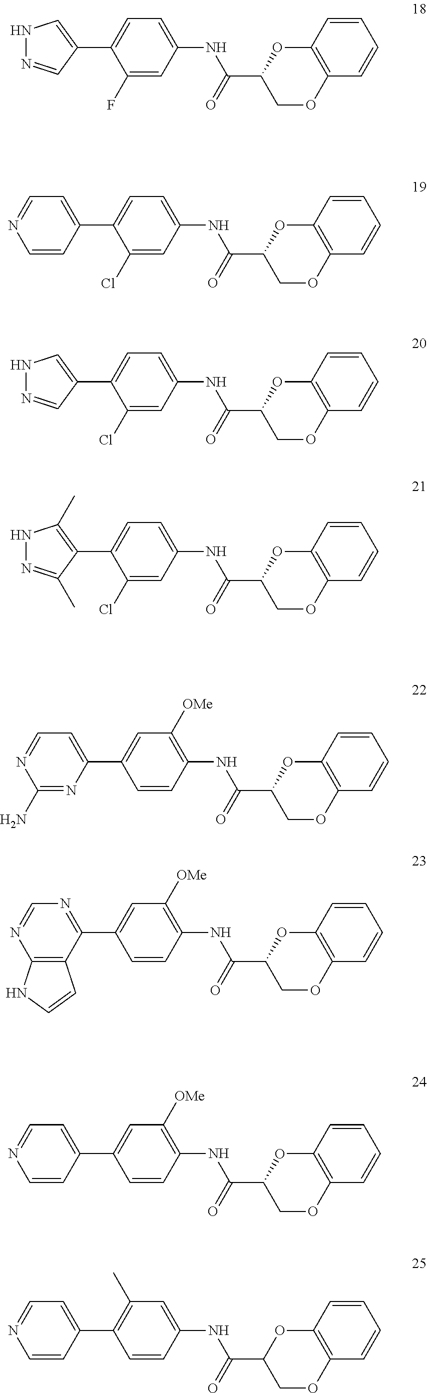 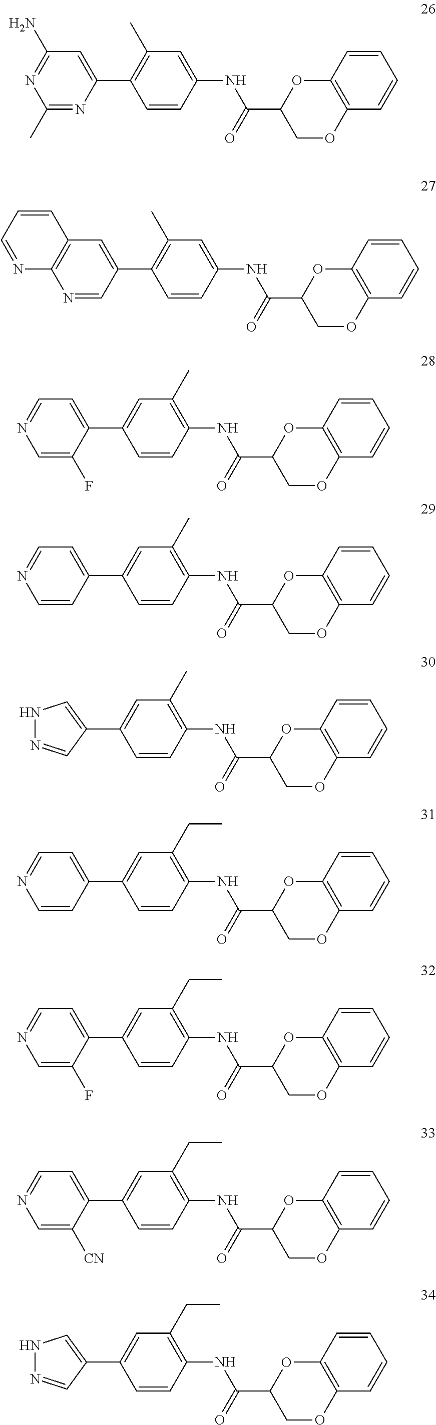 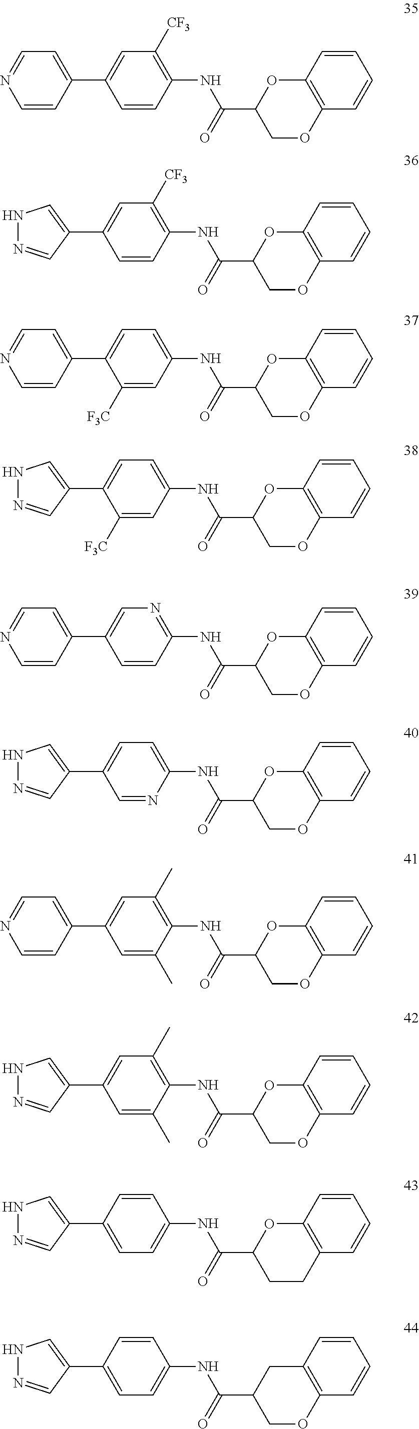 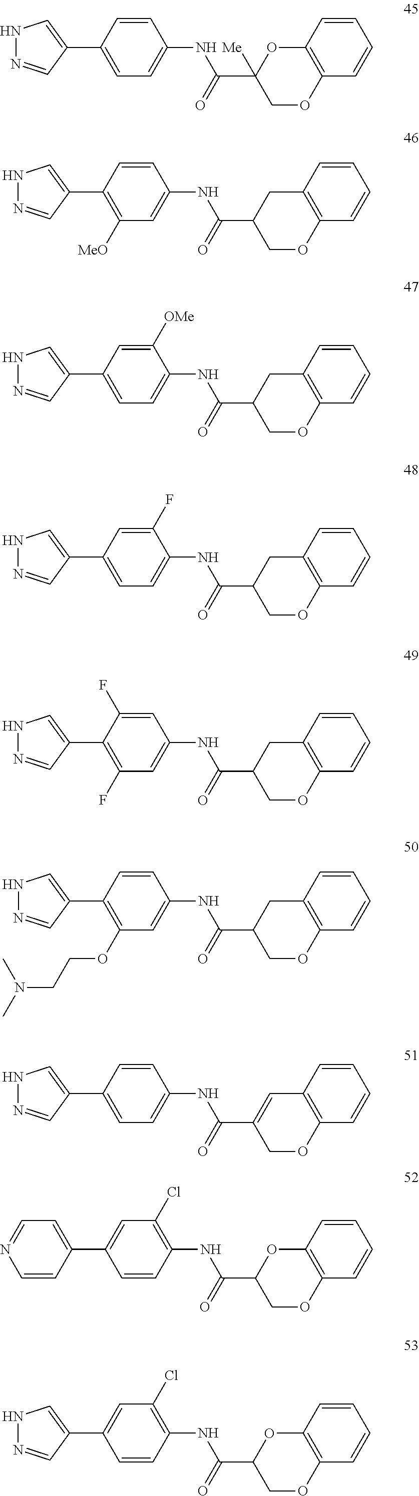 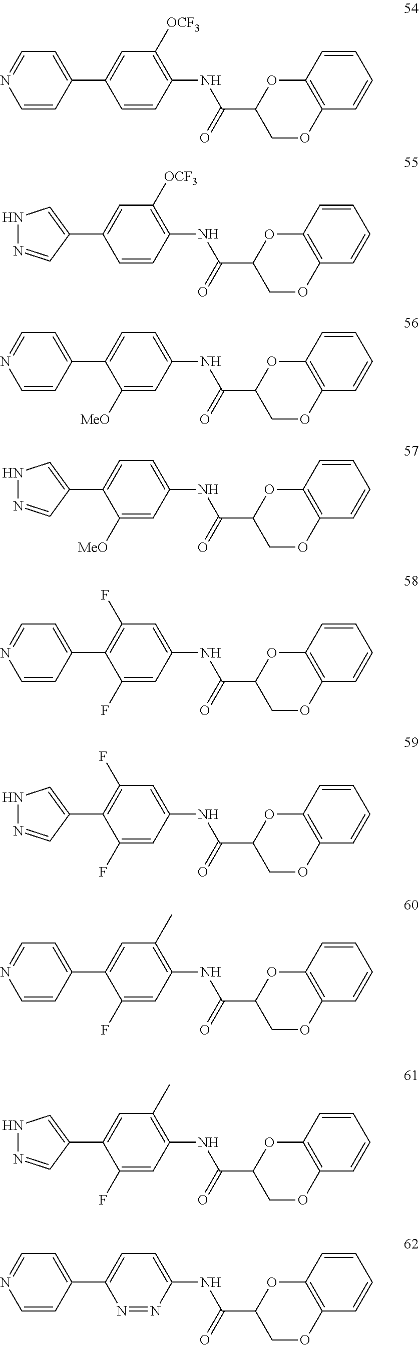 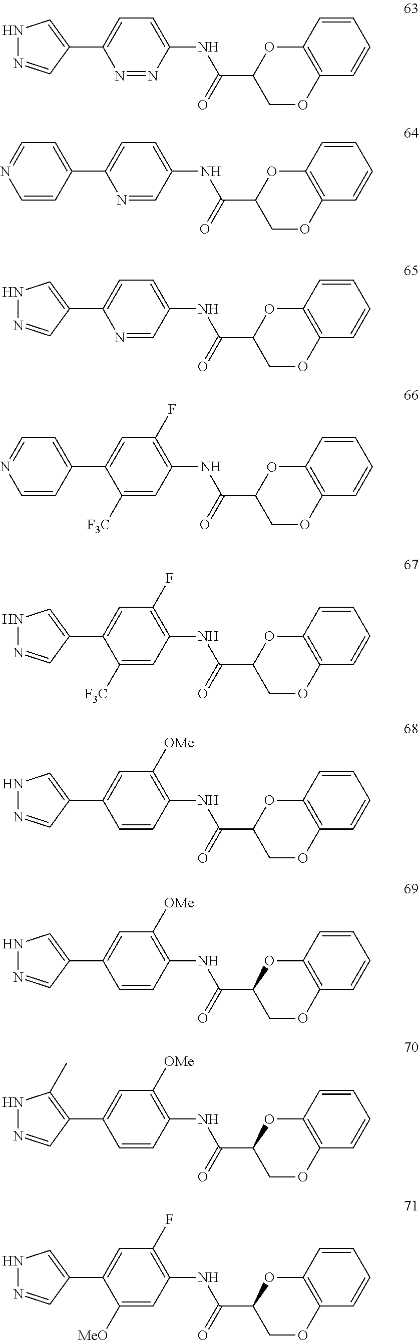 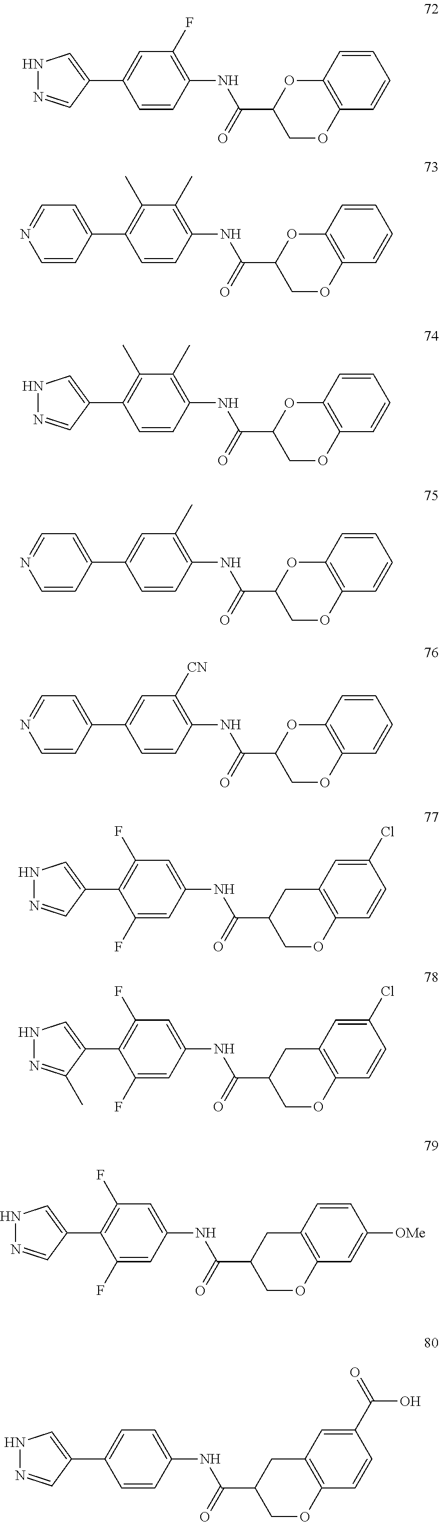 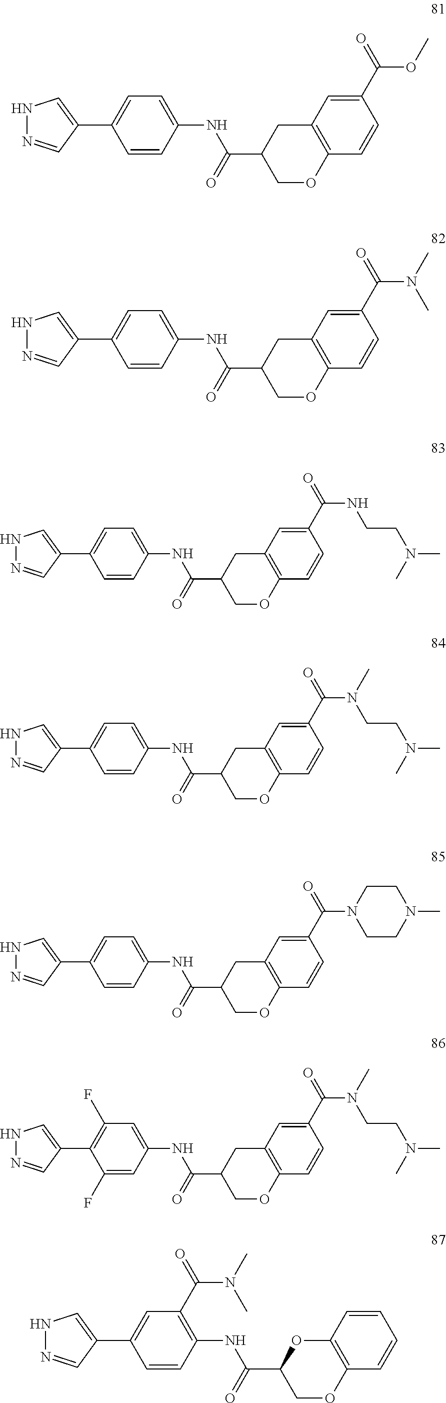 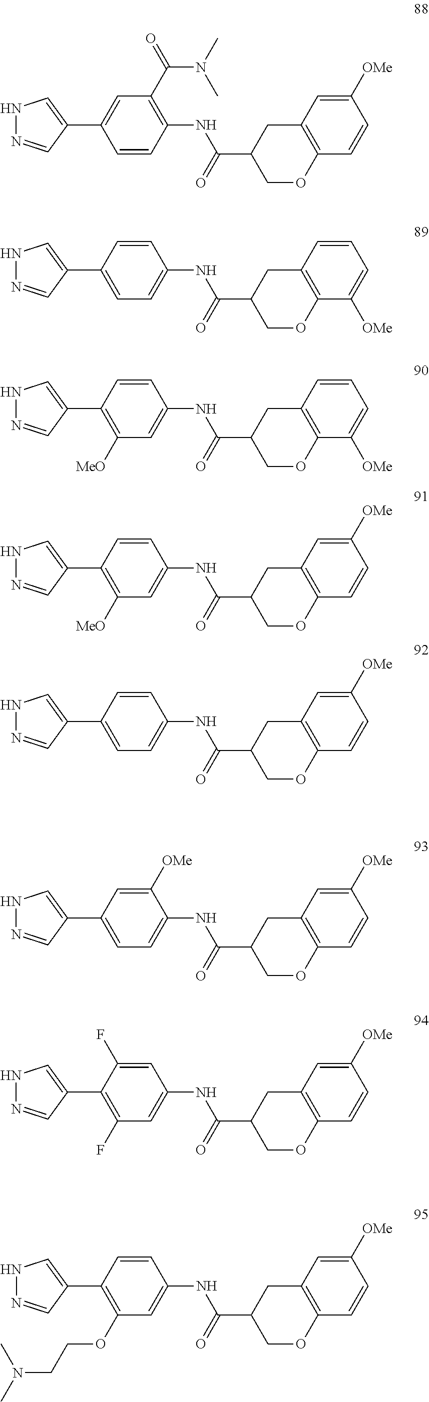 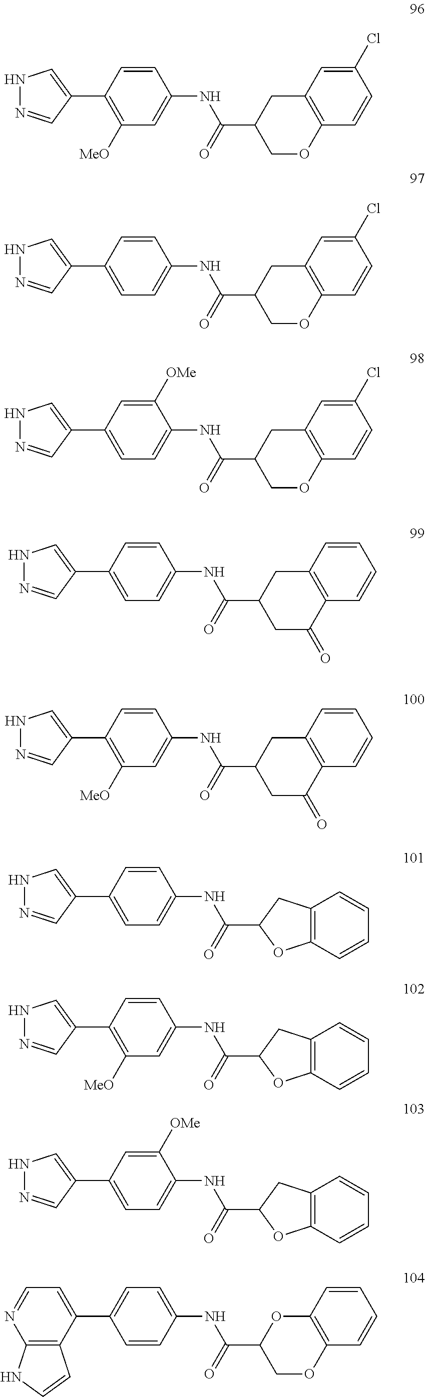 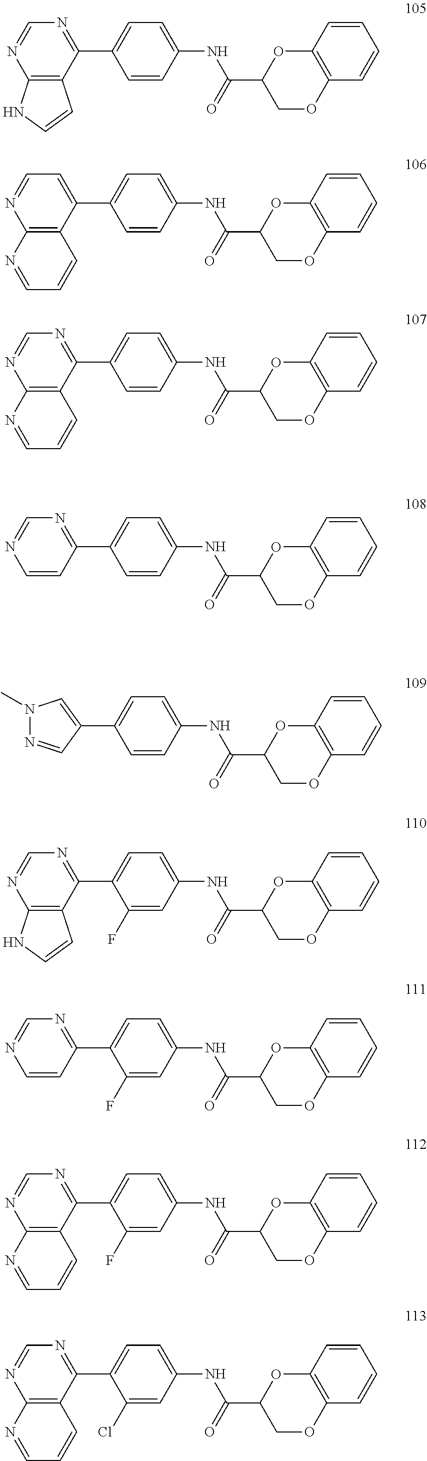 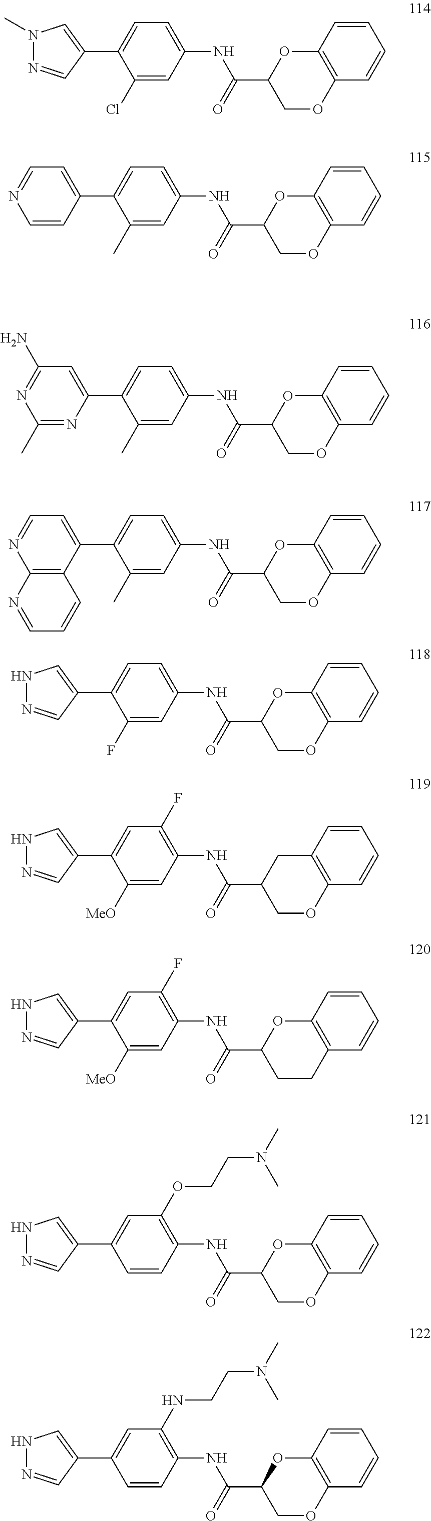 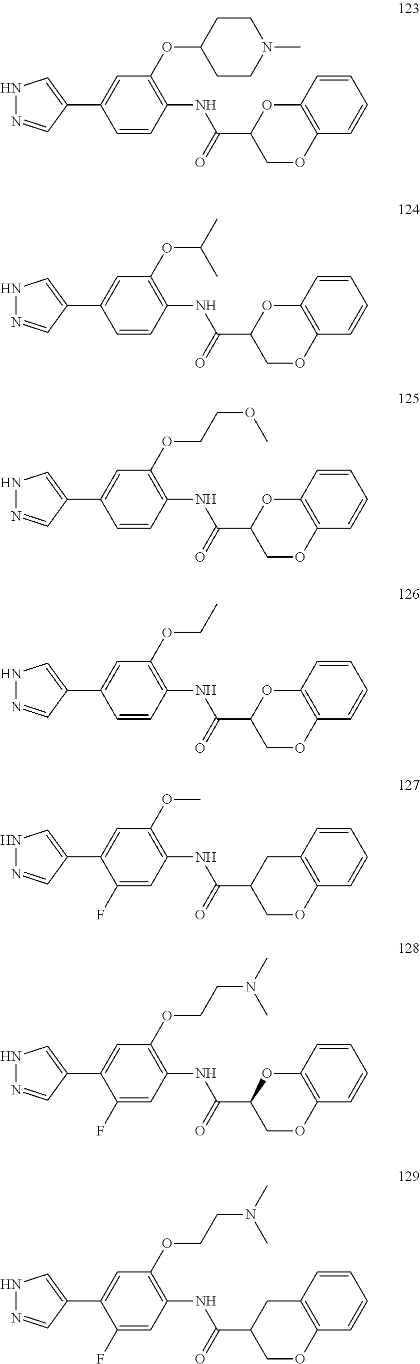 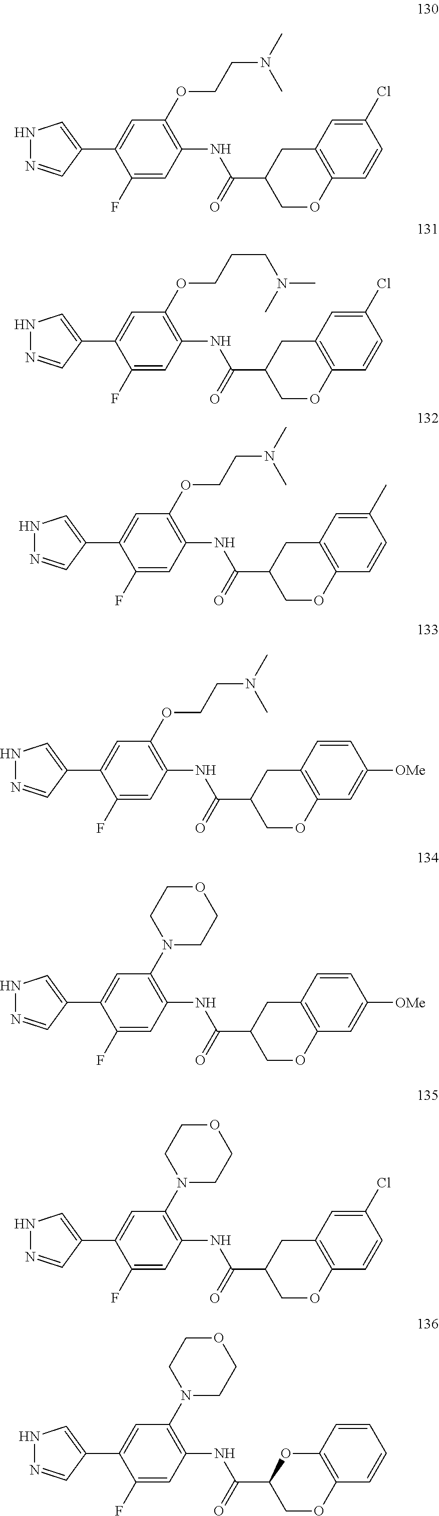 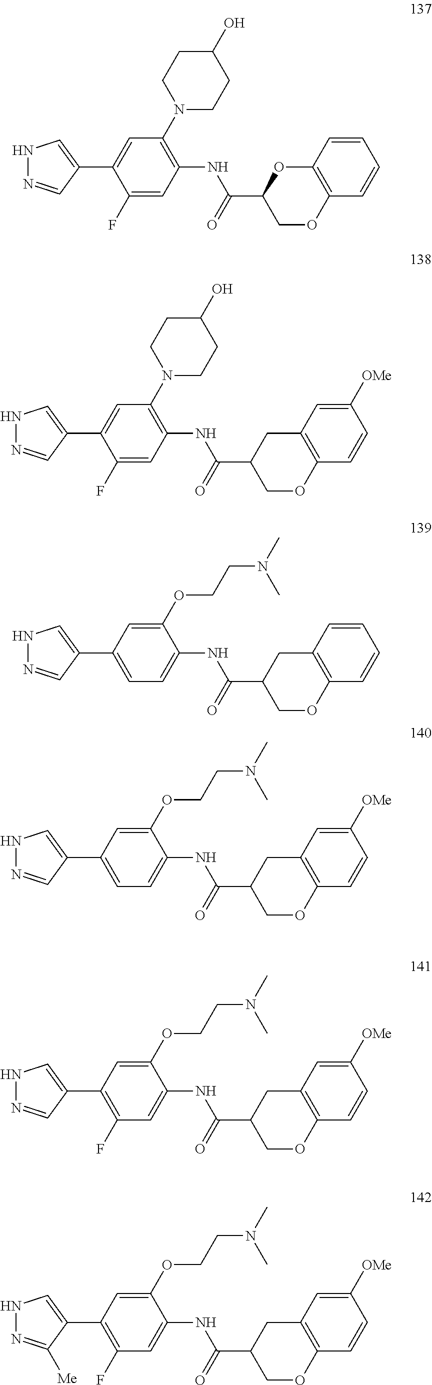 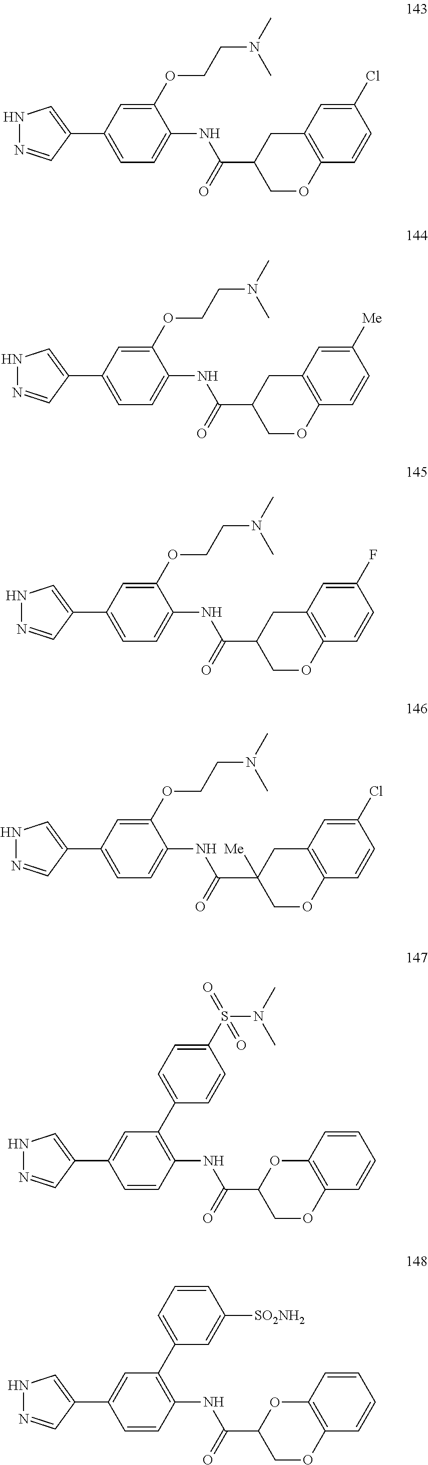 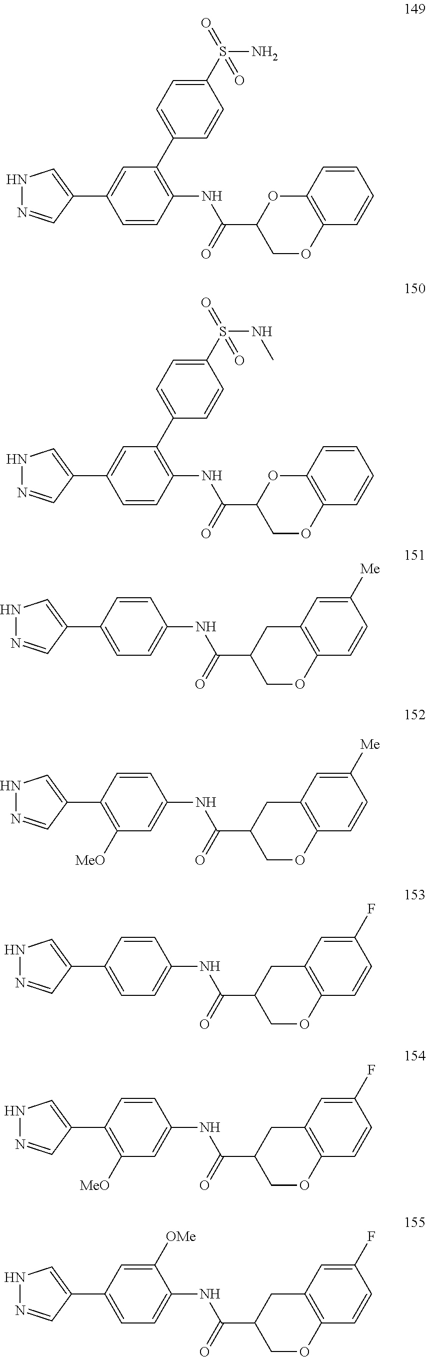 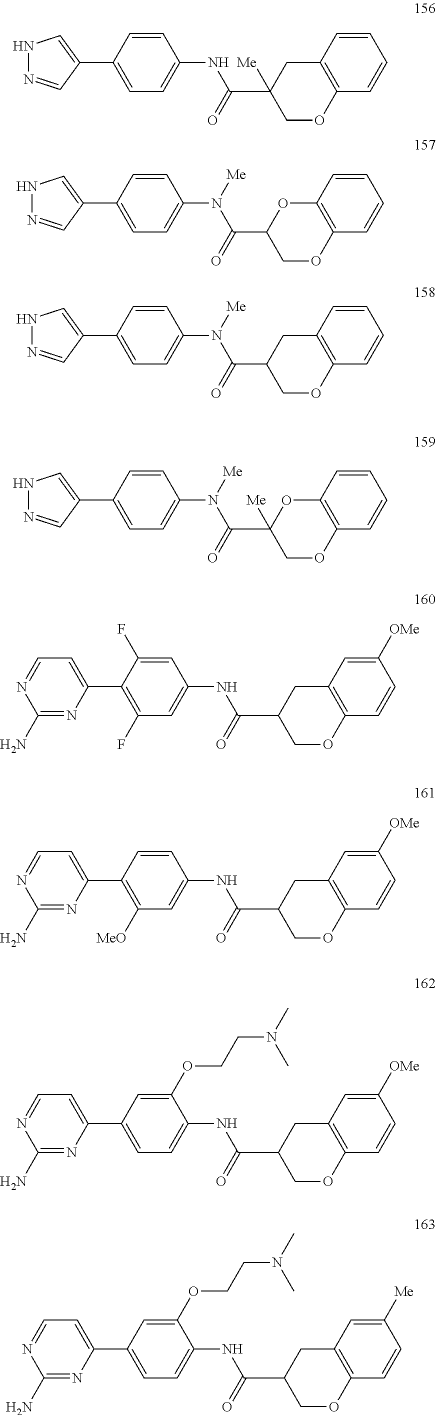 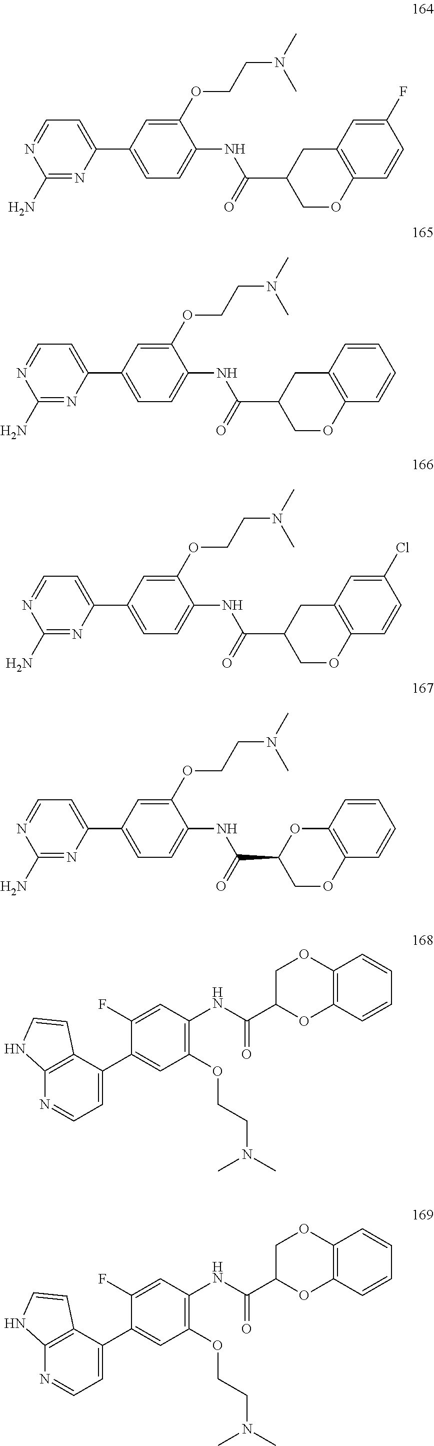 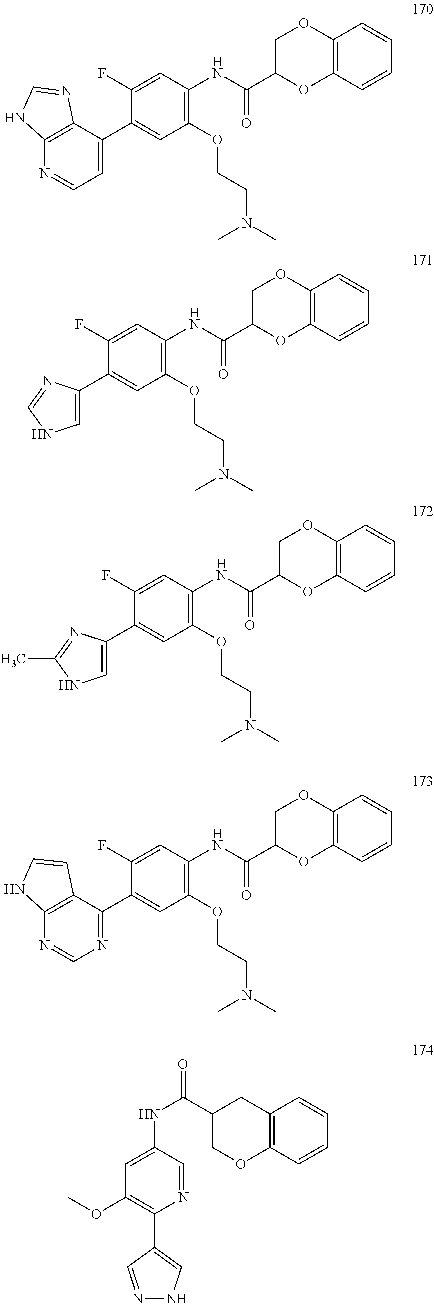 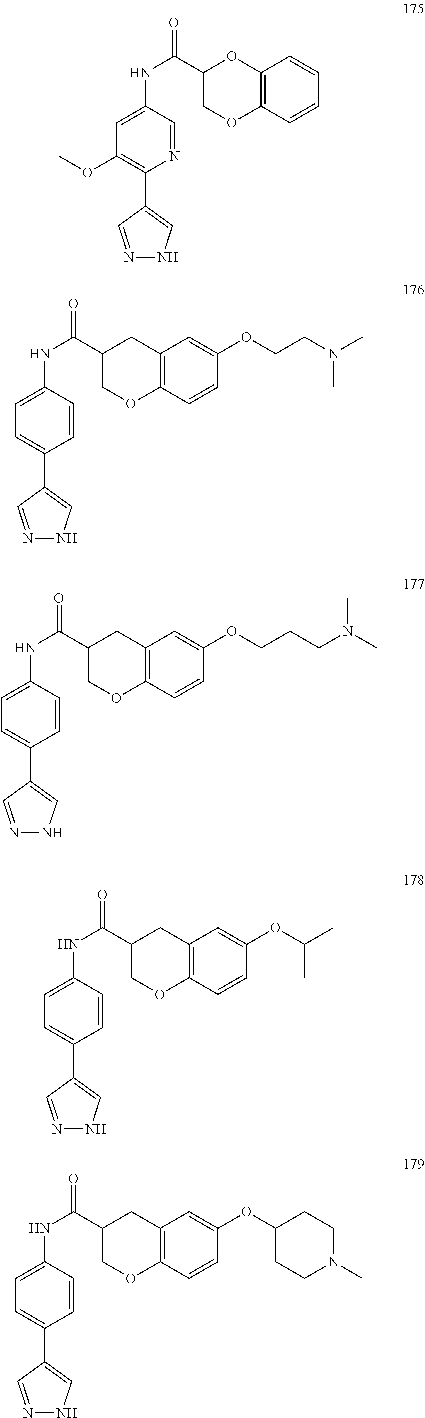 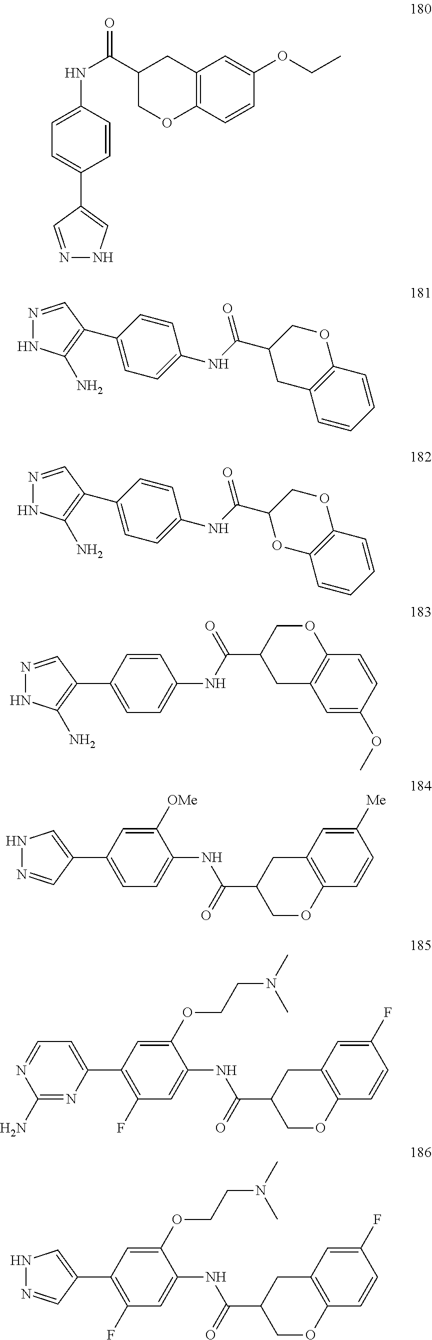 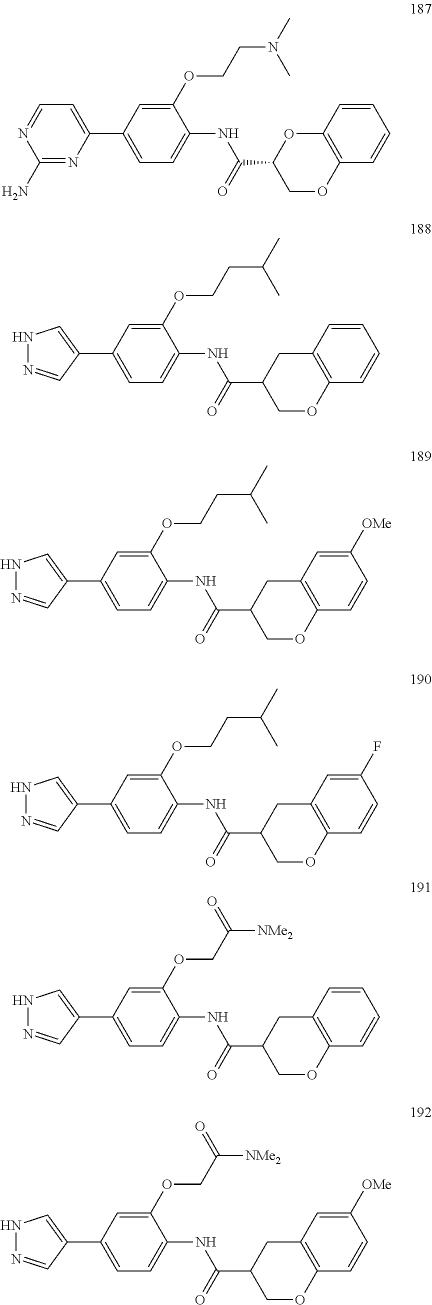 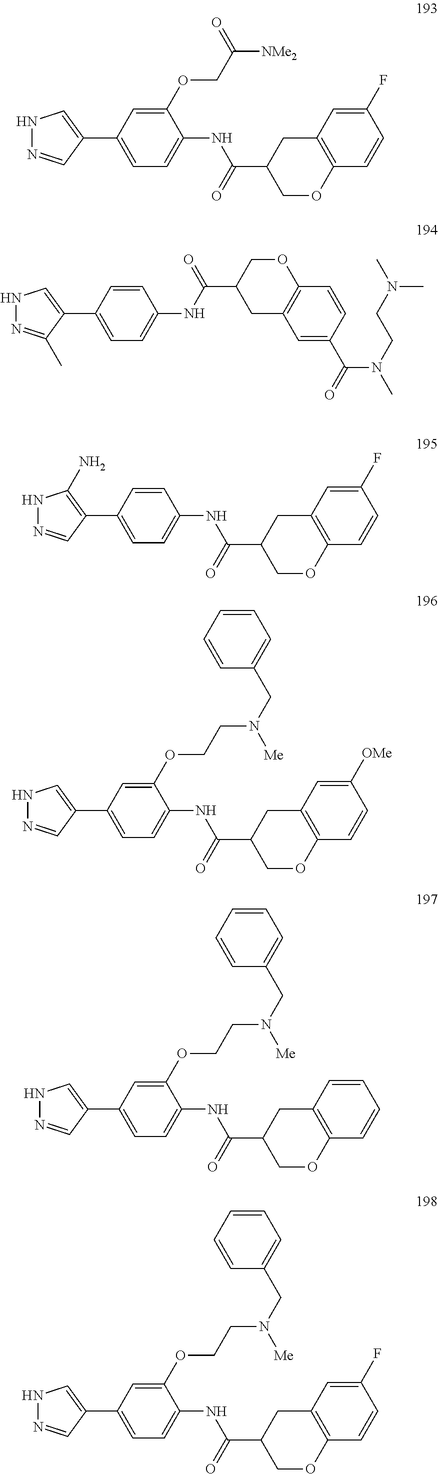 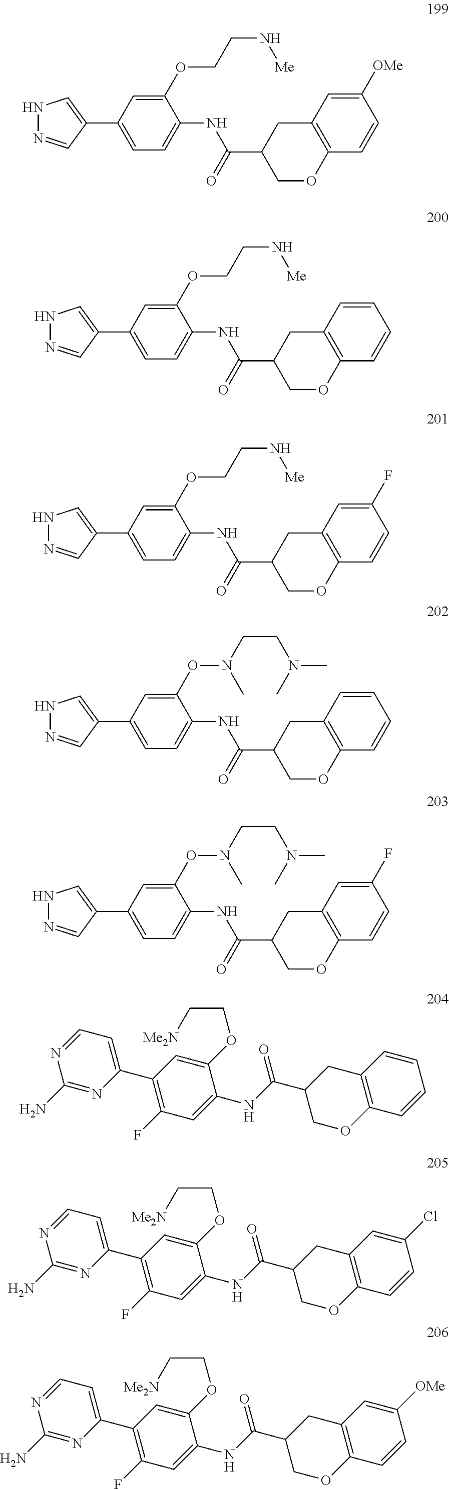 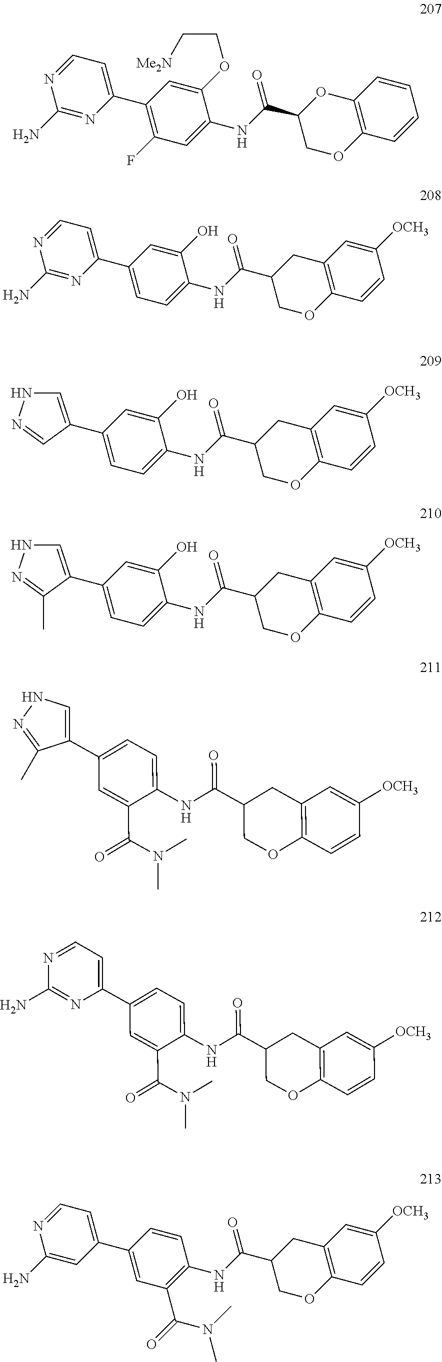 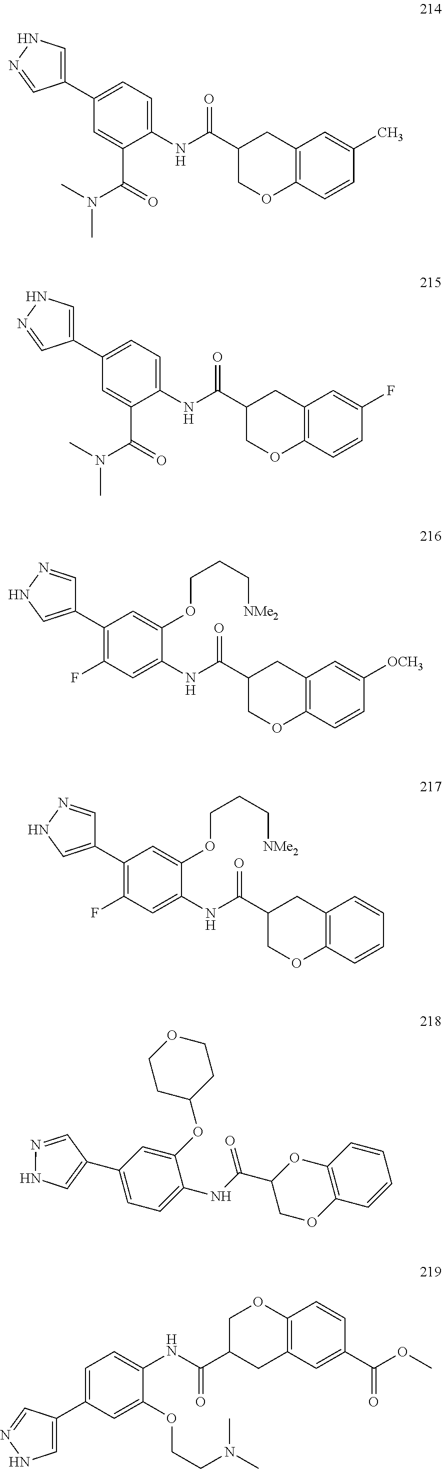 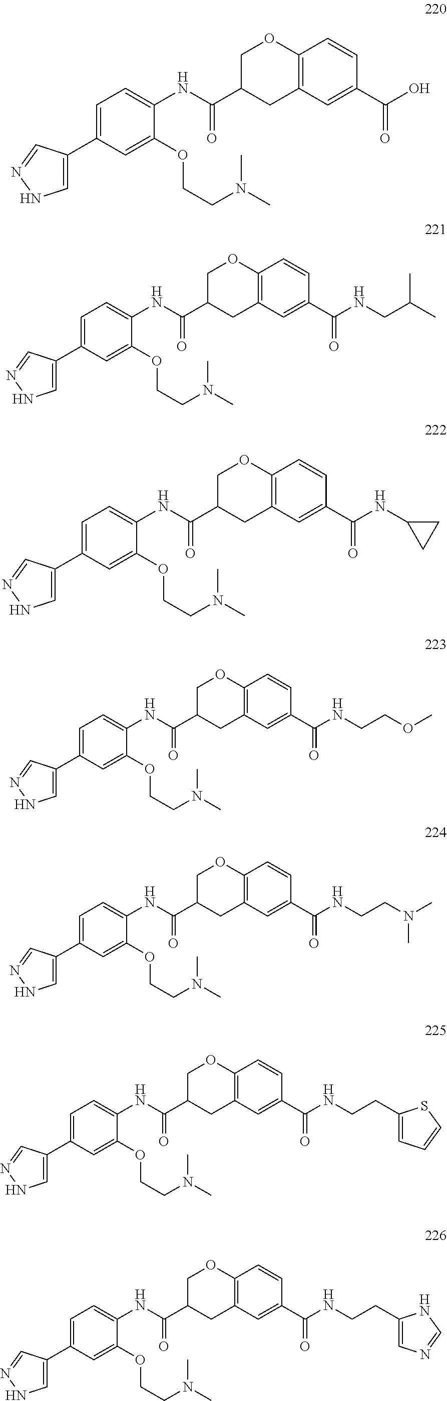 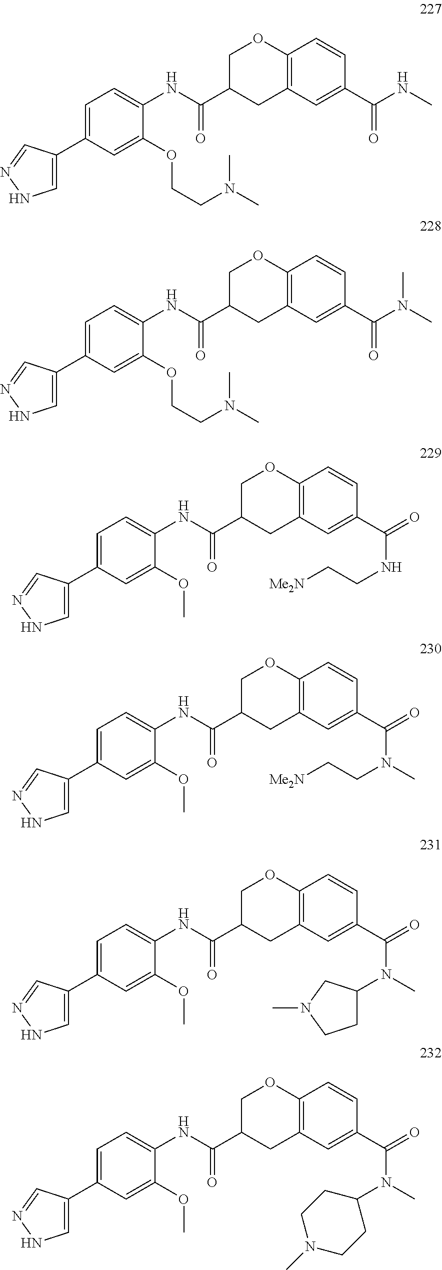 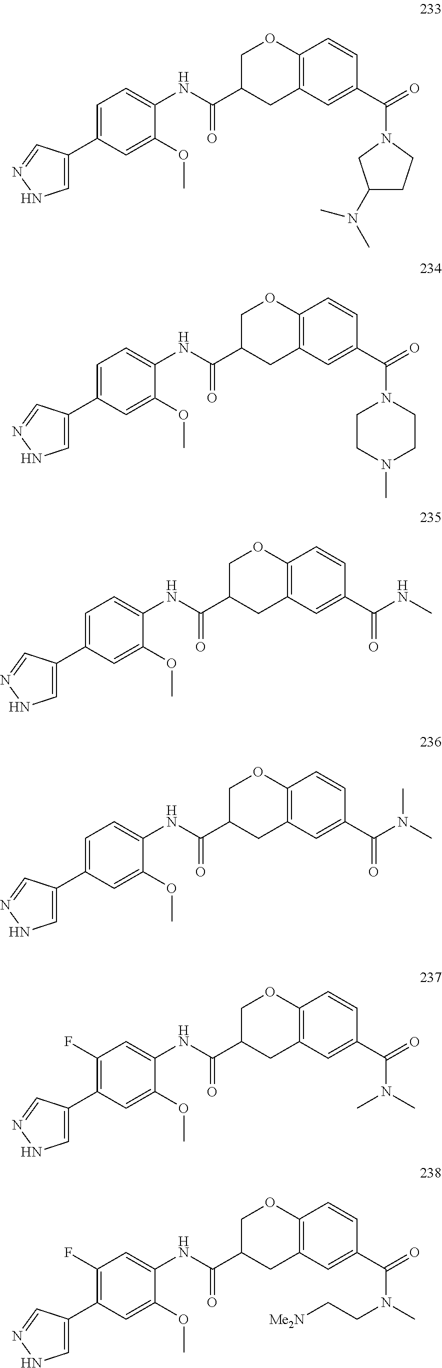 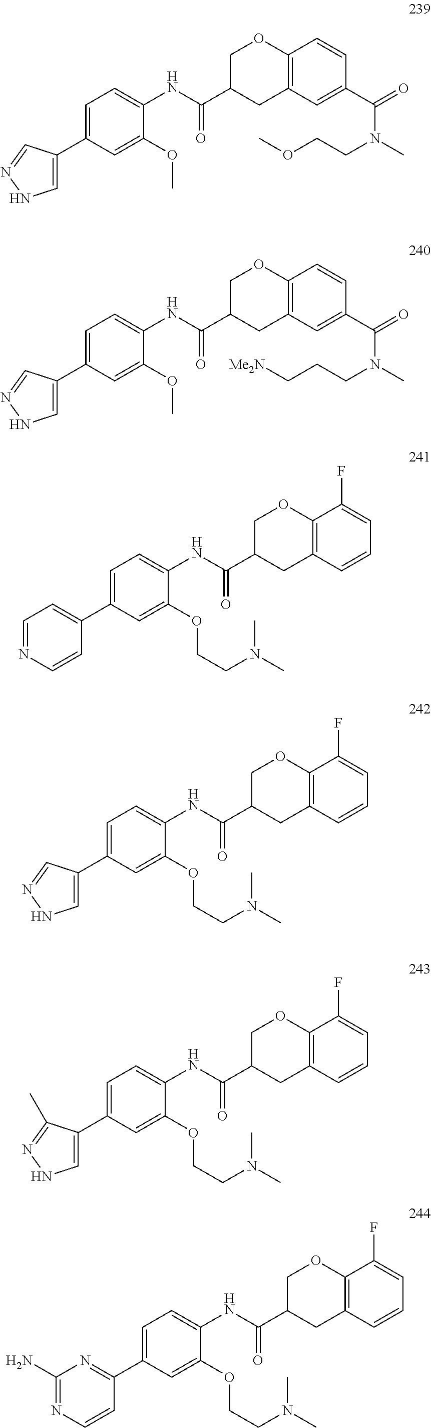 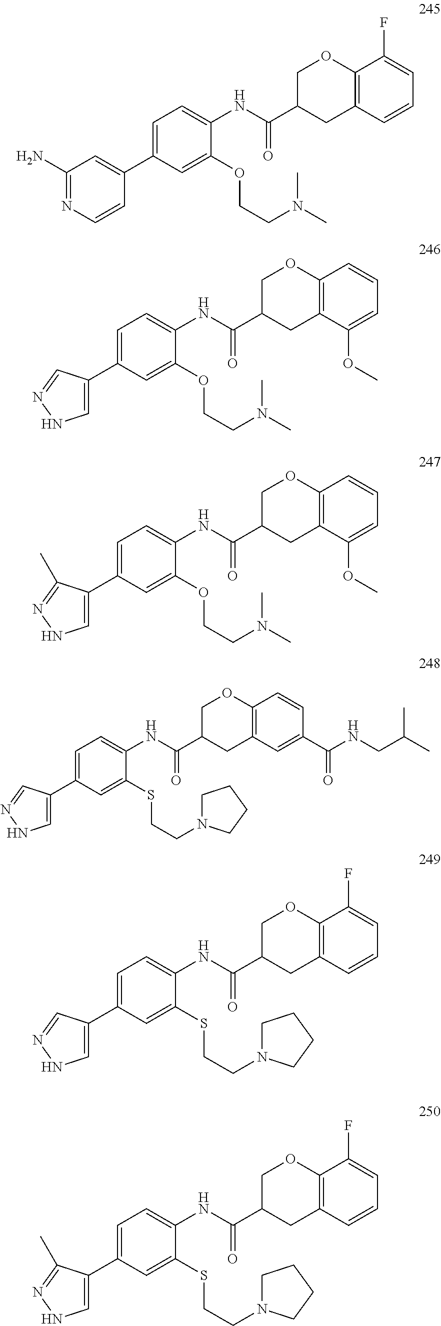 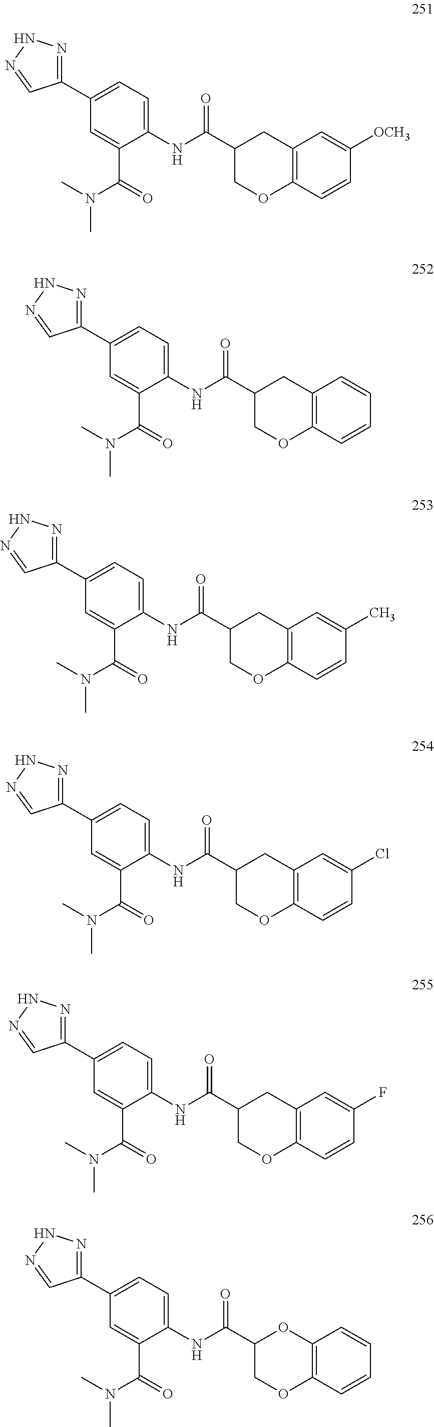 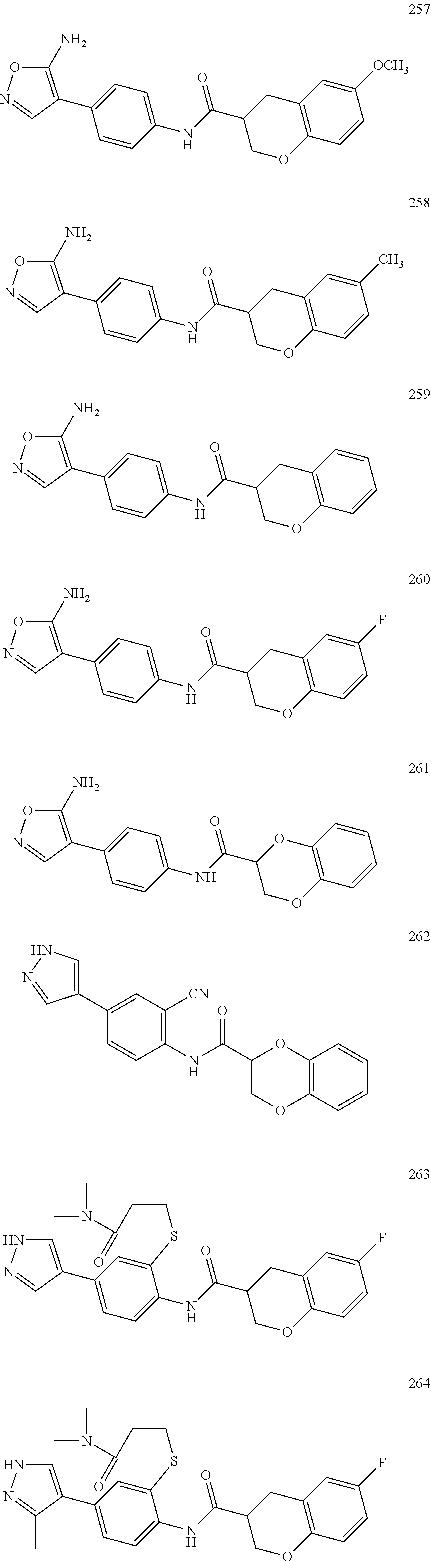 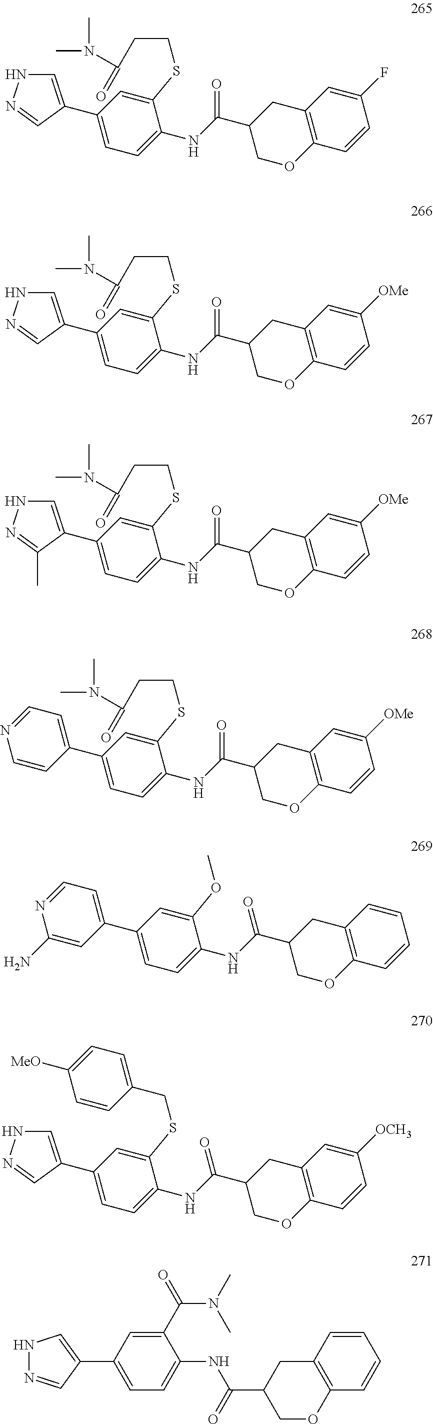 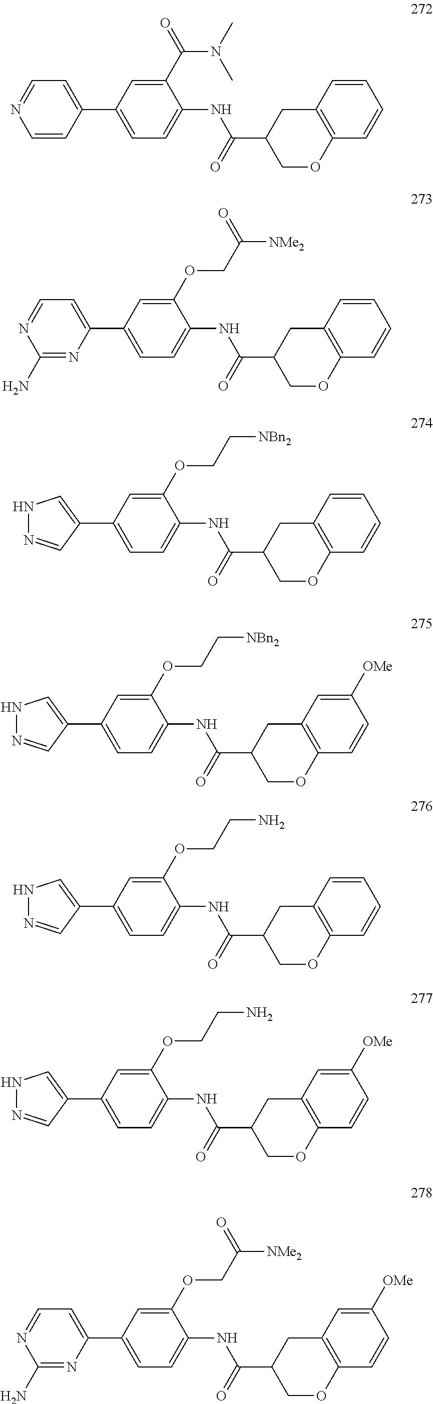 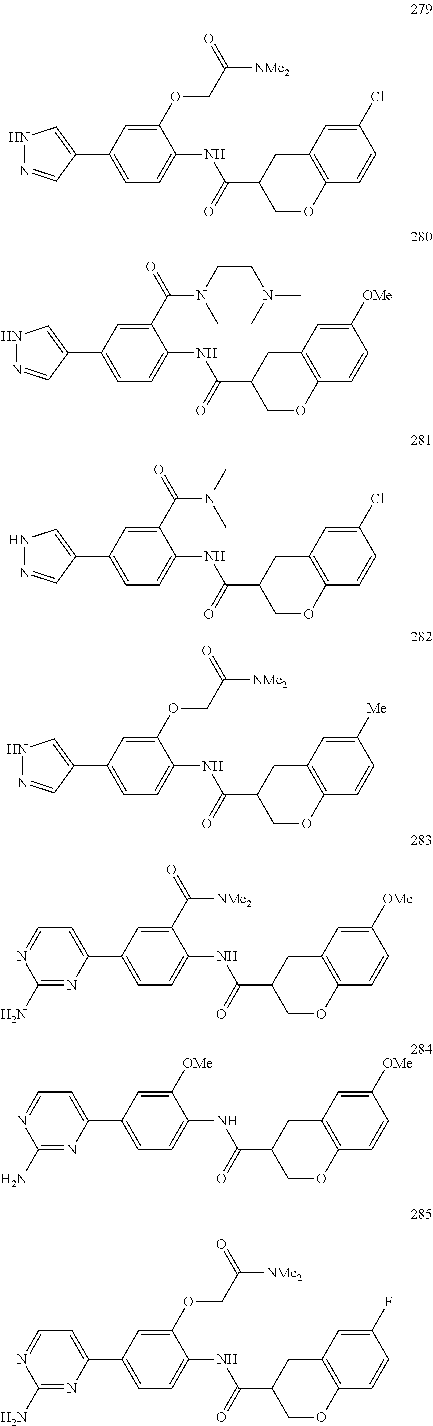 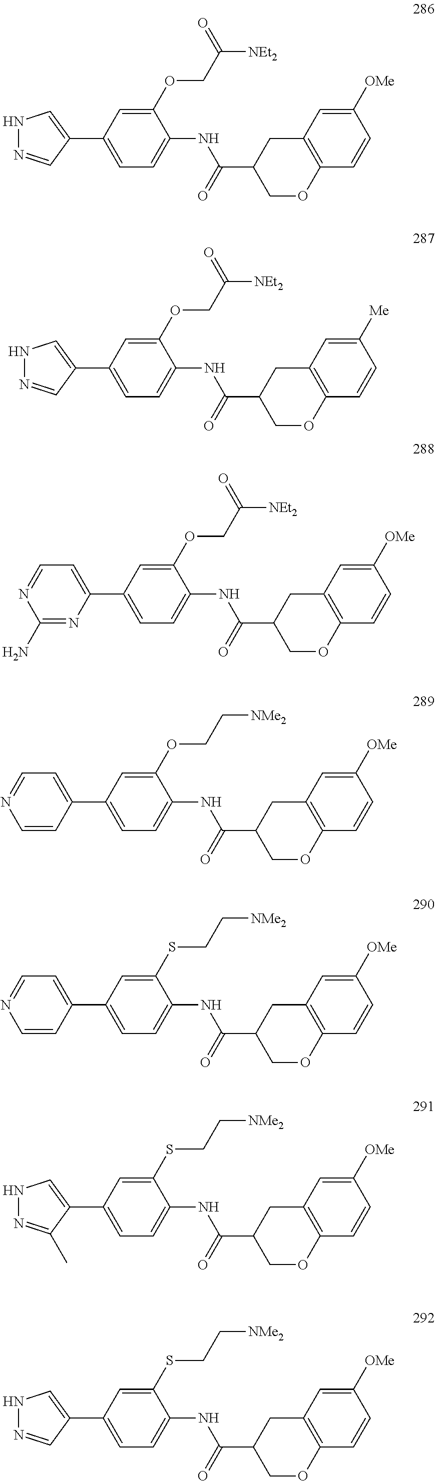 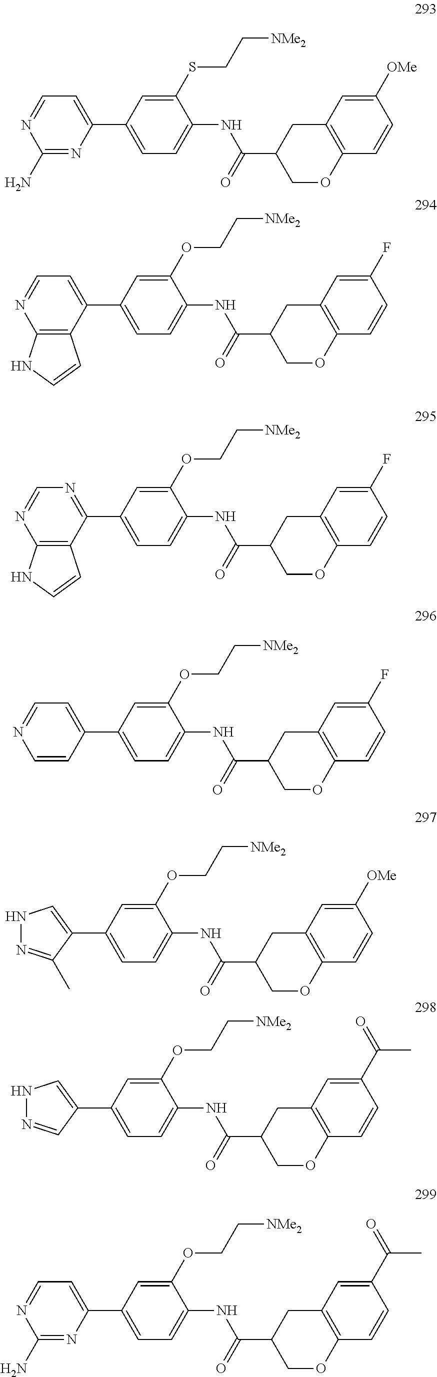 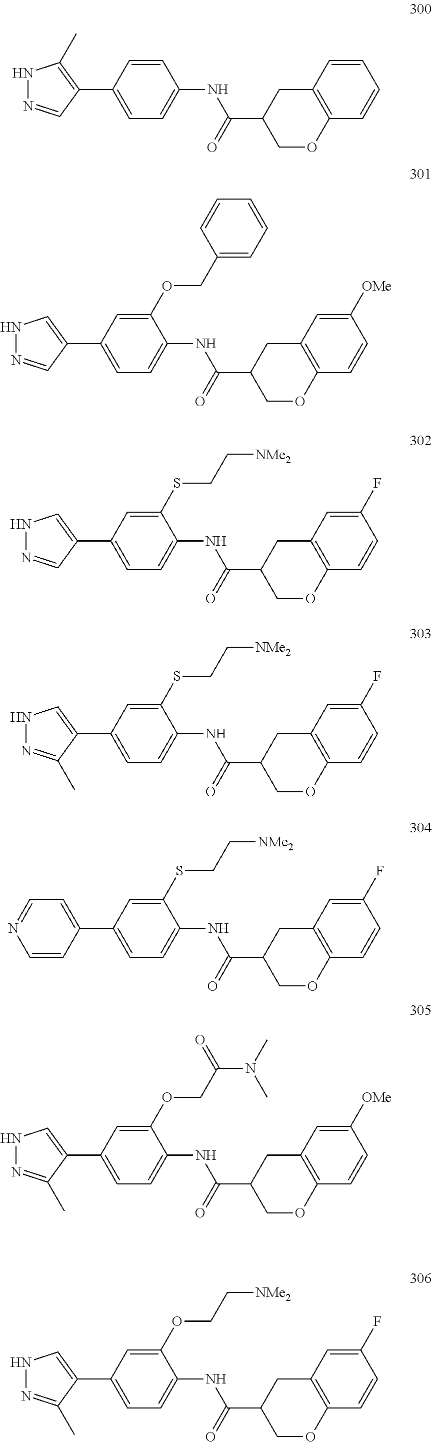 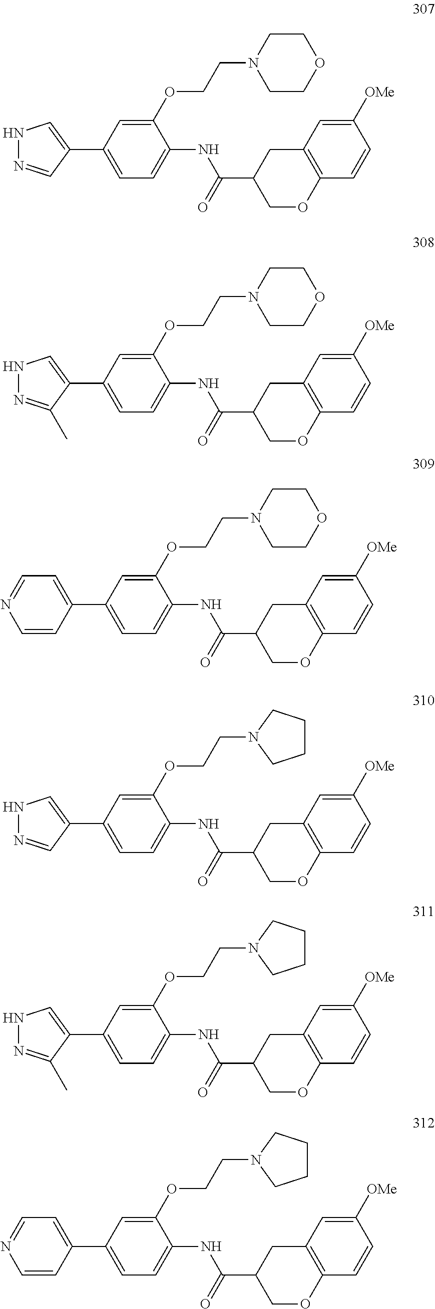 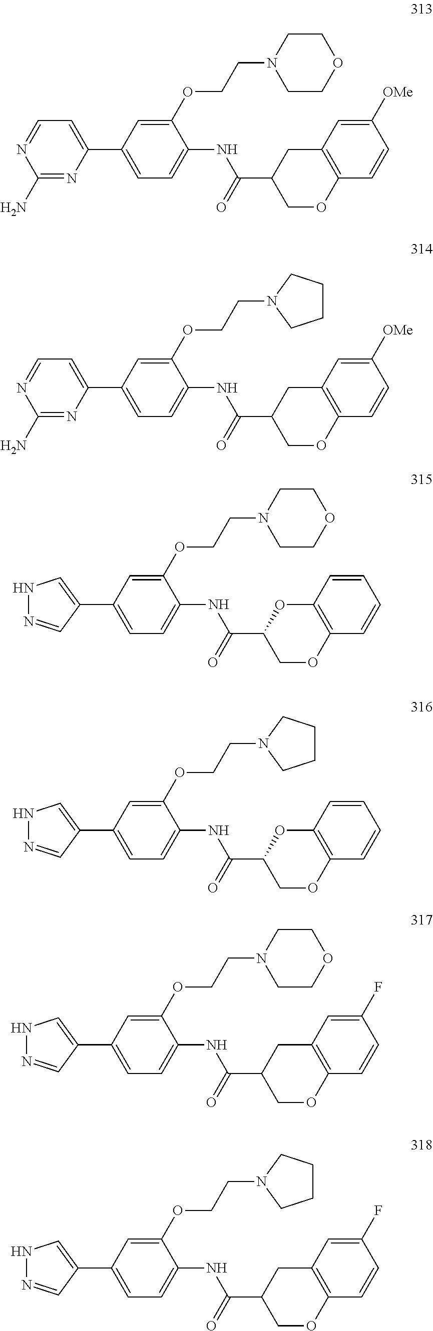 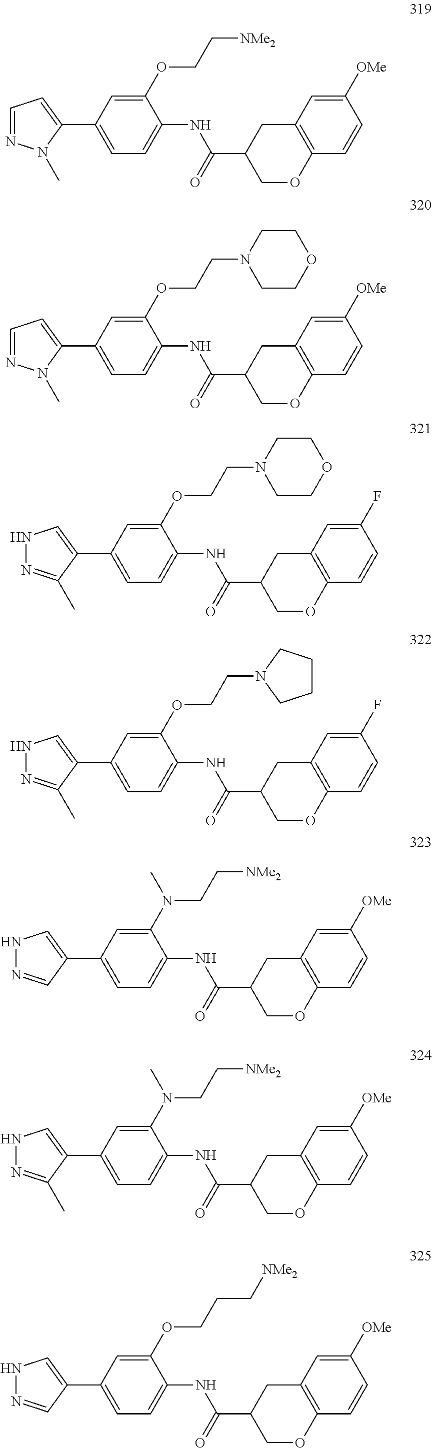 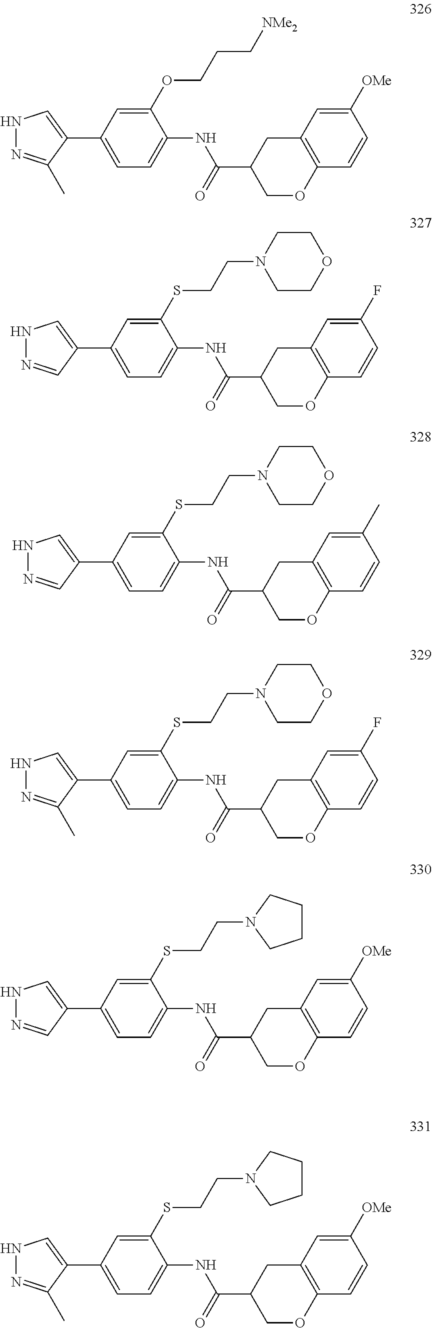 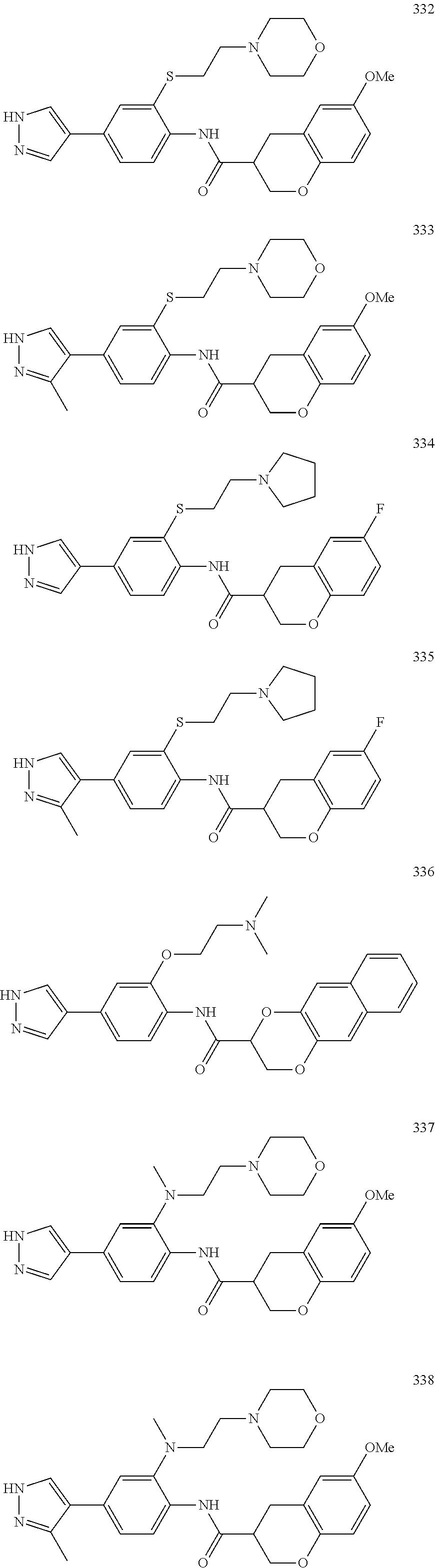 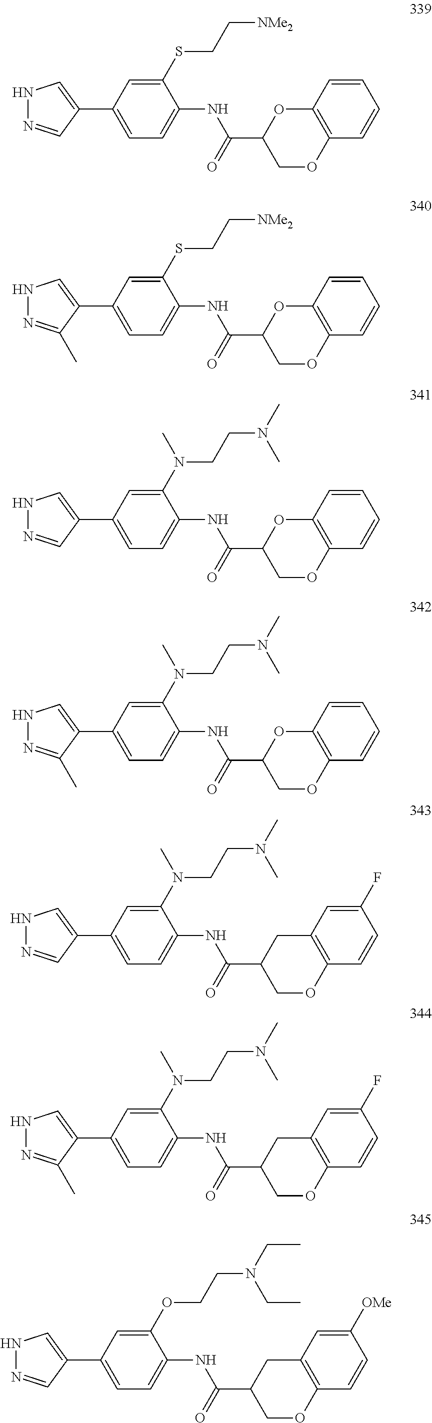 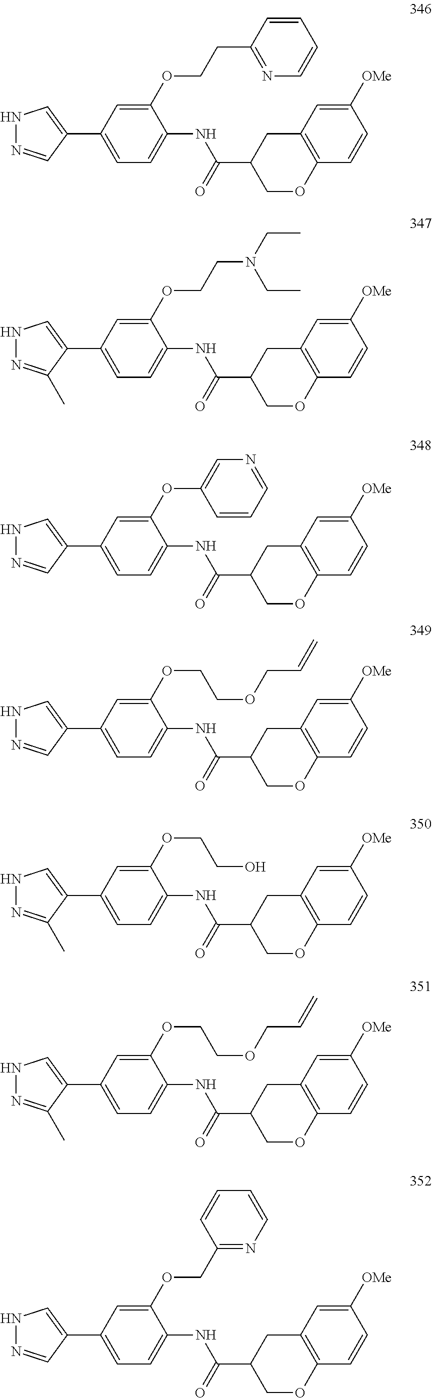 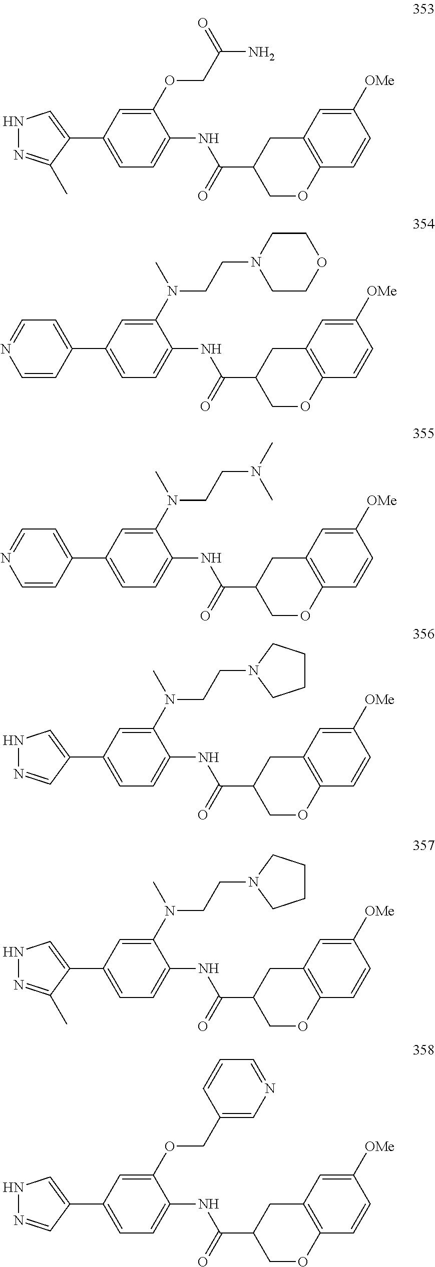 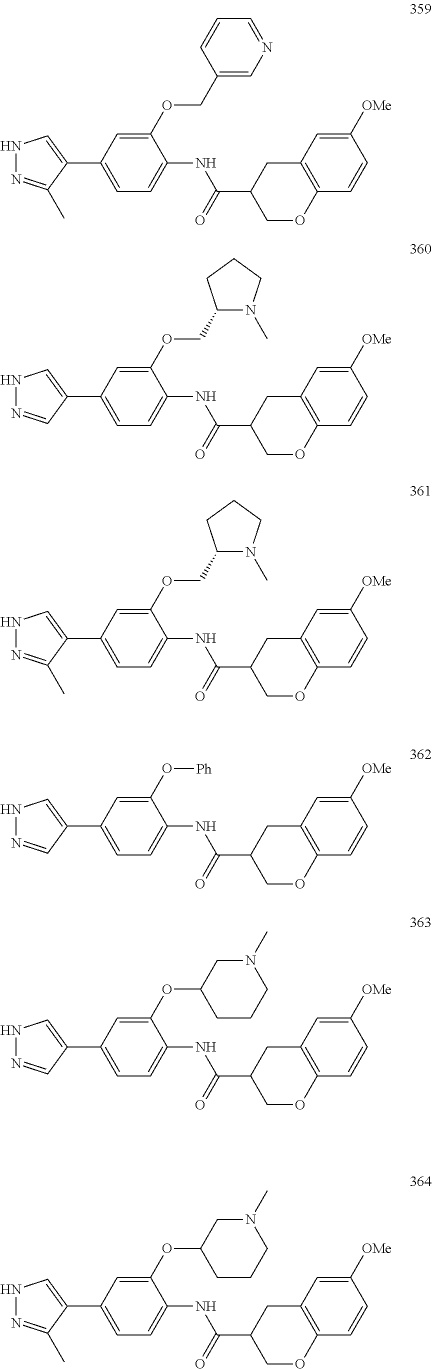 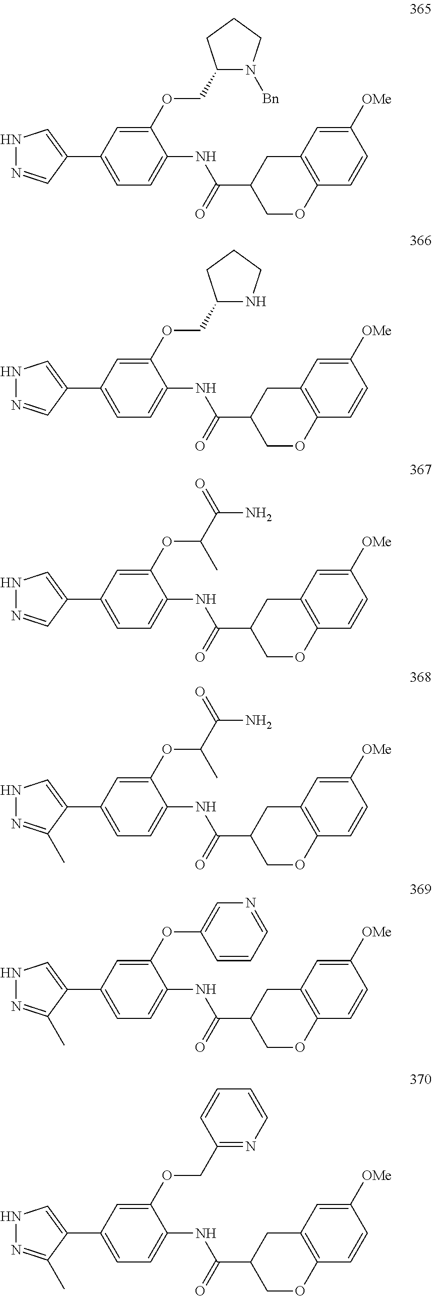 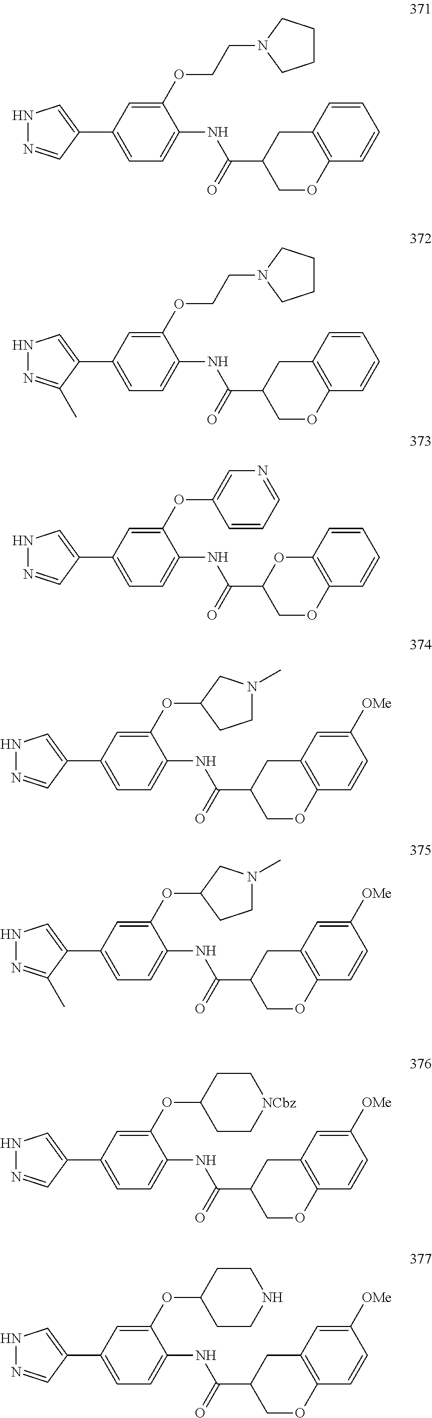 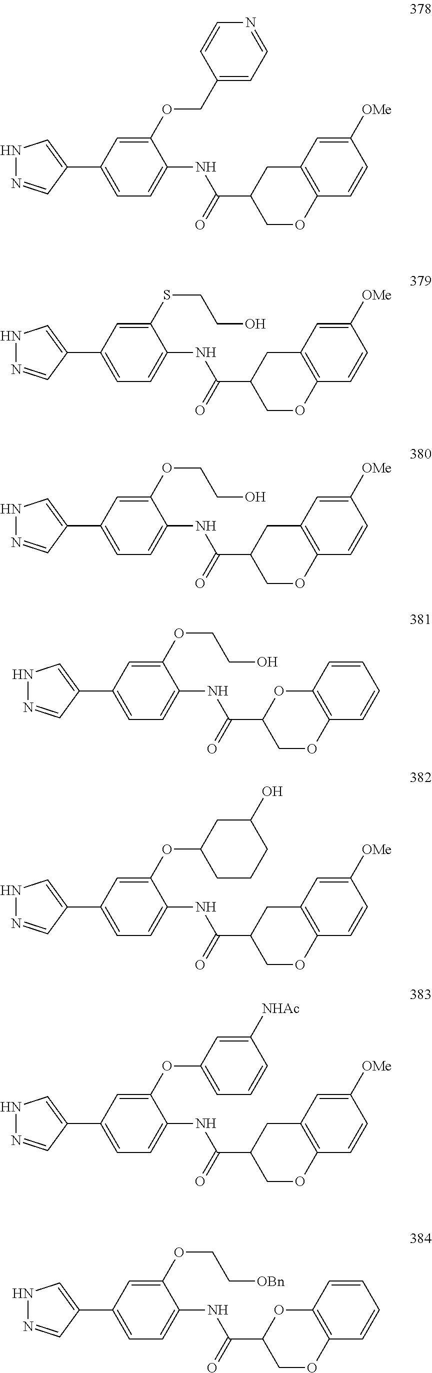 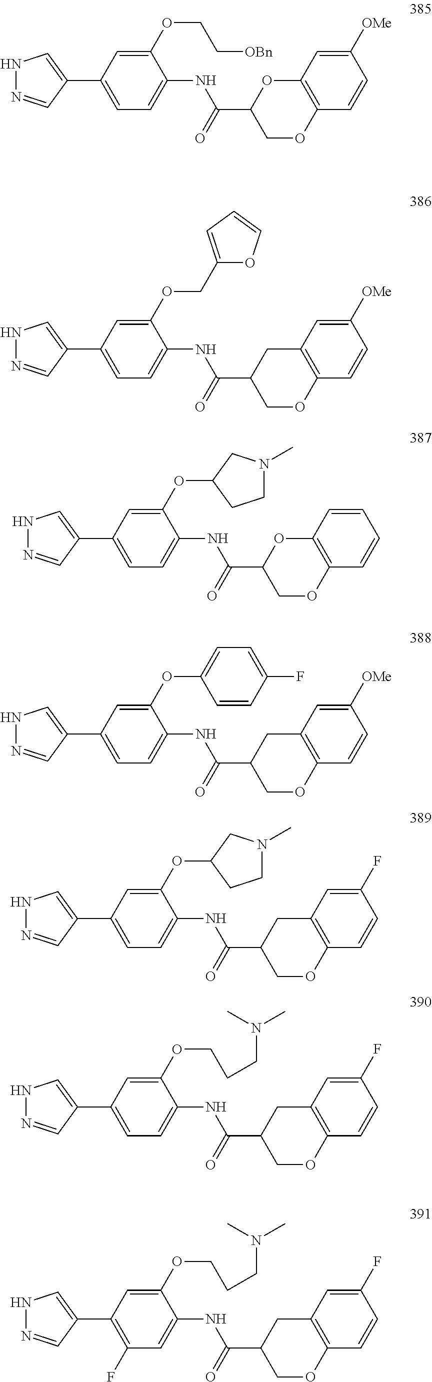 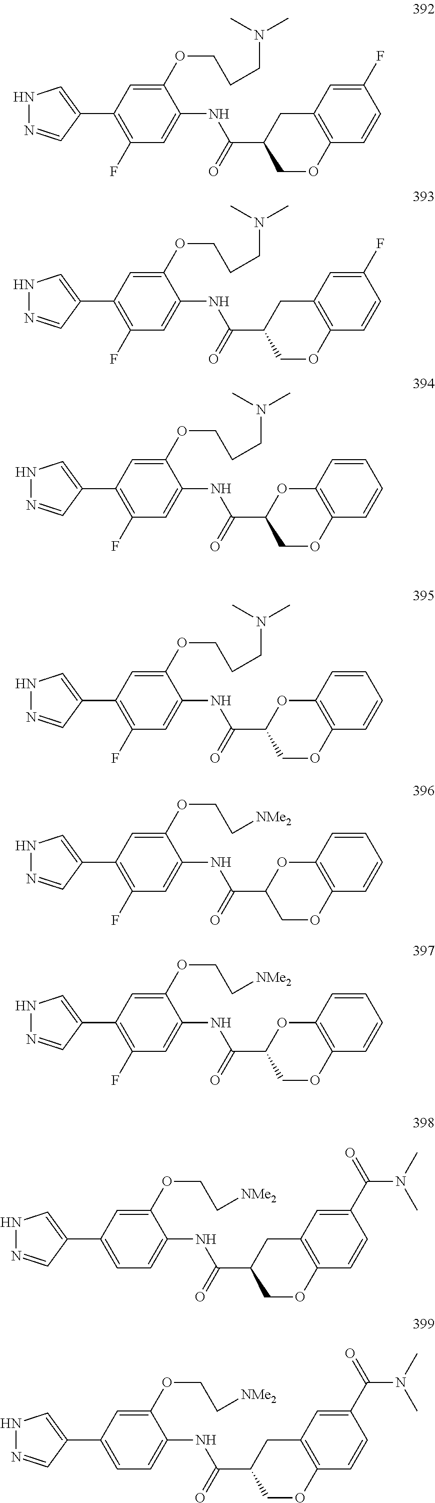 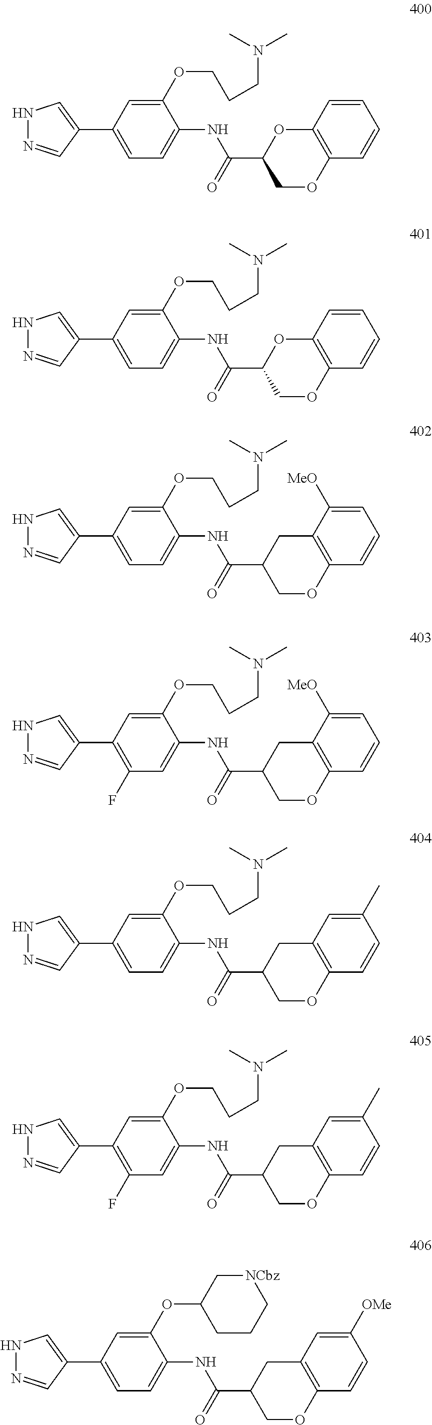 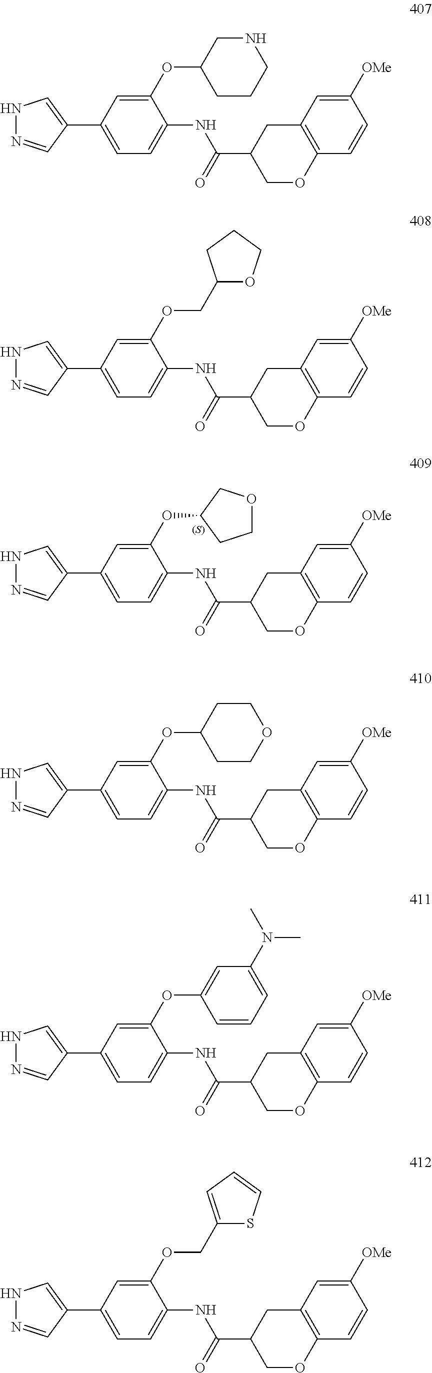 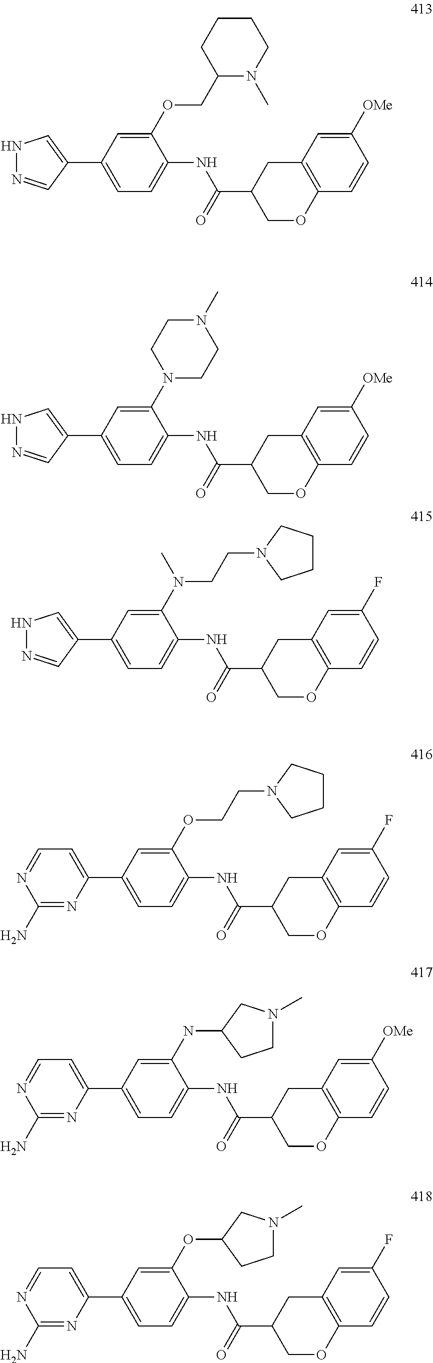 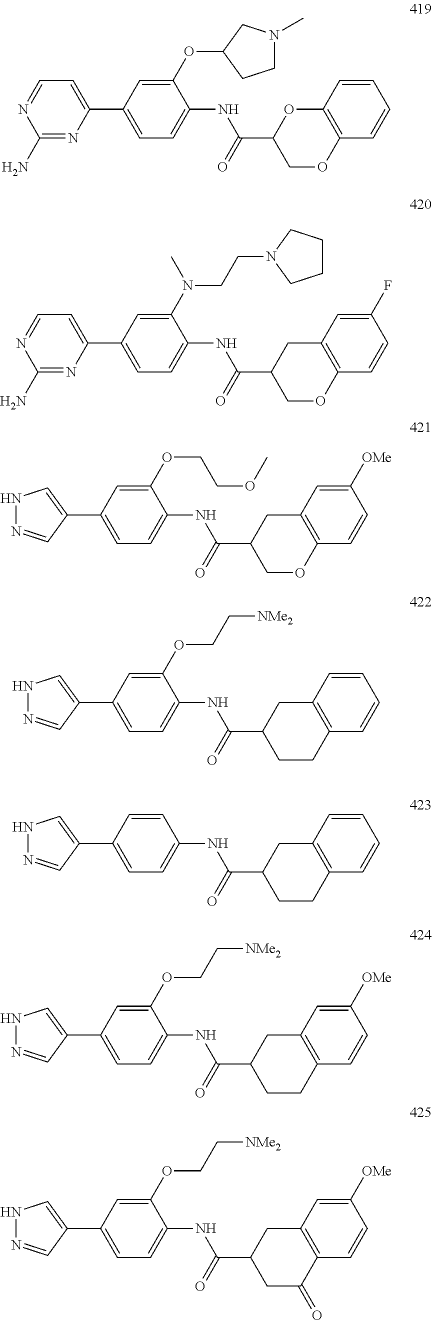 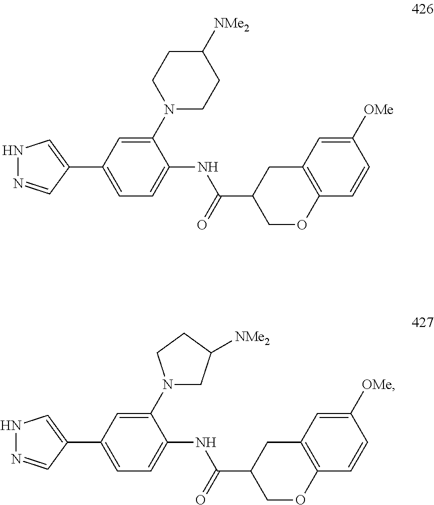
or any salt, stereoisomer, tautomer, hydrate, solvate, or prodrug thereof.

25. A pharmaceutical composition comprising a compound of claim 1 and a pharmaceutically acceptable excipient.

26. A pharmaceutical combination comprising a compound of claim 1 and an effective amount of a second medicament.

27-36. (canceled)

37. A pharmaceutical composition comprising the combination of claim 26 and a suitable excipient.

38. A method of treatment of a malcondition in a patient in need thereof, comprising administering a therapeutically effective amount of the compound of claim 1 to the patient at a frequency of administration and for a duration of time sufficient to provide a beneficial effect to the patient.

39. The method of claim 38 wherein the malcondition comprises cardiovascular disease, neurogenic pain, hypertension, atherosclerosis, angina, stroke, arterial obstruction, peripheral arterial disease, peripheral circulation disorder, erectile dysfunction, acute or chronic pain, dementia, Alzheimer's disease, Parkinson's disease, neuronal degeneration, asthma, amyotrophic lateral sclerosis, spinal cord injury, rheumatoid arthritis, osteoarthritis, osteoporosis, psoriasis, cerebral vasospasm, glaucoma, multiple sclerosis, pulmonary hypertension, acute respiratory distress syndrome, inflammation, diabetes, urinary organ diseases such as overactive bladder (OAB) and benign prostatic hypertrophy (BPH), metastasis, cancer, glaucoma, ocular hypertension, retinopathy, autoimmune disease and viral infection, or myocardial pathology, or any combination thereof.

40. The method of claim 38 for which binding of a ligand to a Rho kinase or inhibition of a bioactivity of a Rho kinase, or both, is medically indicated.

41-43. (canceled)

44. The method of claim 38 further comprising administration of an effective amount of an additional medicament.

45. The method of claim 44 wherein the additional medicament comprises an anti-proliferative agent, an anti-glaucoma agent, an anti-hypertensive agent, an anti-atherosclerotic agent, an anti-multiple sclerosis agent, an anti-angina agent, an anti-erectile dysfunction agent, an anti-stroke agent, or an anti-asthma agent.

46. The method of claim 45 wherein the anti-proliferative agent comprises an alkylating agent, an anti-metabolite, a vinca alkaloid, a terpenoid, a topoisomerase inhibitor, a monoclonal antibody, a kinase inhibitor, carboplatin, cisplatin, taxol, leucovorin, 5-fluorouracil, eloxatin, cyclophosphamide, chlorambucil, avastin, or imatinib mesylate.

47. The method of claim 45 wherein the anti-glaucoma agent comprises a beta receptor-blocker, a prostaglandin, an alpha-adrenergic agonist, a parasympathomimetic (cholinergic agonist), or a carbonic anhydrase inhibitor.

48. The method of claim 45 wherein the anti-hypertensive agent comprises a beta receptor-blocker, a calcium channel blocker, a diueretic, an angiotensin converting enzyme (ACE) inhibitor, a renin inhibitor, or an angiotensin receptor antagonist.

49. The method of claim 45 wherein the anti-atherosclerotic agent comprises a 3-HMG-coA-reductase inhibitor, a statin, atorvastatin, simvastatin, niacin, or a combination drug such as vytorin.

50. The method of claim 45 wherein the anti-multiple sclerosis agent comprises beta-inteferon, tysabri, or glatirimar acetate.

51. The method of claim 45 wherein the anti-angina agent comprises a beta receptor-blocker, a calcium channel blocker, nitroglycerin, isosorbide mononitrate, nicorandil, or ranolanzine.

52. The method of claim 45 wherein the anti-erectile dysfunction agent comprises a phosphodiesterase-5 inhibitor.

53. The method of claim 45 wherein the anti-stroke agent comprises tissue plasminogen activator.

54. The method of claim 45 wherein the anti-asthma agent comprises a bronchodilator, an inhaled corticosteroid, a leukotrine blockers, cromolyn, nedocromil, or theophylline.

55-60. (canceled)
